(19) United States
(12) Patent Application Publication
(10) Pub. No.: US 2007/0197590 A1
(43) Pub. Date: Aug. 23, 2007

(54) SUBSTITUTED DIPIPERIDINE CCR2 ANTAGONISTS

(76) Inventors: Duane E. DeMong, Belle Mead, NJ (US); Mingde Xia, Belle Mead, NJ (US); Scott R. Pollack, Flemington, NJ (US); Xiaoping Zheng, Lawrenceville, NJ (US); James A. Brackley III, Gilbertsville, PA (US); Michael P. Wachter, Bloomsbury, NJ (US); Druie E. Cavender, Flemington, NJ (US); Keith T. Demarest, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/669,284

(22) Filed: Jan. 31, 2007

Related U.S. Application Data
(60) Provisional application No. 60/763,608, filed on Jan. 31, 2006.

(51) Int. Cl. A61K 31/4545; C07D 401/14

(52) U.S. Cl. 514/316; 546/186; 546/187; 546/188

(57) ABSTRACT

Substituted dipiperidine compounds of Formula (I) 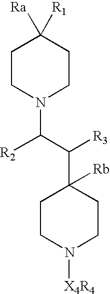 or a form thereof, which are CCR2 antagonists and are useful in preventing, treating or ameliorating CCR2 mediated inflammatory syndromes, disorders or diseases in a subject in need thereof.

### CROSS REFERENCE TO RELATED APPLICATIONS

This present application claims benefit of U.S. Provisional Patent Application Ser. No. 60/763,608, filed Jan. 31, 2006, which is incorporated herein by reference in its entirety and for all purposes.

### FIELD OF THE INVENTION

The invention is directed to substituted dipiperidine compounds that are antagonists to the chemoattractant cytokine receptor 2 (CCR2), pharmaceutical compositions and methods for use thereof. More particularly, the substituted dipiperidine compounds are useful antagonists for preventing, treating or ameliorating a CCR2 mediated inflammatory syndrome, disorder or disease.

### BACKGROUND OF THE INVENTION

CCR2 is a member of the GPCR family of receptors, as are all known chemokine receptors, and are expressed by monocytes and memory T-lymphocytes. The CCR2 signaling cascade involves activation of phospholipases (PLCβ₂), protein kinases (PKC), and lipid kinases (PI-3 kinase).

Chemoattractant cytokines (i.e., chemokines) are relatively small proteins (8-10 kD), which stimulate the migration of cells. The chemokine family is divided into four subfamilies based on the number of amino acid residues between the first and second highly-conserved cysteines.

Monocyte chemotacetic protein-1 (MCP-1) is a member of the CC chemokine subfamily (wherein CC represents the subfamily having adjacent first and second cysteines) and binds to the cell-surface chemokine receptor 2 (CCR2). MCP-1 is a potent chemotacetic factor, which, after binding to CCR2, mediates monocyte and lymphocyte migration (i.e., chemotaxis) toward a site of inflammation. MCP-1 is also expressed by cardiac muscle cells, blood vessel endothelial cells, fibroblasts, chondrocytes, smooth muscle cells, mesangial cells, alveolar cells, T-lymphocytes, marcophages, and the like.

After monocytes enter the inflammatory tissue and differentiate into macrophages, monocyte differentiation provides a secondary source of several proinflammatory modulators, including tumor necrosis factor-α (TNF-α), interleukin-1 (IL-1), IL-8 (a member of the CXC chemokine subfamily, wherein CXC represents one amino acid residue between the first and second cysteines), IL-12, arachidonic acid metabolites (e.g., PGE₂ and LTB₄), oxygen-derived free radicals, matrix metalloproteinases, and complement components.

Animal model studies of chronic inflammatory diseases have demonstrated that inhibition of binding between MCP-1 and CCR2 by an antagonist suppresses the inflammatory response. The interaction between MCP-1 and CCR2 has been implicated (see Rollins B J, Monocyte chemoattractant protein 1: a potential regulator of monocyte recruitment in inflammatory disease, Mol. Med. Today, 1996, 2:198; and Dawson J, et al., Targeting monocyte chemoattractant protein-1 signaling in disease, Expert Opin. Ther. Targets, 2003 February, 7(1):35-48) in inflammatory disease pathologies such as psoriasis, uveitis, atherosclerosis, rheumatoid arthritis, multiple sclerosis, Crohn's Disease, nephritis, organ allograft rejection, fibroid lung, renal insufficiency, diabetes and diabetic complications, diabetic nephropathy, diabetic retinopathy, diabetic retinitis, diabetic microangiopathy, tuberculosis, sarcoidosis, invasive staphylococcia, inflammation after cataract surgery, allergic rhinitis, allergic conjunctivitis, chronic urticaria, Chronic Obstructive Pulmonary Disease (COPD), allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, and stomach.

Monocyte migration is inhibited by MCP-1 antagonists (either antibodies or soluble, inactive fragments of MCP-1), which have been shown to inhibit the development of arthritis, asthma, and uveitis. Both MCP-1 and CCR2 knockout (KO) mice have demonstrated that monocyte infiltration into inflammatory lesions is significantly decreased. In addition, such KO mice are resistant to the development of experimental allergic encephalomyelitis (EAE, a model of human MS), cockroach allergen-induced asthma, atherosclerosis, and uveitis. Rheumatoid arthritis and Crohn's Disease patients have improved during treatment with TNF-α antagonists (e.g., monoclonal antibodies and soluble receptors) at dose levels correlated with decreases in MCP-1 expression and the number of infiltrating macrophages.

MCP-1 has been implicated in the pathogenesis of seasonal and chronic allergic rhinitis, having been found in the nasal mucosa of most patients with dust mite allergies. MCP-1 has also been found to induce histamine release from basophils in vitro. During allergic conditions, both allergens and histamines have been shown to trigger (i.e., to up-regulate) the expression of MCP-1 and other chemokines in the nasal mucosa of people with allergic rhinitis, suggesting the presence of a positive feedback loop in such patients.

CCR2 influences the development of obesity and associated adipose tissue inflammation and systemic insulin resistance and plays a role in the maintenance of adipose tissue macrophages and insulin resistance once obesity and its metabolic consequences are established (J. Clin. Invest., 2006, 116, 115-124).

There remains a need for small molecule CCR2 antagonists for preventing, treating or ameliorating a CCR2 mediated inflammatory syndrome, disorder or disease resulting from MCP-1 induced monocyte and lymphocyte migration to a site of inflammation.

PCT Application WO 02/079190, published on Oct. 10, 2002, describes 3-substituted indoles as chemokine antagonists.

PCT Application WO 04/054974, published on Jul. 1, 2004, describes substituted piperidine compounds as CCR5 antagonists.

United States Patent Publication 2004/0138226, published on Jul. 15, 2004, describes substituted piperidine compounds as 17-beta hydroxysteroid dehydrogenase Type 3 inhibitors for the treatment of androgen related diseases.

United States Patent Publication 2004/0147506, published on Jul. 29, 2004 (Equivalent of PCT Application WO 02/085890, published on Oct. 31, 2002), describes substituted benzimidazolone compounds as muscarinic acetylcholine antagonists.

All documents cited herein are incorporated by reference.

### SUMMARY OF THE INVENTION

The invention provides substituted dipiperidine compounds of Formula (I):

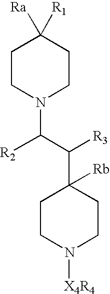

or a form thereof, wherein R₁, Ra, R₂, R₃, Rb and X₄R₄ are as defined herein.

The compounds of the present invention are CCR2 antagonists are useful in preventing, treating or ameliorating CCR2 mediated inflammatory syndromes, disorders or diseases in a subject in need thereof.

The present invention also provides a method for preventing, treating or ameliorating a CCR2 mediated inflammatory syndrome, disorder or disease in a subject in need thereof comprising administering to the subject an effective amount of a compound of Formula (I) or a form thereof.

### DETAILED DESCRIPTION OF THE INVENTION

The present invention is directed to a compound of Formula (I) 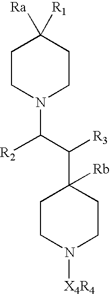
or a form thereof, wherein
R₁ is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino (optionally substituted with one or more of alkyl, alkylcarbonyl, alkoxycarbonyl, alkylsulfonyl, carboxyalkyl or alkoxycarbonylalkyl), carboxyalkyl, alkoxycarbonylalkyl, carboxyalkoxy, alkoxycarbonylalkoxy, aminoalkyl, alkylaminoalkyl, aminosulfonyl, alkylaminosulfonyl, alkylsulfonylamino, aminosulfonylalkyl, alkylaminosulfonylalkyl, carboxy, alkylcarbonyl, alkoxycarbonyl, aminocarbonyl, alkylaminocarbonyl, aryl or heterocyclyl;
Ra and Rb is each hydrogen or hydroxy;
R₂ is hydrogen or is oxo, hydroxyalkyl, haloalkyl, alkylamino, cyano, alkoxy, carboxy or alkoxycarbonyl;
R₃ is hydrogen or is oxo, hydroxy, hydroxyalkyl, halogen, haloalkyl, amino (optionally substituted with one or more of alkyl, formyl, alkylcarbonyl or alkoxycarbonyl), cyano, nitro, alkoxy, carboxy, alkoxycarbonyl, aminocarbonyl, alkylaminocarbonyl, alkoxycarbonylamino, aminocarbonylamino or alkylaminocarbonylamino, with the proviso that R₂ and R₃ are not simultaneously hydrogen;
X₄ is absent or is carbonyl, carboxyl, alkylcarbonyl, alkylcarbonyloxy, acrylyl, alkoxycarbonyl, aminocarbonyl, alkylaminocarbonyl, alkylcarbonylamino, alkylcarbonylaminoalkyl, thiocarbonyl, aminothiocarbonyl, alkylthiocarbonyl or carbonylaminoiminomethyl; and
R₄ is hydrogen or is cycloalkyl, aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl;
R₅ is hydrogen or is one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio or haloalkylthio.

An example of the invention is a compound of Formula (I) or a form thereof, wherein R₁ is aryl or heterocyclyl each optionally substituted with one or more of alkyl, alkoxy, cyano, halogen, haloalkyl, amino, alkylamino, alkylcarbonylamino, alkylsulfonylamino, aryl or heterocyclyl.

An example of the invention is a compound of Formula (I) or a form thereof, wherein R₁ is aryl or heterocyclyl each optionally substituted with one or more of alkyl, alkoxy, cyano, halogen, haloalkyl, amino, alkylcarbonylamino, alkylsulfonylamino or heterocyclyl.

An example of the invention is a compound of Formula (I) or a form thereof, wherein Ra and Rb are each hydroxy.

An example of the invention is a compound of Formula (I) or a form thereof, wherein Ra is hydrogen or hydroxy.

An example of the invention is a compound of Formula (I) or a form thereof, wherein Rb is hydrogen or hydroxy.

An example of the invention is a compound of Formula (I) or a form thereof, wherein R₂ is hydrogen or is oxo, hydroxyalkyl, haloalkyl, cyano, carboxy or alkoxycarbonyl.

An example of the invention is a compound of Formula (I) or a form thereof, wherein R₂ is hydrogen or is oxo, hydroxyalkyl, carboxy or alkoxycarbonyl.

An example of the invention is a compound of Formula (I) or a form thereof, wherein R₃ is hydrogen or is oxo, hydroxy, hydroxyalkyl, halogen, haloalkyl, amino, alkylamino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen.

An example of the invention is a compound of Formula (I) or a form thereof, wherein R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen.

An example of the invention is a compound of Formula (I) or a form thereof, wherein X₄ is absent or is carbonyl, alkylcarbonyl, acrylyl, alkoxycarbonyl, aminocarbonyl, alkylaminocarbonyl, thiocarbonyl, aminothiocarbonyl or carbonylaminoiminomethyl.

An example of the invention is a compound of Formula (I) or a form thereof, wherein X₄ is absent or is carbonyl, alkylcarbonyl, acrylyl, alkoxycarbonyl, aminocarbonyl, aminothiocarbonyl or carbonylaminoiminomethyl.

An example of the invention is a compound of Formula (I) or a form thereof, wherein R₄ is hydrogen or is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl.

An example of the invention is a compound of Formula (I) or a form thereof, wherein R₄ is hydrogen or is aryl or heterocyclyl each optionally substituted with one or more of alkyl, alkoxy, cyano, halogen, haloalkyl, nitro, alkoxycarbonyl or alkylthio.

An example of the invention is a compound of Formula (I) or a form thereof, wherein R₅ is hydrogen or is one or more of alkyl, alkoxy, cyano, halogen, haloalkyl, nitro, alkoxycarbonyl or alkylthio.

An example of the invention is a compound of Formula (I) or a form thereof, wherein
X₄ is absent or is carbonyl, alkylcarbonyl, acrylyl, alkoxycarbonyl, aminocarbonyl, alkylaminocarbonyl, thiocarbonyl, aminothiocarbonyl or carbonylaminoiminomethyl;
R₄ is hydrogen or is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl; and
R₅ is hydrogen or is one or more of alkyl, alkoxy, cyano, halogen, haloalkyl, nitro, alkoxycarbonyl or alkylthio.

An example of the invention is a compound of Formula (I) or a form thereof, wherein
X₄ is absent or is carbonyl, alkylcarbonyl, acrylyl, alkoxycarbonyl, aminocarbonyl, aminothiocarbonyl or carbonylaminoiminomethyl; and
R₄ is hydrogen or is aryl or heterocyclyl each optionally substituted with one or more of alkyl, alkoxy, cyano, halogen, haloalkyl, nitro, alkoxycarbonyl or alkylthio.

An example of the invention is a compound of Formula (I) or a form thereof, wherein
R₁ is aryl or heterocyclyl each optionally substituted with one or more of alkyl, alkoxy, cyano, halogen, haloalkyl, amino, alkylcarbonylamino, alkylsulfonylamino or heterocyclyl;
Ra and Rb is each hydrogen or hydroxy;
R₂ is hydrogen or is oxo, hydroxyalkyl, carboxy or alkoxycarbonyl;
R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen;
X₄ is absent or is carbonyl, alkylcarbonyl, acrylyl, alkoxycarbonyl, aminocarbonyl, aminothiocarbonyl or carbonylaminoiminomethyl;
R₄ is hydrogen or is aryl or heterocyclyl each optionally substituted with one or more of alkyl, alkoxy, cyano, halogen, haloalkyl, nitro, alkoxycarbonyl, alkylthio, R₅-aryl or R₅-heteroaryl; and
R₅ is hydrogen or is one or more of alkyl, alkoxy, cyano, halogen, haloalkyl, nitro, alkoxycarbonyl or alkylthio.

An example of the invention is a compound of Formula (I) or a form thereof, wherein
R₁ is aryl or heterocyclyl each optionally substituted with one or more of alkyl, alkoxy, cyano, halogen, haloalkyl, amino, alkylcarbonylamino, alkylsulfonylamino or heterocyclyl;
Ra and Rb is each hydrogen or hydroxy;
R₂ is hydrogen or is oxo, hydroxyalkyl, carboxy or alkoxycarbonyl;
R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen;
X₄ absent or is carbonyl, alkylcarbonyl, acrylyl, alkoxycarbonyl, aminocarbonyl, aminothiocarbonyl or carbonylaminoiminomethyl; and
R₄ hydrogen or is aryl or heterocyclyl each optionally substituted with one or more of alkyl, alkoxy, cyano, halogen, haloalkyl, nitro, alkoxycarbonyl or alkylthio.

An example of the invention is a compound of Formula (I) or a form thereof, in R₁, Ra, R₂, R₃, Rb and X₄R₄ are dependently selected from

| Cpd | R₁ | Ra | R₂ | R₃ | Rb | X₄R₄ |
|---|---|---|---|---|---|---|
| 1 | (4-Cl)-phenyl | H | H | OH | OH | C(O)CH═CH-3,4- |
| | | | | | | Cl₂-phenyl |
| 2 | (4-Cl)-phenyl | H | oxo | oxo | H | C(O)CH═CH-3,4- |
| | | | | | | Cl₂-phenyl |
| 3 | (4-Cl)-phenyl | H | oxo | OH | H | C(O)CH═CH-3,4- |
| | | | | | | Cl₂-phenyl |
| 4 | indol-3-yl | H | H | OH | H | C(O)NH-3,4-Cl₂- |
| | | | | | | phenyl |
| 5 | indol-3-yl | H | H | OH | H | C(O)NH-3,5-F₂- |
| | | | | | | phenyl |
| 6 | indol-3-yl | H | H | OH | H | C(O)CH═CH-3,4- |
| | | | | | | Cl₂-phenyl |
| 7 | indol-3-yl | H | H | OH | H | C(O)CH═CH-3,5-F₂- |
| | | | | | | phenyl |
| 8 | indol-3-yl | H | H | OH | H | C(O)-3,4,5-F₃-phenyl |
| 9 | indol-3-yl | H | H | OH | H | C(S)NH-3-Br-phenyl |
| 10 | indol-3-yl | H | H | OH | H | C(NH)NHC(O)-3,5- |
| | | | | | | F₂-phenyl |
| 11 | indol-3-yl | H | H | OH | H | C(O)CH═CH-3-CH₃- |
| | | | | | | phenyl |
| 12 | (4-OCH₃)- | H | H | OH | H | C(O)CH═CH-3,5-F₂- |
| | phenyl | | | | | phenyl |
| 13 | (4-OCH₃)- | H | H | OH | H | C(O)CH═CH-3,4,5- |
| | phenyl | | | | | F₃-phenyl |
| 14 | (4-OCH₃)- | H | H | OH | H | C(O)CH═CH-3,4- |
| | phenyl | | | | | Cl₂-phenyl |
| 15 | (4-OCH₃)- | H | H | OH | H | C(O)NH-3,4-Cl₂- |
| | phenyl | | | | | phenyl |
| 16 | (4-OCH₃)- | H | H | oxo | H | C(S)NH-3-Br-phenyl |
| | phenyl | | | | | |
| 17 | 1H- | H | H | oxo | H | C(O)CH═CH-3,4,5- |
| | pyrrolo[2,3- | | | | | F₃-phenyl |
| | b]pyridin-3-yl | | | | | |
| 18 | (5- | H | H | OH | H | C(O)CH═CH-3,4,5- |
| | NHSO₂CH₃)- | | | | | F₃-phenyl |
| | indol-3-yl | | | | | |
| 19 | 1H- | H | H | OH | H | C(O)CH═CH-3-CH₃- |
| | pyrrolo[2,3- | | | | | phenyl |
| | b]pyridin-3-yl | | | | | |
| 20 | 1H- | H | H | OH | H | C(O)CH═CH-3,5-F₂- |
| | pyrrolo[2,3- | | | | | phenyl |
| | b]pyridin-3-yl | | | | | |
| 21 | 1H- | H | H | OH | H | C(O)CH═CH-3,4,5- |
| | pyrrolo[2,3- | | | | | F₃-phenyl |
| | b]pyridin-3-yl | | | | | |
| 22 | 1H- | H | H | OH | H | C(O)CH═CH-3-CF₃- |
| | pyrrolo[2,3- | | | | | phenyl |
| | b]pyridin-3-yl | | | | | |
| 23 | 1H- | H | H | OH | H | C(O)CH═CH-3,4- |
| | pyrrolo[2,3- | | | | | Cl₂-phenyl |
| | b]pyridin-3-yl | | | | | |
| 24 | 1H- | H | H | OH | H | C(O)NH-3,4-Cl₂- |
| | pyrrolo[2,3- | | | | | phenyl |
| | b]pyridin-3-yl | | | | | |
| 25 | 1H- | H | H | OH | H | C(O)NH-4-SCH₃- |
| | pyrrolo[2,3- | | | | | phenyl |
| | b]pyridin-3-yl | | | | | |
| 26 | (5-OCH₃)- | H | H | OH | H | C(O)CH═CH-3,5-F₂- |
| | indol-3-yl | | | | | phenyl |
| 27 | (5- | H | H | OH | H | C(O)CH═CH-3,4,5- |
| | NHSO₂CH₃)- | | | | | F₃-phenyl |
| | indol-3-yl | | | | | |
| 28 | (5- | H | H | NH₂ | H | C(O)CH═CH-3,4,5- |
| | NHSO₂CH₃)- | | | | | F₃-phenyl |
| | indol-3-yl | | | | | |
| 29 | (4-OCH₃)- | H | H | OH | H | C(O)CH═CH-3-CH₃- |
| | phenyl | | | | | phenyl |
| 30 | (4-OCH₃)- | H | H | OH | H | C(O)CH═CH-3-CF₃- |
| | phenyl | | | | | phenyl |
| 31 | (4-OCH₃)- | H | H | OH | H | C(O)NH-3-SCH₃- |
| | phenyl | | | | | phenyl |
| 32 | (4-OCH₃)- | H | H | OH | H | C(O)CH═CH-3,4-F₂- |
| | phenyl | | | | | phenyl |
| 33 | 1H-pyrazol-3- | H | H | OH | H | C(O)CH═CH-3-CH₃- |
| | yl | | | | | phenyl |
| 34 | 1H-pyrazol-3- | H | H | OH | H | C(O)CH═CH-3,5-F₂- |
| | yl | | | | | phenyl |
| 35 | 1H-pyrazol-3- | H | H | OH | H | C(O)CH═CH-3,4,5- |
| | yl | | | | | F₃-phenyl |
| 36 | 1H-pyrazol-3- | H | H | OH | H | C(O)NH-3,4-Cl₂- |
| | yl | | | | | phenyl |
| 37 | 1H-pyrazol-3- | H | H | OH | H | C(O)NH-3,4-F₂- |
| | yl | | | | | phenyl |
| 38 | (4-OCH₃)- | H | H | CO₂—CH₂CH₃ | H | C(O)CH═CH-3,4,5- |
| | phenyl | | | | | F₃-phenyl |
| 39 | (4-OCH₃)- | H | H | CO₂H | H | C(O)CH═CH-3,4,5- |
| | phenyl | | | | | F₃-phenyl |
| 40 | (5- | H | H | OH | H | C(O)CH═CH-3-CH₃- |
| | NHSO₂CH₃)- | | | | | phenyl |
| | indol-3-yl | | | | | |
| 41 | (5- | H | H | OH | H | C(O)CH═CH-3,5-F₂- |
| | NHSO₂CH₃)- | | | | | phenyl |
| | indol-3-yl | | | | | |
| 42 | (4-OCH₃)- | H | H | CH₂OH | H | C(O)CH═CH-3-CH₃- |
| | phenyl | | | | | phenyl |
| 43 | (4-OCH₃)- | H | H | CH₂OH | H | C(O)CH═CH-3,4- |
| | phenyl | | | | | Cl₂-phenyl |
| 44 | (4-OCH₃)- | H | H | CH₂OH | H | C(O)NH-3,4-Cl₂- |
| | phenyl | | | | | phenyl |
| 45 | (4-OCH₃)- | H | H | CH₂OH | H | C(O)CH═CH-3,5-F₂- |
| | phenyl | | | | | phenyl |
| 46 | benzoxazolyl- | H | H | CH₂OH | H | C(O)CH═CH-3,5-F₂- |
| | 2-yl | | | | | phenyl |
| 47 | benzoxazolyl- | H | H | CH₂OH | H | C(O)CH═CH-3-CH₃- |
| | 2-yl | | | | | phenyl |
| 48 | indol-3-yl | H | CO₂—CH₂CH₃ | H | H | C(O)CH═CH-3-CH₃- |
| | | | | | | phenyl |
| 49 | indol-3-yl | H | CO₂—CH₂CH₃ | H | H | C(O)CH═CH-3,4- |
| | | | | | | Cl₂-phenyl |
| 50 | indol-3-yl | H | CO₂—CH₂CH₃ | H | H | C(O)CH═CH-3,4,5- |
| | | | | | | F₃-phenyl |
| 51 | indol-3-yl | H | CO₂—CH₂CH₃ | H | H | C(O)CH═CH-3,5-F₂- |
| | | | | | | phenyl |
| 52 | indol-3-yl | H | CO₂—CH₂CH₃ | H | H | C(O)NH-3,4-Cl₂- |
| | | | | | | phenyl |
| 53 | indol-3-yl | H | CO₂H | H | H | C(O)CH═CH-3-CH₃- |
| | | | | | | phenyl |
| 54 | indol-3-yl | H | CH₂OH | H | H | C(O)CH═CH-3,5-F₂- |
| | | | | | | phenyl |
| 55 | indol-3-yl | H | CH₂OH | H | H | C(O)CH═CH-3,4,5- |
| | | | | | | F₃-phenyl |
| 56 | indol-3-yl | H | CH₂OH | H | H | C(O)CH═CH-3,4- |
| | | | | | | Cl₂-phenyl |
| 57 | indol-3-yl | H | CH₂OH | H | H | C(O)CH═CH-3-CH₃- |
| | | | | | | phenyl |
| 58 | indol-3-yl | H | CH₂OH | H | H | C(O)NH-3,4-Cl₂- |
| | | | | | | phenyl |
| 59 | indol-3-yl | H | CH₂OH | H | H | C(S)NH-3-Br-phenyl |
| 60 | indol-3-yl | H | CO₂H | H | H | C(O)CH═CH-3,4- |
| | | | | | | Cl₂-phenyl |
| 61 | indol-3-yl | H | CO₂H | H | H | C(O)CH═CH-3,5-F₂- |
| | | | | | | phenyl |
| 62 | indol-3-yl | H | CO₂H | H | H | C(O)NH-3,4-Cl₂- |
| | | | | | | phenyl |
| 63 | (5- | H | H | OH | H | C(O)OC(CH₃)₃ |
| | NHSO₂CH₃)- | | | | | |
| | indol-3-yl | | | | | |
| 64 | (4-OCH₃)- | H | H | oxo | H | C(O)CH═CH-3,4,5- |
| | phenyl | | | | | F₃-phenyl |
| 65 | (4-OCH₃)- | H | H | NH₂ | H | C(O)CH═CH-3,4,5- |
| | phenyl | | | | | F₃-phenyl |
| 66 | (4-OCH₃)- | H | H | NHC(O)—CH₃ | H | C(O)CH═CH-3,4,5- |
| | phenyl | | | | | F₃-phenyl |
| 67 | (4-OCH₃)- | H | H | NHC(O)—OCH₃ | H | C(O)CH═CH-3,4,5- |
| | phenyl | | | | | F₃-phenyl |
| 68 | (4-OCH₃)- | H | H | NHC(O)—N(CH₃)₂ | H | C(O)CH═CH-3,4,5- |
| | phenyl | | | | | F₃-phenyl |
| 69 | indol-3-yl | H | H | OH | OH | C(O)O-benzyl |
| 70 | indol-3-yl | H | H | OH | OH | C(O)CH═CH-3,4,5- |
| | | | | | | F₃-phenyl |
| 71 | indol-3-yl | H | H | OH | OH | C(O)CH═CH-3,5-F₂- |
| | | | | | | phenyl |
| 72 | indol-3-yl | H | H | OH | OH | C(O)CH═CH-3,5- |
| | | | | | | (CF₃)₂-phenyl |
| 73 | indol-3-yl | H | H | OH | OH | C(O)CH═CH-3,5- |
| | | | | | | Cl₂-phenyl |
| 74 | indol-3-yl | H | H | OH | OH | C(O)CH═CH-3-F-4- |
| | | | | | | Cl-phenyl |
| 75 | indol-3-yl | H | H | OH | OH | C(O)CH═CH-3,4- |
| | | | | | | Cl₂-phenyl |
| 76 | indol-3-yl | H | H | OH | OH | C(O)CH═CH-3-F- |
| | | | | | | phenyl |
| 77 | indol-3-yl | H | H | OH | OH | C(O)CH═CH-3-CF₃- |
| | | | | | | phenyl |
| 78 | indol-3-yl | H | H | OH | OH | C(O)CH═CH-3,4-F₂- |
| | | | | | | phenyl |
| 79 | indol-3-yl | H | H | OH | OH | C(O)CH═CH-thien-3- |
| | | | | | | yl |
| 80 | indol-3-yl | H | H | OH | OH | C(O)CH═CH-3-Br-4- |
| | | | | | | F-phenyl |
| 81 | indol-3-yl | H | H | OH | OH | C(O)NH-3,5-F₂- |
| | | | | | | phenyl |
| 82 | indol-3-yl | H | H | OH | OH | C(O)NH-3,4-F₂- |
| | | | | | | phenyl |
| 83 | indol-3-yl | H | H | OH | OH | C(S)NH-3,5-Cl₂- |
| | | | | | | phenyl |
| 84 | indol-3-yl | H | H | OH | OH | C(S)NH-3,4-Cl₂- |
| | | | | | | phenyl |
| 85 | 1H- | H | H | OH | H | C(O)CH═CH-3,4- |
| | benzoimidazol- | | | | | Cl₂-phenyl |
| | 2-yl | | | | | |
| 86 | 1H- | H | H | OH | H | C(O)CH═CH-3,4,5- |
| | benzoimidazol- | | | | | F₃-phenyl |
| | 2-yl | | | | | |
| 87 | 1H- | H | H | OH | OH | C(O)CH═CH-3,4,5- |
| | benzoimidazol- | | | | | F₃-phenyl |
| | 2-yl | | | | | |
| 88 | (4-OCH₃)- | H | H | OH | OH | C(O)CH═CH-3,4,5- |
| | phenyl | | | | | F₃-phenyl |
| 89 | (4-OCH₃)- | H | H | OH | H | C(S)NH-3,5-Cl₂- |
| | phenyl | | | | | phenyl |
| 90 | (4-OCH₃)- | H | H | OH | H | C(O)NH-2-F-4,5-Cl₂- |
| | phenyl | | | | | phenyl |
| 91 | (4-OCH₃)- | H | H | OH | H | C(O)NH-3-CF₃-5-F- |
| | phenyl | | | | | phenyl |
| 92 | (4-OCH₃)- | H | H | OH | OH | C(O)CH═CH-4-Cl- |
| | phenyl | | | | | phenyl |
| 93 | (4-OCH₃)- | H | H | OH | OH | C(O)CH═CH-3,4-F₂- |
| | phenyl | | | | | phenyl |
| 94 | (4-OCH₃)- | H | H | OH | OH | C(O)CH═CH-3,5-F₂- |
| | phenyl | | | | | phenyl |
| 95 | (4-OCH₃)- | H | H | OH | OH | C(O)CH═CH-3-CH₃- |
| | phenyl | | | | | phenyl |
| 96 | (4-OCH₃)- | H | H | OH | H | C(S)NH-3-OCH₃- |
| | phenyl | | | | | phenyl |
| 97 | (4-OCH₃)- | H | H | OH | H | C(S)NH-3-CF₃- |
| | phenyl | | | | | phenyl |
| 98 | (4-OCH₃)- | H | H | OH | H | C(S)NH-3,4-Cl₂- |
| | phenyl | | | | | phenyl |
| 99 | (4-OCH₃)- | H | H | OH | H | C(S)NH-3-F-4-Br- |
| | phenyl | | | | | phenyl |
| 100 | (4-OCH₃)- | H | H | OH | H | C(O)NH- |
| | phenyl | | | | | benzo[1,3]dioxol-5-yl |
| 101 | (4-OCH₃)- | H | H | OH | H | C(O)NH-2-Cl-4-CF₃- |
| | phenyl | | | | | phenyl |
| 102 | (4-OCH₃)- | H | H | OH | H | C(S)NH-3-Cl-phenyl |
| | phenyl | | | | | |
| 103 | (4-OCH₃)- | H | H | OH | H | C(S)NH-3-OCH₃- |
| | phenyl | | | | | phenyl |
| 104 | (4-OCH₃)- | H | H | OH | H | C(S)NH-3-CN- |
| | phenyl | | | | | phenyl |
| 105 | (4-OCH₃)- | H | H | OH | H | C(S)NH-3-CF₃- |
| | phenyl | | | | | phenyl |
| 106 | (4-OCH₃)- | H | H | OH | H | C(S)NH-4-CF₃- |
| | phenyl | | | | | phenyl |
| 107 | (4-OCH₃)- | H | H | OH | H | C(S)NH-3,5-F₂- |
| | phenyl | | | | | phenyl |
| 108 | (4-OCH₃)- | H | H | OH | H | C(S)NH-3,4-Cl₂- |
| | phenyl | | | | | phenyl |
| 109 | (4-OCH₃)- | H | H | OH | H | C(S)NH-2,3,5-F₃- |
| | phenyl | | | | | phenyl |
| 110 | (4-OCH₃)- | H | H | OH | H | C(S)NH-3-F-4-Br- |
| | phenyl | | | | | phenyl |
| 111 | (4-OCH₃)- | H | H | OH | H | C(S)NH-3,5- |
| | phenyl | | | | | (OCH₃)₂-phenyl |
| 112 | (4-Cl)-phenyl | H | H | OH | OH | C(O)O-benzyl |
| 113 | (4-OCH₃)- | H | H | OH | OH | C(O)CH═CH-thien-3- |
| | phenyl | | | | | yl |
| 114 | (4-OCH₃)- | H | H | OH | OH | C(O)NH-3,4-F₂- |
| | phenyl | | | | | phenyl |
| 115 | (4-OCH₃)- | H | H | OH | OH | C(O)NH-3,5-F₂- |
| | phenyl | | | | | phenyl |
| 116 | (4-OCH₃)- | H | H | OH | OH | C(O)NH-3,4-Cl₂- |
| | phenyl | | | | | phenyl |
| 117 | (4-OCH₃)- | H | H | OH | OH | C(S)NH-3,4-Cl₂- |
| | phenyl | | | | | phenyl |
| 118 | (4-OCH₃)- | H | H | OH | OH | C(S)NH-3,5-Cl₂- |
| | phenyl | | | | | phenyl |
| 119 | (4-OCH₃)- | H | H | OH | OH | C(S)NH-3,5- |
| | phenyl | | | | | (OCH₃)₂-phenyl |
| 120 | indol-3-yl | H | H | OH | OH | (4-CF₃)-pyrimidin-2- |
| | | | | | | yl |
| 121 | 1H- | H | H | OH | OH | (4-CF₃)-pyrimidin-2- |
| | benzoimidazol- | | | | | yl |
| | 2-yl | | | | | |
| 122 | (5-morpholin- | H | H | OH | OH | (4-CF₃)-pyrimidin-2- |
| | 4-yl)-indol-3- | | | | | yl |
| | yl | | | | | |
| 123 | (5-CH₃)- | H | H | OH | OH | (4-CF₃)-pyrimidin-2- |
| | indol-3-yl | | | | | yl |
| 124 | (5-CN)-indol- | H | H | OH | OH | (4-CF₃)-pyrimidin-2- |
| | 3-yl | | | | | yl |
| 125 | (4-F)-phenyl | H | H | OH | OH | C(O)O-benzyl |
| 126 | (4-CF₃)- | H | H | OH | OH | C(O)O-benzyl |
| | phenyl | | | | | |
| 127 | (4-OCH₃)- | H | H | OH | H | (5-NO₂)-pyridin-2-yl |
| | phenyl | | | | | |
| 128 | (4-F)-phenyl | H | H | OH | OH | C(O)CH═CH-3,4,5- |
| | | | | | | F₃-phenyl |
| 129 | (4-F)-phenyl | H | H | OH | OH | C(O)CH═CH-3,4-F₂- |
| | | | | | | phenyl |
| 130 | (4-F)-phenyl | H | H | OH | OH | C(O)CH═CH-3,5-F₂- |
| | | | | | | phenyl |
| 131 | (4-F)-phenyl | H | H | OH | OH | C(O)CH═CH-3,4- |
| | | | | | | Cl₂-phenyl |
| 132 | (4-F)-phenyl | H | H | OH | OH | C(O)NH-3,4-F₂- |
| | | | | | | phenyl |
| 133 | (4-F)-phenyl | H | H | OH | OH | C(O)NH-3,5-F₂- |
| | | | | | | phenyl |
| 134 | (4-F)-phenyl | H | H | OH | OH | C(O)NH-3,4-Cl₂- |
| | | | | | | phenyl |
| 135 | (4-F)-phenyl | H | H | OH | OH | C(S)NH-3,4-Cl₂- |
| | | | | | | phenyl |
| 136 | (4-CF₃)- | H | H | OH | OH | C(O)CH═CH-3,4,5- |
| | phenyl | | | | | F₃-phenyl |
| 137 | (4-CF₃)- | H | H | OH | OH | C(O)CH═CH-3,4-F₂- |
| | phenyl | | | | | phenyl |
| 138 | (4-CF₃)- | H | H | OH | OH | C(O)CH═CH-3,5-F₂- |
| | phenyl | | | | | phenyl |
| 139 | (4-CF₃)- | H | H | OH | OH | C(O)CH═CH-3,4- |
| | phenyl | | | | | Cl₂-phenyl |
| 140 | (4-CF₃)- | H | H | OH | OH | C(O)CH═CH-3,4- |
| | phenyl | | | | | (OCH₃)₂-phenyl |
| 141 | (4-CF₃)- | H | H | OH | OH | C(O)NH-3,4-F₂- |
| | phenyl | | | | | phenyl |
| 142 | (4-CF₃)- | H | H | OH | OH | C(O)NH-3,5-F₂- |
| | phenyl | | | | | phenyl |
| 143 | (4-CF₃)- | H | H | OH | OH | C(O)NH-3,4-Cl₂- |
| | phenyl | | | | | phenyl |
| 144 | (4-CF₃)- | H | H | OH | OH | C(S)NH-3,4-Cl₂- |
| | phenyl | | | | | phenyl |
| 145 | (4-CF₃)- | H | H | OH | OH | C(S)NH-3,5-Cl₂- |
| | phenyl | | | | | phenyl |
| 146 | (4-Cl)-phenyl | H | H | OH | OH | C(O)OC(CH₃)₃ |
| 147 | (5-NH₂)- | H | H | OH | H | C(O)O-benzyl |
| | indol-3-yl | | | | | |
| 148 | (5-morpholin- | H | H | OH | OH | C(O)CH═CH-3,4,5- |
| | 4-yl)-indol-3- | | | | | F₃-phenyl |
| | yl | | | | | |
| 149 | (5-morpholin- | H | H | OH | OH | C(O)CH═CH-3,5-F₂- |
| | 4-yl)-indol-3- | | | | | phenyl |
| | yl | | | | | |
| 150 | (5-morpholin- | H | H | OH | OH | C(O)CH═CH-3,5- |
| | 4-yl)-indol-3- | | | | | CF₃-phenyl |
| | yl | | | | | |
| 151 | (5-morpholin- | H | H | OH | OH | C(O)CH═CH-3,5- |
| | 4-yl)-indol-3- | | | | | Cl₂-phenyl |
| | yl | | | | | |
| 152 | (4-F)-phenyl | OH | H | OH | OH | C(O)CH═CH-3,4,5- |
| | | | | | | F₃-phenyl |
| 153 | (4-F)-phenyl | OH | H | OH | OH | C(O)CH═CH-3,4-F₂- |
| | | | | | | phenyl |
| 154 | (4-F)-phenyl | OH | H | OH | OH | C(O)CH═CH-3,5-F₂- |
| | | | | | | phenyl |
| 155 | (5-F)-indol-3- | H | H | OH | OH | C(O)CH═CH-3,4,5- |
| | yl | | | | | F₃-phenyl |
| 156 | (5-F)-indol-3- | H | H | OH | OH | C(O)CH═CH-3,4-F₂- |
| | yl | | | | | phenyl |
| 157 | (5-F)-indol-3- | H | H | OH | OH | C(O)CH═CH-3,5-F₂- |
| | yl | | | | | phenyl |
| 158 | (5-F)-indol-3- | H | H | OH | OH | C(O)CH═CH-3,4- |
| | yl | | | | | Cl₂-phenyl |
| 159 | (5-F)-indol-3- | H | H | OH | OH | C(O)CH═CH-3-Br-4- |
| | yl | | | | | F-phenyl |
| 160 | (5-F)-indol-3- | H | H | OH | OH | C(O)(CH₂)₂-4-Cl- |
| | yl | | | | | phenyl |
| 161 | (5-F)-indol-3- | H | H | OH | OH | C(O)NH-2-Cl-5-F- |
| | yl | | | | | phenyl |
| 162 | (5-F)-indol-3- | H | H | OH | OH | C(S)NH-3,4-Cl₂- |
| | yl | | | | | phenyl |
| 163 | (4-Cl)-phenyl | H | H | OH | OH | C(O)CH═CH-3,4,5- |
| | | | | | | F₃-phenyl |
| 164 | (4-Cl)-phenyl | H | H | OH | OH | C(O)CH═CH-3,5-F₂- |
| | | | | | | phenyl |
| 165 | (4-Cl)-phenyl | H | H | OH | OH | C(O)NH-3,4-Cl₂- |
| | | | | | | phenyl |
| 166 | (4-Cl)-phenyl | H | H | OH | OH | C(S)NH-2,3,5-F₃- |
| | | | | | | phenyl |
| 167 | (5-F)-indol-3- | H | H | OH | OH | C(O)NH-3,4-Cl₂- |
| | yl | | | | | phenyl |
| 168 | (5-F)-indol-3- | H | H | OH | OH | C(O)(CH₂)₂-3,4-Cl₂- |
| | yl | | | | | phenyl |
| 169 | (5-morpholin- | H | H | OH | OH | C(O)CH═CH-3,4- |
| | 4-yl)-indol-3- | | | | | Cl₂-phenyl |
| | yl | | | | | |
| 170 | (5-morpholin- | H | H | OH | OH | C(O)CH═CH-4-Cl- |
| | 4-yl)-indol-3- | | | | | phenyl |
| | yl | | | | | |
| 171 | (5-morpholin- | H | H | OH | OH | C(O)CH═CH-4-F- |
| | 4-yl)-indol-3- | | | | | phenyl |
| | yl | | | | | |
| 172 | (5-morpholin- | H | H | OH | OH | C(O)CH═CH-3,4-F₂- |
| | 4-yl)-indol-3- | | | | | phenyl |
| | yl | | | | | |
| 173 | (5-morpholin- | H | H | OH | OH | C(O)CH═CH-3-Br-4- |
| | 4-yl)-indol-3- | | | | | F-phenyl |
| | yl | | | | | |
| 174 | (5-morpholin- | H | H | OH | OH | C(O)CH═CH-3-CH₃- |
| | 4-yl)-indol-3- | | | | | phenyl |
| | yl | | | | | |
| 175 | (5-morpholin- | H | H | OH | OH | C(O)CH═CH-2,4,5- |
| | 4-yl)-indol-3- | | | | | F₃-phenyl |
| | yl | | | | | |
| 176 | (5-morpholin- | H | H | OH | OH | C(O)-3-NO₂-benzyl |
| | 4-yl)-indol-3- | | | | | |
| | yl | | | | | |
| 177 | (5-morpholin- | H | H | OH | OH | C(O)CH═CH-3- |
| | 4-yl)-indol-3- | | | | | OCH₃-phenyl |
| | yl | | | | | |
| 178 | (5-morpholin- | H | H | OH | OH | C(O)CH═CH-thien-3- |
| | 4-yl)-indol-3- | | | | | yl |
| | yl | | | | | |
| 179 | (5-morpholin- | H | H | OH | OH | C(O)NH-3,4-F₂- |
| | 4-yl)-indol-3- | | | | | phenyl |
| | yl | | | | | |
| 180 | (5-morpholin- | H | H | OH | OH | C(O)NH-3-F-5-CF₃- |
| | 4-yl)-indol-3- | | | | | phenyl |
| | yl | | | | | |
| 181 | (5-morpholin- | H | H | OH | OH | C(O)NH-3,4-Cl₂- |
| | 4-yl)-indol-3- | | | | | phenyl |
| | yl | | | | | |
| 182 | (5-morpholin- | H | H | OH | OH | C(O)NH-3,5-Cl₂- |
| | 4-yl)-indol-3- | | | | | phenyl |
| | yl | | | | | |
| 183 | (5-morpholin- | H | H | OH | OH | C(O)NH-3,5-F₂- |
| | 4-yl)-indol-3- | | | | | phenyl |
| | yl | | | | | |
| 184 | (5-NHC(O)- | H | H | OH | H | C(O)O-benzyl |
| | CH₃)-indol-3- | | | | | |
| | yl | | | | | |
| 185 | (5-NHC(O)- | H | H | OH | H | H |
| | CH₃)-indol-3- | | | | | |
| | yl | | | | | |

An example of the invention is a compound of Formula (I) or a form thereof, wherein
R₁ is selected from (4-Cl)-phenyl, (4-OCH₃)-phenyl, (4-F)-phenyl, (4-CF₃)-phenyl, indol-3-yl, (5-CH₃)-indol-3-yl, (5-OCH₃)-indol-3-yl, (5-CN)-indol-3-yl, (5-NH₂)-indol-3-yl, (5-F)-indol-3-yl, (5-NHC(O)—CH₃)-indol-3-yl, (5-NHSO₂CH₃)-indol-3-yl, (5-morpholin-4-yl)-indol-3-yl, 1H-pyrazol-3-yl, benzoxazolyl-2-yl, 1H-benzoimidazol-2-yl, or 1H-pyrrolo[2,3-b]pyridin-3-yl;
Ra is selected from hydrogen or hydroxy;
Rb is selected from hydrogen or hydroxy;
R₂ is selected from hydrogen, oxo, CO₂H, CO₂CH₂CH₃ or CH₂OH;
R₃ is selected from hydrogen, oxo, hydroxy, NH₂, CO₂H, CO₂CH₂CH₃, CH₂OH, NHC(O)CH₃, NHC(O)OCH₃ or NHC(O)N(CH₃)₂, with the proviso that R₂ and R₃ are not simultaneously hydrogen; and
X₄R₄ is selected from hydrogen, C(O)CH═CH-3,4-Cl₂-phenyl, C(O)CH═CH-3,5-Cl₂-phenyl, C(O)CH═CH-3,4-F₂-phenyl, C(O)CH═CH-3,5-F₂-phenyl, C(O)CH═CH-3-CH₃-phenyl, C(O)CH═CH-3-OCH₃-phenyl, C(O)CH═CH-3-CF₃-phenyl, C(O)CH═CH-3-F-phenyl, C(O)CH═CH-4-F-phenyl, C(O)CH═CH-4-Cl-phenyl, C(O)CH═CH-3,5-(CF₃)-2-phenyl, C(O)CH═CH-3-F-4-Cl-phenyl, C(O)CH═CH-3,4-(OCH₃)-2-phenyl, C(O)CH═CH-3,5-CF₃-phenyl, C(O)CH═CH-2,4,5-F₃-phenyl, C(O)CH═CH-3,4,5-F₃-phenyl, C(O)CH═CH-3-Br-4-F-phenyl, C(O)CH═CH-thien-3-yl, C(O)NH-3,4-Cl₂-phenyl, C(O)NH-3,5-Cl₂-phenyl, C(O)NH-3,4-F₂-phenyl, C(O)NH-3,5-F₂-phenyl, C(O)NH-3-SCH₃-phenyl, C(O)NH-4-SCH₃-phenyl, C(O)NH-2-F-4,5-Cl₂-phenyl, C(O)NH-2-Cl₅-F-phenyl, C(O)NH-3-CF₃-5-F-phenyl, C(O)NH-3-F-5-CF₃-phenyl, C(O)NH-2-Cl₄-CF₃-phenyl, C(O)NH-benzo[1,3]dioxol-5-yl, C(S)NH-3-Br-phenyl, C(S)NH-3,5-Cl₂-phenyl, C(S)NH-3,4-Cl₂-phenyl, C(S)NH-3-OCH₃-phenyl, C(S)NH-3-CF₃-phenyl, C(S)NH-3-F-4-Br-phenyl, C(S)NH-3-Cl-phenyl, C(S)NH-3-CN-phenyl, C(S)NH-4-CF₃-phenyl, C(S)NH-3,5-F₂-phenyl, C(S)NH-2,3,5-F₃-phenyl, C(S)NH-3,5-(OCH₃)-2-phenyl, C(O)-3,4,5-F₃-phenyl, C(NH)NHC(O)-3,5-F₂-phenyl, C(O)OC(CH₃)₃, C(O)O-benzyl, (4-CF₃)-pyrimidin-2-yl, (5-NO₂)-pyridin-2-yl, C(O)(CH₂)₂-4-Cl-phenyl, C(O)(CH₂)₂-3,4-Cl₂-phenyl or C(O)-3-NO₂-benzyl.

An example of the invention is a compound of Formula (I) or a form thereof represented as follows: 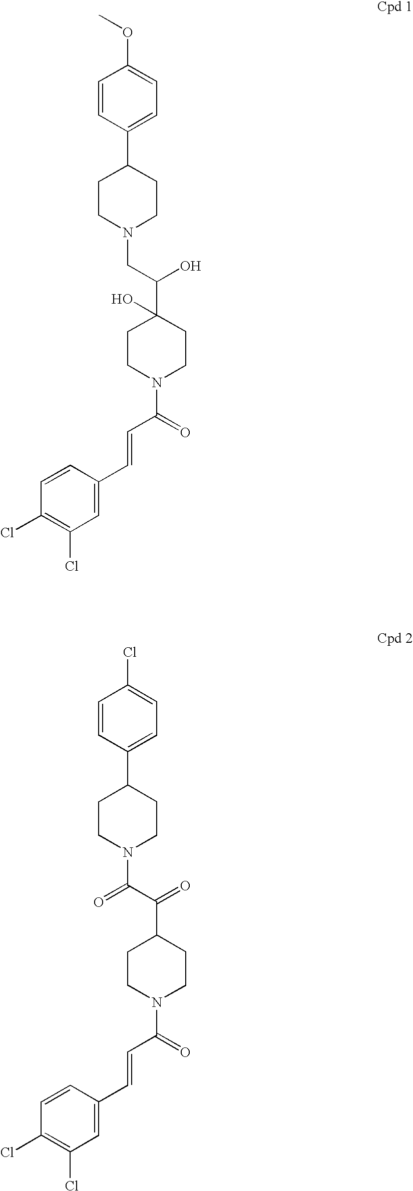 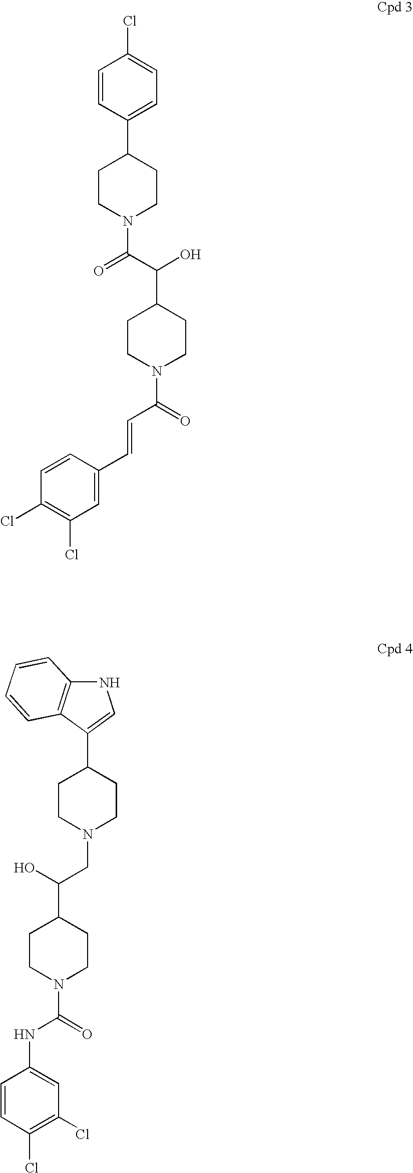 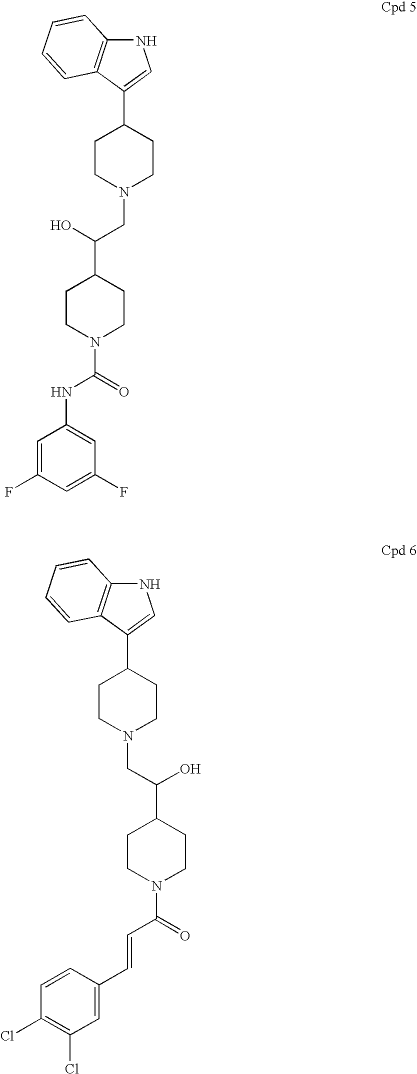 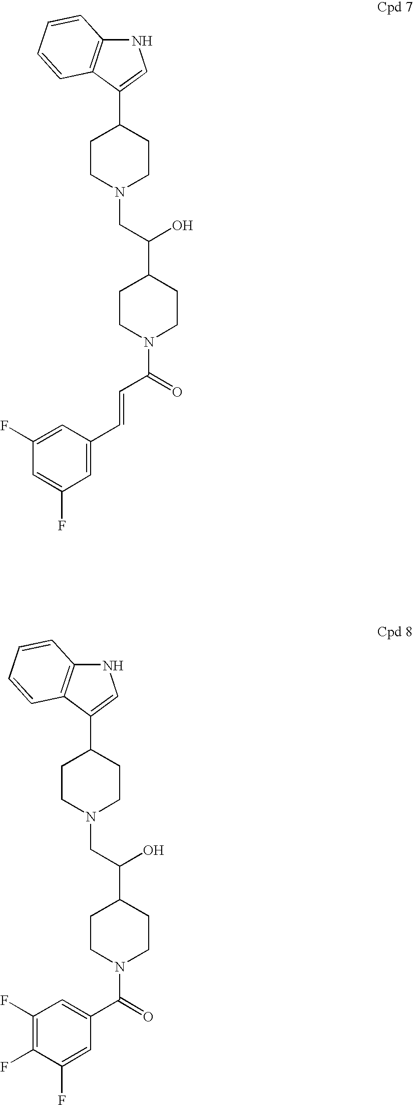 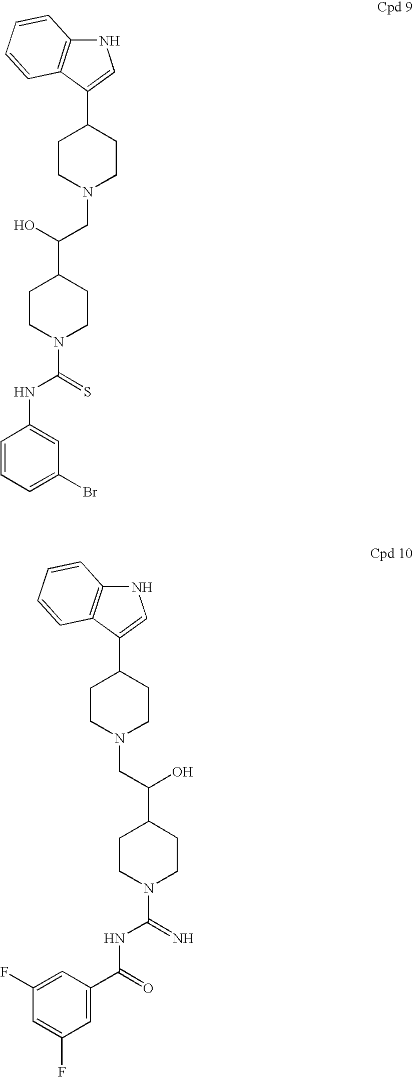 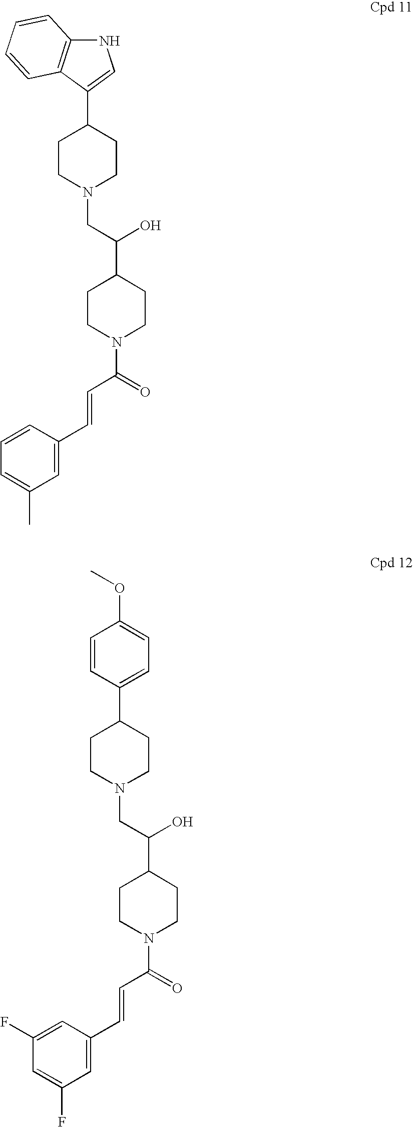 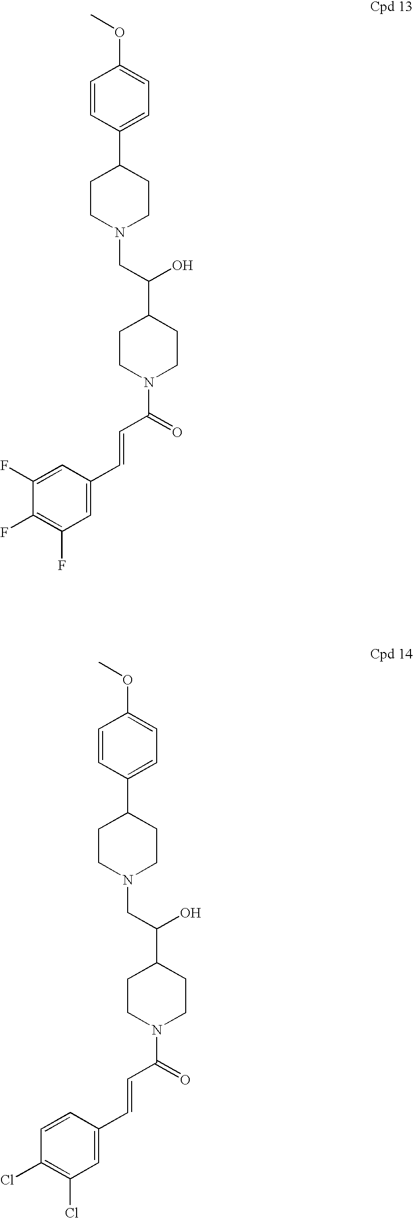 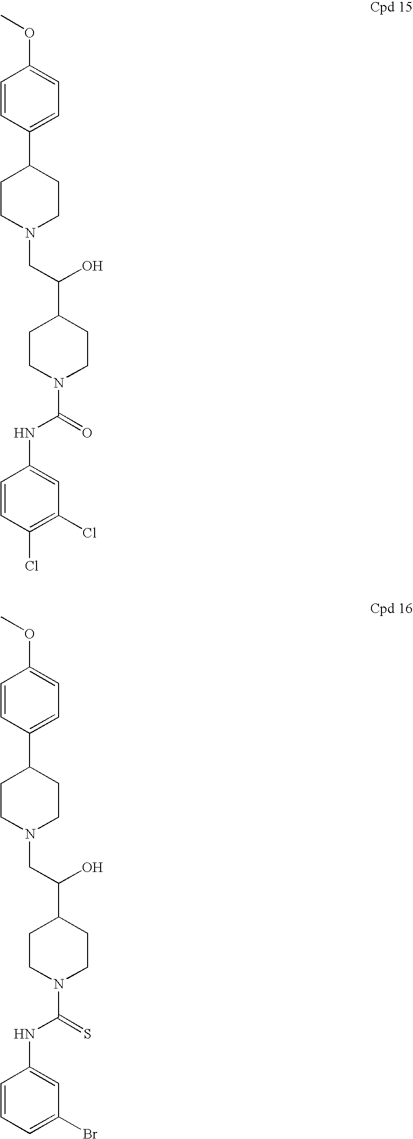 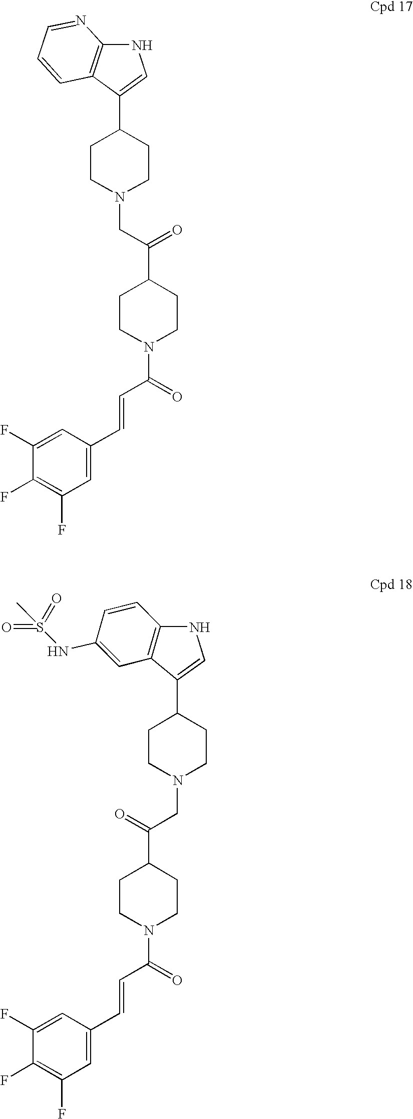 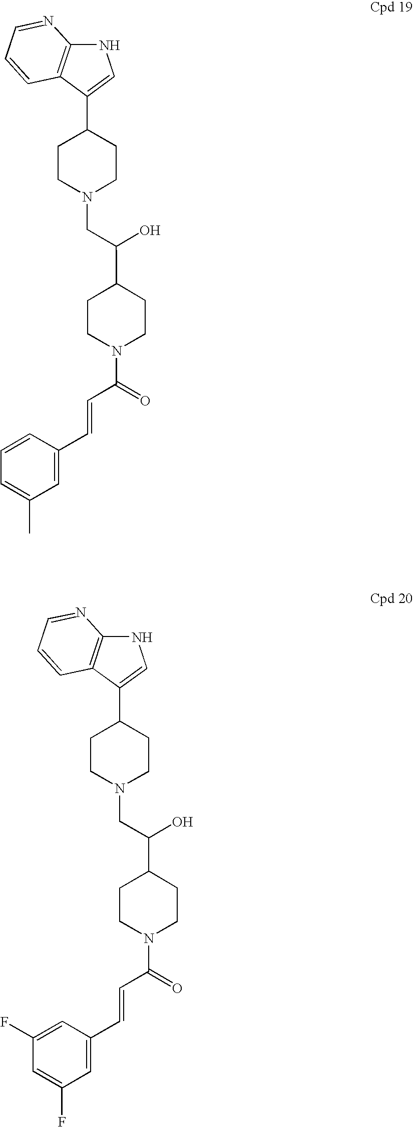 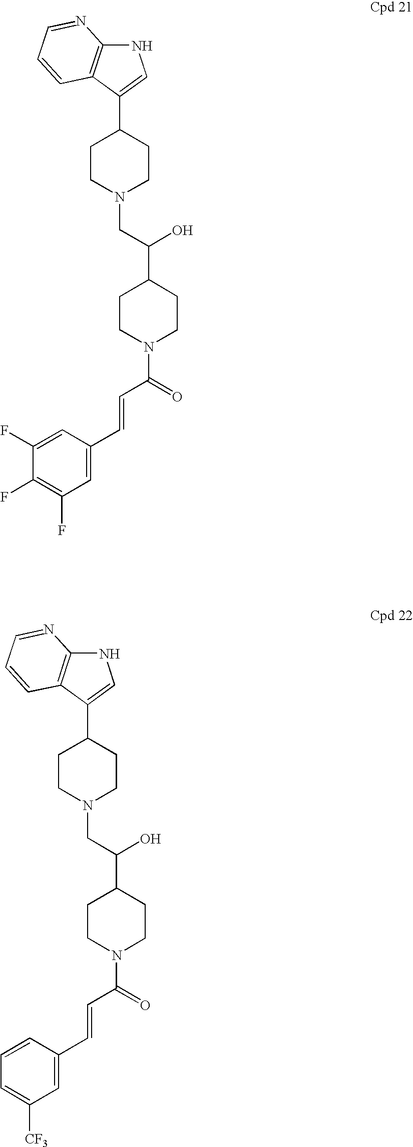 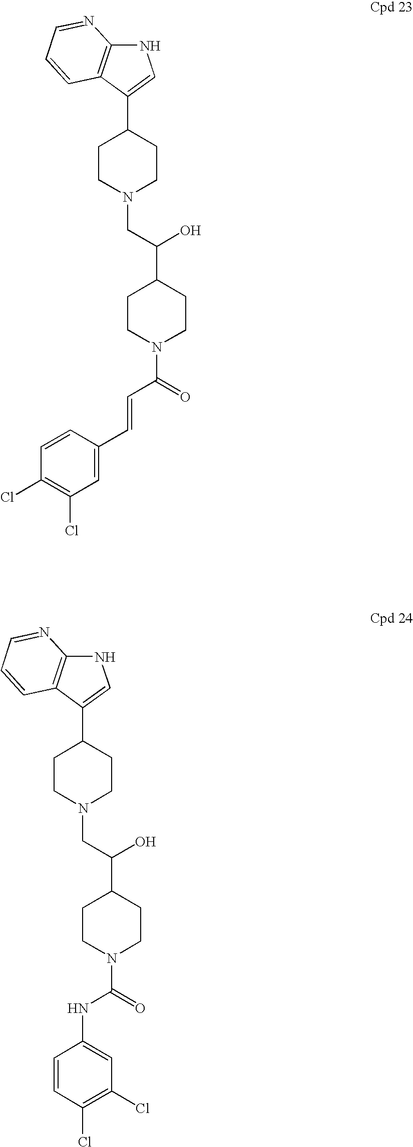 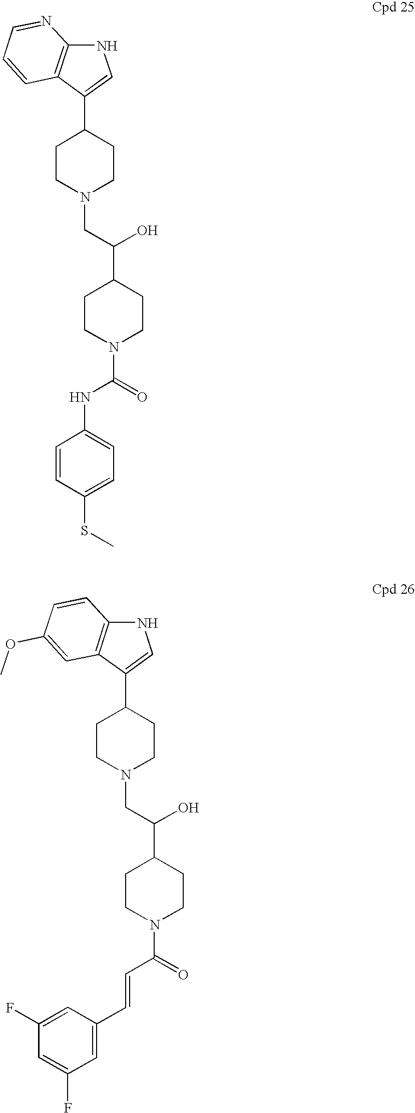 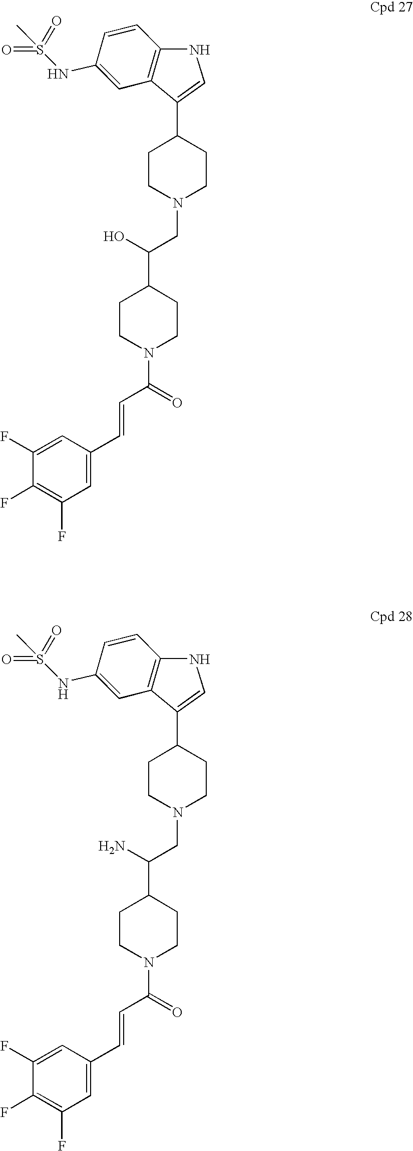 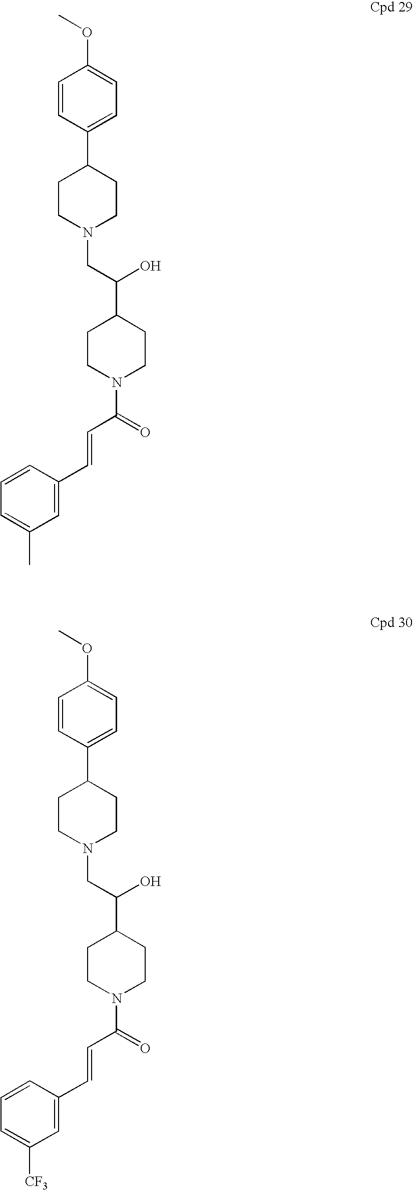 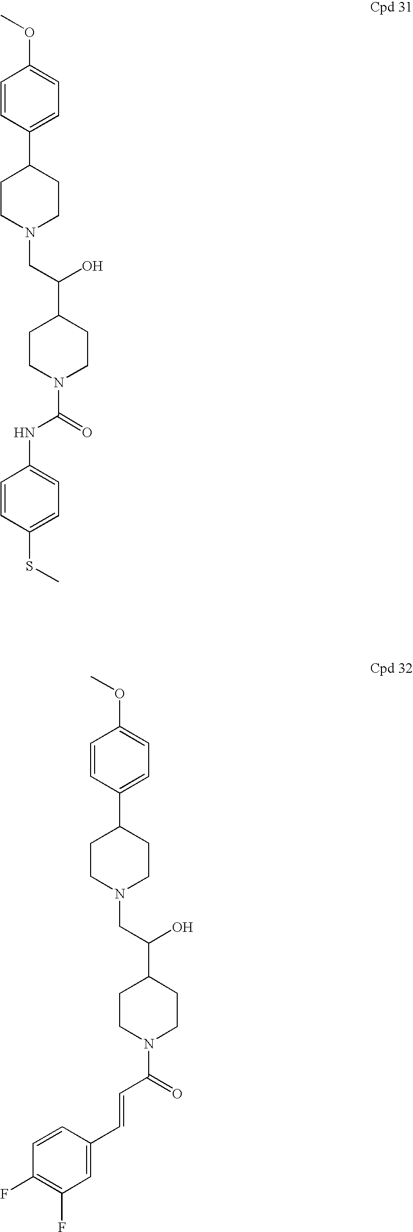 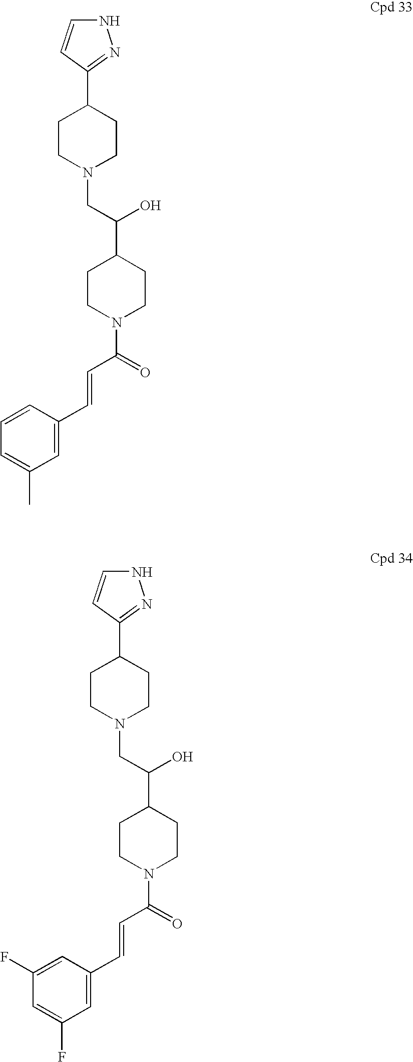 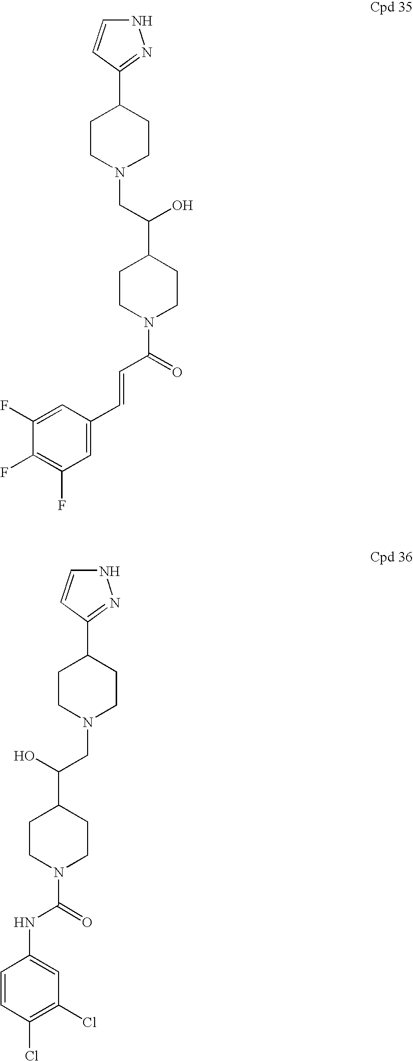 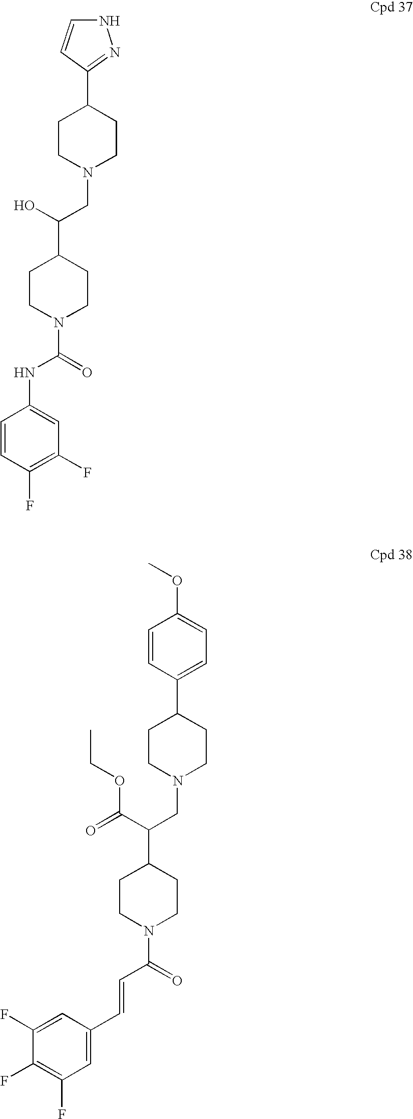 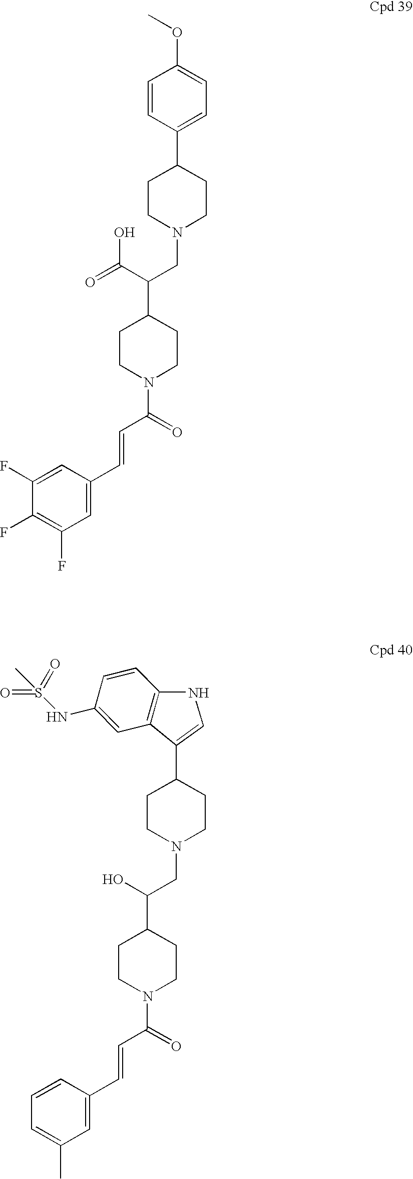 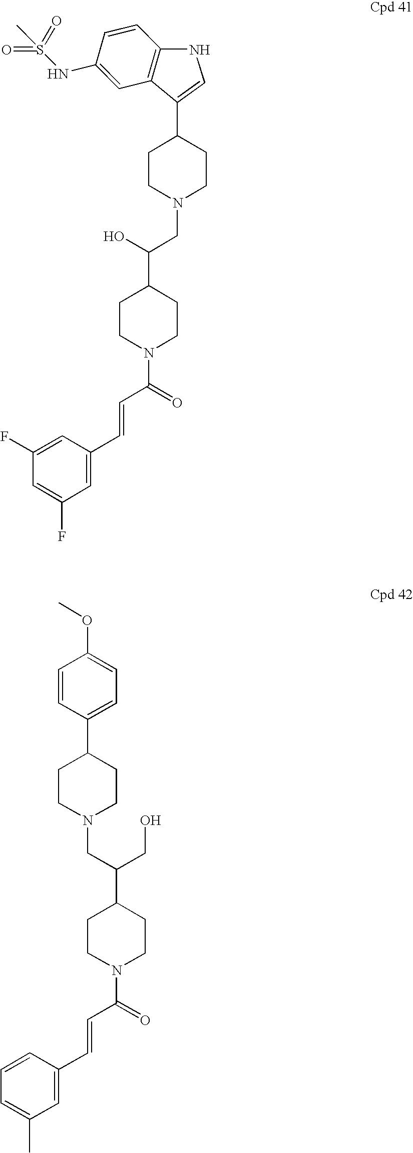 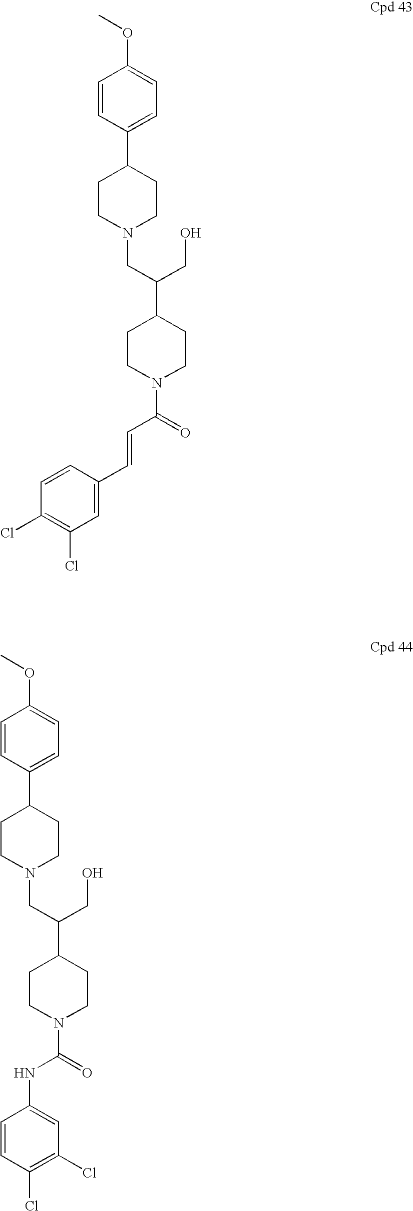 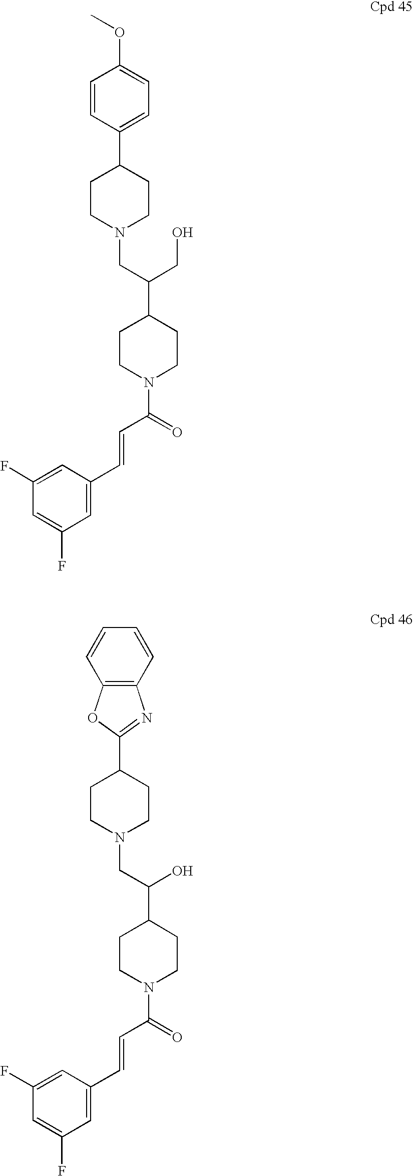 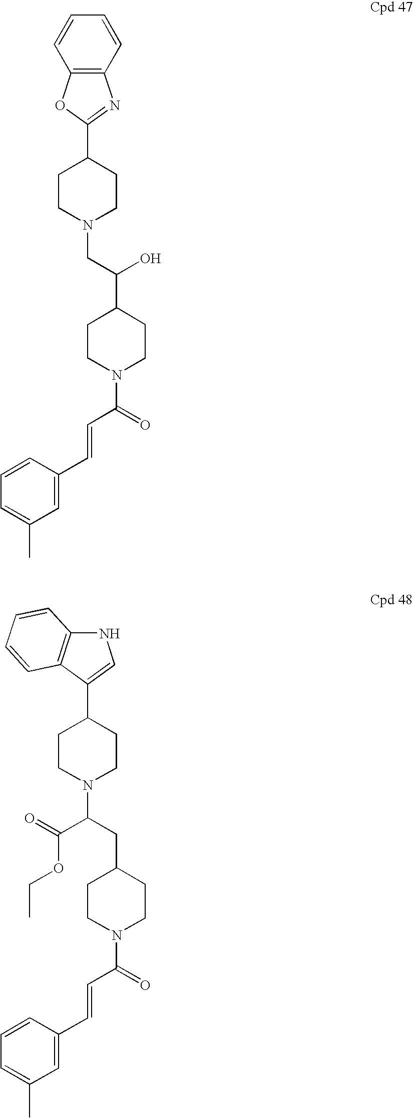 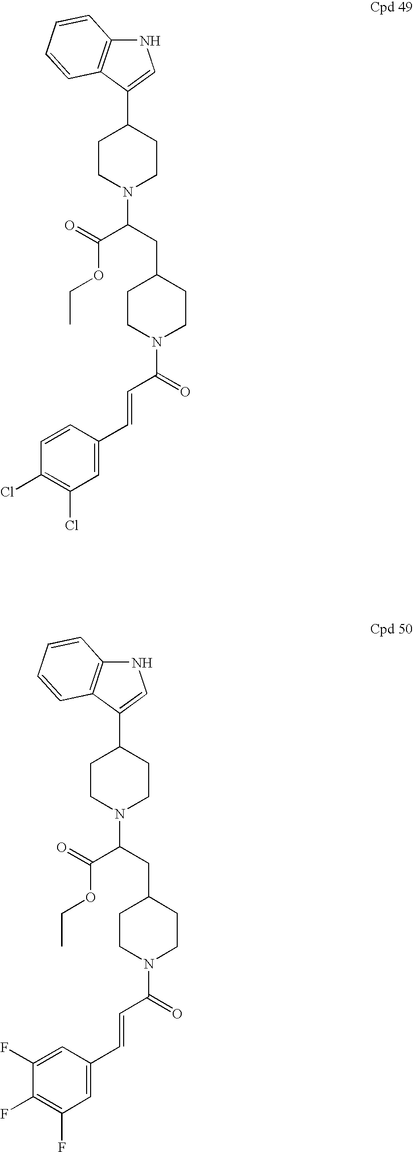 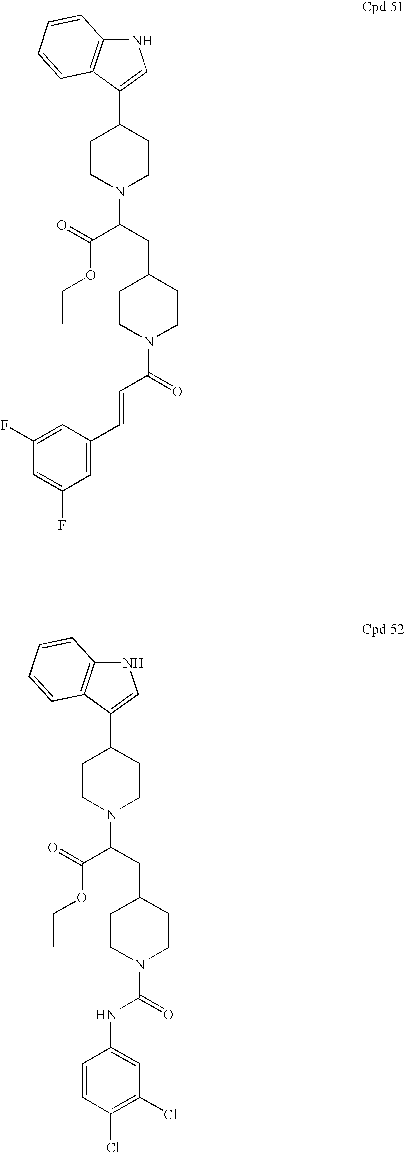 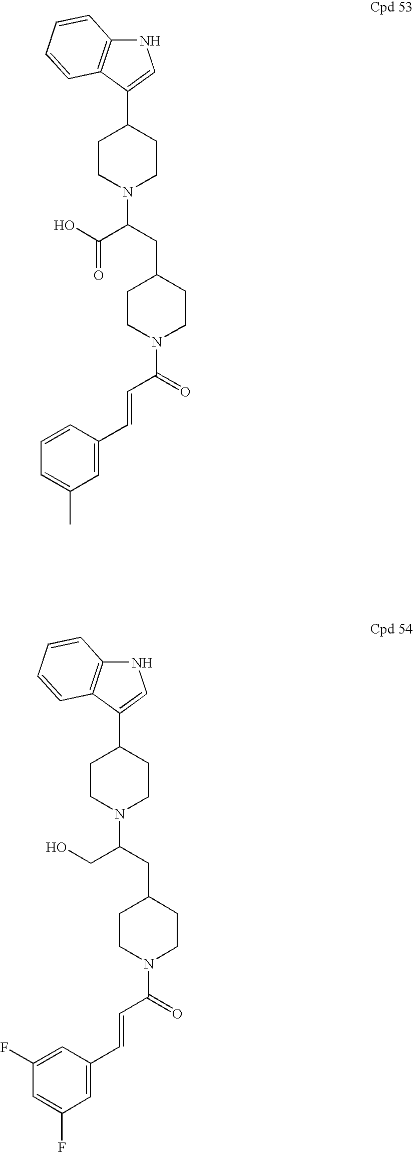 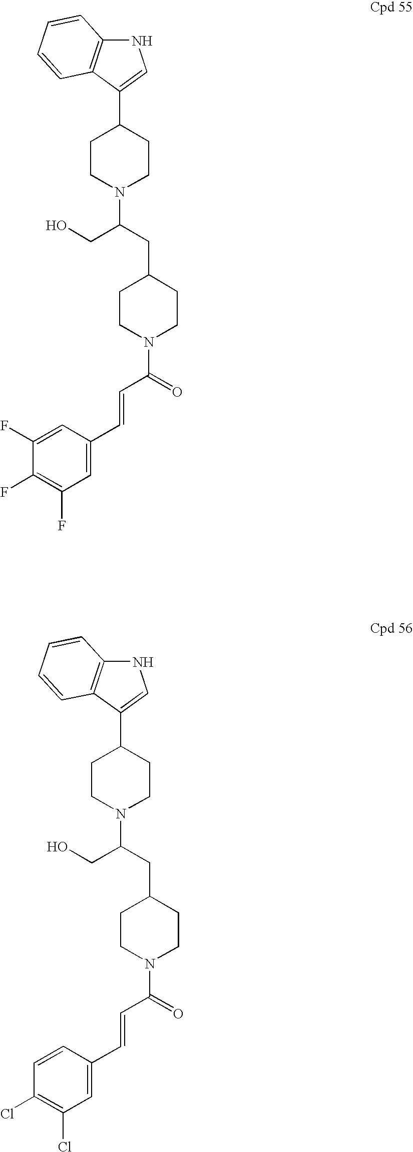 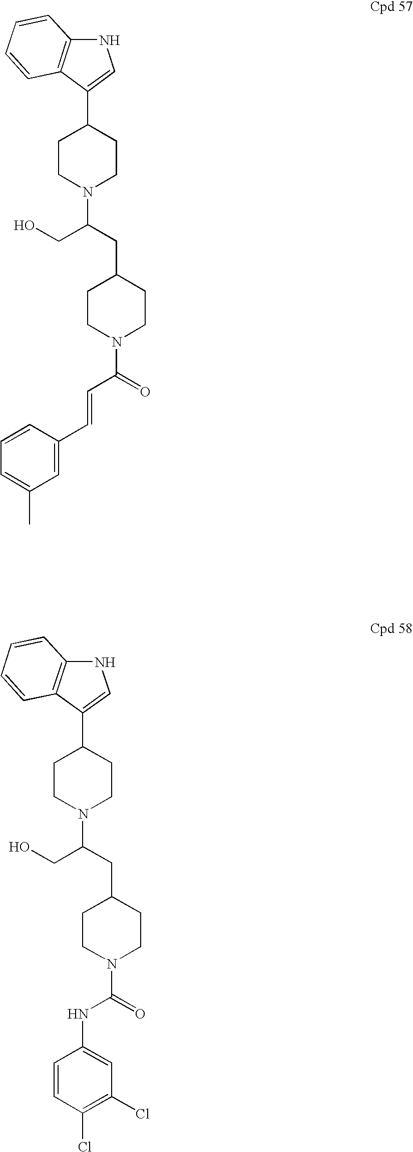 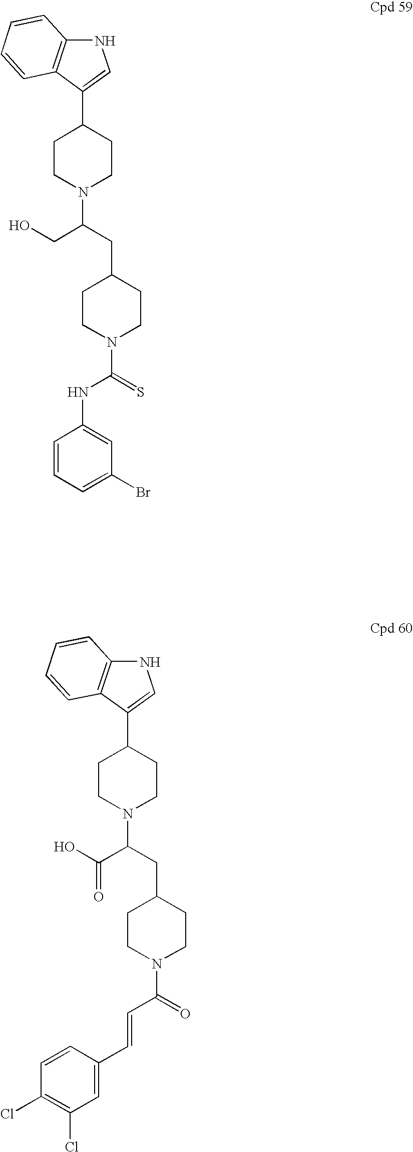 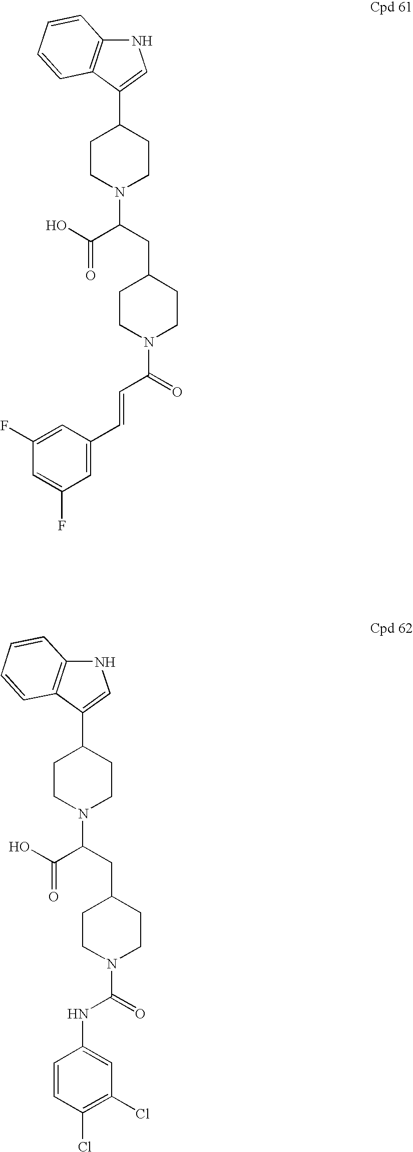 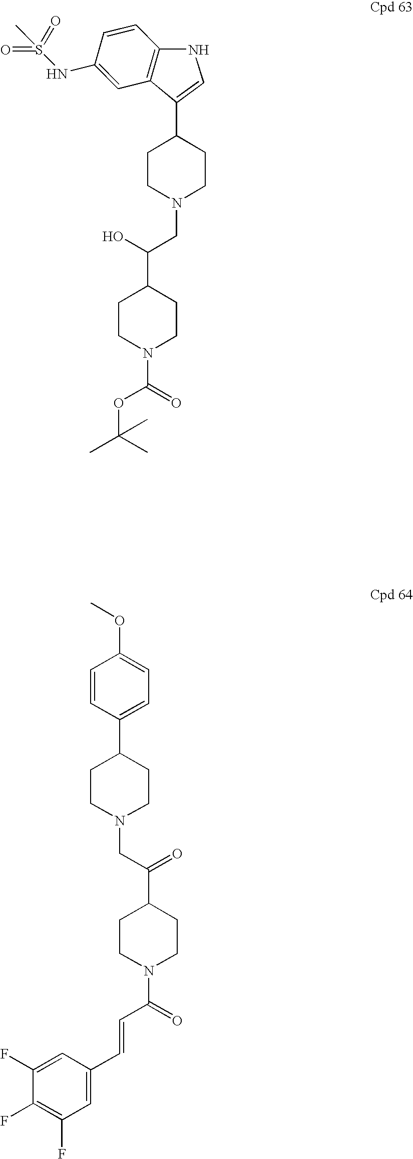 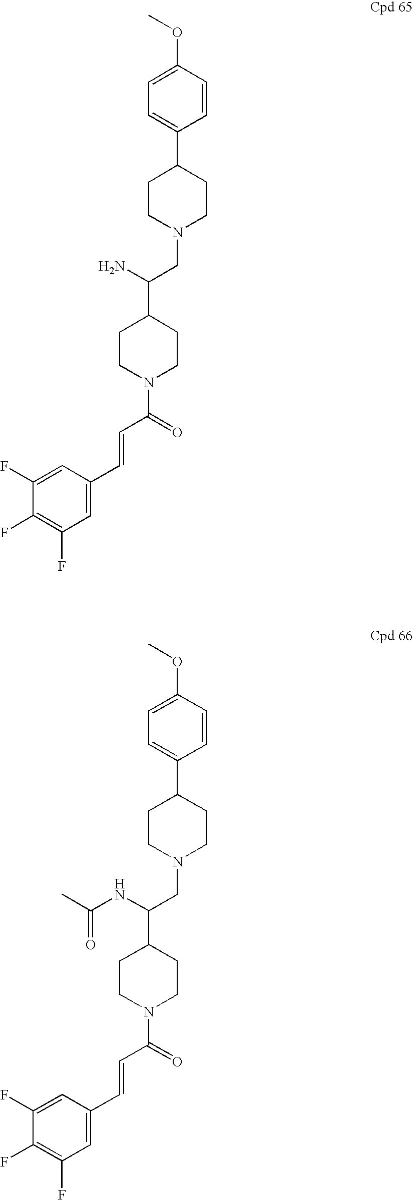 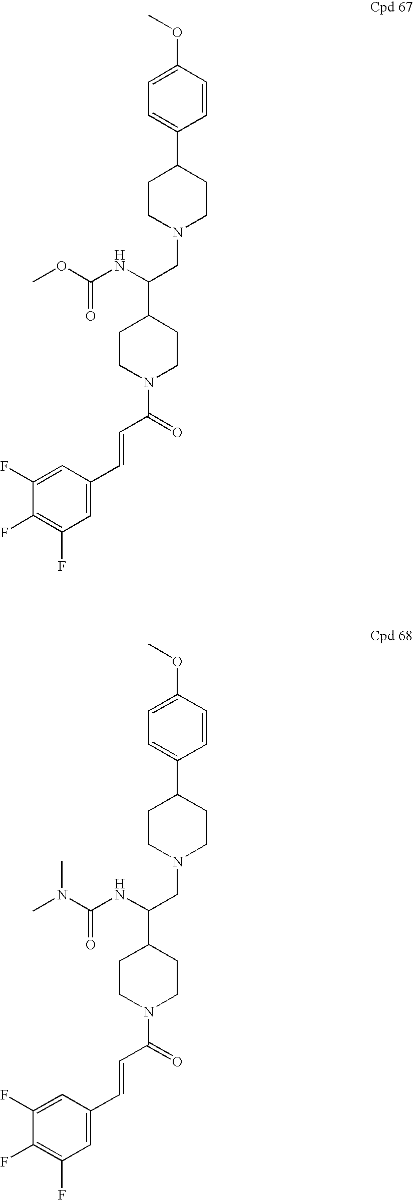 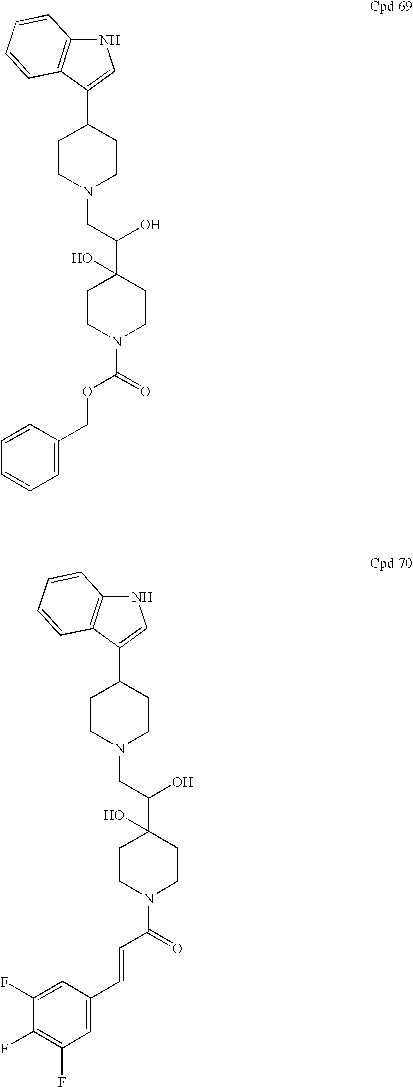 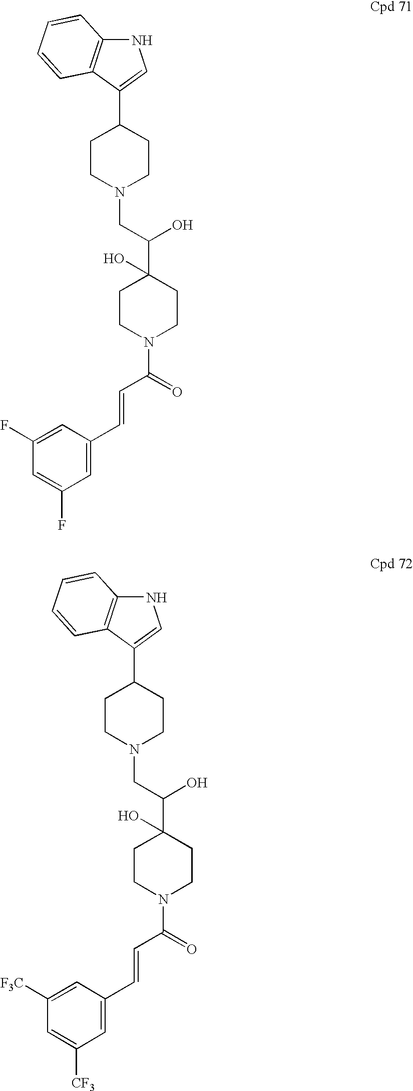 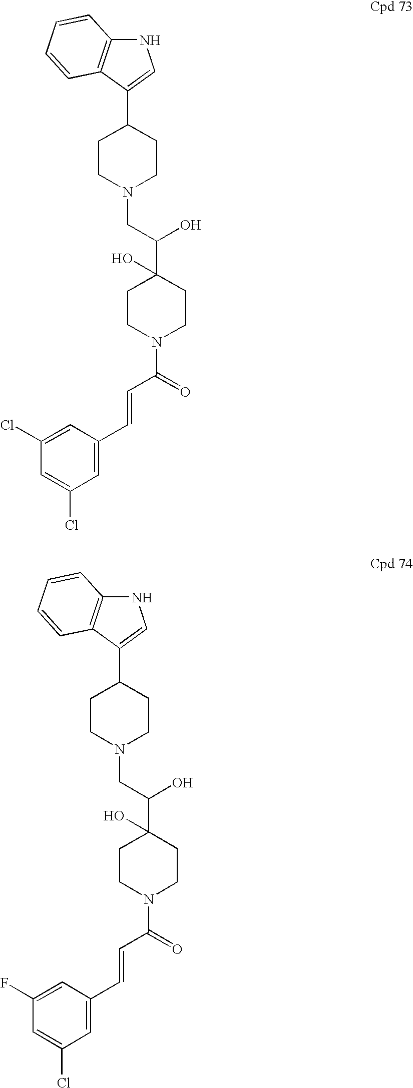 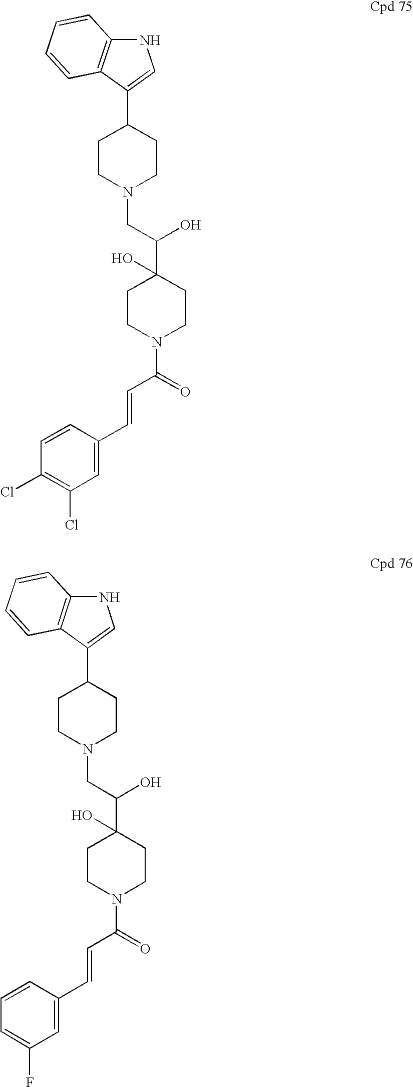 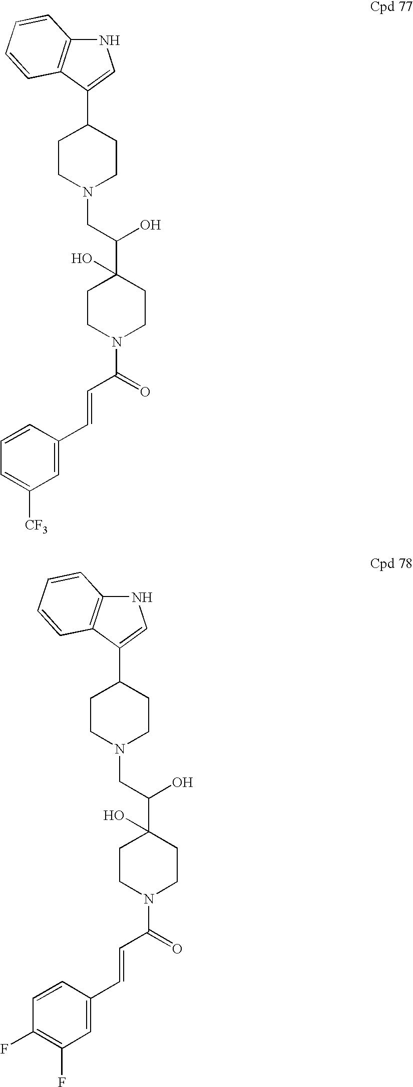 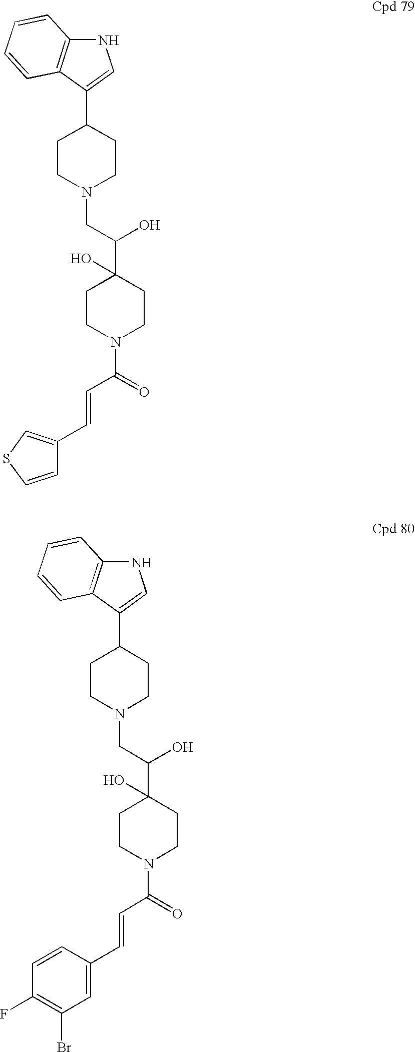 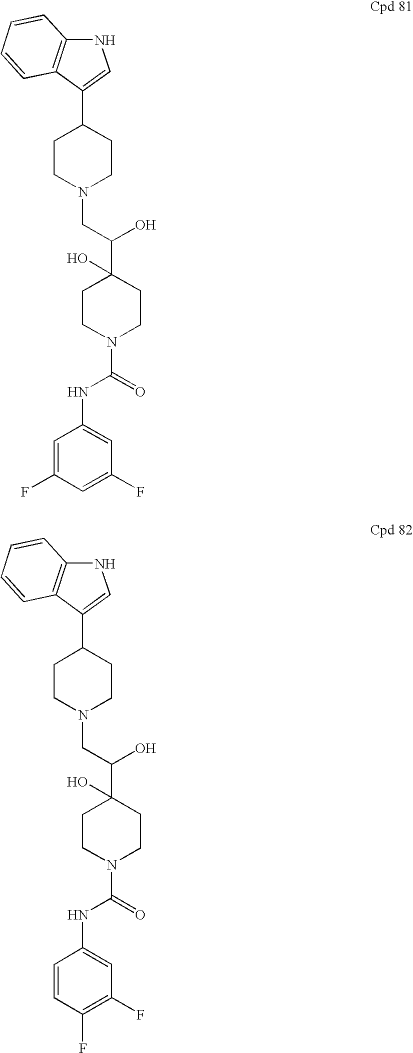 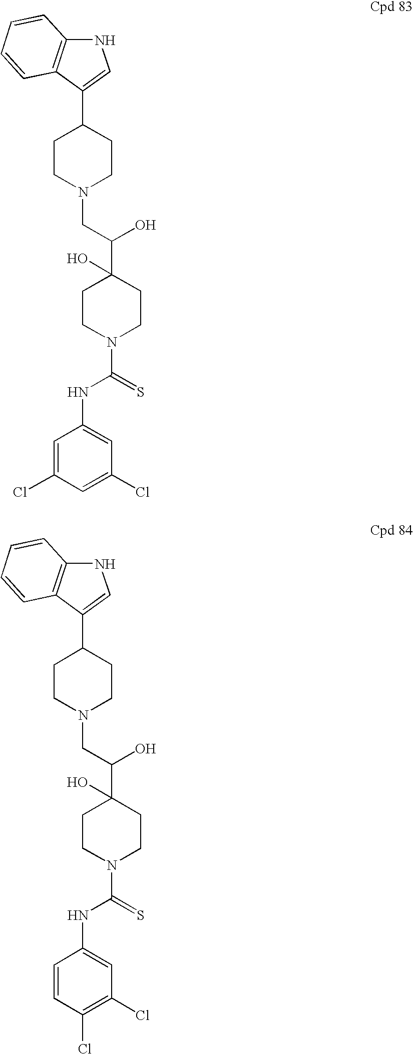 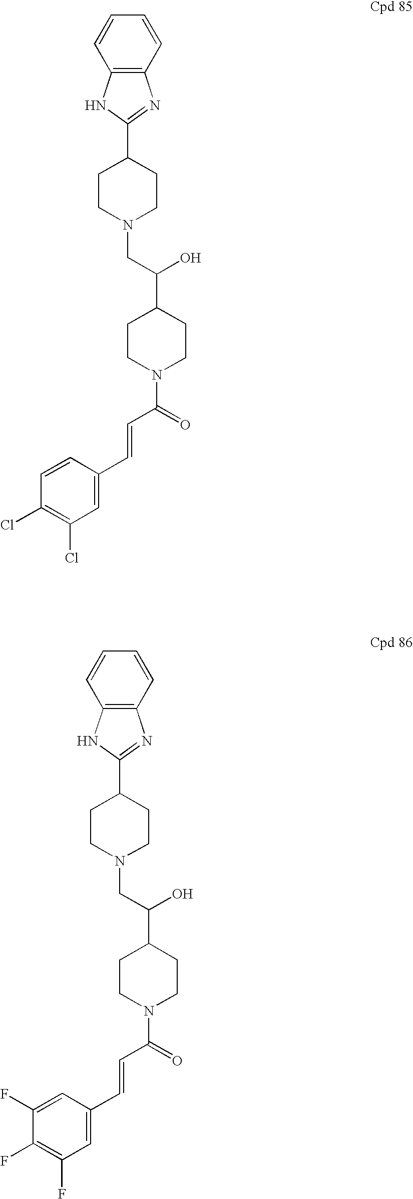 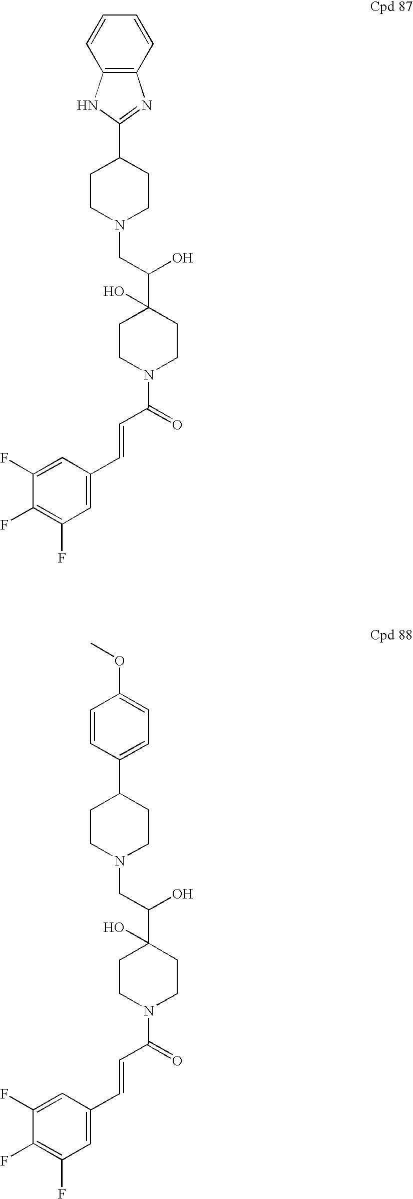 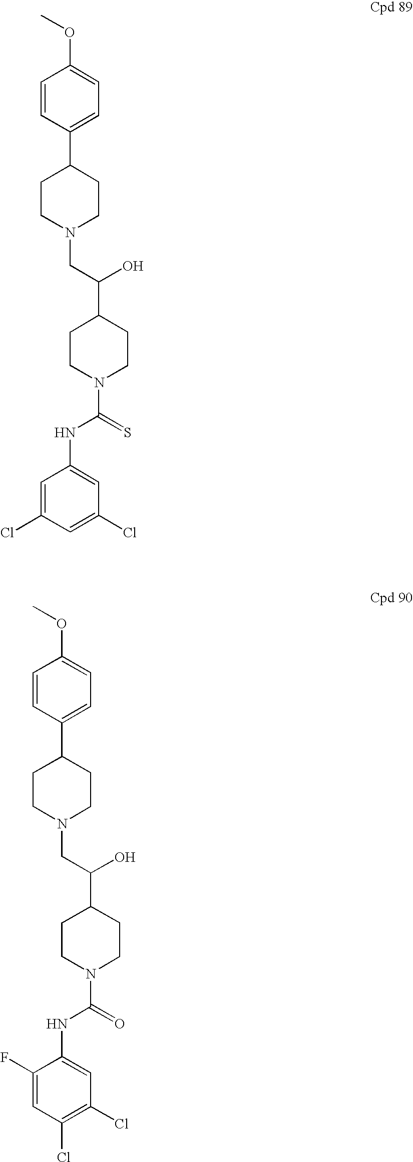 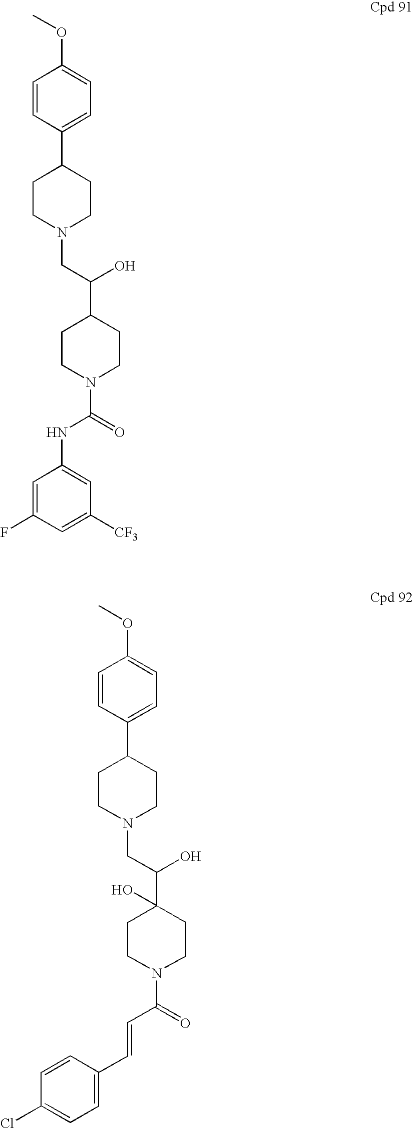 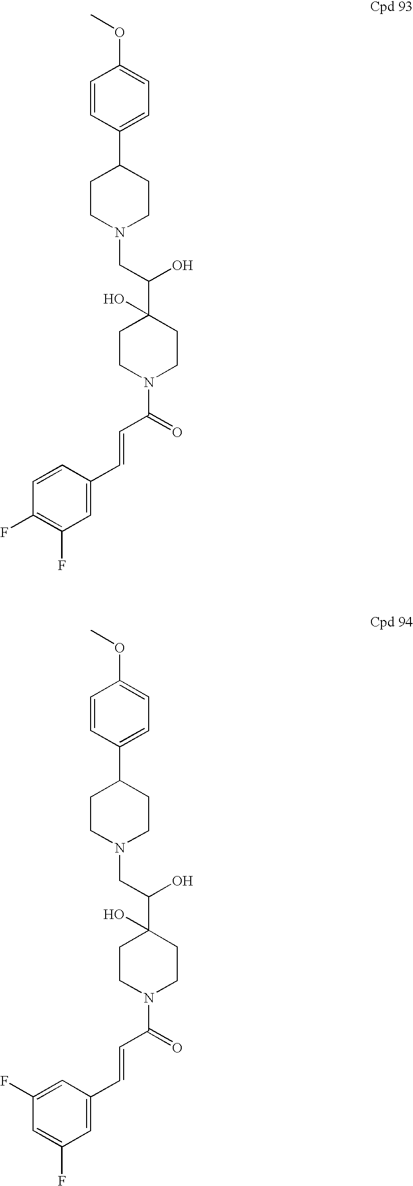 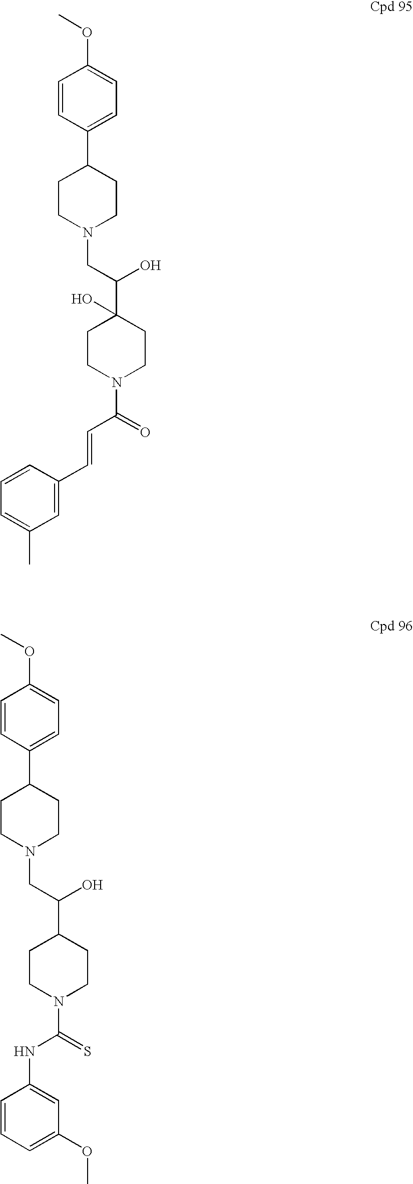 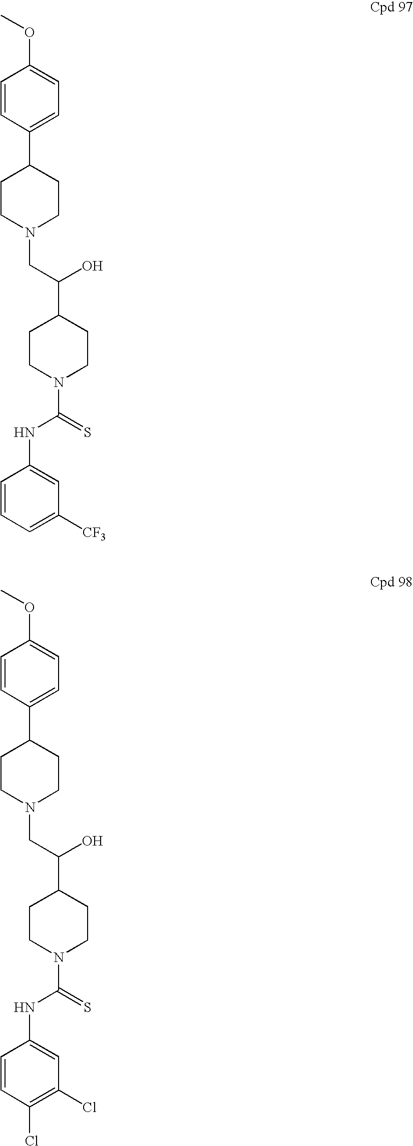 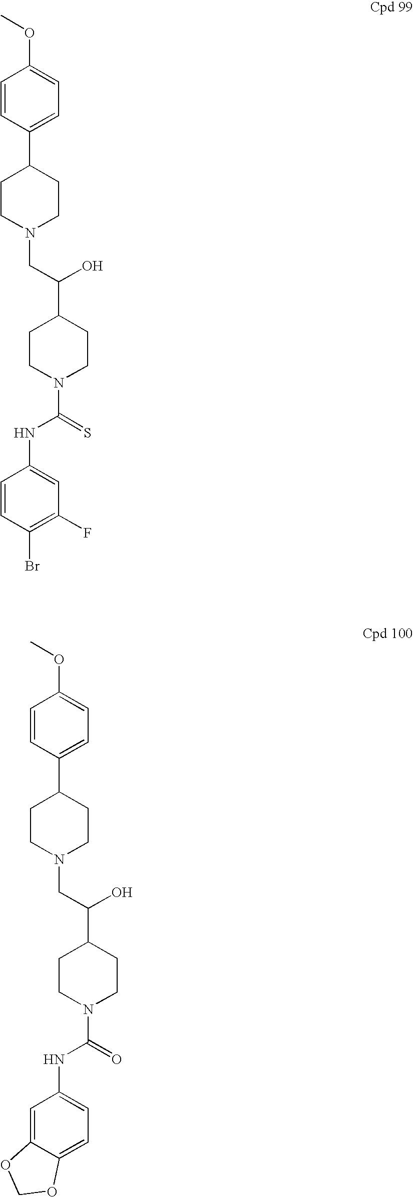 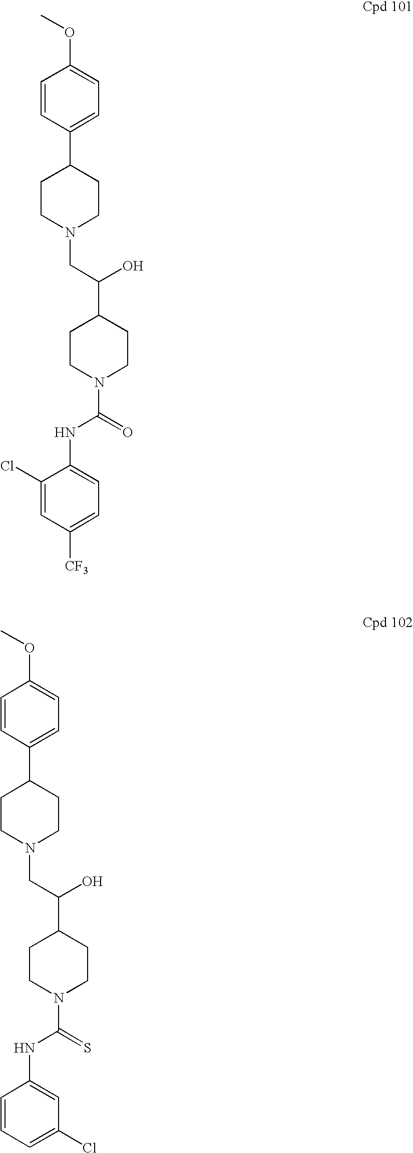 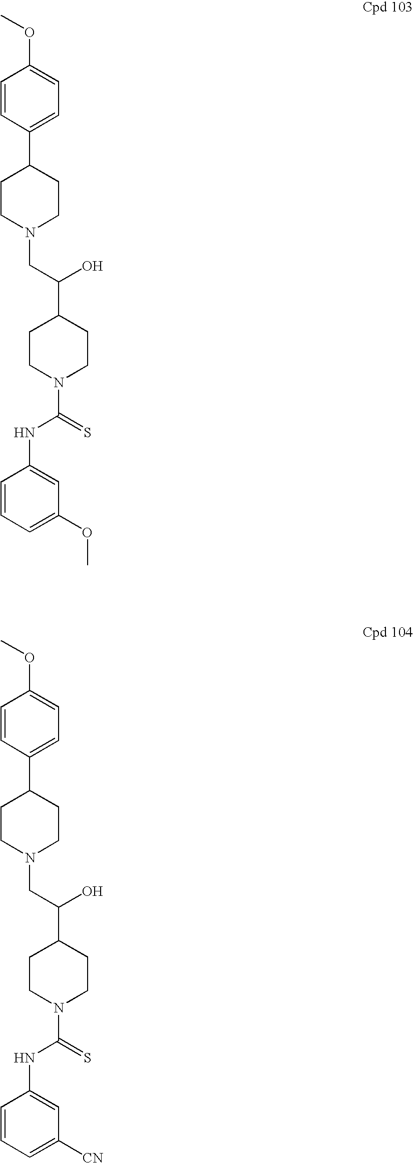 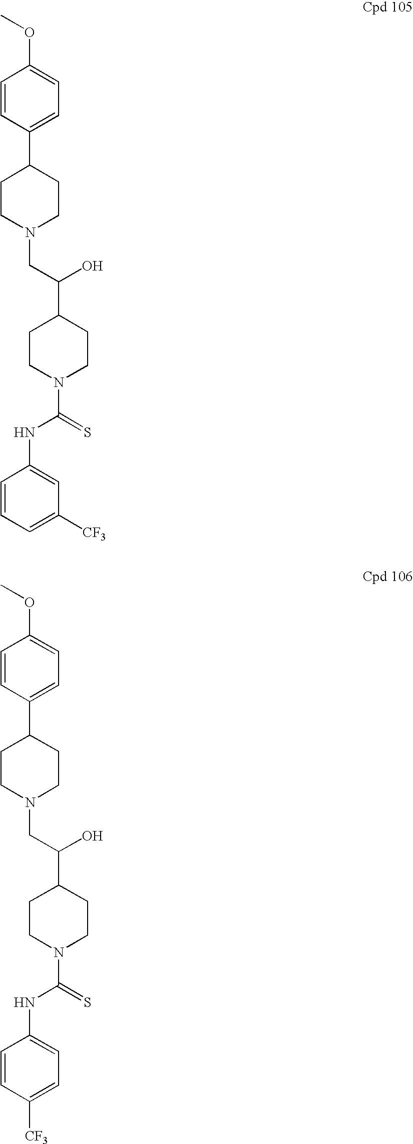 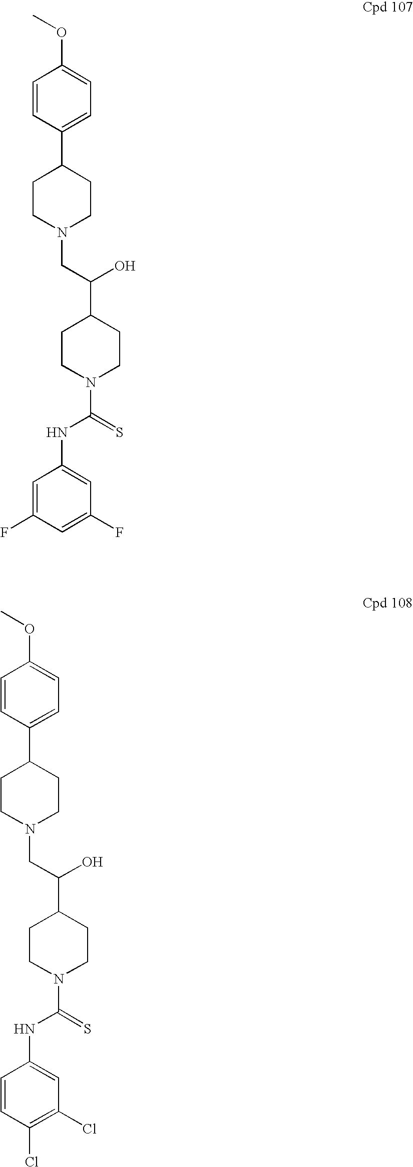 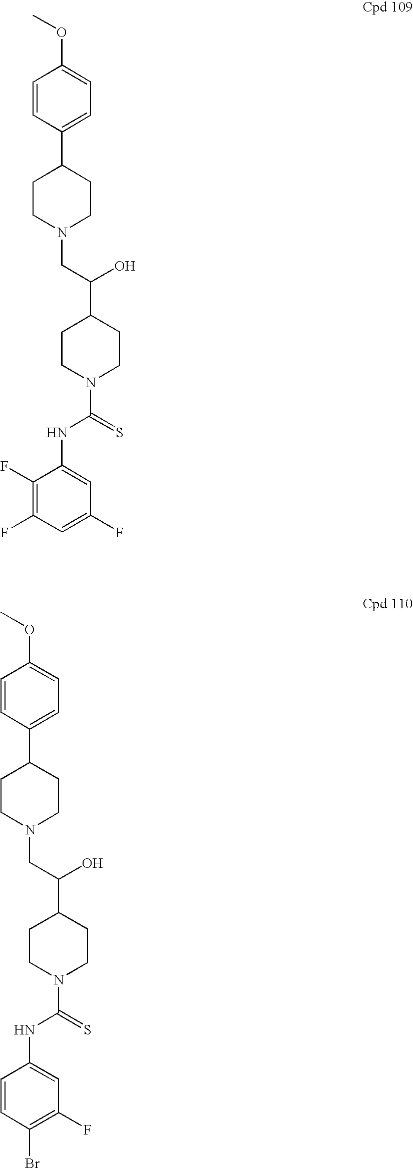 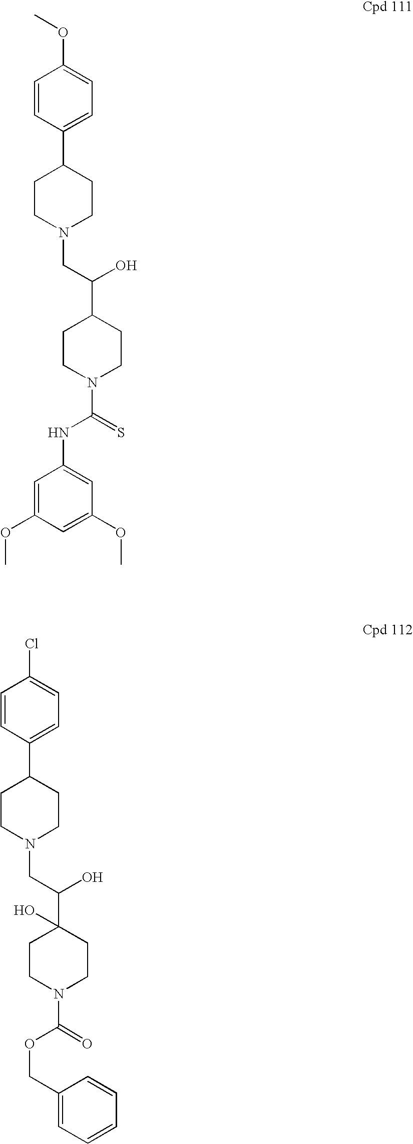 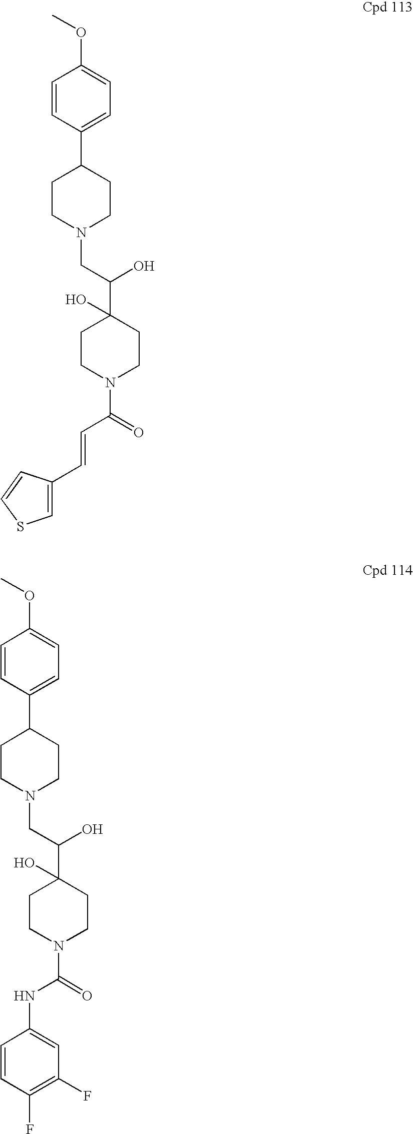 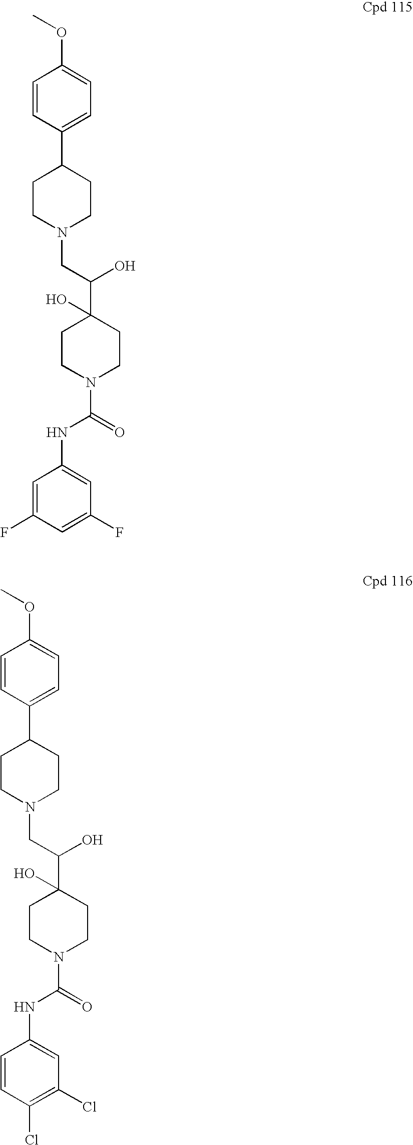 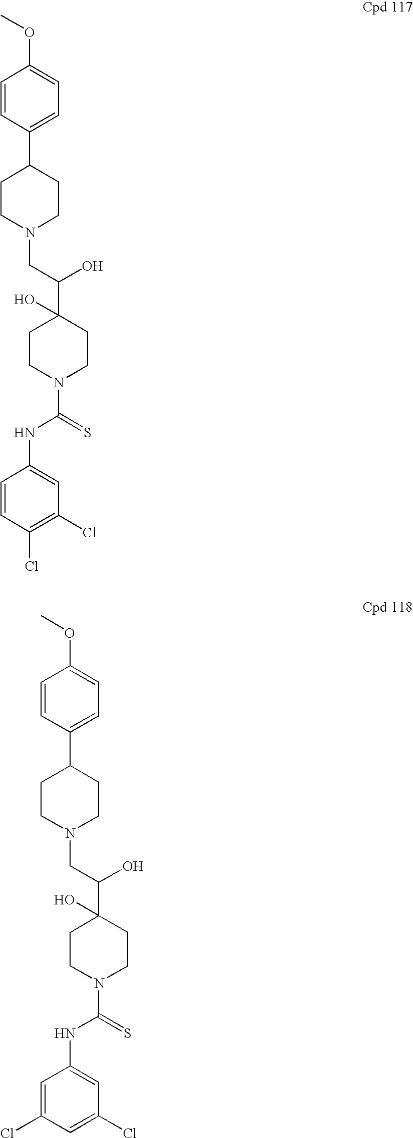 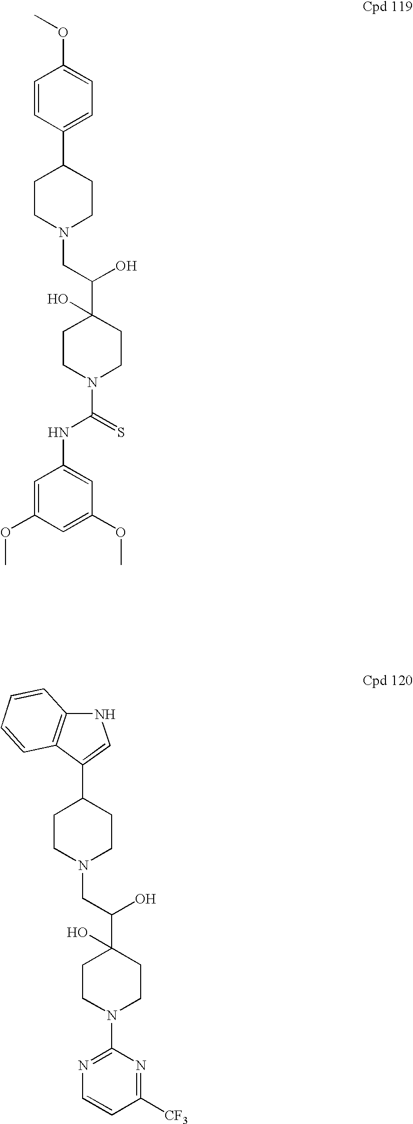 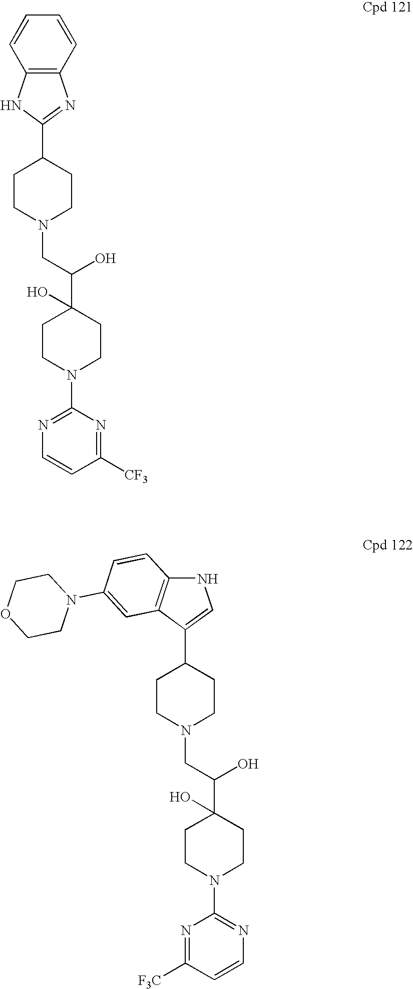 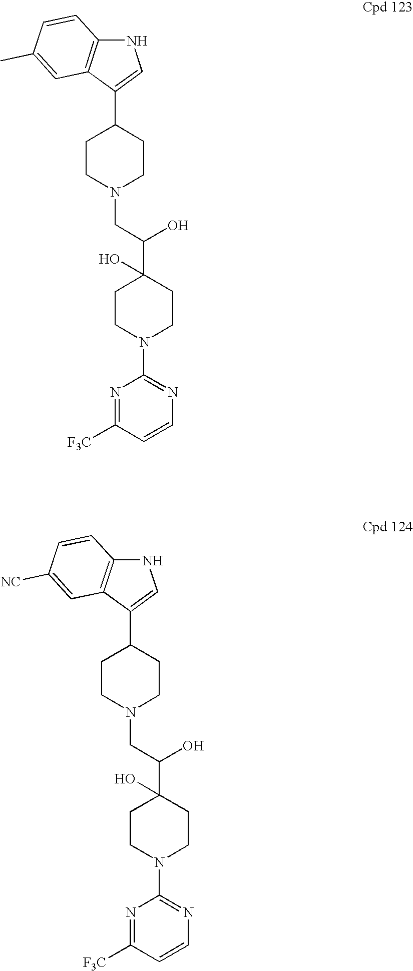 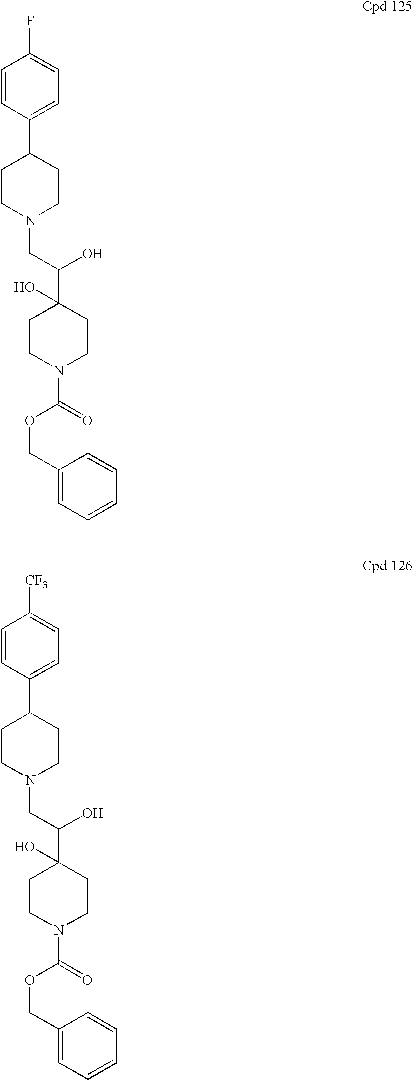 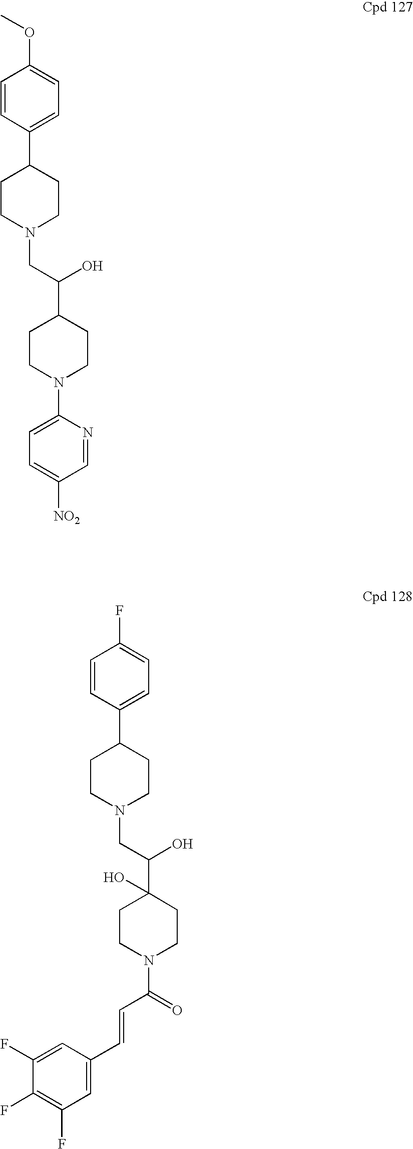 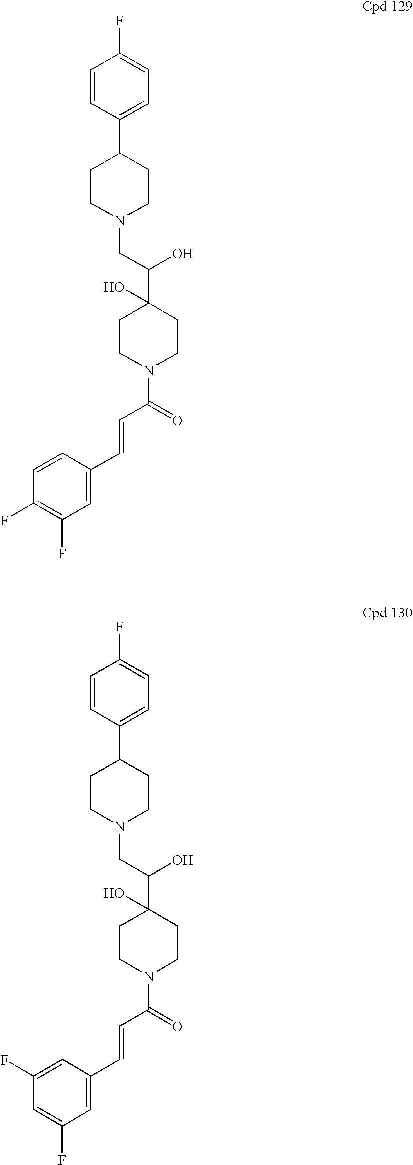 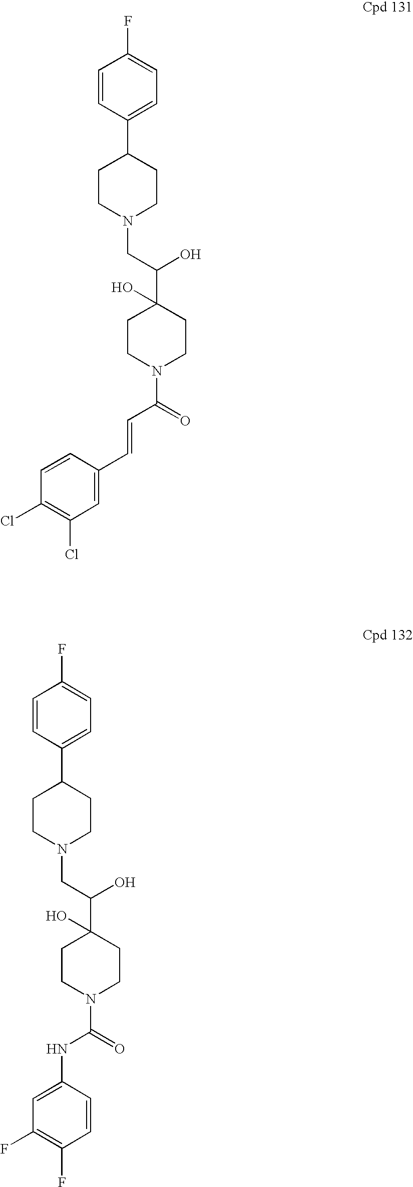 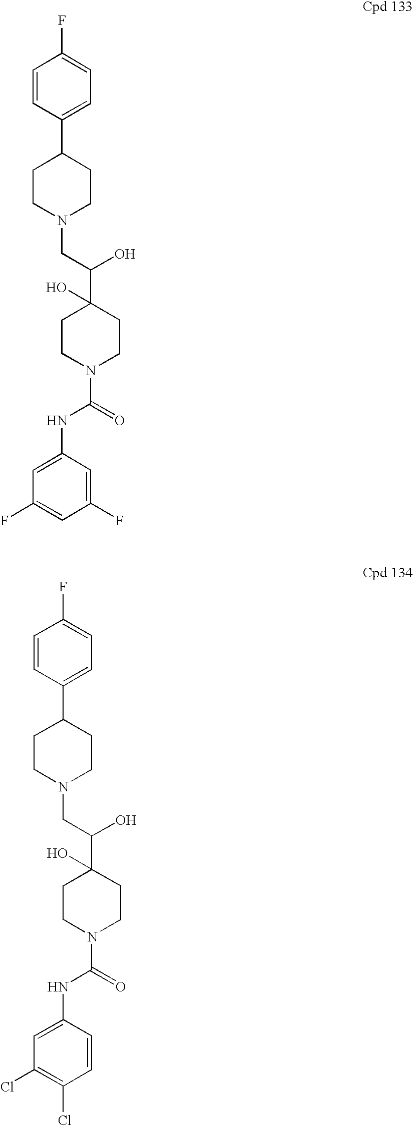 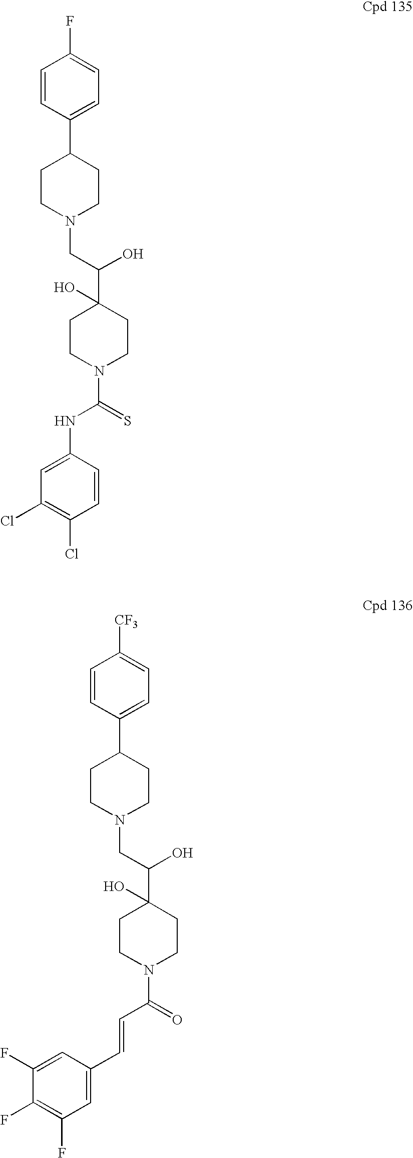 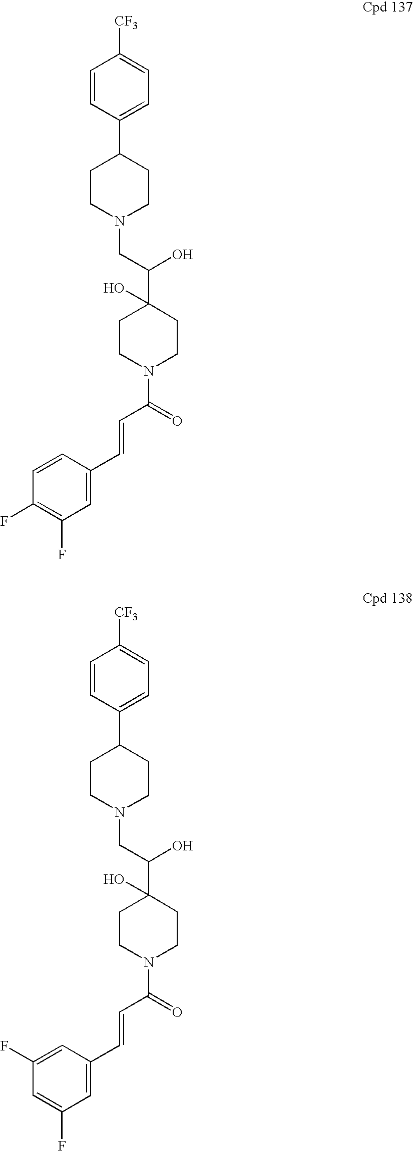 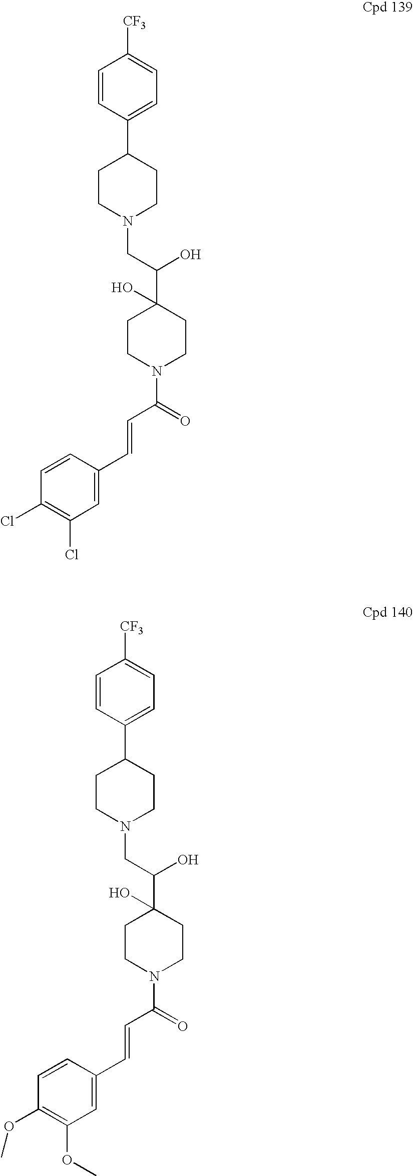 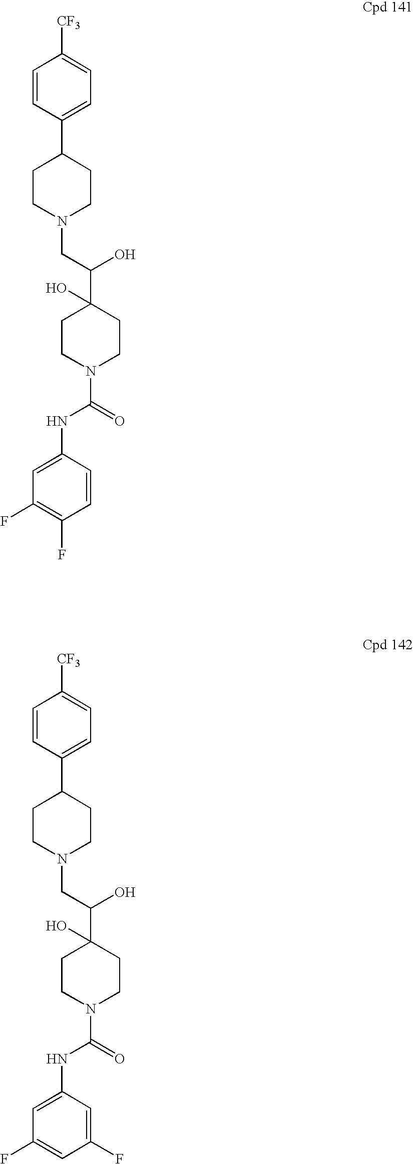 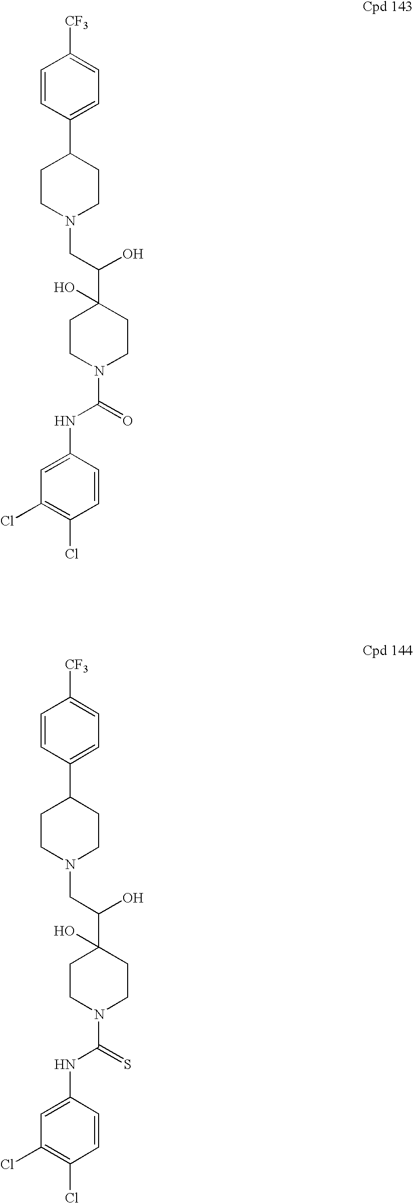 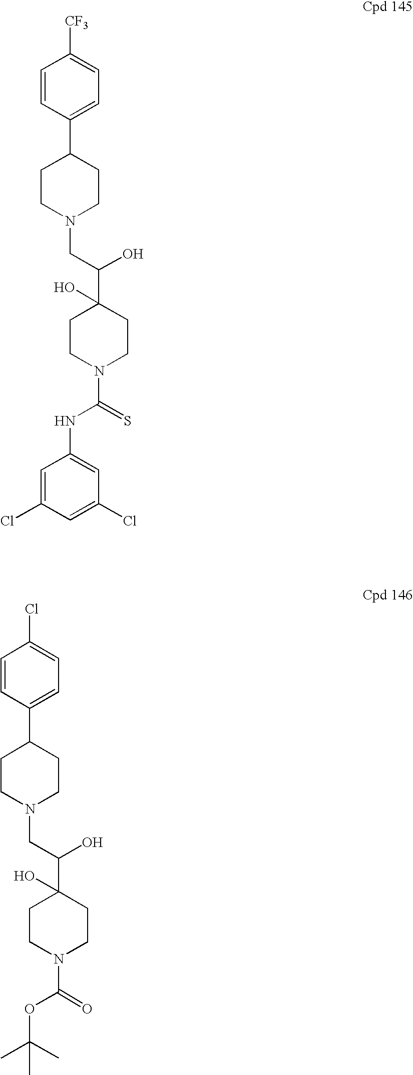 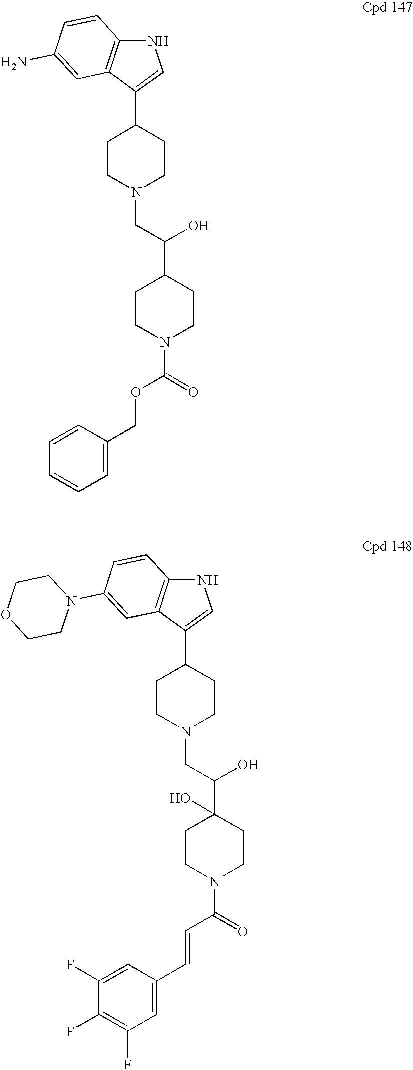 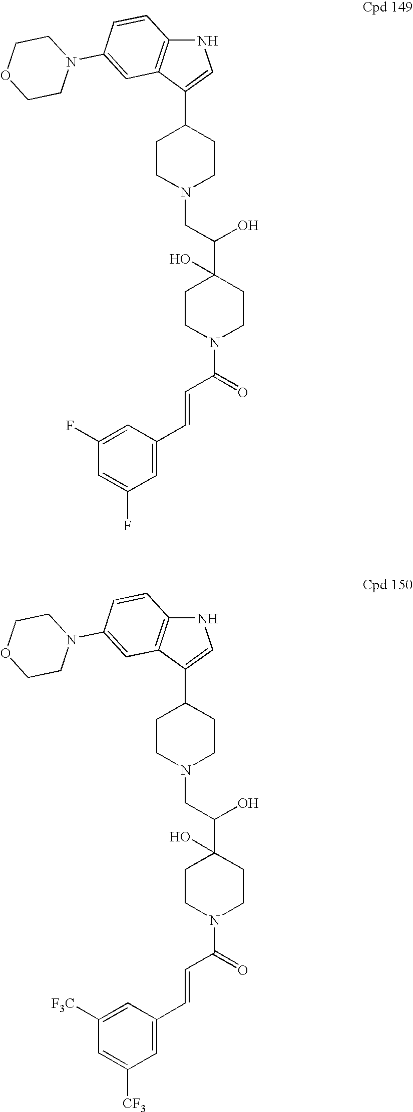 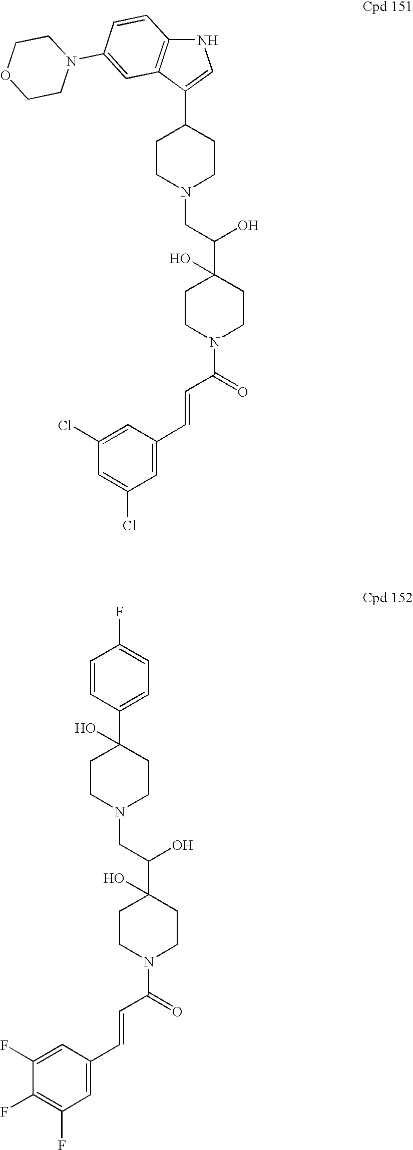 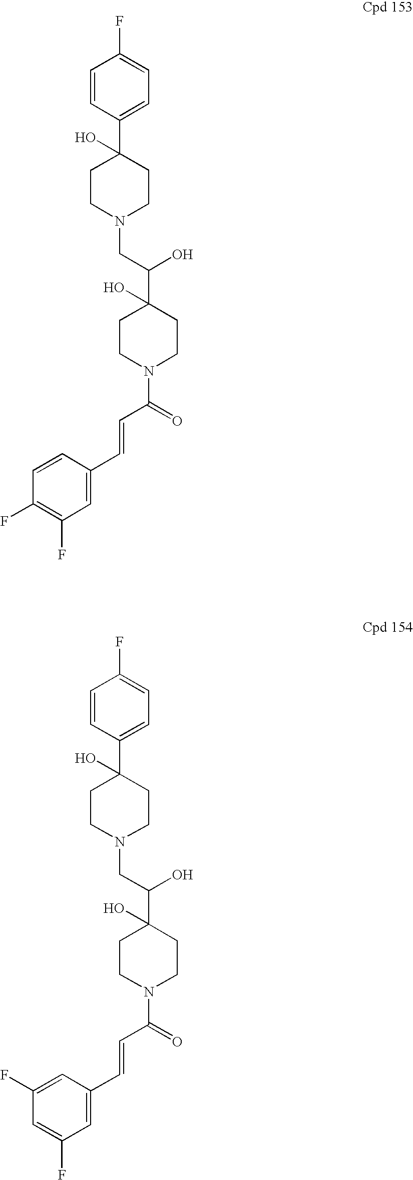 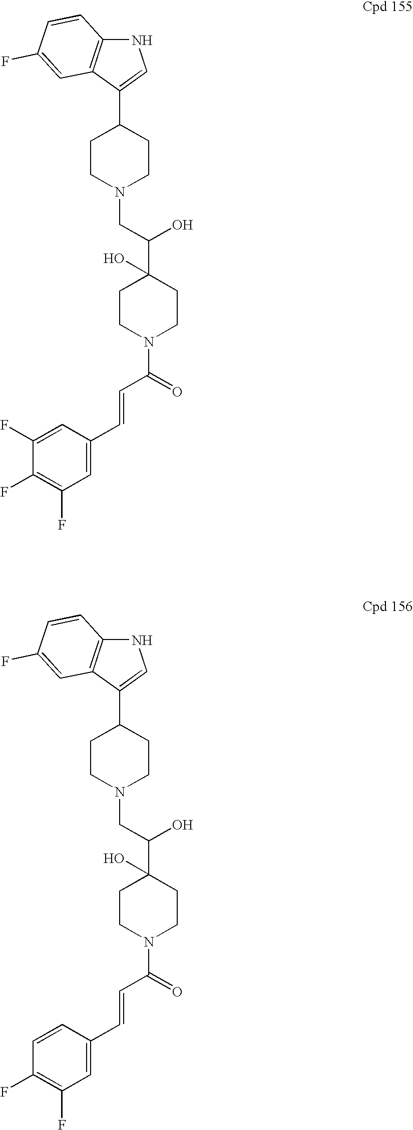 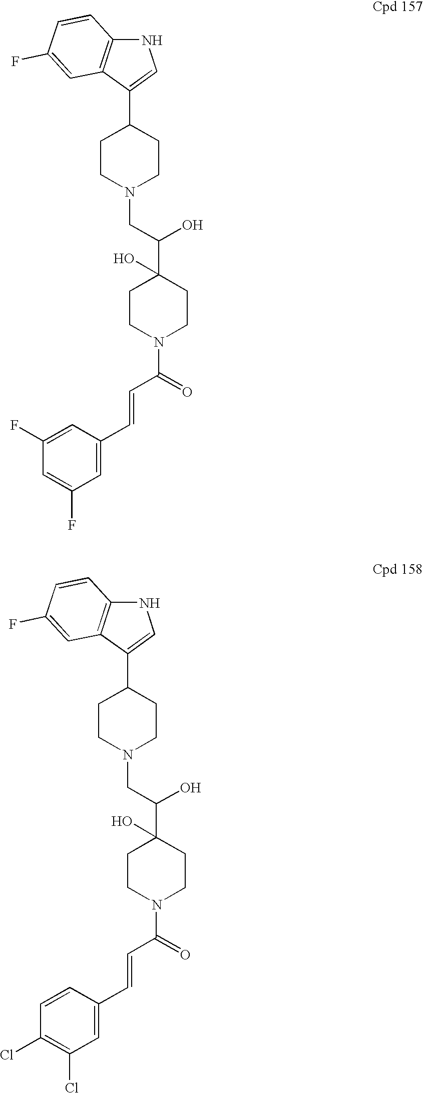 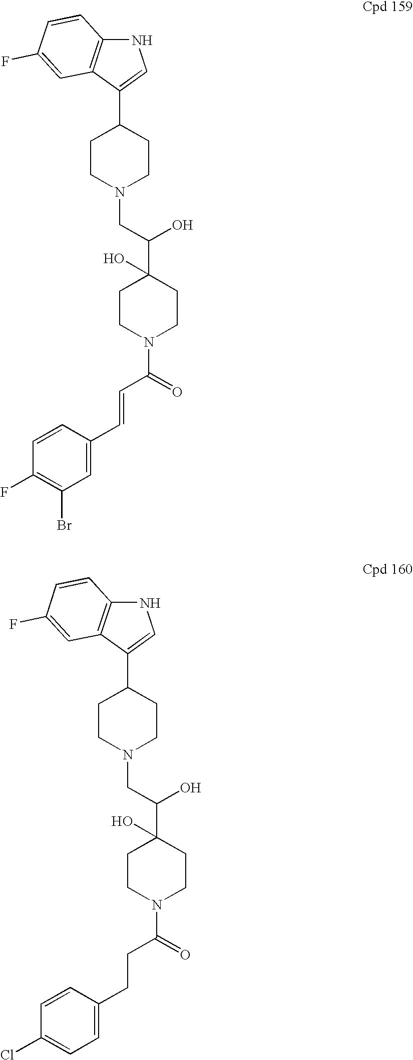 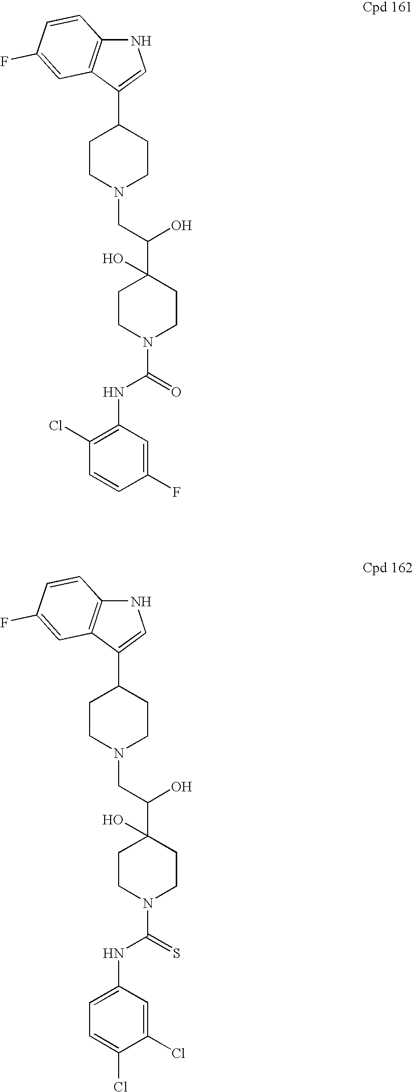 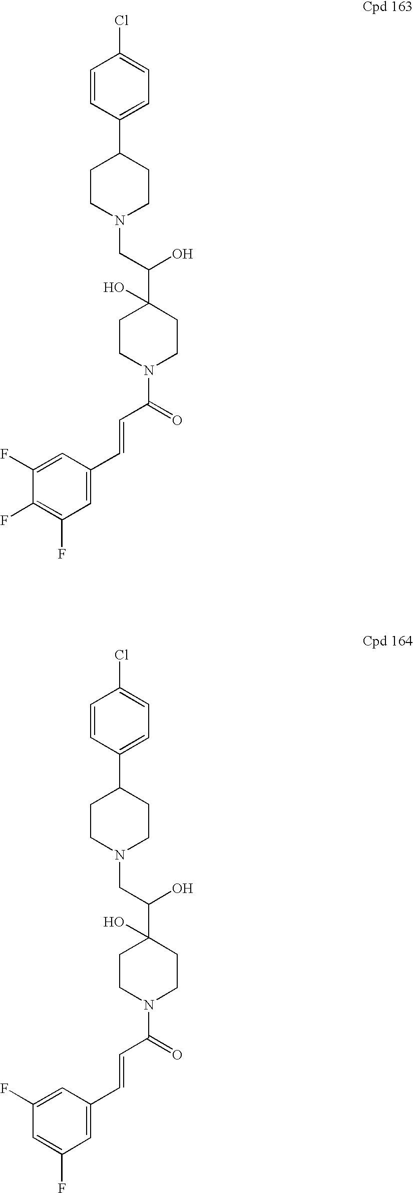 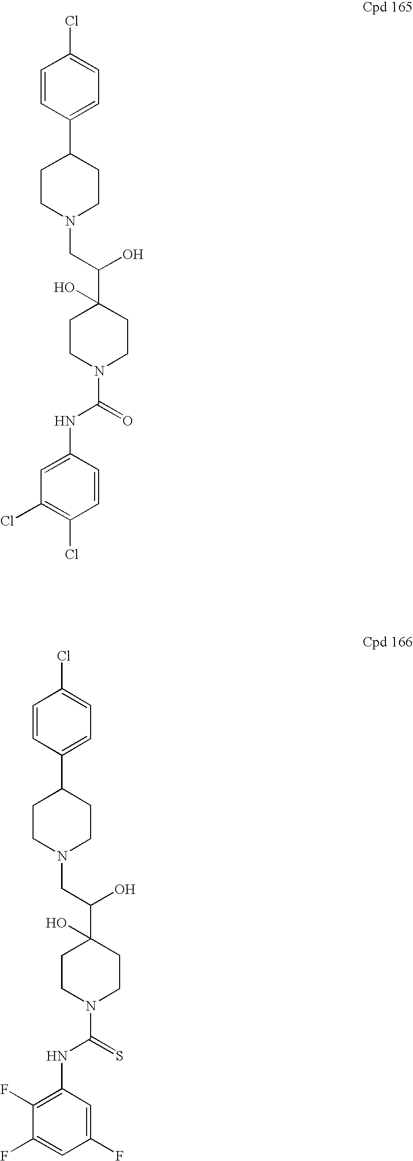 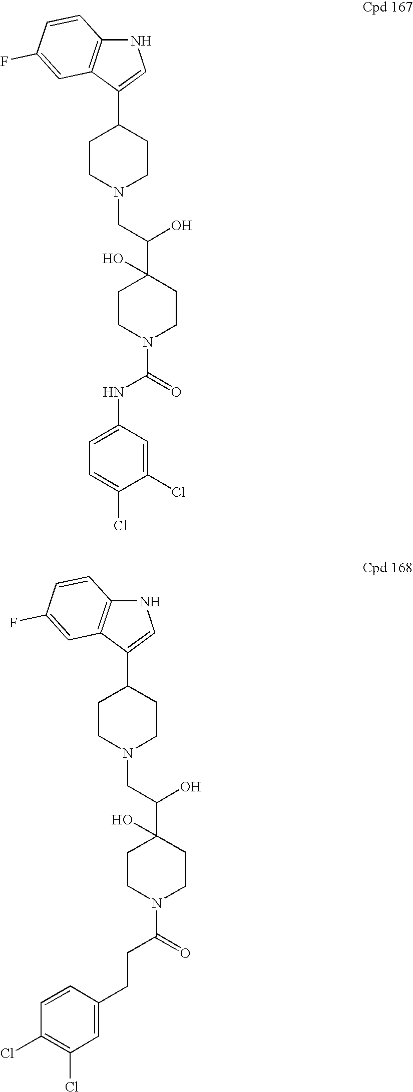 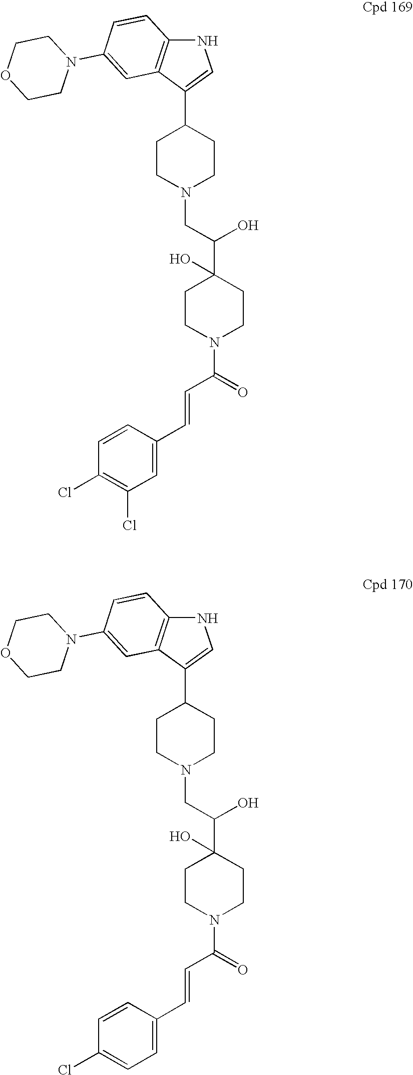 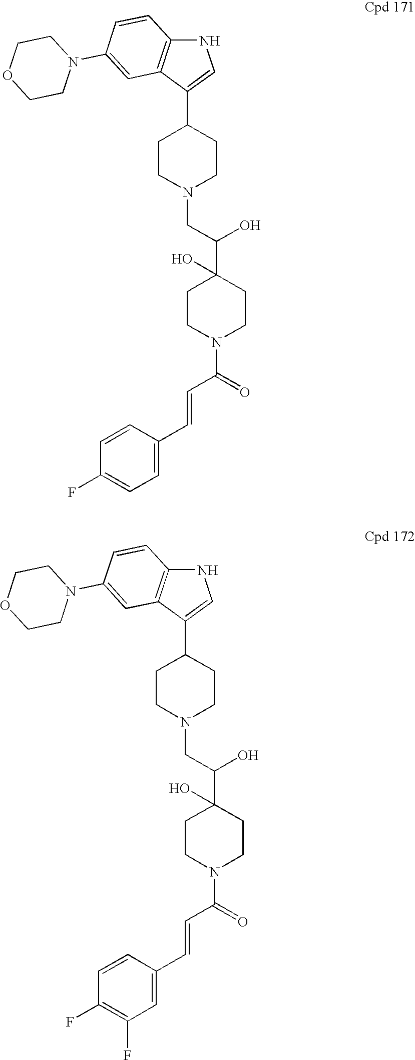 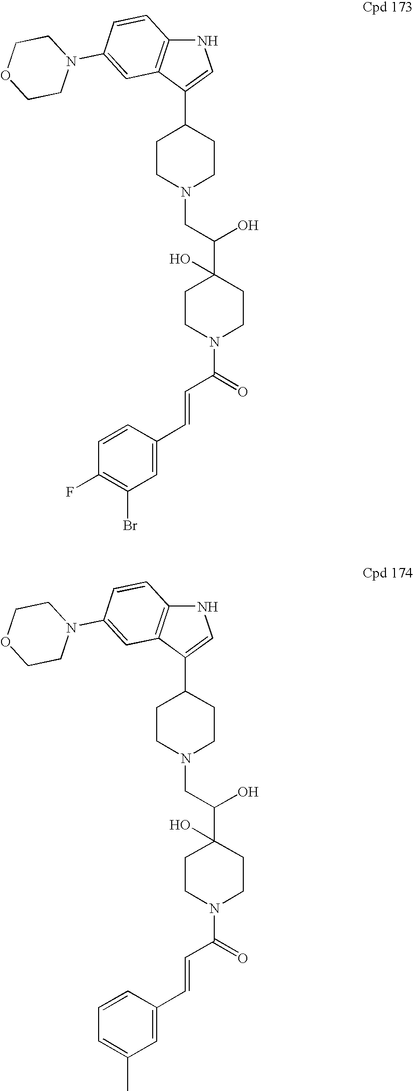 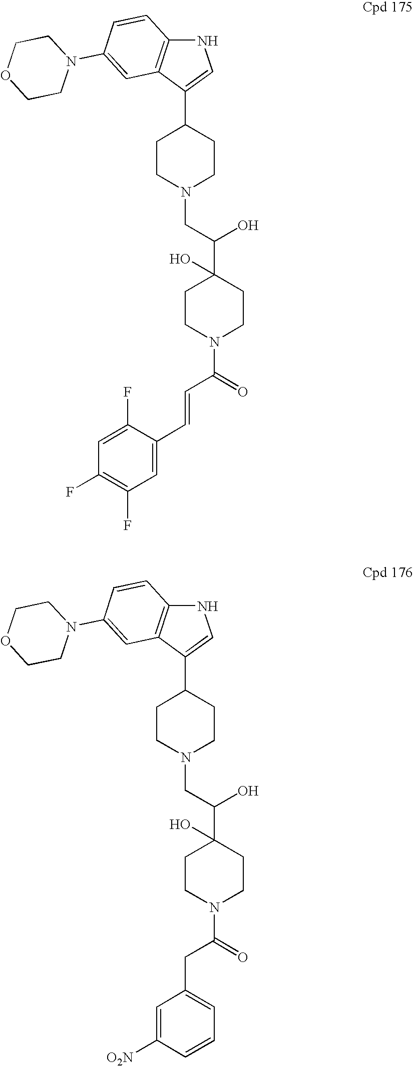 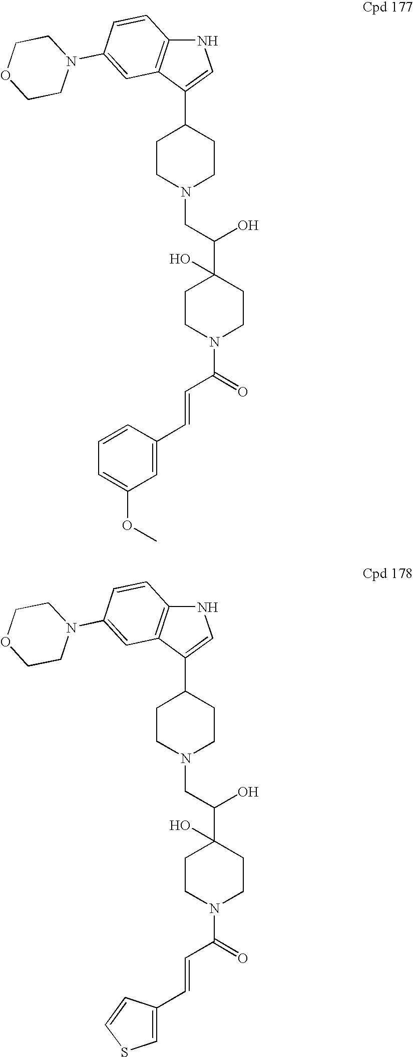 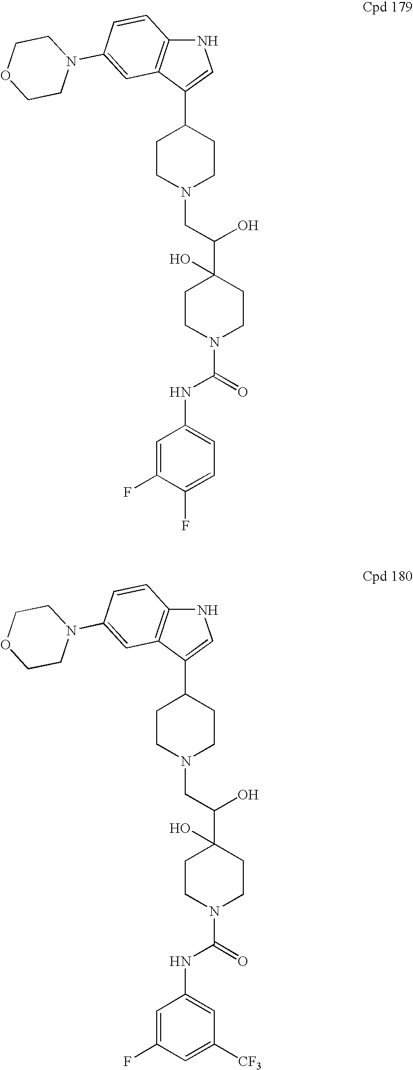 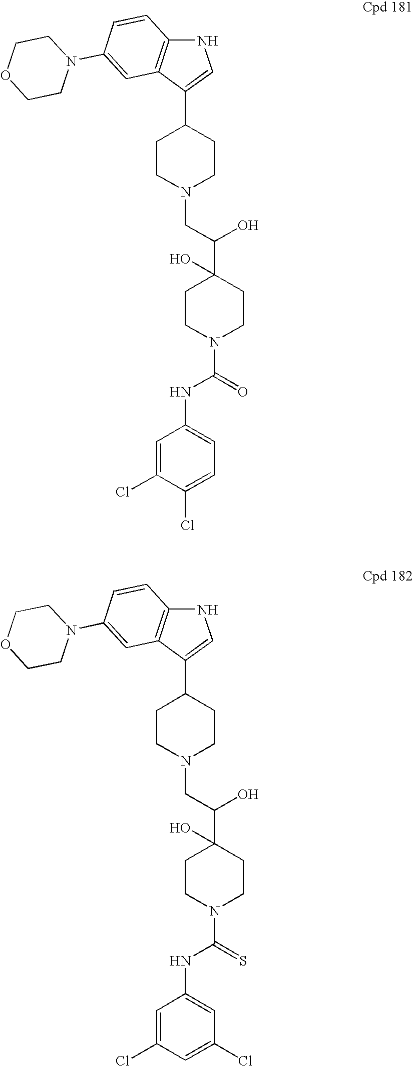 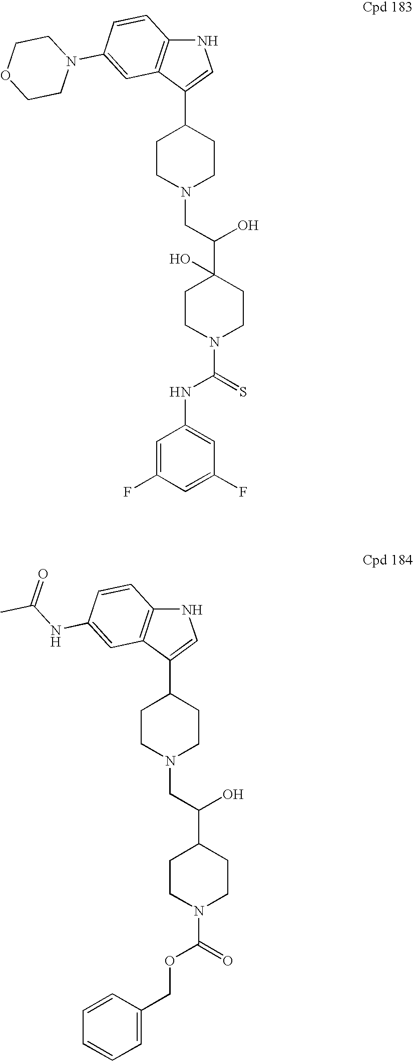 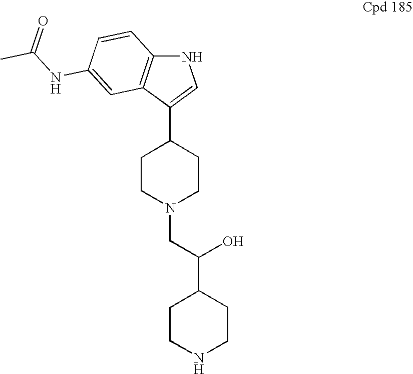
Chemical Definitions

Bond lines drawn into a ring system from a substitutent variable indicate that the substitutent may be attached to any of the substitutable ring atoms.

As used herein, the following terms are intended to have the following definitions. The definitions herein may specify that a chemical term has an indicated formula. The particular formula provided is not intended to limit the scope of the invention, but is provided as an illustration of the term.

The term “alkyl” means a saturated branched or straight-chain hydrocarbon radical or linking group substitutent having from 1-8 carbon atoms, wherein the radical is derived by the removal of one hydrogen atom from a carbon atom and the linking group is derived by the removal of one hydrogen atom from each of two carbon atoms in the chain. An alkyl radical or linking group includes, without limitation, methyl, methylene, ethyl, ethylene, propyl, propylene, isopropyl, isopropylene, n-butyl, n-butylene, t-butyl, t-butylene, pentyl, pentylene, hexyl, hexylene and the like. An alkyl radical may be attached to a core molecule and further substituted on any atom when allowed by available valences.

The term “alkenyl” means a partially unsaturated alkyl radical or linking group substitutent having at least one double bond, wherein the double bond is derived by the removal of one hydrogen atom from each of two adjacent carbon atoms in the chain. An alkenyl radical or linking group includes, without limitation, vinyl, propenyl, propenylene, isopropenyl, isopropenylene, n-butenyl (1-butenyl), n-butenylene, crotyl (2-butenyl) and the like. An alkenyl radical may be attached to a core molecule and further substituted on any atom when allowed by available valences.

The term “alkoxy” means an alkyl radical or linking group substitutent attached through an oxygen-linking atom, wherein a radical is of the formula —O-alkyl and a linking group is of the formula —O-alkyl-terminal group, and includes, without limitation, methoxy, ethoxy, propoxy, butoxy and the like. An alkoxy radical may be attached to a core molecule and further substituted on any atom when allowed by available valences.

The term “cycloalkyl” means a saturated or partially unsaturated hydrocarbon ring system radical or linking group such as a C₃₋₈cycloalkyl, C₃₋₁₀cycloalkyl, C₅₋₈cycloalkyl, C₅₋₁₂cycloalkyl or C₉₋₁₂cycloalkyl ring system radical and the like, and includes, without limitation, cyclopropyl, cyclobutyl, cyclopentyl, cyclohexyl, cyclohexenyl, cycloheptyl, cyclooctyl, indanyl, indenyl, 1,2,3,4-tetrahydro-naphthalenyl, 5,6,7,8-tetrahydro-naphthalenyl, 6,7,8,9-tetrahydro-5H-benzocycloheptenyl, 5,6,7,8,9,10-hexahydro-benzocyclooctenyl, fluorenyl, bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantanyl, octahydro-4,7-methano-1H-indenyl, octahydro-2,5-methano-pentalenyl (also referred to as hexahydro-2,5-methano-pentalenyl) and the like. A cycloalkyl radical may be attached to a core molecule and further substituted on any ring atom when allowed by available valences.

The term “heterocyclyl” means a saturated, partially unsaturated (such as those named with the prefix dihydro, trihydro, tetrahydro, hexahydro and the like) or unsaturated ring system radical, wherein at least one ring carbon atom has been replaced with one or more heteroatoms independently selected from N, O, S, S(O) or SO₂. A heterocyclyl ring system further includes a ring system having 1, 2, 3, or 4 carbon atom members replaced by a nitrogen atom. Alternatively, a ring may have 0, 1, 2, or 3 nitrogen atom members and 1 oxygen or sulfur atom member. Alternatively, up to two adjacent ring members may be heteroatoms, wherein one heteroatom is nitrogen and the other heteroatom is selected from N, O, S, S(O) or SO₂. A heterocyclyl radical is derived by the removal of one hydrogen atom from a single carbon or nitrogen ring atom. A heterocyclyl linking group is derived by the removal of one hydrogen atom from two of either a carbon or nitrogen ring atom. A heterocyclyl radical may be attached to a core molecule and further substituted on any ring atom when allowed by available valences.

The term heterocyclyl includes, without limitation, furanyl, thienyl, 2H-pyrrolyl, 2-pyrrolinyl, 3-pyrrolinyl, pyrrolidinyl, pyrrolyl, 1,3-dioxolanyl, oxazolyl, thiazolyl, imidazolyl, 2-imidazolinyl (also referred to as 4,5-dihydro-1H-imidazolyl), imidazolidinyl, 2-pyrazolinyl, pyrazolidinyl, pyrazolyl, isoxazolyl, isothiazolyl, oxadiazolyl, triazolyl, thiadiazolyl, tetrazolyl, tetrazolinyl, tetrazolidinyl, 2H-pyranyl, 4H-pyranyl, thiopyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azetidinyl, azepanyl, indolizinyl, indolyl, 4-aza-indolyl (also referred to as 1H-pyrrolo[3,2-b]pyridinyl), 6-aza-indolyl (also referred to as 1H-pyrrolo[2,3-c]pyridinyl), 7-aza-indolyl (also referred to as 1H-pyrrolo[2,3-b]pyridinyl), isoindolyl, 3H-indolyl, indolinyl (also referred to as 2,3-dihydro-indolyl), benzo[b]furanyl, furo[2,3-b]pyridinyl, benzo[b]thienyl, indazolyl (also referred to as 1H-indazolyl), benzoimidazolyl, benzothiazolyl, benzoxazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl, quinuclidinyl, 2H-chromenyl, 3H-benzo[f]chromenyl, tetrahydro-furanyl, tetrahydro-thienyl, tetrahydro-pyranyl, tetrahydro-thiopyranyl, tetrahydro-pyridazinyl, hexahydro-1,4-diazepinyl, hexahydro-1,4-oxazepanyl, 2,3-dihydro-benzo[b]oxepinyl, 1,3-benzodioxolyl (also known as 1,3-methylenedioxyphenyl or benzo[1,3]dioxolyl), 2,3-dihydro-1,4-benzodioxinyl (also known as 1,4-ethylenedioxyphenyl or benzo[1,4]dioxinyl), benzo-dihydro-furanyl (also known as 2,3-dihydro-benzofuranyl), benzo-tetrahydro-pyranyl, benzo-dihydro-thienyl, 5,6,7,8-tetrahydro-4H-cyclohepta[b]thienyl, 5,6,7-trihydro-4H-cyclohexa[b]thienyl, 5,6-dihydro-4H-cyclopenta[b]thienyl, 2-aza-bicyclo[2.2.1]heptyl, 1-aza-bicyclo[2.2.2]octyl, 8-aza-bicyclo[3.2.1]octyl, 7-oxa-bicyclo[2.2.1]heptyl, pyrrolidinium, piperidinium, piperazinium, morpholinium and the like.

The term “aryl” means an unsaturated aromatic hydrocarbon ring system radical, and includes, without limitation, phenyl, naphthalenyl, azulenyl, anthracenyl and the like. An aryl radical may be attached to a core molecule and further substituted on any ring atom when allowed by available valences.

The term “acrylyl” means a radical of the formula —C(O)CH═CH-terminal group.

The term “alkoxycarbonyl” means a radical of the formula: —C(O)—O-alkyl or —C(O)—O-alkyl-(terminal group).

The term “alkoxycarbonylalkoxy” means a radical of the formula: —O-alkyl-C(O)—O-alkyl.

The term “alkoxycarbonylalkyl” means a radical of the formula: -alkyl-C(O)—O-alkyl.

The term “alkoxycarbonylalkylamino” means a radical of the formula: —NH-alkyl-C(O)—O-alkyl or —N[alkyl-C(O)—O-alkyl]₂.

The term “alkoxycarbonylamino” means a radical of the formula: —NH—C(O)—O-alkyl or —N[C(O)—O-alkyl]₂.

The term “alkylamino” means a radical of the formula: —NH-alkyl or —N(alkyl)₂.

The term “alkylaminoalkyl” means a radical of the formula: -alkyl-NH-alkyl, -alkyl-N(alkyl)₂, -alkyl-NH-alkyl-(terminal group), -alkyl-N(alkyl)-alkyl-(terminal group) or -alkyl-N(alkyl-terminal group)₂.

The term “alkylaminocarbonyl” means a radical of the formula: —C(O)—NH-alkyl, —C(O)—N(alkyl)₂, —C(O)—NH-alkyl-(terminal group), —C(O)—N(alkyl)-alkyl-(terminal group) or —C(O)—N(alkyl-terminal group)₂.

The term “alkylaminocarbonylamino” means a radical of the formula: —NH—C(O)—NH-alkyl or —NH—C(O)—N(alkyl)₂.

The term “alkylaminosulfonyl” means a radical of the formula: —SO₂—NH-alkyl or —SO₂—N(alkyl)₂.

The term “alkylaminosulfonylalkyl” means a radical of the formula: -alkyl-SO₂—NH-alkyl or -alkyl-SO₂—N(alkyl)₂.

The term “alkylcarbonyl” means a radical of the formula: —C(O)-alkyl or —C(O)-alkyl-(terminal group).

The term “alkylcarbonylamino” means a radical of the formula: —NH—C(O)-alkyl, —N[C(O)-alkyl]₂, —NH—C(O)-alkyl-(terminal group) or —N[C(O)-alkyl-(terminal group)]₂.

The term “alkylcarbonylaminoalkyl” means a radical of the formula: -alkyl-NH—C(O)-alkyl, -alkyl-N[C(O)-alkyl]₂, -alkyl-NH—C(O)-alkyl-(terminal group) or -alkyl-N[C(O)-alkyl-(terminal group)]₂.

The term “alkylcarbonyloxy” means a radical of the formula: —O—C(O)-alkyl or —O—C(O)-alkyl-(terminal group).

The term “alkylsulfonyl” means a radical of the formula: —SO₂-alkyl.

The term “alkylsulfonylamino” means a radical of the formula: —NH—SO₂-alkyl.

The term “alkylthio” means a radical of the formula —S-alkyl or —S-alkyl-(terminal group).

The term “alkylthiocarbonyl” means a radical of the formula —C(S)-alkyl or —C(S)-alkyl-(terminal group).

The term “amino” means a radical of the formula: —NH₂.

The term “aminoalkyl” means a radical of the formula: -alkyl-NH₂, -alkyl-NH-terminal group or -alkyl-N(terminal group)₂.

The term “aminocarbonyl” means a radical of the formula: —C(O)—NH₂, —C(O)—NH(terminal group) or —C(O)—N(terminal group)₂.

The term “aminocarbonylamino” means a radical of the formula: —NH—C(O)—NH₂.

The term “aminosulfonyl” means a radical of the formula: —SO₂—NH₂.

The term “aminosulfonylalkyl” means a radical of the formula: -alkyl-SO₂—NH₂.

The term “aryloxy” means a radical of the formula: —O-aryl.

The term “carbonyl” means a radical of the formula —C(O)-(terminal group).

The term “carbonylaminoiminomethyl” means a radical of the formula —C(NH)NHC(O)-terminal group.

The term “carboxy” means a radical of the formula —C(O)—OH.

The term “carboxyalkoxy” means a radical of the formula —O-alkyl-C(O)—OH.

The term “carboxyalkyl” means a radical of the formula -alkyl-C(O)—OH.

The term “carboxyalkylamino” means a radical of the formula —NH-alkyl-C(O)—OH.

The term “carboxyl” means a radical of the formula —C(O)—O-(terminal group).

The term “formyl” means a radical of the formula: —C(O)—H.

The term “formylamino” means a radical of the formula: —NH—C(O)—H.

The term “halogen” or “halo” means the group chloro, bromo, fluoro or iodo.

The term “haloalkoxy” means a radical of the formula: —O-alkyl-(halo)ₙ, wherein one or more halogen atoms may be substituted on alkyl when allowed by available valences (wherein n represents that amount of available valences based on the number of carbon atoms in the chain), and includes monofluoromethoxy, difluoromethoxy, trifluoromethoxy, trifluoroethoxy and the like.

The term “haloalkyl” means a radical of the formula: -alkyl-(halo)ₙ, wherein one or more halogen atoms may be substituted on alkyl when allowed by available valences (wherein n represents that amount of available valences based on the number of carbon atoms in the chain), and includes monofluoromethyl, difluoromethyl, trifluoromethyl, trifluoroethyl and the like.

The term “haloalkylthio” means a radical of the formula: —S-alkyl-(halo)ₙ, wherein one or more halogen atoms may be substituted on alkyl when allowed by available valences (wherein n represents that amount of available valences based on the number of carbon atoms in the chain), and includes monofluoromethyl, difluoromethyl, trifluoromethyl, trifluoroethyl and the like.

The term “hydroxyalkoxy” means a radical of the formula: —O-alkyl-hydroxy, wherein alkyl is substituted on one or more available carbon chain atoms with one or more hydroxy radicals.

The term “hydroxyalkyl” means a radical of the formula: -alkyl-hydroxy, wherein alkyl is substituted on one or more available carbon chain atoms with one or more hydroxy radicals.

The term “thiocarbonyl” means a radical of the formula —C(S)-(terminal group).

The term “aminothiocarbonyl” means a radical of the formula —C(S)—NH₂, —C(S)—NH-(terminal group) or —C(S)—N(terminal group)₂.

The term “substituted” means the independent replacement of one or more hydrogen atoms within a radical with that amount of substitutents allowed by available valences.

The term “dependently selected” means that the structure variables are specified in an indicated combination.

The term “terminal group” means a substitutent attached to a radical, wherein the radical functions as a linking group. When used in a radical formula, the atom to which the terminal group is attached is opposite the atom with an open dash. The open dash accordingly represents the point of attachment for the radical to the core molecule.

In general, IUPAC nomenclature rules are used throughout this disclosure.

Compound Forms

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Suitable salt forms include acid addition salts which may, for example, be formed by mixing a solution of the compound according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulfuric acid, tartaric acid, trifluoroacetic acid and the like.

Furthermore when the compounds of the present invention carry an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts include the following: acetate, adipate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

The term “prodrug” means a compound of Formula (I) or a form thereof that is converted in vivo into a functional derivative form that may contribute to therapeutic biological activity, wherein the converted form may be: 1) a relatively active form; 2) a relatively inactive form; 3) a relatively less active form; or, 4) any form which results, directly or indirectly, from such in vivo conversions.

Prodrugs are useful when said compound may be either too toxic to administer systemically, absorbed poorly by the digestive tract or broken down by the body before it reaches its target. Conventional procedures for the selection and preparation of suitable prodrug derivatives are described in, for example, “Design of Prodrugs”, ed. H. Bundgaard, Elsevier, 1985.

The term “metabolite” means a prodrug form of a compound of Formula (I) or a form thereof converted by in vivo metabolism or a metabolic process to a relatively less active functional derivative of said compound.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of isolated specie rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule, which in a given configuration, cannot be superimposed on its mirror image. This is in contrast to achiral molecules, which can be superimposed on their mirror images.

The two distinct mirror image versions of the chiral molecule are also known as levo (left-handed), abbreviated L, or dextro (right handed), abbreviated D, depending on which way they rotate polarized light. The symbols “R” and “S” represent the configuration of groups around a stereogenic carbon atom(s).

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levorotatory = \frac{( mass levorotatory )}{( mass dextrorotatory ) + ( mass levorotatory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( mass dextrorotatory )}{( mass dextrorotatory ) + ( mass levorotatory )} \times 100$$

The term “geometric isomer” means isomers that differ in the orientation of substitutent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substitutents are on opposite sides in relationship to the carbon-carbon double bond. In the “Z” configuration, the substitutents are oriented on the same side in relationship to the carbon-carbon double bond.

Substituent atoms (other than hydrogen) attached to a ring system may be in a cis or trans configuration. In the “cis” configuration, the substitutents are on the same side in relationship to the plane of the ring; in the “trans” configuration, the substitutents are on opposite sides in relationship to the plane of the ring. Compounds having a mixture of “cis” and “trans” species are designated “cis/trans”.

The isomeric descriptors (“R,” “S,” “E,” and “Z”) indicate atom configurations and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, a compound of the present invention may have at least one crystalline, polymorph or amorphous form. The plurality of such forms is intended to be included in the scope of the invention. In addition, a compound of the present invention may form a solvate with water (i.e., hydrates) or common organic solvents (e.g., organic esters such as ethanolate and the like). The plurality of such solvates is also intended to be encompassed within the scope of this invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

Therapeutic Use

The compounds of Formula (I) or a form thereof in accordance with the present invention are CCR2 antagonists.

A compound of Formula (I) or a form thereof may have a mean inhibition constant (IC₅₀) against MCP-1 binding to CCR2 of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

A compound of Formula (I) or a form thereof reduces MCP-1 induced monocyte chemotaxis. A compound of Formula (I) or a form thereof may have an IC₅₀ for reduction in MCP-1 induced monocyte chemotaxis of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

A compound of Formula (I) or a form thereof reduces MCP-1 intracellular calcium mobilization. A compound of Formula (I) or a form thereof may have an IC₅₀ for reduction in MCP-1 induced intracellular calcium mobilization of between about 50 μM to about 0.01 nM; between about 25 μM to about 0.01 nM; between about 10 μM to about 0.01 nM; between about 5 μM to about 0.01 nM; between about 1 μM to about 0.01 nM; between about 800 nM to about 0.01 nM; between about 200 nM to about 0.01 nM; between about 100 nM to about 0.01 nM; or, between about 10 nM to about 0.01 nM.

The present invention includes use of a compound of Formula (I) or a form thereof as a CCR2 antagonist comprising contacting the receptor with the compound.

The use of the compound of Formula (I) or a form thereof further comprises use of the compound in a pharmaceutical composition, medicine or medicament for preventing, treating or ameliorating a CCR2 mediated inflammatory syndrome, disorder or disease.

The compound of Formula (I) or a form thereof may also be used in the manufacture of a medicament for preventing, treating or ameliorating a CCR2 mediated inflammatory syndrome, disorder or disease.

The present invention also includes a medicament comprising an effective amount of a compound of Formula (I) or a form thereof.

The present invention also includes the use of a compound of Formula (I) or a form thereof in a method for preventing, treating or ameliorating a CCR2 mediated inflammatory syndrome, disorder or disease in a subject in need thereof comprising administering to the subject an effective amount of the compound of Formula (I) or form thereof.

The present invention is directed to a method for preventing, treating or ameliorating a CCR2 mediated inflammatory syndrome, disorder or disease in a subject in need thereof comprising administering to the subject an effective amount of a compound of Formula (I) or a form thereof.

The term “administering” with respect to the methods of the invention, means a method for therapeutically or prophylactically preventing, treating or ameliorating a syndrome, disorder or disease as described herein by using a compound of Formula (I) or a form thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment and is at risk of (or susceptible to) developing a syndrome, disorder or disease that is associated with elevated MCP-1 expression or MCP-1 overexpression, or a patient with an inflammatory condition that accompanies syndromes, disorders or diseases associated with elevated MCP-1 expression or MCP-1 overexpression.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The effective amount of a compound of the invention in such a therapeutic method is from about 0.001 mg/kg/day to about 300 mg/kg/day.

An example of a compound of Formula (I) or a form thereof is selected from the

| Cpd | Name |
|---|---|
| 1 | 3-(3,4-dichloro-phenyl)-1-(4-hydroxy-4-{1-hydroxy-2-[4-(4-methoxy-phenyl)- |
| | piperidin-1-yl]-ethyl}-piperidin-1-yl)-propenone, |
| 2 | 1-[4-(4-chloro-phenyl)-piperidin-1-yl]-2-{1-[3-(3,4-dichloro-phenyl)-acryloyl]- |
| | piperidin-4-yl}-ethane-1,2-dione, |
| 3 | 1-(4-{2-[4-(4-chloro-phenyl)-piperidin-1-hydroxy-2-oxo-ethyl}-piperidin- |
| | 1-yl)-3-(3,4-dichloro-phenyl)-propenone, |
| 4 | 4-{1-hydroxy-2-[4-(1H-indol-3-yl)-piperidin-1-yl]-ethyl}-piperidine-1- |
| | carboxylic acid (3,4-dichloro-phenyl)-amide, |
| 5 | 4-{1-hydroxy-2-[4-(1H-indol-3-yl)-piperidin-1-yl]-ethyl}-piperidine-1- |
| | carboxylic acid (3,5-difluoro-phenyl)-amide, |
| 6 | 3-(3,4-dichloro-phenyl)-1-(4-{1-hydroxy-2-[4-(1H-indol-3-yl)-piperidin-1-yl]- |
| | ethyl}-piperidin-1-yl)-propenone, |
| 7 | 3-(3,5-difluoro-phenyl)-1-(4-{1-hydroxy-2-[4-(1H-indol-3-yl)-piperidin-1-yl]- |
| | ethyl}-piperidin-1-yl)-propenone, |
| 8 | (4-{1-hydroxy-2-[4-(1H-indol-3-yl)-piperidin-1-yl]-ethyl}-piperidin-1-yl)- |
| | (3,4,5-trifluoro-phenyl)-methanone, |
| 9 | 4-{1-hydroxy-2-[4-(1H-indol-3-yl)-piperidin-1-yl]-ethyl}-piperidine-1- |
| | carbothioic acid (3-bromo-phenyl)-amide, |
| 10 | 3,5-difluoro-N-[(4-{1-hydroxy-2-[4-(1H-indol-3-yl)-piperidin-1-yl]-ethyl}- |
| | piperidin-1-yl)-imino-methyl]-benzamide, |
| 11 | 1-(4-{1-hydroxy-2-[4-(1H-indol-3-yl)-piperidin-1-yl]-ethyl}-piperidin-1-yl)-3- |
| | m-tolyl-propenone, |
| 12 | 3-(3,5-difluoro-phenyl)-1-(4-[1-hydroxy-2-[4-(4-methoxy-phenyl)-piperidin-1- |
| | yl]-ethyl}-piperidin-1-yl)-propenone, |
| 13 | 1-(4-{1-hydroxy-2-[4-(4-methoxy-phenyl)-piperidin-1-yl]-ethyl}-piperidin-1- |
| | yl)-3-(3,4,5-trifluoro-phenyl)-propenone, |
| 14 | 3-(3,4-dichloro-phenyl)-1-(4-{1-hydroxy-2-[4-(4-methoxy-phenyl)-piperidin-1- |
| | yl]-ethyl}-piperidin-1-yl)-propenone, |
| 15 | 4-{1-hydroxy-2-[4-(4-methoxy-phenyl)-piperidin-1-yl]-ethyl}-piperidine-1- |
| | carboxylic acid (3,4-dichloro-phenyl)-amide, |
| 16 | 4-{1-hydroxy-2-[4-(4-methoxy-phenyl)-piperidin-1-yl]-ethyl}-piperidine-1- |
| | carbothioic acid (3-bromo-phenyl)-amide, |
| 17 | 1-(4-{2-[4-(1H-pyrrolo[2,3-b]pyridin-3-yl)-piperidin-1-yl]-acetyl}-piperidin-1- |
| | yl)-3-(3,4,5-trifluoro-phenyl)-propenone, |
| 18 | N-{3-[1-(2-oxo-2-{1-[3-(3,4,5-trifluoro-phenyl)-acryloyl]-piperidin-4-yl}- |
| | ethyl)-piperidin-4-yl]-1H-indol-5-yl}-methanesulfonamide, |
| 19 | 1-(4-{1-hydroxy-2-[4-(1H-pyrrolo[2,3-b]pyridin-3-yl)-piperidin-1-yl]-ethyl}- |
| | piperidin-1-yl)-3-m-tolyl-propenone, |
| 20 | 3,(3,5-difluoro-phenyl)-1-(4-{1-hydroxy-2-[4-(1H-pyrrolo[2,3-b]pyridin-3yl)- |
| | piperidin-1-yl]-ethyl}-piperidin-1-yl)-propenone, |
| 21 | 1-(4-{1-hydroxy-2-[4-(1H-pyrrolo[2,3-b]pyridin-3-yl)-piperidin-1-yl]-ethyl}- |
| | piperidin-1-yl)-3-(3,4,5-trifluoro-phenyl)-propenone, |
| 22 | 1-(4-{1-hydroxy-2-[4-(1H-pyrrolo[2,3-b]pyridin-3-yl)-piperidin-1-yl]-ethyl}- |
| | piperidin-1-yl)-3-(3-trifluoromethyl-phenyl)-propenone, |
| 23 | 3-(3,4-dichloro-phenyl)-1-(4-{1-hydroxy-2-[4-(1H-pyrrolo[2,3-b]pyridin-3-yl)- |
| | piperidin-1-yl]-ethyl}-piperidin-1-yl)-propenone, |
| 24 | 4-{1-hydroxy-2-[4-(1H-pyrrolo[2,3-b]pyridin-3-yl)-piperidin-1-yl]-ethyl}- |
| | piperidine-1-carboxylic acid (3,4-dichloro-phenyl)amide, |
| 25 | 4-{1-hydroxy-2-[4-(1H-pyrrolo[2,3-b]pyridin-3-yl)-piperidin-1-yl]-ethyl}- |
| | piperidine-1-carboxylic acid (4-methylsulfanyl-phenyl)-amide, |
| 26 | 3-(3,5-difluoro-phenyl)-1-(4-{1-hydroxy-2-[4-(5-methoxy-1H-indol-3-yl)- |
| | piperidin-1-yl]-ethyl}-piperidin-1-yl)-propenone, |
| 27 | N-{3-[1-(2-hydroxy-2-{1-[3-(3,4,5-trifluoro-phenyl)-acryloyl]-piperidin-4-yl}- |
| | ethyl)-piperidin-4-yl]-1H-indol-5-yl}-mathanesulfonamide, |
| 28 | N-{3-[1-(2-amino-2-{1-[3-(3,4,5-trifluoro-phenyl)-acryloyl]-piperidin-4-yl}- |
| | ethyl)-piperidin-4-yl]-1H-indol-5-yl}-methanesulfonamide, |
| 29 | 1-(4-{1-hydroxy-2-[(4-methoxy-phenyl)-piperidin-1-yl[-ethyl}-piperidin-1- |
| | yl)-3-m-tolyl-propenone, |
| 30 | 1-(4-{1-hydroxy-2-[4-(4-methyoxy-phenyl)-piperidin-1-yl]-ethyl}-piperidin-1- |
| | yl)-3-(3-trifluoromethyl-phenyl)-propenone, |
| 31 | 4-{1-hydroxy-2-[4-(4-methoxy-phenyl)-piperidin-1-yl]-ethyl}-piperidine-1- |
| | carboxylic acid (4-methylsulfanyl-phenyl)-amide, |
| 32 | 3-(3,4-difluoro-phenyl)-1-(4-{1-hydroxy-2-[4-(4-methoxy-phenyl)-piperidin-1- |
| | yl]-ethyl}-piperidin-1-yl)-propenone, |
| 33 | 1-(4-{1-hydroxy-2[4-(1H-pyrazol-3-yl)-piperidin-1-yl]-ethyl}-piperidin-1-yl)- |
| | 3-m-tolyl-propenone, |
| 34 | 3-(3,5-difluoro-phenyl)-1-(4-{1-hydroxy-2-[4-(1H-pyrazol-3-yl)-piperidin-1-yl]- |
| | ethyl}-piperidin-1-yl)-propenone, |
| 35 | 1-(4-{1-hydroxy-2-[4-(1H-pyrazol-3-yl)-piperidin-1-yl]-ethyl}-piperidin-1-yl)- |
| | 3-(3,4,5-trifluoro-phenyl)-propenone, |
| 36 | 4-{1-hydroxy-2-[4-(1H-pyrazol-3-yl)-piperadin-1-yl]-ethyl}-piperidine-1- |
| | carboxylic acid (3,4-dichloro-phenyl)-amide, |
| 37 | 4-{1-hydroxy-2-[4-(1H-pyrazol-3-yl)-piperidin-1-yl]-ethyl}-piperidine-1- |
| | carboxylic acid (3,4-difluoro-phenyl)-amide, |
| 38 | 3-[4-(4-methoxy-phenyl)-piperidin-1-yl]-2-{1-[3-(3,4,5-trifluoro-phenyl)- |
| | acryloyl]-piperidin-4-yl}-propionic acid ethyl ester, |
| 39 | 3-[4-(4-methoxy-phenyl)-piperidin-1-yl]-2-{1-[3-(3,4,5-trifluoro-phenyl)- |
| | acryloyl]-piperidin-4-yl}-propionic acid, |
| 40 | N-[3-(1-{2-hydroxy-2-[1-(3-m-tolyl-acryloyl)-piperidin-4-yl]-ethyl}-piperidin- |
| | 4-yl)-1H-indol-5-yl]-methanesulfonamide, |
| 41 | N-{3-[1-(2-{1-[3-(3,5-difluoro-phenyl)-acrloyl]-piperidin-4-yl}-2-hydroxy- |
| | ethyk)-piperidin-4-yl]-1H-indol-5-yl}-methanesulfonamide, |
| 42 | 1-(4-{1-hydroxymethyl-2-[4-(4-methoxy-phenyl)-piperidin-1-yl]-ethyl}- |
| | piperidin-1-yl)-3-m-tolyl-propenone, |
| 43 | 3-(3,4-dichloro-phenyl)-1-(4-{1-hydroxymethyl-2-[4-(4-methoxy-phenyl)- |
| | piperidin-1-yl]-ethyl}-piperidin-1-yl)-propenone, |
| 44 | 4-{1-hydroxymethyl-2-[4-(4-methoxy-phenyl)-piperidin-1-yl]-ethyl}-piperidine- |
| | 1-carboxylic acid (3,4-dichloro-phenyl)-amide, |
| 45 | 3-(3,5-difluoro-phenyl)-1-(4-{1-hydroxymethyl-2-[4-(4-methoxy-phenyl)- |
| | piperidin-1-yl]-ethyl{-piperidin-1-yl)-propenone, |
| 46 | 1-{4-[2-(4-benzoxazol-2-yl-piperidin-1-yl)-1-hydroxy-ethyl]-piperidin-1-yl}-3- |
| | (3,5-difluoro-phenyl)-propenone, |
| 47 | 1-{4-[2-(4-benzoxazol-2-yl-piperidin-1-yl)-1-hydroxy-ethyl]-piperidin-1-yl}-3- |
| | m-tolyl-propenone, |
| 48 | 2-[4-(1H-indol-3-yl)-piperidin-1-yl]-3-[1-(3-m-tolyl-acryloyl)-piperidin-4-yl]- |
| | propionic acid ethyl ester, |
| 49 | 3-{1-[3-(3,4-dichloro-phenyl)-acryloyl]-piperidin-4-yl}-2-[4-(1H-indol-3-yl)- |
| | piperidin-1-yl]-propionic acid ethyl ester, |
| 50 | 2-[4-(1H-indol-3-yl)-piperidin-1-yl]-3-{1-[3-(3,4,5-trifluoro-phenyl)-acryloyl]- |
| | piperidin-4-yl}-propionic acid ethyl ester, |
| 51 | 3-{1-[3-(3,5-difluoro-phenyl)-acryloyl]-piperidin-4-yl}-2-[4-(1H-indol-3-yl)- |
| | piperidin-1-yl]-propionic acid ethyl ester, |
| 52 | 3-[1-(3,4-dichloro-phenylcarbamoyl)-piperidin-4-yl]-2-[4-(1H-indol-3-yl)- |
| | piperidin-1-yl]-propionic acid ethyl ester, |
| 53 | 2-[4-(1H-indol-3-yl)-piperidin-1-yl]-3-[1-(3-m-tolyl-acryloyl)-piperidin-4-yl]- |
| | propionic acid, |
| 54 | 3-(3,5-difluoro-phenyl)-1-(4-[3-hydroxy-2-[4-(1H-indol-3-yl)-piperidin-1-yl]- |
| | propyl}-piperidin-1-yl)-propenone, |
| 55 | 1-(4-{3-hydroxy-2-[4-(1H-indol-3-yl)-piperidin-1-yl]-propyl}-piperidin-1-yl)-3- |
| | (3,4,5-trifluoro-phenyl)-propenone, |
| 56 | 3-(3,4-dichloro-phenyl)-1-(4-{3-hydroxy-2-[4-(1H-indol-3-yl)-piperidin-1-yl]- |
| | propyl}-piperidin-1-yl)-propenone, |
| 57 | 1-(4-{3-hydroxy-2-[4-(1H-indol-3-yl)-piperidin-1-yl]-propyl}-piperidin-1-yl)-3- |
| | m-tolyl-propenone, |
| 58 | 4-{3-hydroxy-2-[4-(1H-indol-3-yl)-piperidin-1-yl]-propyl}-piperidine-1- |
| | carboxylic acid (3,4-dichloro-phenyl)-amide, |
| 59 | 4-{3-hydroxy-2-[4-(1H-indol-3-yl)-piperidin-1-yl]-propyl}-piperidine-1- |
| | carbothioic acid (3-bromo-phenyl)-amide, |
| 60 | 3-{1-[3-(3,4-dichloro-phenyl)-acryloyl]-piperidin-4-yl}-2-[4-(1H-indol-3-yl)- |
| | piperidin-1-yl]-propionic acid, |
| 61 | 3-{1-[3-(3,5-difluoro-phenyl)-acryloyl]-piperidin-4-yl}-2-[4-(1H-indol-3-yl)- |
| | piperidin-1-yl]-propionic acid, |
| 62 | 3-[1-(3,4-dichloro-phenylcarbamoyl)-piperidin-4-yl]-2-[4-(1H-indol-3-yl)- |
| | piperidin-1-yl]-propionic acid, |
| 63 | 4-{1-hydroxy-2-[4-(5-methanesulfonylamino-1H-indol-3-yl)-piperidin-1-yl]- |
| | ethyl}-piperidine-1-carboxylic acid tert-butyl ester, |
| 64 | 1-(4-{2-[4-(4-methoxy-phenyl)-piperidin-1-yl]-acetyl}-piperidin-1-yl)-3-(3,4,5- |
| | trifluoro-phenyl)-propenone, |
| 65 | 1-(4-{1-amino-2-[4-(4-methoxy-phenyl)-piperidin-1-yl]-ethyl}-piperidin-1-yl)- |
| | 3-(3,4,5-trifluoro-phenyl)-propenone, |
| 66 | N-(2-[4-(4-methoxy-phenyl)-piperidin-1-yl]-1-{1-[3-(3,4,5-trifluoro-phenyl)- |
| | acryloyl]-piperidin-4-yl}-ethyl)-acetamide, |
| 67 | (2-[4-(4-methoxy-phenyl)-piperidin-1-yl]-1-{1-[3-(3,4,5-trifluoro-phenyl)- |
| | acryloyl]-piperidin-4-yl}-ethyl)-carbamic acid methyl ester, |
| 68 | 3-(2-[4-(4-methoxy-phenyl)-piperidin-1-yl]-1-{1-[3-(3,4,5-trifluoro-phenyl)- |
| | acryloyl]-piperidin-4-yl}-ethyl)-1,1-dimethyl-urea, |
| 69 | 4-hydroxy-4-{1-hydroxy-2-[4-(1H-indol-3-yl)-piperidin-1-yl]-ethyl}-piperidine- |
| | 1-carboxylic acid benzyl ester, |
| 70 | 1-(4-hydroxy-4-{1-hydroxy-2-[4-(1H-indol-3-yl)-piperidin-1-yl]-ethyl}- |
| | piperidin-1-yl)-3-(3,4,5-trifluoro-phenyl)-propenone, |
| 71 | 3-(3,5-difluoro-phenyl)-1-(4-hydroxy-4-{1-hydroxy-2-[4-(1H-indol-3-yl)- |
| | piperidin-1-yl]-ethyl}-piperidin-1-yl)-propenone, |
| 72 | 3-(3,5-bis-trifluoromethyl-phenyl)-1-(4-hydroxy-4-{1-hydroxy-2-[4-(1H-indol- |
| | 3-yl)-piperidin-1-yl]-ethyl}-piperidin-1-yl)-propenone, |
| 73 | 3-(3,5-dichloro-phenyl)-1-(4-hydroxy-4-{1-hydroxy-2-[4-(1H-indol-3-yl)- |
| | piperidin-1-yl]-ethyl}-piperidin-1-yl)-propenone, |
| 74 | 3-(3-chloro-5-fluoro-phenyl)-1-(4-hydroxy-4-{1-hydroxy-2-[4-(1H-indol-3-yl)- |
| | piperidin-1-yl]-ethyl]-piperidin-1-yl)-propenone, |
| 75 | 3-(3,4-dichloro-phenyl)-1-(4-hydroxy-4-[1-hydroxy-2-[4-(1H-indol-3-yl)- |
| | piperidin-1-yl]-ethyl}-piperidin-1-yl)-propenone, |
| 76 | 3-(3-fluoro-phenyl)-1-(4-hydroxy-4-{1-hydroxy-2-[4-(1H-indol-3-yl)-piperidin- |
| | 1-yl]-ethyl}-piperidin-1-yl)-propenone, |
| 77 | 1-(4-hydroxy-4-{1-hydroxy-2-[4-(1H-indol-3-yl)-piperidin-1-yl]-ethyl}- |
| | piperidin-1-yl)-3-(3-trifluoromethyl-phenyl)-propenone, |
| 78 | 3-(3,4-difluoro-phenyl)-1-(4-hydroxy-4-{1-hydroxy-2-[4-(1H-indol-3-yl)- |
| | piperidin-1-yl]-ethyl}-piperdin-1-yl)-propenone, |
| 79 | 1-(4-hydroxy-4-{1-hydroxy-2-[4-(1H-indol-3-yl)-piperidin-1-yl]-ethyl}- |
| | piperidin-1-yl)-3-thiophen-3-yl-propenone, |
| 80 | 3-(3-bromo-4-fluoro-phenyl)-1-(4-hydroxy-4-{1-hydroxy-2-[4-(1H-indol-3-yl)- |
| | piperidin-1-yl]-ethyl}-piperidin-1-yl)-propenone, |
| 81 | 4-hydroxy-4-{1-hydroxy-2-hydroxy-2-[4-(1H-indol-3-yl)-piperidin-1-yl]-ethyl}-piperidine- |
| | 1-carboxylic acid (3,5-difluoro-phenyl)-amide, |
| 82 | 4-hydroxy-4-{1-hydroxy-2-[4-(1H-indol-3-yl)-piperidin-1-yl]-ethyl}-piperidine- |
| | 1-carboxylic acid (3,4-difluoro-phenyl)-amide, |
| 83 | 4-hydroxy-4-{1-hydroxy-2-[4-(1H-indol-3-yl)-piperidin-1-yl]-ethyl}-piperidine- |
| | 1-carbothioic acid (3,5-dichloro-phenyl)-amide, |
| 84 | 4-hydroxy-4-{1-hydroxy-2-[4-(1H-indol-3-yl)-piperidin-1-yl]-ethyl}-piperidine- |
| | 1-carbothioic acid (3,4-dichloro-phenyl)-amide, |
| 85 | 1-(4-{2-[4-(1H-benzoimidazol-2-yl)-piperidin-1-yl]-1-hydroxy-ethyl}-piperidin- |
| | 1-yl)-3-(3,4-dichloro-phenyl)-propenone, |
| 86 | 1-(4-{2-[4-(1H-benzoimidazol-2-yl)-piperidin-1-yl]-1-hydroxy-ethyl}-piperidin- |
| | 1-yl)-3-(3,4,5-trifluoro-phenyl)-propenone, |
| 87 | 1-(4-{2-[4-(1H-benzoimidazol-2-yl)-piperidin-1-yl]-1-hydroxy-ethyl}-4- |
| | hydroxy-piperidin-1-yl)-3-(3,4,5-trifluoro-phenyl)-propenone, |
| 88 | 1-(4-hydroxy-4-{1-hydroxy-2-[4-(4-methoxy-phenyl)-piperidin-1-yl]-ethyl}- |
| | piperidin-1-yl)-3-(3,4,5-trifluoro-phenyl)-propenone, |
| 89 | 4-{1-hydroxy-2-[4-(4-methoxy-phenyl)-piperidin-1-yl]-ethyl}-piperidine-1- |
| | carbothioic acid (3,5-dichloro-phenyl)-amide, |
| 90 | 4-{1-hydroxy-2-[4-(4-methoxy-phenyl)-piperidin-1-yl]-ethyl}-piperidine-1- |
| | carboxylic acid (4,5-dichloro-2-fluoro-phenyl)-amide, |
| 91 | 4-{1-hydroxy-2-[4-(4-methoxy-phenyl)-piperidin-1-yl]-ethyl}-piperidine-1- |
| | carboxylic acid (3-fluoro-5-trifluoromethyl-phenyl)-amide, |
| 92 | 3-(4-chloro-phenyl)-1-(4-hydroxy-4-{hydroxy-2-[4-(4-methoxy-phenyl)- |
| | piperidin-1-yl]-ethyl}-piperidin-1-yl)-propenone, |
| 93 | 3-(3,4-difluoro-phenyl)-1-(4-hydroxy-4-{1-hydroxy-2-[4-(4-methoxy-phenyl)- |
| | piperidin-1-yl]-ethyl}-piperidin-1-yl)-propenone, |
| 94 | 3-(3,5-difluoro-phenyl)-1-(4-hydroxy-4-{1-hydroxy-2-[4-(4-methoxy-phenyl)- |
| | piperidin-1-yl]-ethyl}-piperidin-1-yl)-propenone, |
| 95 | 1-(4-hydroxy-4-{1-hydroxy-2-[4-(4-methoxy-phenyl)-piperidin-1-yl]-ethyl}- |
| | piperidin-1-yl)-3-m-tolyl-propenone, |
| 96 | 4-{1-hydroxy-2-[4-(4-methoxy-phenyl)-piperidin-1-yl]-ethyl}-piperidine-1- |
| | carbothioic acid (3-methoxy-phenyl)-amide, |
| 97 | 4-{1-hydroxy-2-[4-(4-methoxy-phenyl)-piperidin-1-yl]-ethyl}-piperidine-1- |
| | carbothioic acid (3-trifluoromethyl-phenyl)-amide, |
| 98 | 4-{1-hydroxy-2-[4-(4-methoxy-phenyl)-piperidin-1-yl]-ethyl}-piperidine-1- |
| | carbothioic acid (3,4-dichloro-phenyl)-amide, |
| 99 | 4-{1-hydroxy-2-[4-(4-methoxy-phenyl)-piperidin-1-yl]-ethyl}-piperidine-1- |
| | carbothioic acid (4-bromo-3-fluoro-phenyl)-amide, |
| 100 | 4-{1-hydroxy-2-[4-(4-methoxy-phenyl)-piperidin-1-yl]-ethyl}-piperidine-1- |
| | carboxylic acid benzo[1,3]dioxol-5-ylamide, |
| 101 | 4-{1-hydroxy-2-[4-(4-methoxy-phenyl)-piperidin-1-yl]-ethyl}-piperidine-1- |
| | carboxylic acid (2-chloro-4-trifluoromethyl-phenyl)-amide, |
| 102 | 4-{1-hydroxy-2-[4-(4-methoxy-phenyl)-piperidin-1-yl]-ethyl}-piperidine-1- |
| | carbothioic acid (3-chloro-phenyl)-amide, |
| 103 | 4-{1-hydroxy-2-[4-(4-methoxy-phenyl)-piperidin-1-yl]-ethyl}-piperidine-1- |
| | carbothioic acid (3-methoxy-phenyl)-amide, |
| 104 | 4-{1-hydroxy-2-[4-(4-methoxy-phenyl)-piperidin-1-yl]-ethyl}-piperidine-1- |
| | carbothioic acid (3-cyano-phenyl)-amide, |
| 105 | 4-{1-hydroxy-2-[4-(4-methoxy-phenyl)-piperidin-1-yl]-ethyl}-piperidine-1- |
| | carbothioic acid (3-trifluoromethyl-phenyl)-amide, |
| 106 | 4-{1-hydroxy-2-[4-(4-methoxy-phenyl)-piperidin-1-yl]-ethyl}-piperidine-1- |
| | carbothioic acid (4-trifluoromethyl-phenyl)-amide, |
| 107 | 4-{1-hydroxy-2-[4-(4-methoxy-phenyl)-piperidin-1-yl]-ethyl}-piperidine-1- |
| | carbothioic acid (3,5-difluoro-phenyl)-amide, |
| 108 | 4-{1-hydroxy-2-]4-(4-methoxy-phenyl)-piperidin-1-yl]-ethyl}-piperidine-1- |
| | carbothioic acid (3,4-dichloro-phenyl)-amide, |
| 109 | 4-{1-hydroxy-2-[4-(4-methoxy-phenyl)-piperidin-1-yl]-ethyl}-piperidine-1- |
| | carbothioic acid (2,3,5-trifluoro-phenyl)-amide, |
| 110 | 4-{1-hydroxy-2-[4-(4-methoxy-phenyl)-piperidin-1-yl]-ethyl}-piperidine-1- |
| | carbothioic acid (4-bromo-3-fluoro-phenyl)-amide, |
| 111 | 4-{1-hydroxy-2-[4-(4-methoxy-phenyl)-piperidin-1-yl]-ethyl}-piperidine-1- |
| | carbothioic acid (3,5-dimethoxy-phenyl)-amide, |
| 112 | 4-{2-[4-(4-chloro-phenyl)-piperidin-1-yl]-1-hydroxy-ethyl}-4-hydroxy- |
| | piperidine-1-carboxylic acid benzyl ester, |
| 113 | 1-(4-hydroxy-4-{1-hydroxy-2-[4-(4-methoxy-phenyl)-piperidin-1-yl]-ethyl}- |
| | piperidin-1-yl)-3-thiophen-3-yl-propenone, |
| 114 | 4-hydroxy-4-{1-hydroxy-2-[4-(4-methoxy-phenyl)-piperidin-1-yl]-ethyl}- |
| | piperidine-1-carboxylic acid (3,4-difluoro-phenyl)-amide, |
| 115 | 4-hydroxy-4-{1-hydroxy-2-[4-(4-methoxy-phenyl)-piperidin-1-yl]-ethyl}- |
| | piperidine-1-carboxylic acid (3,5-difluoro-phenyl)-amide, |
| 116 | 4-hydroxy-4-{1-hydroxy-2-[4-(4-methoxy-phenyl)-piperidin-1-yl]-ethyl}- |
| | piperidine-1-carboxylic acid (3,4-dichloro-phenyl)-amide, |
| 117 | 4-hydroxy-4-{1-hydroxy-2-[4-(4-methoxy-phenyl)-piperidin-1-yl]-ethyl}- |
| | piperidine-1-carbothioic acid (3,4-dichloro-phenyl)-amide, |
| 118 | 4-hydroxy-4-{1-hydroxy-2-[4-(4-methoxy-phenyl)-piperidin-1-yl]-ethyl}- |
| | piperidine-1-carbothioic acid (3,5-dichloro-phenyl)-amide, |
| 119 | 4-hydroxy-4-{1-hydroxy-2-[4-(4-methoxy-phenyl)-piperidin-1-yl]-ethyl}- |
| | piperidine-1-carbothioic acid (3,5-dimethoxy-phenyl)-amide, |
| 120 | 4-{1-hydroxy-2-[4-(1H-indol-3-yl)-piperidin-1-yl]-ethyl}-1-(4-trifluoromethyl- |
| | pyrimidin-2-yl)-piperidin-4-ol, |
| 121 | 4-{2-[4-1H-benzoimidazol-2-yl)-piperidin-1-yl]-1-hydroxy-ethyl}-1-(4- |
| | trifluoromethyl-pyrimidin-2-yl)-piperidin-4-ol, |
| 122 | 4-{1-hydroxy-2-[4-(5-morpholin-4-yl-1H-indol-3-yl)-piperidin-1-yl]-ethyl}-1- |
| | (4-trifluoromethyl-pyrimidin-2-yl)-piperidin-4-ol, |
| 123 | 4-{1-hydroxy-2-[4-(5-methyl-1H-indol-3-yl)-piperidin-1-yl]-ethyl}-1-(4- |
| | trifluoromethyl-pyrimidin-2-yl)-piperidin-4-ol, |
| 124 | 3-(1-{2-hydroxy-2-[4-hydroxy-1-(4-trifluoromethyl-pyrimidin-2-yl)-piperidin-4- |
| | yl]-ethyl}-piperidin-4-yl)-1H-indole-5-carbonitrile, |
| 125 | 4-{2-[4-(4-fluoro-phenyl)-piperidin-1-yl]-1-hydroxy-ethyl}-4-hydroxy- |
| | piperidine-1-carboxylic acid benzyl ester, |
| 126 | 4-hydroxy-4-{1-hydroxy-2-[4-(4-trifluoromethyl-phenyl)-piperidin-1-yl]-ethyl}- |
| | piperidine-1-carboxylic acid benzyl ester, |
| 127 | 2-[4-(4-methoxy-phenyl)-piperidin-1-yl]-1-(5′-nitro-3,4,5,6-tetrahydro-2H- |
| | [1,2′]bipyridinyl-4-yl)-ethanol, |
| 128 | 1-(4-{2-[4-(4-fluoro-phenyl)-piperidin-1-yl]-1-hydroxy-ethyl}-4-hydroxy- |
| | piperidin-1-yl)-3-(3,4,5-trifluoro-phenyl)-propenone, |
| 129 | 3-(3,4-difluoro-phenyl)-1-(4-{2-[4-(4-fluoro-phenyl)-piperidin-1-yl]-1-hydroxy- |
| | ethyl}-4-hydroxy-piperidin-1-yl)-propenone, |
| 130 | 3-(3,5-difluoro-phenyl)-1-(4-{2-[4-(4-fluoro-phenyl)-piperidin-1-yl]-1-hydroxy- |
| | ethyl}-4-hydroxy-piperidin-1-yl)-propenone, |
| 131 | 3-(3,4-dichloro-phenyl)-1-(4-{2-[4-(4-fluoro-phenyl)-piperidin-1-yl]-1-hydroxy- |
| | ethyl}-4-hydroxy-piperidin-1-yl)-propenone, |
| 132 | 4-{2-[4-(4-fluoro-phenyl)-piperidin-1-yl]-1-hydroxy-ethyl}-4-hydroxy- |
| | piperidine-1-carboxylic acid (3,4-difluoro-phenyl)-amide, |
| 133 | 4-{2-[4-(4-fluoro-phenyl)-piperidin-1-yl]-1-hydroxy-ethyl}-4-hydroxy- |
| | piperidine-1-carboxylic acid (3,5-difluoro-phenyl)-amide, |
| 134 | 4-{2-[4-(4-fluoro-phenyl)-piperidin-1-yl]-1-hydroxy-ethyl}-4-hydroxy- |
| | piperidine-1-carboxylic acid (3,4-dichloro-phenyl)-amide, |
| 135 | 4-{2-[4-(4-fluoro-phenyl)-piperidin-1-yl]-1-hydroxy-ethyl}-4-hydroxy- |
| | piperidine-1-carbothioic acid (3,4-dichloro-phenyl)-amide, |
| 136 | 1-(4-hydroxy-4-{1-hydroxy-2-[4-(4-trifluoromethyl-phenyl)-piperidin-1-yl]- |
| | ethyl}-piperidin-1-yl)-3-(3,4,5-trifluoro-phenyl)-propenone, |
| 137 | 3-(3,4-difluoro-phenyl)-1-(4-hydroxy-4-{1-hydroxy-2-[4-(4-trifluoromethyl- |
| | phenyl)-piperidin-1-yl]-ethyl}-piperidin-1-yl)-propenone, |
| 138 | 3-(3,5-difluoro-phenyl)-1-(4-hydroxy-4-{1-hydroxy-2-[4-(4-trifluoromethyl- |
| | phenyl)-piperidin-1-yl]-ethyl}-piperidin-1-yl)-propenone, |
| 139 | 3-(3,4-dichloro-phenyl)-1-(4-hydroxy-4-{1-hydroxy-2-[4-(4-trifluoromethyl- |
| | phenyl)-piperidin-1-yl]-ethyl}-piperidin-1-yl)-propenone, |
| 140 | 3-(3,4-dimethoxy-phenyl)-1-(4-hydroxy-4-{1-hydroxy-2-[4(4-trifluoromethyl- |
| | phenyl)-piperidin-1-yl]-ethyl}-piperidin-1-yl)-propenone, |
| 141 | 4-hydroxy-4-{1-hydroxy-2-[4-(4-trifluoromethyl-phenyl)-piperidin-1-yl]-ethyl}- |
| | piperidine-1-carboxylic acid (3,4-difluoro-phenyl)-amide, |
| 142 | 4-hydroxy-4-{1-hydroxy-2-[4-(4-trifluoromethyl-phenyl)-piperidin-1 yl]-ethyl}- |
| | piperidine-1-carboxylic acid (3,5-difluoro-phenyl)-amide, |
| 143 | 4-hydroxy-4-{1-hydroxy-2-[4-(4-trifluoromethyl-phenyl)-piperidin-1-yl]-ethyl}- |
| | piperidine-1-carboxylic acid (3,4-dichloro-phenyl)-amide, |
| 144 | 4-hydroxy-4-{1-hydroxy-2-[4-(4-trifluoromethyl-phenyl)-piperidin-1-yl]-ethyl}- |
| | piperidine-1-carbothioic acid (3,4-dichloro-phenyl)-amide, |
| 145 | 4-hydroxy-4-{1-hydroxy-2-[4-(4-trifluoromethyl-phenyl)-piperidin-1-yl]-ethyl}- |
| | piperidine-1-carbothioic acid (3,5-dichloro-phenyl)-amide, |
| 146 | 4-{2-[4-(4-chloro-phenyl)-piperidin-1-yl]-1-hydroxy-ethyl}-4-hydroxy- |
| | piperidine-1-carboxylic acid tert-butyl ester, |
| 147 | 4-{2-[4-(5-amino-1H-indol-3-yl)-piperidin-1-yl]-1-hydroxy-ethyl}-piperidine-1- |
| | carboxylic acid benzyl ester, |
| 148 | 1-(4-hydroxy-4-{1-hydroxy-2-[4-(5-morpholin-4-yl-1H-indol-3-yl)-piperidin-1- |
| | yl]-ethyl}-piperidin-1-yl)-3-(3,4,5-trifluoro-phenyl)propenone, |
| 149 | 3-(3,5-difluoro-phenyl)1-(4-hydroxy-4-{1-hydroxy-2-[4-(5-morpholin-4-yl-1H- |
| | indol-3-yl)-piperidin-1-yl]-ethyl}-piperidin-1-yl)-propenone, |
| 150 | 3-(3,5-bis-trifluoromethyl-phenyl)-1-(4-hydroxy-4-{1-hydroxy-2-[4-(5- |
| | morpholin-4-yl-1H-indol-3-yl)-piperidin-1-yl]-ethyl}-piperidin-1-yl)-propenone, |
| 151 | 3-(3,5-dichloro-phenyl)-1-(4-hydroxy-4-{1-hydroxy-2[4-(5-morpholin-4-yl-1H- |
| | indol-3-yl)-piperidin-1-yl]-ethyl}-piperidin-1-yl)-propenone, |
| 152 | 1-(4-{2-[4-(4-fluoro-phenyl)-4-hydroxy-piperidin-1-yl]-1-hydroxy-ethyl}-4- |
| | hydroxy-piperidin-1-yl)-3-(3,4,5-trifluoro-phenyl)-propenone, |
| 153 | 3-(3,4-difluoro-phenyl)-1-(4-{2-[4-(4-fluoro-phenyl)-4-hydroxy-piperidin-1-yl]- |
| | 1-hydroxy-ethyl}-4-hydroxy-piperidin-1-yl)-propenone, |
| 154 | 3-(3,5-difluoro-phenyl)-1-(4-{2-[4-(4-fluoro-phenyl)-4-hydroxy-piperidin-1-yl]- |
| | 1-hydroxy-ethyl}-4-hydroxy-piperidin-1-yl)-propenone, |
| 155 | 1-(4-{2-[4-(5-fluoro-1H-indol-3-yl)-piperidin-1-yl]-1-hydroxy-ethyl}-4- |
| | hydroxy-piperidin-1-yl)-3-(3,4,5-trifluoro-phenyl)-propenone, |
| 156 | 3-(3,4-difluoro-phenyl)-1-(4-{2-[4-(5-fluoro-1H-indol-3-yl)-piperidin-1-yl]-1- |
| | hydroxy-ethyl}-4-hydroxy-piperidin-1-yl)-propenone, |
| 157 | 3-(3,5-difluoro-phenyl)-1-(4-{2-[4-(5-fluoro-1H-indol-3-yl)-piperidin-1-yl]-1- |
| | hydroxy-ethyl}-4-hydroxy-piperidin-1-yl)-propenone, |
| 158 | 3-(3,4-dichloro-phenyl)-1-(4-{2-[4-(5-fluoro-1H-indol-3-yl)-piperidin-1-yl]-1- |
| | hydroxy-ethyl}-4-hydroxy-piperidin-1-yl)-propenone, |
| 159 | 3-(3-bromo-4-fluoro-phenyl)-1-(4-{2-[4-(5-fluoro-1H-indol-3-yl)-piperidin-1- |
| | yl[-1-hydroxy-ethyl}-4-hydroxy-piperidin-1-yl)-propenone, |
| 160 | 3-(4-chloro-phenyl)-1-(4-{2-[4-(5-fluoro-1H-indol-3-yl)-piperidin-1-yl]-1- |
| | hydroxy-ethyl}-4-hydroxy-piperidine-1-yl)-propan-1-one, |
| 161 | 2-(2-chloro-5-fluoro-phenyl)-1-(4-{2-[4-(5-fluoro-1H-indol-3-yl)-piperidin-1- |
| | yl]1-1hydroxy-ethyl}-4-hydroxy-piperidin-1-yl)-ethanone, |
| 162 | 2-(3,4-dichloro-phenyl)-1-(4-{2-[4(5-fluoro-1H-indol-3-yl)-piperidin-1-yl]-1- |
| | hydroxy-ethyl}-4-hydroxy-piperidin-1-yl)-ethanethione, |
| 163 | 1-(4-{2-[4-(4-chloro-phenyl)-piperidin-1-yl]-1-hydroxy-ethyl}-4-hydroxy- |
| | piperidin-1-yl)-3-(3,4,5-trifluoro-phenyl)-propenone, |
| 164 | 1-(4-{2-[4-(4-chloro-phenyl)-piperidin-1-yl]-1-hydroxy-ethyl}-4-hydroxy- |
| | piperidin-1-yl)-3-(3,5-difluoro-phenyl)-propenone, |
| 165 | 4-{2-[4-(4-chloro-phenyl)-piperidin-1-yl]-1-hydroxy-ethyl}-4-hydroxy- |
| | piperidine-1-carboxylic acid (3,4-dichloro-phenyl)-amide, |
| 166 | 4-{2-[4-(4-chloro-phenyl)-piperidin-1-yl]-1-hydroxy-ethyl}-4-hydroxy- |
| | piperidine-1-carbothioic acid (2,3,5-trifluoro-phenyl)-amide, |
| 167 | 4-{2-[4-(5-fluoro-1H-indol-3-yl)-piperidin-1-yl]-1-hydroxy-ethyl}-4-hydroxy- |
| | piperidine-1-carboxylic acid (3,4-dichloro-phenyl)-amide, |
| 168 | 3-(3,4-dichloro-phenyl)-1-(4-{2-[4-(5-fluoro-1H-indol-3-yl)-piperidin-1-yl]-1- |
| | hydroxy-ethyl}-4-hydroxy-piperidini1iyl)-propan-1-one, |
| 169 | 3-(3,4-dichloro-phenyl)-1-(4-hydroxy-4-{1-hydroxy-2-[4-(5-morpholin-4-yl-1H- |
| | indol-3-yl)-piperidin-1-yl]-ethyl}-piperidin-1-yl)-propenone, |
| 170 | 3-(4-chloro-phenyl)-1-(4-hydroxy-4-{1-hydroxy-2-[4-(5-morpholin-4-yl-1H- |
| | indol-3-yl)-piperidin-1-yl]-ethyl}-piperidin-1-yl)-propenone, |
| 171 | 3-(4-fluoro-phenyl)-1-(4-hydroxy-4-{1-hydroxy-2-[4-(5-morpholin-4-yl-1H- |
| | indol-3-yl)-piperidin-1-yl]-ethyl}-piperidin-1-yl)-propenone, |
| 172 | 3-(3,4-difluoro-phenyl)-1-(4-hydroxy-4-{1-hydroxy-2-[4-(5-morpholin-4-yl-1H- |
| | indol-3-yl)-piperidin-1-yl]-ethyl}-piperidin-1-yl)-propenone, |
| 173 | 3-(3-bromo-4-fluoro-phenyl)-1-(4-hydroxy-4-{1-hydroxy-2-[4 (5- morpholin-4- |
| | yl-1H-indol-3-yl)-piperidin-1-yl]-ethyl}-piperidin-1-yl)-propenone, |
| 174 | 1-(4-hydroxy-4-{1-hydroxy-2-[4-(5-morpholin-4-yl-1H-indol-3-yl)-piperidin-1- |
| | yl]-ethyl}-piperidin-1-yl)-3-m-tolyl-propenone, |
| 175 | 1-(4-hydroxy-4-{1-hydroxy-2-[4-(5-morpholin-4-yl-1H-indol-3-yl)-piperidin-1- |
| | yl[-ethyl}-piperidin-1-yl)-3-(2,4,5-trifluoro-phenyl)-propenone, |
| 176 | 1-(4-hydroxy-4-{1-hydroxy-2-[4-(5-morpholin-4-yl-1H-indol-3-yl)-piperidin-1- |
| | yl]-ethyl}-piperidin-1-yl)-2-(3-nitro-phenyl)-ethanone, |
| 177 | 1-(4-hydroxy-4-{1-hydroxy-2-[4-(5-morpholin-4-yl-1H-indol-3-yl)-piperidin-1- |
| | yl]-ethyl}-piperidin-1-yl)-3-(3-methoxy-phenyl)-propenone, |
| 178 | 1-(4-hydroxy-4-{1-hydroxy-2-[4-(5-morpholin-4-yl-1H-indol-3-yl)-piperidin-1- |
| | yl]-ethyl}-piperidin-1-yl)-3-thiophen-3-yl-propenone, |
| 179 | 4-hydroxy-4-{1-hydroxy-2-[4-(5-morpholin-4 yl-1H-indol-3-yl)-piperidin-1-yl]- |
| | ethyl}-piperidine-1-carboxylic acid (3,4-difluoro-phenyl)-amide, |
| 180 | 4-hydroxy-4-{1-hydroxy-2-[4-(5-morpholin-4-yl-1H-indol-3-yl)-piperidin-1-yl]- |
| | ethyl}-piperidine-1-carboxylic acid (3-fluoro-5-trifluoromethyl-phenyl)-amide, |
| 181 | 4-hydroxy-4-{1-hydroxy-2-[4-(5-morpholin-4-yl-1H-indol-3-yl)-piperidin-1-yl]- |
| | ethyl}-piperidine-1-carboxylic acid (3,4-dichloro-phenyl)-amide, |
| 182 | 4-hydroxy-4-{1-hydroxy-2-[4-(5-morpholin-4-yl-1H-indol-3-yl)-piperidin-1-yl]- |
| | ethyl}-piperidine-1-carbothioic acid (3,5-dichloro-phenyl)-amide, |
| 183 | 4-hydroxy-4-{1-hydroxy-2-[4-(5-morpholin-4-yl-1H-indol-3-yl)-piperidin-1-yl]- |
| | ethyl}-piperidine-1-carbothioic acid (3,5-difluoro-phenyl)-amide, |
| 184 | 4-{2-[4-(5-acetylamino-1H-indol-3-yl)-piperidin-1-yl]-1-hydroxy-ethyl}- |
| | piperidine-1-carboxylic acid benzyl easter, and |
| 185 | N-{3-[1-(2-hydroxy-2-piperidin-4-yl-ethyl)-piperidin-4-yl]-1H-indol-5-yl}- |
| | acetamide. |

An example of a compound of Formula (I) or a form thereof is selected from the consisting of:

| Cpd | Name |
|---|---|
| 4 | 4-{1-hydroxy-2-[4-(1H-indol-3-yl)-piperidin-1-yl]-ethyl}- |
| | piperidine-1-carboxylic acid (3,4-dichloro-phenyl)-amide, |
| 5 | 4-{1-hydroxy-2-[4-(1H-indol-3-yl)-piperidin-1-yl]-ethyl}- |
| | piperidine-1-carboxylic acid (3,5-difluoro-phenyl)-amide, |
| 6 | 3-(3,4-dichloro-phenyl)-1-(4-{1-hydroxy-2-[4-(1H-indol-3-yl)- |
| | piperidin-1-yl]-ethyl}-piperidin-1-yl)-propenone, |
| 7 | 3-(3,5-difluoro-phenyl)-1-(4-{1-hydroxy-2-[4-(1H-indol-3-yl)- |
| | piperidin-1-yl]-ethyl}-piperidin-1-yl)-propenone, |
| 9 | 4-{1-hydroxy-2-[4-(1H-indol-3-yl)-piperidin-1-yl]-ethyl}- |
| | piperidine-1-carbothioic acid (3-bromo-phenyl)-amide, |
| 10 | 3,5-difluoro-N-[(4-{1-hydroxy-2-[4-(1H-indol-3-yl)-piperidin-1- |
| | yl]-ethyl}-piperidin-1-yl)-imino-methyl]-benzamide, |
| 11 | 1-(4-{1-hydroxy-2-[4-(1H-indol-3-yl)-piperidin-1-yl]-ethyl}- |
| | piperidin-1-yl)-3-m-tolyl-propenone, |
| 12 | 3-(3,5-difluoro-phenyl)-1-(4-[1-hydroxy-2-[4-(4-methoxy-phenyl)- |
| | piperidin-1-yl]-ethyl}-piperidin-1-yl)-propenone, |
| 13 | 1-(4-{1-hydroxy-2-[4-(4-methoxy-phenyl)-piperidin-1-yl]-ethyl}- |
| | piperidin-1-yl)-3-(3,4,5-trifluoro-phenyl)-propenone, |
| 14 | 3-(3,4-dichloro-phenyl)-1-(4-{1-hydroxy-2-[4-(4-methoxy-phenyl)- |
| | piperidin-1-yl]-ethyl}-piperidin-1-yl)-propenone, |
| 15 | 4-{1-hydroxy-2-[4-(4-methoxy-phenyl)-piperidin-1-yl]-ethyl}- |
| | piperidine-1-carboxylic acid (3,4-dichloro-phenyl)-amide, |
| 16 | 4-{1-hydroxy-2-[4-(4-methoxy-phenyl)-piperidin-1-yl]-ethyl}- |
| | piperidine-1-carbothioic acid (3-bromo-phenyl)-amide, |
| 19 | 1-(4-{1-hydroxy-2-[4-(1H-pyrrolo[2,3-b]pyridin-3-yl)-piperidin-1- |
| | yl]-ethyl}-piperidin-1-yl)-3-m-tolyl-propenone, |
| 20 | 3,(3,5-difluoro-phenyl)-1-(4-{1-hydroxy-2-[4-(1H-pyrrolo[2,3- |
| | b]pyridin-3yl)-piperidin-1-yl]-ethyl}-piperidin-1-yl)-propenone, |
| 21 | 1-(4-{1-hydroxy-2-[4-(1H-pyrrolo[2,3-b]pyridin-3-yl)-piperidin-1- |
| | yl]-ethyl}-piperidin-1-yl)-3-(3,4,5-trifluoro-phenyl)-propenone, |
| 22 | 1-(4-{1-hydroxy-2-[4-(1H-pyrrolo[2,3-b]pyridin-3-yl)-piperidin-1- |
| | yl]-ethyl}-piperidin-1-yl)-3-(3-trifluoromethyl-phenyl)-propenone, |
| 23 | 3-(3,4-dichloro-phenyl)-1-(4-{1-hydroxy-2-[4-(1H-pyrrolo[2,3- |
| | b]pyridin-3-yl)-piperidin-1-yl]-ethyl}-piperidin-1-yl)-propenone, |
| 24 | 4-{1-hydroxy-2-[4-(1H-pyrrolo[2,3-b]pyridin-3-yl)-piperidin-1-yl]- |
| | ethyl}-piperidine-1-carboxylic acid (3,4-dichloro-phenyl)amide, |
| 26 | 3-(3,5-difluoro-phenyl)-1-(4-{1-hydroxy-2-[4-(5-methoxy-1H- |
| | indol-3-yl)-piperidin-1-yl]-ethyl}-piperidin-1-yl)-propenone, |
| 27 | N-{3-[1-(2-hydroxy-2-{1-[3-(3,4,5-trifluoro-phenyl)-acryloyl]- |
| | piperidin-4-yl}-ethyl)-piperidin-4-yl]-1H-indol-5-yl}- |
| | mathanesulfonamide, |
| 29 | 1-(4-{1-hydroxy-2-[(4-methoxy-phenyl)-piperidin-1-yl[-ethyl}- |
| | piperidin-1-yl)-3-m-tolyl-propenone, |
| 30 | 1-(4-{1-hydroxy-2-[4-(4-methyoxy-phenyl)-piperidin-1-yl]-ethyl}- |
| | piperidin-1-yl)-3-(3-trifluoromethyl-phenyl)-propenone, |
| 32 | 3-(3,4-difluoro-phenyl)-1-(4-{1-hydroxy-2-[4-(4-methoxy-phenyl)- |
| | piperidin-1-yl]-ethyl}-piperidin-1-yl)-propenone, |
| 39 | 3-[4-(4-methoxy-phenyl)-piperidin-1-yl]-2-{1-[3-(3,4,5-trifluoro- |
| | phenyl)-acryloyl]-piperidin-4-yl}-propionic acid, |
| 40 | N-[3-(1-{2-hydroxy-2-[1-(3-m-tolyl-acryloyl)-piperidin-4- |
| | yl]-ethyl}-piperidin-4-yl)-1H-indol-5-yl]-methanesulfonamide, |
| 41 | N-{3-[1-(2-{1-[3-(3,5-difluoro-phenyl)-acrloyl]-piperidin-4-yl}-2- |
| | hydroxy-ethyl)-piperidin-4-yl]-1H-indol-5-yl}- |
| | methanesulfonamide, |
| 43 | 3-(3,4-dichloro-phenyl)-1-(4-{1-hydroxymethyl-2-[4-(4-methoxy- |
| | phenyl)-piperidin-1-yl]-ethyl}-piperidin-1-yl)-propenone, |
| 45 | 3-(3,5-difluoro-phenyl)-1-(4-{1-hydroxymethyl-2-[4-(4-methoxy- |
| | phenyl)-piperidin-1-yl]-ethyl{-piperidin-1-yl)-propenone, |
| 48 | 2-[4-(1H-indol-3-yl)-piperidin-1-yl]-3-[1-(3-m-tolyl-acryloyl)- |
| | piperidin-4-yl]-propionic acid ethyl ester, |
| 49 | 3-{1-[3-(3,4-dichloro-phenyl)-acryloyl]-piperidin-4-yl}-2-[4-(1H- |
| | indol-3-yl)-piperidin-1-yl]-propionic acid ethyl ester, |
| 50 | 2-[4-(1H-indol-3-yl)-piperidin-1-yl]-3-{1-[3-(3,4,5-trifluoro- |
| | phenyl)-acryloyl]-piperidin-4-yl}-propionic acid ethyl ester, |
| 51 | 3-{1-[3-(3,5-difluoro-phenyl)-acryloyl]-piperidin-4-yl}-2-[4-(1H- |
| | indol-3-yl)-piperidin-1-yl]-propionic acid ethyl ester, |
| 52 | 3-[1-(3,4-dichloro-phenylcarbamoyl)-piperidin-4-yl]-2-[4-(1H- |
| | indol-3-yl)-piperidin-1-yl]-propionic acid ethyl ester, |
| 53 | 2-[4-(1H-indol-3-yl)-piperidin-1-yl]-3-[1-(3-m-tolyl-acryloyl)- |
| | piperidin-4-yl]-propionic acid, |
| 58 | 4-{3-hydroxy-2-[4-(1H-indol-3-yl)-piperidin-1-yl]-propyl}- |
| | piperidine-1-carboxylic acid (3,4-dichloro-phenyl)-amide, |
| 62 | 3-[1-(3,4-dichloro-phenylcarbamoyl)-piperidin-4-yl]-2-[4-(1H- |
| | indol-3-yl)-piperidin-1-yl]-propionic acid, |
| 63 | 4-{1-hydroxy-2-[4-(5-methanesulfonylamino-1H-indol-3-yl)- |
| | piperidin-1-yl]-ethyl}-piperidine-1-carboxylic acid tert-butyl ester, |
| 65 | 1-(4-{1-amino-2-[4-(4-methoxy-phenyl)-piperidin-1-yl]-ethyl}- |
| | piperidin-1-yl)-3-(3,4,5-trifluoro-phenyl)-propenone, |
| 70 | 1-(4-hydroxy-4-{1-hydroxy-2-[4-(1H-indol-3-yl)-piperidin-1- |
| | yl]-ethyl}-piperidin-1-yl)-3-(3,4,5-trifluoro-phenyl)-propenone, |
| 71 | 3-(3,5-difluoro-phenyl)-1-(4-hydroxy-4-{1-hydroxy-2-[4-(1H- |
| | indol-3-yl)-piperidin-1-yl]-ethyl}-piperidin-1-yl)-propenone, |
| 73 | 3-(3,5-dichloro-phenyl)-1-(4-hydroxy-4-{1-hydroxy-2-[4-(1H- |
| | indol-3-yl)-piperidin-1-yl]-ethyl}-piperidin-1-yl)-propenone, |
| 74 | 3-(3-chloro-5-fluoro-phenyl)-1-(4-hydroxy-4-{1-hydroxy-2-[4-(1H- |
| | indol-3-yl)-piperidin-1-yl]-ethyl]-piperidin-1-yl)-propenone, |
| 75 | 3-(3,4-dichloro-phenyl)-1-(4-hydroxy-4-[1-hydroxy-2-[4-(1H- |
| | indol-3-yl)-piperidin-1-yl]-ethyl}-piperidin-1-yl)-propenone, |
| 77 | 1-(4-hydroxy-4-{1-hydroxy-2-[4-(1H-indol-3-yl)-piperidin-1- |
| | yl]-ethyl}-piperidin-1-yl)-3-(3-trifluoromethyl-phenyl)-propenone, |
| 78 | 3-(3,4-difluoro-phenyl)-1-(4-hydroxy-4-{1-hydroxy-2-[4-(1H- |
| | indol-3-yl)-piperidin-1-yl]-ethyl}-piperdin-1-yl)-propenone, |
| 80 | 3-(3-bromo-4-fluoro-phenyl)-1-(4-hydroxy-4-{1-hydroxy-2-[4-(1H- |
| | indol-3-yl)-piperidin-1-yl]-ethyl}-piperidin-1-yl)-propenone, |
| 84 | 4-hydroxy-4-{1-hydroxy-2-[4-(1H-indol-3-yl)-piperidin-1-yl]- |
| | ethyl}-piperidine-1-carbothioic acid (3,4-dichloro-phenyl)-amide, |
| 85 | 1-(4-{2-[4-(1H-benzoimidazol-2-yl)-piperidin-1-yl]-1-hydroxy- |
| | ethyl}-piperidin-1-yl)-3-(3,4-dichloro-phenyl)-propenone, |
| 86 | 1-(4-{2-[4-(1H-benzoimidazol-2-yl)-piperidin-1-yl]-1-hydroxy- |
| | ethyl}-piperidin-1-yl)-3-(3,4,5-trifluoro-phenyl)-propenone, |
| 88 | 1-(4-hydroxy-4-{1-hydroxy-2-[4-(4-methoxy-phenyl)-piperidin-1- |
| | yl]-ethyl}-piperidin-1-yl)-3-(3,4,5-trifluoro-phenyl)-propenone, |
| 90 | 4-{1-hydroxy-2-[4-(4-methoxy-phenyl)-piperidin-1-yl]-ethyl}- |
| | piperidine-1-carboxylic acid (4,5-dichloro-2-fluoro-phenyl)-amide, |
| 93 | 3-(3,4-difluoro-phenyl)-1-(4-hydroxy-4-{1-hydroxy-2-[4-(4- |
| | methoxy-phenyl)-piperidin-1-yl]-ethyl}-piperidin-1-yl)-propenone, |
| 94 | 3-(3,5-difluoro-phenyl)-1-(4-hydroxy-4-{1-hydroxy-2-[4-(4- |
| | methoxy-phenyl)-piperidin-1-yl]-ethyl}-piperidin-1-yl)-propenone, |
| 95 | 1-(4-hydroxy-4-{1-hydroxy-2-[4-(4-methoxy-phenyl)-piperidin-1- |
| | yl]-ethyl}-piperidin-1-yl)-3-m-tolyl-propenone, |
| 100 | 4-{1-hydroxy-2-[4-(4-methoxy-phenyl)-piperidin-1-yl]-ethyl}- |
| | piperidine-1-carboxylic acid benzo[1,3]dioxol-5-ylamide, |
| 101 | 4-{1-hydroxy-2-[4-(4-methoxy-phenyl)-piperidin-1-yl]-ethyl}- |
| | piperidine-1-carboxylic acid (2-chloro-4-trifluoromethyl-phenyl)- |
| | amide, |
| 107 | 4-{1-hydroxy-2-[4-(4-methoxy-phenyl)-piperidin-1-yl]-ethyl}- |
| | piperidine-1-carbothioic acid (3,5-difluoro-phenyl)-amide, |
| 110 | 4-{1-hydroxy-2-[4-(4-methoxy-phenyl)-piperidin-1-yl]-ethyl}- |
| | piperidine-1-carbothioic acid (4-bromo-3-fluoro-phenyl)-amide, |
| 114 | 4-hydroxy-4-{1-hydroxy-2-[4-(4-methoxy-phenyl)-piperidin-1-yl]- |
| | ethyl}-piperidine-1-carboxylic acid (3,4-difluoro-phenyl)-amide, |
| 115 | 4-hydroxy-4-{1-hydroxy-2-[4-(4-methoxy-phenyl)-piperidin-1-yl]- |
| | ethyl}-piperidine-1-carboxylic acid (3,5-difluoro-phenyl)-amide, |
| 119 | 4-hydroxy-4-{1-hydroxy-2-[4-(4-methoxy-phenyl)-piperidin-1-yl]- |
| | ethyl}-piperidine-1-carbothioic acid (3,5-dimethoxy-phenyl)- |
| | amide, |
| 121 | 4-{2-[4-1H-benzoimidazol-2-yl)-piperidin-1-yl]-1-hydroxy- |
| | ethyl}-1-(4-trifluoromethyl-pyrimidin-2-yl)-piperidin-4-ol, |
| 122 | 4-{1-hydroxy-2-[4-(5-morpholin-4-yl-1H-indol-3-yl)-piperidin-1- |
| | yl]-ethyl}-1-(4-trifluoromethyl-pyrimidin-2-yl)-piperidin-4-ol, |
| 123 | 4-{1-hydroxy-2-[4-(5-methyl-1H-indol-3-yl)-piperidin-1-yl]- |
| | ethyl}-1-(4-trifluoromethyl-pyrimidin-2-yl)-piperidin-4-ol, |
| 124 | 3-(1-{2-hydroxy-2-[4-hydroxy-1-(4-trifluoromethyl-pyrimidin-2- |
| | yl)-piperidin-4-yl]-ethyl}-piperidin-4-yl)-1H-indole-5-carbonitrile, |
| 125 | 4-{2-[4-(4-fluoro-phenyl)-piperidin-1-yl]-1-hydroxy-ethyl}-4- |
| | hydroxy-piperidine-1-carboxylic acid benzyl ester, |
| 126 | 4-hydroxy-4-{1-hydroxy-2-[4-(4-trifluoromethyl-phenyl)-piperidin- |
| | 1-yl]-ethyl}-piperidine-1-carboxylic acid benzyl ester, |
| 127 | 2-[4-(4-methoxy-phenyl)-piperidin-1-yl]-1-(5′-nitro-3,4,5,6- |
| | tetrahydro-2H-[1,2′]bipyridinyl-4-yl)-ethanol, |
| 128 | 1-(4-{2-[4-(4-fluoro-phenyl)-piperidin-1-yl]-1-hydroxy-ethyl}- |
| | 4-hydroxy-piperidin-1-yl)-3-(3,4,5-trifluoro-phenyl)-propenone, |
| 130 | 3-(3,5-difluoro-phenyl)-1-(4-{2-[4-(4-fluoro-phenyl)-piperidin-1- |
| | yl]-1-hydroxy-ethyl}-4-hydroxy-piperidin-1-yl)-propenone, |
| 131 | 3-(3,4-dichloro-phenyl)-1-(4-{2-[4-(4-fluoro-phenyl)-piperidin-1- |
| | yl]-1-hydroxy-ethyl}-4-hydroxy-piperidin-1-yl)-propenone, |
| 134 | 4-{2-[4-(4-fluoro-phenyl)-piperidin-1-yl]-1-hydroxy-ethyl}-4- |
| | hydroxy-piperidine-1-carboxylic acid (3,4-dichloro-phenyl)-amide, |
| 136 | 1-(4-hydroxy-4-{1-hydroxy-2-[4-(4-trifluoromethyl-phenyl)- |
| | piperidin-1-yl]-ethyl}-piperidin-1-yl)-3-(3,4,5-trifluoro-phenyl)- |
| | propenone, |
| 137 | 3-(3,4-difluoro-phenyl)-1-(4-hydroxy-4-{1-hydroxy-2-[4-(4- |
| | trifluoromethyl-phenyl)-piperidin-1-yl]-ethyl}-piperidin-1-yl)- |
| | propenone, |
| 138 | 3-(3,5-difluoro-phenyl)-1-(4-hydroxy-4-{1-hydroxy-2-[4-(4- |
| | trifluoromethyl-phenyl)-piperidin-1-yl]-ethyl}-piperidin-1-yl)- |
| | propenone, |
| 142 | 4-hydroxy-4-{1-hydroxy-2-[4-(4-trifluoromethyl-phenyl)-piperidin- |
| | 1 yl]-ethyl}-piperidine-1-carboxylic acid (3,5-difluoro-phenyl)- |
| | amide, |
| 146 | 4-{2-[4-(4-chloro-phenyl)-piperidin-1-yl]-1-hydroxy-ethyl}-4- |
| | hydroxy-piperidine-1-carboxylic acid tert-butyl ester, |
| 151 | 3-(3,5-dichloro-phenyl)-1-(4-hydroxy-4-{1-hydroxy-2[4-(5- |
| | morpholin-4-yl-1H-indol-3-yl)-piperidin-1-yl]-ethyl}-piperidin-1- |
| | yl)-propenone, |
| 152 | 1-(4-{2-[4-(4-fluoro-phenyl)-4-hydroxy-piperidin-1-yl]-1-hydroxy- |
| | ethyl}-4-hydroxy-piperidin-1-yl)-3-(3,4,5-trifluoro-phenyl)- |
| | propenone, |
| 153 | 3-(3,4-difluoro-phenyl)-1-(4-{2-[4-(4-fluoro-phenyl)-4-hydroxy- |
| | piperidin-1-yl]-1-hydroxy-ethyl}-4-hydroxy-piperidin-1-yl)- |
| | propenone, |
| 155 | 1-(4-{2-[4-(5-fluoro-1H-indol-3-yl)-piperidin-1-yl]-1-hydroxy- |
| | ethyl}-4-hydroxy-piperidin-1-yl)-3-(3,4,5-trifluoro-phenyl)- |
| | propenone, |
| 156 | 3-(3,4-difluoro-phenyl)-1-(4-{2-[4-(5-fluoro-1H-indol-3-yl)- |
| | piperidin-1-yl]-1-hydroxy-ethyl}-4-hydroxy-piperidin-1-yl)- |
| | propenone, |
| 157 | 3-(3,5-difluoro-phenyl)-1-(4-{2-[4-(5-fluoro-1H-indol-3-yl)- |
| | piperidin-1-yl]-1-hydroxy-ethyl}-4-hydroxy-piperidin-1-yl)- |
| | propenone, |
| 163 | 1-(4-{2-[4-(4-chloro-phenyl)-piperidin-1-yl]-1-hydroxy-ethyl}-4- |
| | hydroxy-piperidin-1-yl)-3-(3,4,5-trifluoro-phenyl)-propenone, |
| 164 | 1-(4-{2-[4-(4-chloro-phenyl)-piperidin-1-yl]-1-hydroxy-ethyl}-4- |
| | hydroxy-piperidin-1-yl)-3-(3,5-difluoro-phenyl)-propenone, |
| 165 | 4-{2-[4-(4-chloro-phenyl)-piperidin-1-yl]-1-hydroxy-ethyl}-4- |
| | hydroxy-piperidine-1-carboxylic acid (3,4-dichloro-phenyl)-amide, |
| 167 | 4-{2-[4-(5-fluoro-1H-indol-3-yl)-piperidin-1-yl]-1-hydroxy- |
| | ethyl}-4- |
| | hydroxy-piperidine-1-carboxylic acid (3,4-dichloro-phenyl)-amide, |
| 172 | 3-(3,4-difluoro-phenyl)-1-(4-hydroxy-4-{1-hydroxy-2-[4-(5- |
| | morpholin-4-yl-1H-indol-3-yl)-piperidin-1-yl]-ethyl}-piperidin- |
| | 1-yl)-propenone, |
| 174 | 1-(4-hydroxy-4-{1-hydroxy-2-[4-(5-morpholin-4-yl-1H-indol-3-yl)- |
| | piperidin-1-yl]-ethyl}-piperidin-1-yl)-3-m-tolyl-propenone, |
| 175 | 1-(4-hydroxy-4-{1-hydroxy-2-[4-(5-morpholin-4-yl-1H-indol-3-yl)- |
| | piperidin-1-yl[-ethyl}-piperidin-1-yl)-3-(2,4,5-trifluoro-phenyl)- |
| | propenone, |
| 176 | 1-(4-hydroxy-4-{1-hydroxy-2-[4-(5-morpholin-4-yl-1H-indol-3-yl)- |
| | piperidin-1-yl]-ethyl}-piperidin-1-yl)-2-(3-nitro-phenyl)-ethanone, |
| 177 | 1-(4-hydroxy-4-{1-hydroxy-2-[4-(5-morpholin-4-yl-1H-indol-3-yl)- |
| | piperidin-1-yl]-ethyl}-piperidin-1-yl)-3-(3-methoxy-phenyl)- |
| | propenone, |
| 178 | 1-(4-hydroxy-4-{1-hydroxy-2-[4-(5-morpholin-4-yl-1H-indol-3-yl)- |
| | piperidin-1-yl]-ethyl}-piperidin-1-yl)-3-thiophen-3-yl-propenone, |
| 181 | 4-hydroxy-4-{1-hydroxy-2-[4-(5-morpholin-4-yl-1H-indol-3-yl)- |
| | piperidin-1-yl]-ethyl}-piperidine-1-carboxylic acid (3,4-dichloro- |
| | phenyl)-amide, |
| 183 | 4-hydroxy-4-{1-hydroxy-2-[4-(5-morpholin-4-yl-1H-indol-3-yl)- |
| | piperidin-1-yl]-ethyl}-piperidine-1-carbothioic acid (3,5-difluoro- |
| | phenyl)-amide, |
| 184 | 4-{2-[4-(5-acetylamino-1H-indol-3-yl)-piperidin-1-yl]-1-hydroxy- |
| | ethyl}-piperidine-1-carboxylic acid benzyl easter, and |
| 185 | N-{3-[1-(2-hydroxy-2-piperidin-4-yl-ethyl)-piperidin-4-yl]-1H- |
| | indol-5-yl}-acetamide. |

The invention includes the use of an instant compound for the preparation of a composition or medicament for preventing, treating or ameliorating a CCR2 mediated inflammatory syndrome, disorder or disease in a subject in need thereof, wherein the composition or medicament comprises a mixture one or more compounds of the invention and an optional pharmaceutically acceptable carrier.

The term “composition” means a product comprising at least a compound of the invention, such as a product comprising the specified ingredients in the specified amounts, as well as any product which results, directly or indirectly, from such combinations of the specified ingredients in the specified amounts and one or more pharmaceutically acceptable carriers or any such alternatives to a compound of the invention and a pharmaceutically acceptable carrier therefor.

The term “medicament” means a product for use in preventing, treating or ameliorating a CCR2 mediated inflammatory syndrome, disorder or disease.

The term “pharmaceutically acceptable” means molecular entities and compositions that are of sufficient purity and quality for use in the formulation of a composition or medicament of the invention and that, when appropriately administered to an animal or a human, do not produce an adverse, allergic, or other untoward reaction. Since both human and veterinary use is included within the scope of the invention, a pharmaceutically acceptable formulation includes a compound of Formula (I) or a form, composition or medicament thereof for either human or veterinary use.

The term “CCR2 mediated inflammatory syndrome, disorder or disease” means, without limitation, syndromes, disorders or diseases associated with elevated MCP-1 expression, MCP-1 overexpression or inflammatory conditions that accompany syndromes, disorders or diseases associated with elevated MCP-1 expression or MCP-1 overexpression.

The terms “elevated MCP-1 expression” or “MCP-1 overexpression” mean unregulated or up-regulated CCR2 activation as a result of MCP-1 binding.

The term “unregulated” means unwanted CCR2 activation in a multicellular organism resulting in harm (such as discomfort or decreased life expectancy) to the multicellular organism.

The term “up-regulated” means: 1). increased or unregulated CCR2 activity or expression, or 2). increased CCR2 expression leading to unwanted monocyte and lymphocyte migration. The existence of an inappropriate or abnormal level of MCP-1 or activity of CCR2 is determined by procedures well known in the art.

CCR2 mediated inflammatory syndromes, disorders or diseases include, without limitation, ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, diabetes and diabetic complications, diabetic nephropathy, diabetic retinopathy, diabetic retinitis, diabetic microangiopathy, obesity, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, or stomach.

The term “uveitis” generically refers to any inflammatory disease involving the eye. Uveitis can be divided into clinically distinct subtypes based on the part of the eye in which the inflammation is present (percentages correspond to patients known to fit these categories): anterior (51%), intermediate (13%), posterior (20%), or panuveitis (16%) and, according to the course of the disease, as either acute (16%), recurring (26%), or chronic (58%). Those with anterior uveitis (19%) eventually develop irreparable vision damage despite aggressive treatment such as unilateral blindness (9%), bilateral blindness (2%), or unilateral or bilateral vision impairment (8%). Most cases of uveitis are idiopathic, but known causes include infection (e.g., toxoplasmosis, cytomegalovirus, and the like) or development as a component of a systemic inflammatory and/or autoimmune disorder (e.g., juvenile RA, HLA-B27-associated spondyloarthropathies, sarcoidosis, and the like).

Patients with anterior uveitis have MCP-1 present in large quantities in the aqueous humor of the eye. The amount of MCP-1 correlates with the severity of the clinical symptoms and the large number of mononuclear cells present in the cellular infiltrate. Uveitis is also a potential complication resulting from cataract surgery and prophylactic use of antibiotics and corticosteroids is common for such patients. Currently, most patients with anterior uveitis are first treated with topical corticosteroids. Injected or oral steroids may be used in severe cases, or if the disease is recurrent or chronic. If steroids are ineffective, immunosuppressive agents (e.g., cyclosporine, methotrexate, azathioprine, cyclophosphamide, and the like) are used, particularly if the patient's vision is in danger. All of these drugs have potentially severe side-effects, particularly in children, and there is general agreement that there is an unmet medical need for safe and effective steroid substitutes or steroid-sparing agents.

An example of the invention is a method for preventing, treating or ameliorating CCR2 mediated ophthalmic disorders (such as uveitis, allergic conjunctivitis and the like), rheumatoid arthritis, psoriasis, psoriatic arthritis, atopic dermatitis, obesity, chronic obstructive pulmonary disease, allergic rhinitis, asthma, allergic asthma, periodontal diseases (such as periodonitis, gingivitis, gum disease and the like) in a subject in need thereof comprising administering to the subject an effective amount of a compound of Formula (I) or a form, composition or medicament thereof.

Another example of the invention is a method for preventing, treating or ameliorating CCR2 mediated uveitis, wherein uveitis includes, without limitation, acute, recurring or chronic uveitis (such as anterior uveitis, intermediate uveitis, posterior uveitis, panuveitis and the like) in a subject in need thereof comprising administering to the subject an effective amount of a compound of Formula (I) or a form, composition or medicament thereof.

An example of the invention is a method for preventing, treating or ameliorating CCR2 mediated acute uveitis, recurring uveitis, chronic uveitis, allergic conjunctivitis, rheumatoid arthritis, psoriasis, psoriatic arthritis, atopic dermatitis, obesity, chronic obstructive pulmonary disease, allergic rhinitis, asthma, allergic asthma, periodonitis, gingivitis or gum disease in a subject in need thereof comprising administering to the subject an effective amount of a compound of Formula (I) or a form, composition or medicament thereof.

Another example of the invention is a method for preventing, treating or ameliorating CCR2 mediated obesity in a subject in need thereof comprising administering to the subject an effective amount of a compound of Formula (I) or a form, composition or medicament thereof.

The invention includes a method for preventing, treating or ameliorating a CCR2 mediated inflammatory syndrome, disorder or disease in a subject in need thereof comprising administering to the subject an effective amount of a compound of Formula (I) or a form, composition or medicament thereof in a combination product with one or more therapeutic agents.

The term “combination product” refers to a compound of Formula (I) or a form, composition or medicament thereof in admixture with a therapeutic agent and an optional carrier for preventing, treating or ameliorating a CCR2 mediated inflammatory syndrome, disorder or disease.

The term “therapeutic agent” refers to one or more anti-inflammatory agents (such as a small molecule, antibiotic, corticosteroid, steroid, and the like), anti-infective agents or immunosuppressive agents.

The term “administering,” in the context of a combination product includes, without limitation, co-administration of the compound and the agent, sequential administration of the compound and the agent, administration of a composition containing of the compound and the agent or simultaneous administration of separate compositions containing of the compound and the agent.

As those skilled in the art will appreciate, the effective amounts of the components comprising the combination product may be independently optimized and combined to achieve a synergistic result whereby the pathology is reduced more than it would be if the components of the combination product were used alone.

The present invention includes a compound of Formula (I) or a form thereof, wherein the compound is labeled with a ligand for use as a marker, and wherein the ligand is a radioligand selected from deuterium, tritium and the like.

Pharmaceutical Compositions

The present invention includes a pharmaceutical composition or medicament comprising one or more of the instant compounds and an optional pharmaceutically acceptable carrier.

The present invention further includes a process for making a pharmaceutical composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration ocularly, intranasally (by inhalation or insufflation), sublingually, orally, parenterally or rectally including, without limitation, ocular (via a delivery device such as a contact lens and the like), intranasal (via a delivery device), transdermal, topical with or without occlusion, intravenous (both bolus and infusion), injection (intraperitoneally, subcutaneously, intramuscularly, intratumorally, or parenterally) and the like.

The composition or medicament may be in a dosage unit such as a tablet, pill, capsule, powder, granule, liposome, biodegradable carrier, ion exchange resin, sterile solution and the like (facilitating immediate release, timed release, or sustained release), parenteral solution or suspension, metered aerosol or liquid spray, drop, ampoule, auto-injector device or suppository.

Compositions or medicaments suitable for oral administration include solid forms such as pills, tablets, caplets, capsules (each including immediate release, timed release, and sustained release formulations), granules and powders and liquid forms such as solutions, syrups, elixirs, emulsions and suspensions. Forms useful for nasal administration include sterile solutions or nasal delivery devices. Forms useful for ocular administration include sterile solutions or ocular delivery devices. Forms useful for parenteral administration include sterile solutions, emulsions and suspensions.

Alternatively, the composition or medicament may be administered in a form suitable for once-weekly or once-monthly administration. For example, an insoluble salt of the active compound may be adapted to provide a depot preparation for intramuscular injection (e.g., a salt form) or to provide a solution for nasal or ocular administration (e.g., a quaternary ammonium salt).

The dosage form (tablet, capsule, powder, solution, contact lens, patch, liposome, ion exchange resin, suppository, teaspoonful, and the like) containing the composition or medicament thereof contains an effective amount of the active ingredient necessary to provide a therapeutic effect.

The composition or medicament may contain an effective amount of from about 0.001 mg to about 5000 mg (preferably, from about 0.001 to about 500 mg) of a compound of the present invention or a pharmaceutically acceptable form thereof and may be constituted into any form suitable for the mode of administration selected for a subject in need.

A contemplated range of the effective amount includes from about 0.001 mg to about 300 mg/kg of body weight per day. A contemplated range also includes from about 0.003 to about 100 mg/kg of body weight per day. Another contemplated range includes from about 0.005 to about 15 mg/kg of body weight per day. Another contemplated range includes from about 0.01 to about 15 mg/kg of body weight per day. Another contemplated range includes from about 0.1 to about 10 mg/kg of body weight per day. The composition or medicament may be administered according to a dosage regimen of from about 1 to about 5 times per day.

For oral administration, the composition or medicament is preferably in the form of a tablet containing, e.g., 0.001, 0.005, 0.01, 0.05, 0.1, 0.5, 1.0, 2.5, 5.0, 10.0, 15.0, 25.0, 50.0, 100, 150, 200, 250, and 500 milligrams of the active ingredient for the symptomatic adjustment of the dosage to the patient to be treated.

Optimal dosages to be administered may be readily determined by those skilled in the art, and will vary with the particular compound used, the mode of administration, the strength of the preparation and the advancement of the disease condition. In addition, factors associated with the particular patient being treated, including patient's sex, age, weight, diet, time of administration and concomitant diseases, will result in the need to adjust dosages. The use of either daily administration or post-periodic dosing may be employed.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette.

Synthetic Methods

Representative compounds of the present invention can be synthesized in accordance with the general synthetic schemes described below and are illustrated more particularly in the specific examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art.

The following abbreviations and formulas have the indicated meanings:

| | |
|---|---|
| HOAc or AcOH | acetic acid |
| CH₃COCl | acetyl chloride |
| atm | atmosphere |
| Boc | tert-butoxy carbonyl or t-butoxy carbonyl |
| (Boc)₂O | di-tert-butyl dicarbonate |
| Cbz | benzyloxycarbonyl |
| (CH₂O)ₙ | paraformaldehyde |
| Cpd | compound |
| DBU | 1,8-diazabicyclo[5.4.0]undec-7-ene |
| DCM or CH₂Cl₂ | methylene chloride or dichloromethane |
| DIPEA or i-Pr₂NEt | diisopropylethylamine |
| DMAP | 4-dimethylaminopyridine |
| DMF | N,N-dimethyl formamide |
| EDCI | 1-(3-dimethylaminopropyl)-3-ethylcarbodiimide |
| | hydrochloride |
| Et₂O | diethyl ether |
| EtOAc or CH₃CO₂Et | ethyl acetate |
| FLIPR | fluorometric imaging plate reader |
| HBr | hydrobromic acid |
| HOBt | 1-hydroxy-benzotriazole |
| HBTU | O-(7-benzotriazol-1-yl)-N,N,N′,N′- |
| | tetramethyluronium hexafluorophosphate |
| K₂CO₃ | potassium carbonate |
| KHMDS | potassium bis(trimethylsilyl)amide |
| LiAlH₄ | lithium aluminum hydride |
| LHMDS | lithium bis(trimethylsilyl)amide |
| LiOH | lithium hydroxide |
| MeCN or CH₃CN | acetonitrile |
| MeOH or CH₃OH | methanol |
| Me₃SiCHN₂ | trimethylsilyl-diazomethane |
| min/hr(s)/dy | minute(s)/hour(s)/day(s) |
| MS | mass spectrum, refers to data |
| | shown as m/z (M + H)⁺ |
| NBS | N-bromo-succinimide |
| NCS | N-chloro-succinimide |
| NH₄Cl | ammonium chloride |
| HCO₂NH₄ | ammonium formate |
| NaBH₄ | sodium borohydride |
| NaB(OAc)₃H | sodium triacetoxyborohydride |
| NaCNBH₃ | sodium cyanoborohydride |
| NaHCO₃ | sodium bicarbonate |
| NaOH | sodium hydroxide |
| Na₂SO₄ | sodium sulfate |
| Pd(OH)₂ | palladium hydroxide |
| psi | pounds per square inch |
| PTLC | preparative thin layer chromatography |
| RPMI | Roswell Park Memorial Institute |
| RT/rt/r.t. | room temperature |
| SOCl₂ | thionyl chloride |
| t-BuOOH | tert-butyl-hydroxy-peroxide |
| TEA or Et₃N | triethylamine |
| TFA | trifluoroacetic acid |
| THF | tetrahydrofuran |
| TLC | thin layer chromatography |
| TMSCl | chlorotrimethylsilane or trimethylsilyl chloride |
| VO(acac)₂ | vanadyl acetylacetonate |

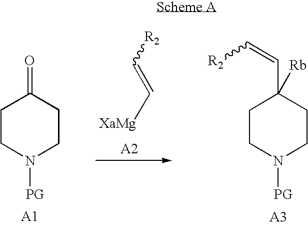

Compound A1 (wherein PG is a suitable protecting group such as benzyl, benzyloxycarbonyl, tert-butoxycarbonyl and the like) is reacted with a solution of Compound A2 (in a solvent such as diethyl ether, tetrahydrofuran and the like or a mixture thereof, wherein Xa is a halogen such as chlorine, bromine, or iodine) to give a Compound A3.

For purposes of Scheme A, Compound A2 represents a compound wherein R₂ is hydrogen. A Compound A2 having a plurality of R₂ substitutents as defined herein is either commercially available or may be prepared according to methods well known to one skilled in the art.

For purposes of Scheme A, as well, Compound A3 represents a compound wherein Rb is hydroxy. A Compound A3 having an Rb substitutent as further defined herein is either commercially available or may be prepared according to methods well known to one skilled in the art. For example, a Compound A3 wherein Rb is hydrogen may be prepared as described in U.S. Pat. No. 6,004,982.

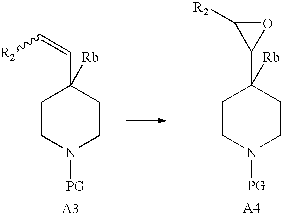

Compound A3 is reacted with a suitable oxidant in solution (such as tert-butyl-hydroxy-peroxide, m-chloro-perbenzoic acid and the like in a solvent such as toluene, benzene, DCM and the like or a mixture thereof) to give a Compound A4.

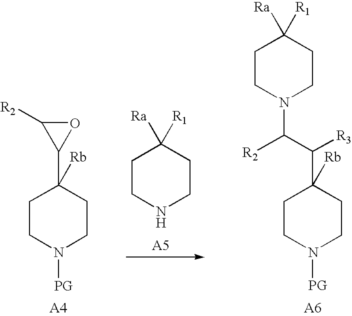

Compound A4 is reacted with a solution of a Compound A5 (in a solvent such as isopropanol, acetonitrile and the like or a mixture thereof) at reflux to give a Compound A6.

A Compound A5 having a plurality of Ra and R₁ substitutents as defined herein is either commercially available or may be prepared according to methods well known to one skilled in the art.

For purposes of Scheme A, Compound A6 represents an intermediate compound of Formula (I) wherein R₃ is hydroxy.

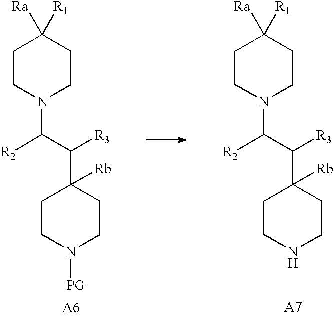

The Compound A6 protecting group may be removed at a suitable point using means known to those skilled in the art to provide a Compound A7 free base or salt form that is amendable for further substitution.

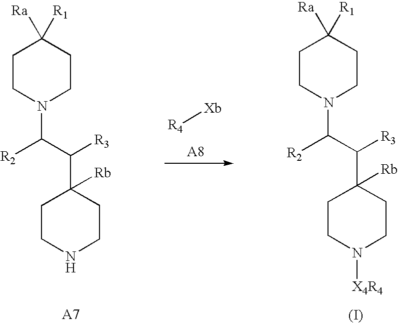

A solution of Compound A7 (in a suitable solvent such as CH₂Cl₂, CH₃CN, DMF and the like or a mixture thereof) is reacted with a Compound A8 (wherein Xb is a suitable leaving group such as halogen, or a suitable reaction group such as isocyanato, isothiocyanato, N-(imino-pyrazol-1-yl-methyl)-aminoacyl, acrylyl-chloride, acrylyl-carboxy and the like) to provide a compound of Formula (I) (wherein certain portions of Xb are incorporated into X₄ as a product of the reaction).

When the Compound A8 Xb reaction group is an acrylyl-carboxy reaction group, Compound A8 is reacted in conjunction with coupling reagents such as EDCl, HBTU and the like.

When Compound A7 is a salt form, Compound A7 is reacted with Compound A8 in the presence of a suitable base such as Et₃N, DIPEA and the like.
Scheme B

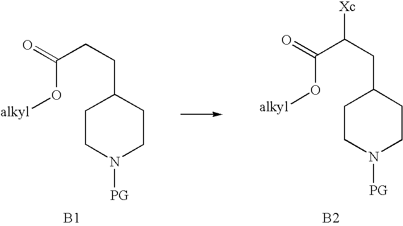

A solution of Compound B1 (wherein PG is a suitable protecting group such as tert-butoxycarbonyl and the like) is reacted with a reagent solution (such as LHMDS in a solvent such as THF and the like). A second reagent (such as TMSCl and the like) is added to the mixture and the mixture is stirred. A halogen reagent solution is added (such as NBS, NCS, bromine and the like in a solvent such as THF and the like) and the mixture is stirred. The reaction provides Compound B2 as a racemate (wherein Xc is a suitable leaving group such as halogen).

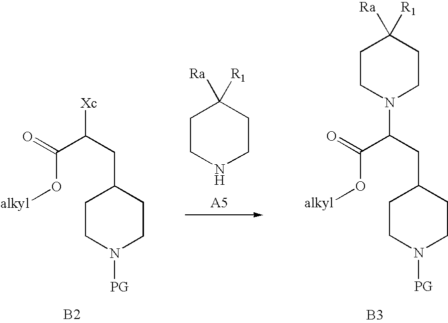

A solution of Compound A5 (in a solvent such as CH₃CN and the like) is reacted with a solution of Compound B2 (in a solvent such as acetonitrile and the like) in the presence of a suitable base (such as Et₃N, DIPEA and the like) to provide Compound B3, representative of a racemate form of an intermediate compound of Formula (I). The racemate Compound B3 may be chromatographically separated into its constituent enantiomers using conventional resolution techniques known to those skilled in the art. Using the procedure of Scheme A, Compound B3 may be carried forward in place of Compound A6 to provide other compounds representative of the present invention.

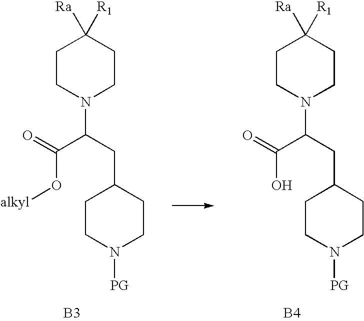

Further, Compound B3 may be reacted with a reducing agent (such as lithium aluminum hydride and the like) to provide a Compound B4, representative of a compound of Formula (I).

Using the procedure of Scheme A, Compound B4 may be further carried forward in place of Compound A6 to provide other compounds representative of the present invention.

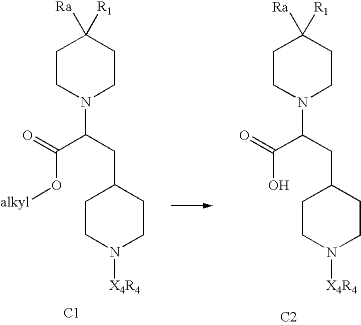

A solution of a Compound C1 (prepared by carrying forward Compound B3 using the procedure of Scheme A) is reacted with an aqueous reagent solution (such as LiOH in a solvent such as MeOH, and the like) to provide a Compound C2, representative of other compounds of the present invention.

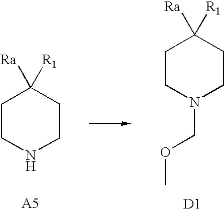

A solution of Compound A5 (in a solvent such as methanol and the like stirred in the presence of paraformaldehyde; wherein R₁ is aryl or heteroaryl) is reacted with a base (such as potassium carbonate and the like) to provide a Compound D1.

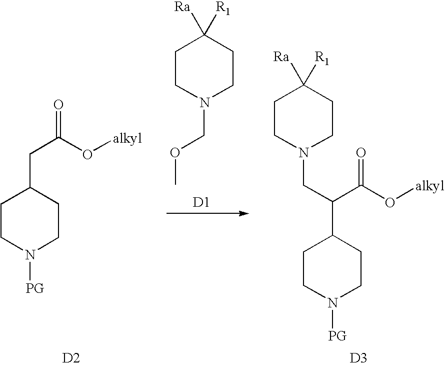

A solution of Compound D2 (in solvent such as DCM and the like; wherein PG is a suitable protecting group such as benzyloxycarbonyl and the like) is reacted with a reagent solution (such as KHMDS in a solvent such as toluene, DCM and the like and mixtures thereof) and the mixture is stirred. A second reagent (such as TMSCl and the like) is added to the mixture and the mixture is stirred. Compound D1 is added followed by a Lewis Acid (such as TiCl₄ and the like) and the mixture is stirred to provide Compound D3.

Using the procedure of Scheme A, Compound D3 may be carried forward in place of Compound A6 to provide other compounds representative of the present invention.

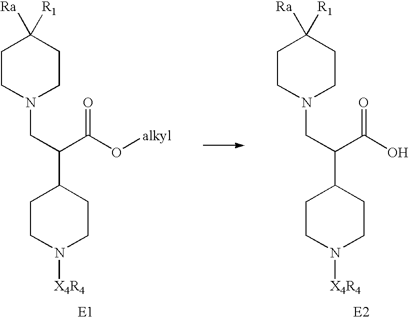

A solution of a Compound E1 is reacted with an aqueous reagent solution (such as LiOH in a solvent such as MeOH, and the like) to provide a Compound E2.

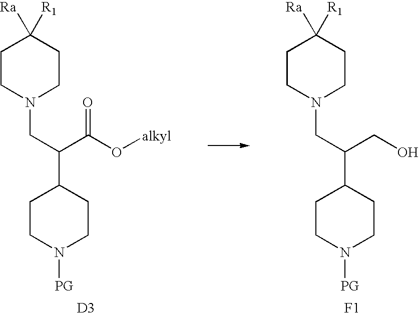

Compound D3 (wherein PG is tert-butoxycarbonyl and the like) is reacted with a reducing agent (such as lithium aluminum hydride and the like) to provide Compound F1.

Using the procedure of Scheme A, Compound F1 is carried forward in place of Compound A6 to provide other compounds representative of the present invention.

The invention is further defined by reference to the following examples, which are merely intended to be illustrative and not limiting.

### EXAMPLE 1

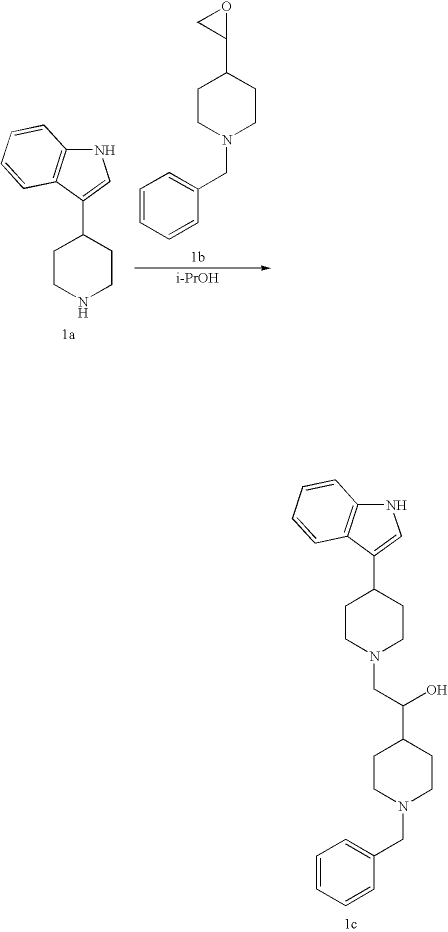

A solution of 3-piperidin-4-yl-1H-indole Compound 1a (200 mg, 1.00 mmol, 1 eq) and 1-benzyl-4-oxiranyl-piperidine Compound 1b (217 mg, 1.00 mmol, 1 eq) in isopropanol (5 mL) was heated at reflux for 48 hrs. The solvents were evaporated and the residue subjected to silica gel chromatography (gradient 10% to 20% 2M MeOH_NH₃ in CH₂Cl₂) to provide 1-(1-benzyl-piperidin-4-yl)-2-[4-(1H-indol-3-yl)-piperidin-1-yl]-ethanol Compound 1c (338 mg, 81%) as a pale foam. MS m/z 418 (M+H)⁺

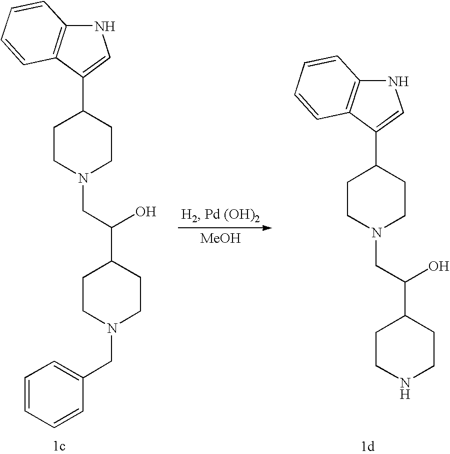

Palladium hydroxide (110 mg, 0.16 mmol, 0.2 eq) was added to a solution of Compound 1c (327 mg, 0.78 mmol, 1 eq) in methanol. The solution was purged with nitrogen, then filled to 50 psi with hydrogen and shaken for 16 hrs. The solution was purged with nitrogen, filtered through Celite and evaporated to provide 2-[4-(1H-indol-3-yl)-piperidin-1-yl]-1-piperidin-4-yl-ethanol Compound 1d (243 mg, 95%) as a viscous oil that was used without further purification. MS m/z 328 (M+H)⁺

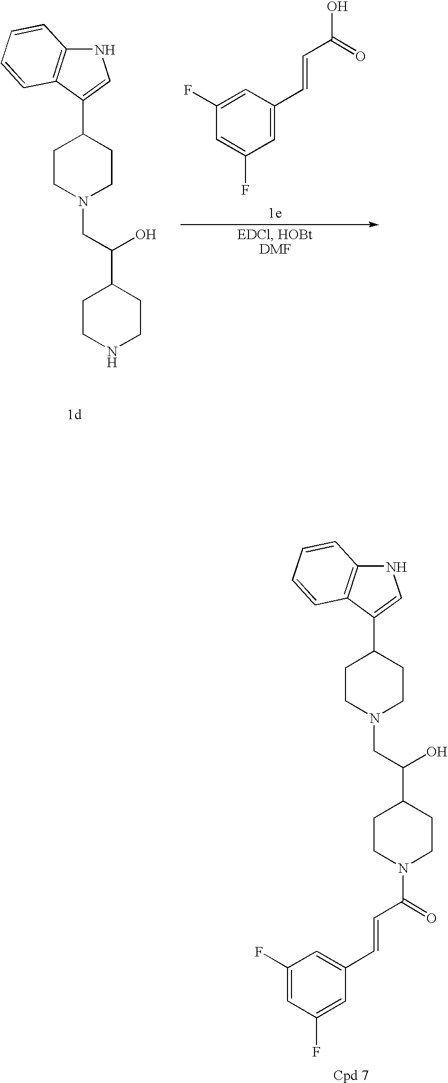

Compound 1d (29 mg, 0.09 mmol, 1 eq) was combined with 3-(3,5-difluoro-phenyl)-acrylic acid Compound 1e (17 mg, 0.09 mmol, 1 eq) and HOBt (13 mg, 0.099 mmol, 1.1 eq) in DMF (1 mL). EDCl (19 mg, 0.099 mmol, 1.1 eq) was added, and the reaction stirred for 16 hrs. The volatiles were removed in vacuo, to provide a residue that was purified via silica gel chromatography (gradient 2% to 10% 2M MeOH.NH₃ in CH₂Cl₂) to provide Compound 7 (41 mg, 92%) as a pale foam. MS m/z 494 (M+H)⁺

Using the procedure of Example 1 and known appropriate reagents and starting materials, the following compounds of the invention were prepared:

| Cpd | Name | MS |
|---|---|---|
| 6 | 3-(3,4-dichloro-phenyl)-1-(4-{1-hydroxy-2-[4-(1H-indol-3-yl)- | 526 |
| | piperidin-1-yl]-ethyl}-piperidin-1-yl)-propenone | |
| 8 | (4-{1-hydroxy-2-[4-(1H-indol-3-yl)-piperidin-1-yl]-ethyl}-piperidin-1-yl)- | 486 |
| | (3,4,5-trifluoro-phenyl)-methanone | |
| 11 | 1-(4-{1-hydroxy-2-[4-(1H-indol-3-yl)-piperidin-1-yl]-ethyl}- | 472 |
| | piperidin-1-yl)-3-m-tolyl-propenone | |
| 12 | 3-(3,5-difluoro-phenyl)-1-(4-{1-hydroxy-2-[4-(4-methoxy-phenyl)- | 485 |
| | piperidin-1-yl]-ethyl}-piperidin-1-yl)-propenone | |
| 13 | 1-(4-{1-hydroxy-2-[4-(4-methoxy-phenyl)-piperidin-1-yl]-ethyl}- | 503 |
| | piperidin-1-yl)-3-(3,4,5-trifluoro-phenyl)-propenone | |
| 14 | 3-(3,4-dichloro-phenyl)-1-(4-{1-hydroxy-2-[4-(4-methoxy-phenyl)- | 517 |
| | piperidin-1-yl]-ethyl}-piperidin-1-yl)-propenone | |
| 19 | 1-(4-{1-hydroxy-2-[4-(1H-pyrrolo[2,3-b]pyridin-3-yl)-piperidin-1- | 473 |
| | yl]-ethyl}-piperidin-1-yl)-3-m-tolyl-propenone | |
| 20 | 3-(3,5-difluoro-phenyl)-1-(4-{1-hydroxy-2-[4-(1H-pyrrolo[2,3- | 495 |
| | b]pyridin-3-yl)-piperidin-1-yl]-ethyl}-piperidin-1-yl)-propenone | |
| 21 | 1-(4-{1-hydroxy-2-[4-(1H-pyrrolo[2,3-b]pyridin-3-yl)-piperidin-1- | 512 |
| | yl]-ethyl}-piperidin-1-yl)-3-(3,4,5-trifluoro-phenyl)-propenone | |
| 22 | 1-(4-{1-hydroxy-2-[4-(1H-pyrrolo[2,3-b]pyridin-3-yl)-piperidin-1- | 527 |
| | yl]-ethyl}-piperidin-1-yl)-3-(3-trifluoromethyl-phenyl)-propenone | |
| 23 | 3-(3,4-dichloro-phenyl)-1-(4-{1-hydroxy-2-[4-(1H-pyrrolo[2,3- | 527 |
| | b]pyridin-3-yl)-piperidin-1-yl]-ethyl}-piperidin-1-yl)-propenone | |
| 26 | 3-(3,5-difluoro-phenyl)-1-(4-{1-hydroxy-2-[4-(5-methoxy-1H-indol- | 524 |
| | 3-yl)-piperidin-1-yl]-ethyl}-piperidin-1-yl)-propenone | |
| 29 | 1-(4-{1-hydroxy-2-[4-(4-methoxy-phenyl)-piperidin-1-yl]-ethyl}- | 463 |
| | piperidin-1-yl)-3-m-tolyl-propenone | |
| 30 | 1-(4-{1-hydroxy-2-[4-(4-methoxy-phenyl)-piperidin-1-yl]-ethyl}- | 517 |
| | piperidin-1-yl)-3-(3-trifluoromethyl-phenyl)-propenone | |
| 32 | 3-(3,4-difluoro-phenyl)-1-(4-{1-hydroxy-2-[4-(4-methoxy-phenyl)- | 485 |
| | piperidin-1-yl]-ethyl}-piperidin-1-yl)-propenone | |
| 33 | 1-(4-{1-hydroxy-2-[4-(1H-pyrazol-3-yl)-piperidin-1-yl]-ethyl}- | 423 |
| | piperidin-1-yl)-3-m-tolyl-propenone | |
| 34 | 3-(3,5-difluoro-phenyl)-1-(4-{1-hydroxy-2-[4-(1H-pyrazol-3-yl)- | 445 |
| | piperidin-1-yl]-ethyl}-piperidin-1-yl)-propenone | |
| 35 | 1-(4-{1-hydroxy-2-[4-(1H-pyrazol-3-yl)-piperidin-1-yl]-ethyl}- | 463 |
| | piperidin-1-yl)-3-(3,4,5-trifluoro-phenyl)-propenone | |
| 40 | N-[3-(1-{2-hydroxy-2-[1-(3-m-tolyl-acryloyl)-piperidin-4-yl]-ethyl}- | 565 |
| | piperidin-4-yl)-1H-indol-5-yl]-methanesulfonamide | |
| 41 | N-{3-[1-(2-{1-[3-(3,5-difluoro-phenyl)-acryloyl]-piperidin-4-yl}-2- | 587 |
| | hydroxy-ethyl)-piperidin-4-yl]-1H-indol-5-yl}-methanesulfonamide | |
| 46 | 1-{4-[2-(4-benzoxazolyl-2-yl-piperidin-1-yl)-1-hydroxy-ethyl]- | 496 |
| | piperidin-1-yl}-3-(3,5-difluoro-phenyl)-propenone | |
| 47 | 1-{4-[2-(4-benzoxazolyl-2-yl-piperidin-1-yl)-1-hydroxy-ethyl]- | 474 |
| | piperidin-1-yl}-3-m-tolyl-propenone | |
| 63 | 4-{1-hydroxy-2-[4-(5-methanesulfonylamino-1H-indol-3-yl)- | 521 |
| | piperidin-1-yl]-ethyl}-piperidine-1-carboxylic acid tert-butyl ester | |
| 85 | 1-(4-{2-[4-(1H-benzoimidazol-2-yl)-piperidin-1-yl]-1-hydroxy- | 527 |
| | ethyl}-piperidin-1-yl)-3-(3,4-dichloro-phenyl)-propenone | |
| 86 | 1-(4-{2-[4-(1H-benzoimidazol-2-yl)-piperidin-1-yl]-1-hydroxy- | 513 |
| | ethyl}-piperidin-1-yl)-3-(3,4,5-trifluoro-phenyl)-propenone | |
| 147 | 4-{2-[4-(5-amino-1H-indol-3-yl)-piperidin-1-yl]-1-hydroxy-ethyl}- | 477 |
| | piperidine-1-carboxylic acid benzyl ester | |
| 184 | 4-{2-[4-(5-acetylamino-1H-indol-3-yl)-piperidin-1-yl]-1-hydroxy- | 519 |
| | ethyl}-piperidine-1-carboxylic acid benzyl ester | |
| 185 | N-{3-[1-(2-hydroxy-2-piperidin-4-yl-ethyl)-piperidin-4-yl]-1H-indol- | 385 |
| | 5-yl}-acetamide | |

### EXAMPLE 2

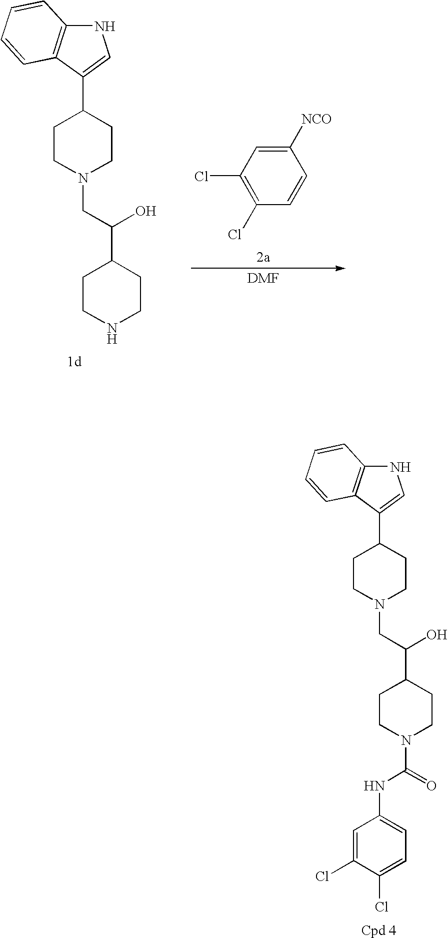

1,2-dichloro-4-isocyanato-benzene Compound 2a (17 mg, 0.09 mmol, 1 eq) was added to a solution of 2-[4-(1H-indol-3-yl)-piperidin-1-yl]-1-piperidin-4-yl-ethanol Compound 1d (29 mg, 0.09 mmol, 1 eq) in DMF (1 mL) and the mixture was stirred overnight. The volatile solvents were removed in vacuo to provide a crude residue that was subjected to silica gel chromatography (gradient 2% to 10% 2M MeOH.NH₃ in CH₂Cl₂) to provide a product with minor impurities. The product was suspended in CH₂Cl₂ and filtered to provide pure Compound 4 (27 mg, 59%) as a white solid. MS m/z 515 (MH⁺)

Using the procedure of Example 2 and known appropriate reagents and starting materials, the following compounds of the invention were prepared:

| Cpd | Name | MS |
|---|---|---|
| 5 | 4-{1-hydroxy-2-[4-(1H-indol-3-yl)-piperidin-1-yl]-ethyl}- | 483 |
| | piperidine-1-carboxylic acid (3,5-difluoro-phenyl)-amide | |
| 15 | 4-{1-hydroxy-2-[4-(4-methoxy-phenyl)-piperidin-1-yl]- | 506 |
| | ethyl}-piperidine-1-carboxylic | |
| | acid (3,4-dichloro-phenyl)-amide | |
| 24 | 4-{1-hydroxy-2-[4-(1H-pyrrolo[2,3-b]pyridin-3-yl)- | 516 |
| | piperidin-1-yl]-ethyl}-piperidine-1-carboxylic | |
| | acid (3,4-dichloro-phenyl)-amide | |
| 25 | 4-{1-hydroxy-2-[4-(1H-pyrrolo[2,3-b]pyridin-3-yl)- | 494 |
| | piperidin-1-yl]-ethyl}-piperidine-1-carboxylic acid (4- | |
| | methylsulfanyl-phenyl)-amide | |
| 31 | 4-{1-hydroxy-2-[4-(4-methoxy-phenyl)-piperidin-1-yl]- | 484 |
| | ethyl}-piperidine-1-carboxylic acid (4- | |
| | methylsulfanyl-phenyl)-amide | |
| 36 | 4-{1-hydroxy-2-[4-(1H-pyrazol-3-yl)-piperidin-1-yl]-ethyl}- | 466 |
| | piperidine-1-carboxylic acid (3,4-dichloro-phenyl)-amide | |
| 37 | 4-{1-hydroxy-2-[4-(1H-pyrazol-3-yl)-piperidin-1-yl]-ethyl}- | 434 |
| | piperidine-1-carboxylic acid (3,4-difluoro-phenyl)-amide | |
| 90 | 4-{1-hydroxy-2-[4-(4-methoxy-phenyl)-piperidin-1-yl]- | 524 |
| | ethyl}-piperidine-1-carboxylic acid (4,5-dichloro- | |
| | 2-fluoro-phenyl)-amide | |
| 91 | 4-{1-hydroxy-2-[4-(4-methoxy-phenyl)-piperidin-1-yl]- | 524 |
| | ethyl}-piperidine-1-carboxylic acid (3-fluoro- | |
| | 5-trifluoromethyl-phenyl)-amide | |
| 100 | 4-{1-hydroxy-2-[4-(4-methoxy-phenyl)-piperidin-1-yl]- | 482 |
| | ethyl}-piperidine-1-carboxylic acid | |
| | benzo[1,3]dioxol-5-ylamide | |
| 101 | 4-{1-hydroxy-2-[4-(4-methoxy-phenyl)-piperidin-1-yl]- | 540 |
| | ethyl}-piperidine-1-carboxylic acid (2-chloro-4- | |
| | trifluoromethyl-phenyl)-amide | |

### EXAMPLE 3

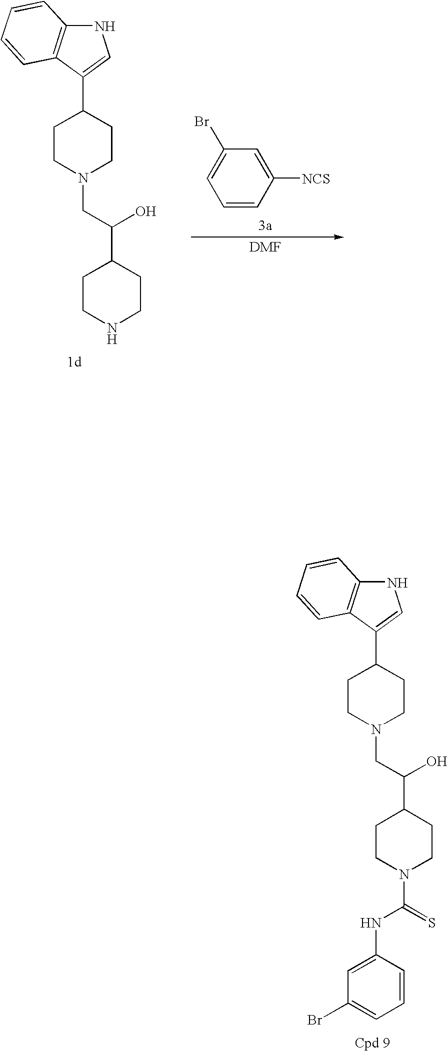

A solution of 2-[4-(1H-indol-3-yl)-piperidin-1-yl]-1-piperidin-4-yl-ethanol Compound 1d (29 mg, 0.09 mmol, 1 eq) and Et₃N (38 μL, 0.27 mmol, 3 eq) in DMF (1 mL) was treated with 1-bromo-3-isothiocyanato-benzene Compound 3a (19 mg, 0.09 mmol, 1 eq) and the mixture was stirred for 16 hrs. The volatile solvents were removed in vacuo, to provide a crude residue that was subjected to silica gel chromatography (gradient 2% to 10% 2M MeOH.NH₃ in CH₂Cl₂) to provide Compound 9 (44 mg, 90%) as a pale foam. MS m/z 541 (MH⁺)

Using the procedure of Example 3 and known appropriate reagents and starting materials, the following compounds of the invention were prepared:

| Cpd | Name | MS |
|---|---|---|
| 16 | 4-{1-hydroxy-2-[4-(4-methoxy-phenyl)-piperidin-1-yl]-ethyl}- | 532 |
| | piperidine-1-carbothioic acid (3-bromo-phenyl)-amide | |
| 89 | 4-{1-hydroxy-2-[4-(4-methoxy-phenyl)-piperidin-1-yl]-ethyl}- | 522 |
| | piperidine-1-carbothioic acid (3,5-dichloro-phenyl)-amide | |
| 96 | 4-{1-hydroxy-2-[4-(4-methoxy-phenyl)-piperidin-1-yl]-ethyl}- | 484 |
| | piperidine-1-carbothioic acid (3-methoxy-phenyl)-amide | |
| 97 | 4-{1-hydroxy-2-[4-(4-methoxy-phenyl)-piperidin-1-yl]-ethyl}- | 522 |
| | piperidine-1-carbothioic acid (3-trifluoromethyl-phenyl)-amide | |
| 98 | 4-{1-hydroxy-2-[4-(4-methoxy-phenyl)-piperidin-1-yl]-ethyl}-piperidine- | 522 |
| | 1-carbothioic acid (3,4-dichloro-phenyl)-amide | |
| 99 | 4-{1-hydroxy-2-[4-(4-methoxy-phenyl)-piperidin-1-yl]-ethyl}- | 550 |
| | piperidine-1-carbothioic acid (4-bromo-3-fluoro-phenyl)-amide | |
| 102 | 4-{1-hydroxy-2-[4-(4-methoxy-phenyl)-piperidin-1-yl]-ethyl}- | 488 |
| | piperidine-1-carbothioic acid (3-chloro-phenyl)-amide | |
| 103 | 4-{1-hydroxy-2-[4-(4-methoxy-phenyl)-piperidin-1-yl]-ethyl}- | 484 |
| | piperidine-1-carbothioic acid (3-methoxy-phenyl)-amide | |
| 104 | 4-{1-hydroxy-2-[4-(4-methoxy-phenyl)-piperidin-1-yl]-ethyl}- | 479 |
| | piperidine-1-carbothioic acid (3-cyano-phenyl)-amide | |
| 105 | 4-{1-hydroxy-2-[4-(4-methoxy-phenyl)-piperidin-1-yl]-ethyl}- | 522 |
| | piperidine-1-carbothioic acid (3-trifluoromethyl-phenyl)-amide | |
| 106 | 4-{1-hydroxy-2-[4-(4-methoxy-phenyl)-piperidin-1-yl]-ethyl}- | 522 |
| | piperidine-1-carbothioic acid (4-trifluoromethyl-phenyl)-amide | |
| 107 | 4-{1-hydroxy-2-[4-(4-methoxy-phenyl)-piperidin-1-yl]-ethyl}- | 490 |
| | piperidine-1-carbothioic acid (3,5-difluoro-phenyl)-amide | |
| 108 | 4-{1-hydroxy-2-[4-(4-methoxy-phenyl)-piperidin-1-yl]-ethyl}- | 522 |
| | piperidine-1-carbothioic acid (3,4-dichloro-phenyl)-amide | |
| 109 | 4-{1-hydroxy-2-[4-(4-methoxy-phenyl)-piperidin-1-yl]-ethyl}- | 508 |
| | piperidine-1-carbothioic acid (2,3,5-trifluoro-phenyl)-amide | |
| 110 | 4-{1-hydroxy-2-[4-(4-methoxy-phenyl)-piperidin-1-yl]-ethyl}-piperidine- | 550 |
| | 1-carbothioic acid (4-bromo-3-fluoro-phenyl)-amide | |
| 111 | 4-{1-hydroxy-2-[4-(4-methoxy-phenyl)-piperidin-1-yl]-ethyl}- | 514 |
| | piperidine-1-carbothioic acid (3,5-dimethoxy-phenyl)-amide | |

### EXAMPLE 4

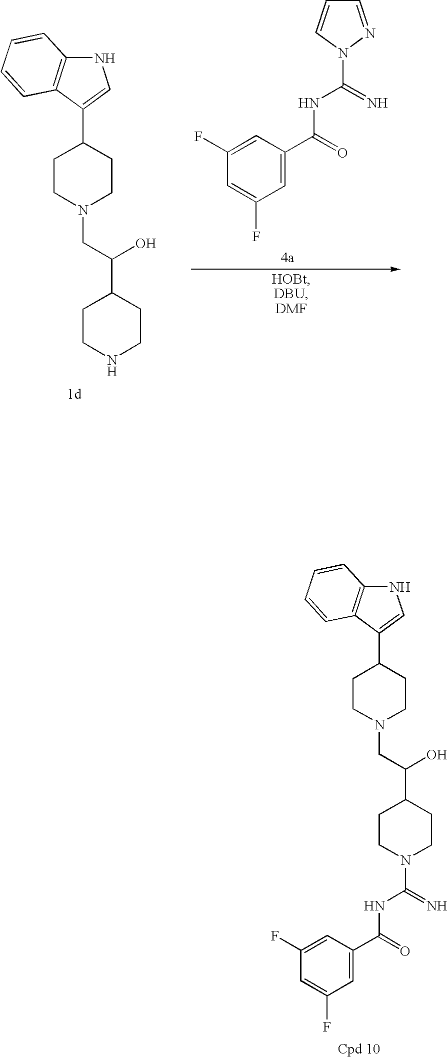

A solution of 2-[4-(1H-indol-3-yl)-piperidin-1-yl]-1-piperidin-4-yl-ethanol Compound 1d (29 mg, 0.09 mmol, 1 eq), DBU (40 μL, 0.27 mmol, 3 eq) and HOBt (13 mg, 0.09 mmol, 1 eq) in DMF (1 mL) was treated with 3,5-difluoro-N-(imino-pyrazol-1-yl-methyl)-benzamide Compound 4a (25 mg, 0.099 mmol, 1.1 eq) and stirred for 16 hrs. The volatile solvents were removed in vacuo, to provide a crude residue that was subjected to silica gel chromatography (gradient 2% to 10% 2M MeOH:NH₃ in CH₂Cl₂) to provide Compound 10 (36 mg, 90%) as a pale foam. MS m/z 510 (M+H)⁺

### EXAMPLE 5

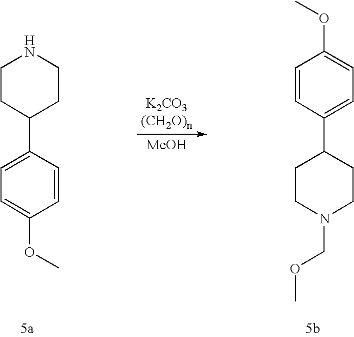

Paraformaldehyde (120 mg, 4.00 mmol, 1 eq) was added to a solution of potassium carbonate (553 mg, 4.00 mmol, 1 eq) and 4-(4-methoxy-phenyl)-piperidine Compound 5a (766 mg, 4.00 mmol, 1 eq) in methanol (5 mL) and the mixture was stirred for 72 hrs. The reaction mixture was filtered through Celite and the filtrate evaporated. The resulting residue was dissolved in diethyl ether and filtered through Celite, then the filtrate was evaporated to provide 1-methoxymethyl-4-(4-methoxy-phenyl)-piperidine Compound 5b (239 mg, 25%) as a clear oil that was used in the next step without further purification.

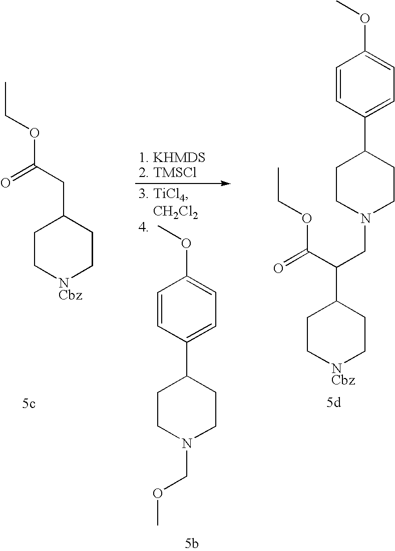

A solution of 4-ethoxycarbonylmethyl-piperidine-1-carboxylic acid benzyl ester Compound 5c (200 mg, 0.65 mmol, 1 eq) was dissolved in DCM (4 mL) and cooled to −78° C. Potassium bis(trimethylsilyl)amide (0.5 M in toluene, 1.3 mL, 1.3 mmol, 2 eq) was added dropwise to the solution of Compound 5c and the mixture was stirred for 1 hr at −78° C. Trimethylsilyl chloride (164 μL, 1.3 mmol, 2 eq) was added and the mixture was stirred for 1 hr at −78° C. A solution of Compound 5b (120 mg, 0.51 mmol, 0.78 eq) in DCM (1 mL) was added, followed by the addition of TiCl₄ (108 μL, 0.99 mmol, 1.5 eq). The mixture was slowly warmed to 0° C. and stirred over 2 hrs, then the reaction was carefully quenched with saturated sodium bicarbonate solution and stirred for 1.5 hrs. The solids were removed by filtration through Celite. After washing the filter pad with EtOAc, the combined organic filtrates were washed with saturated sodium bicarbonate, dried over anhydrous sodium sulfate, then filtered and evaporated to provide a residue that was subjected to silica gel chromatography (gradient 1:1 EtOAc:hexanes to 2:1 EtOAc:hexanes) to provide 4-{1-ethoxycarbonyl-2-[4-(4-methoxy-phenyl)-piperidin-1-yl]-ethyl}-piperidine-1-carboxylic acid benzyl ester Compound 5d (115 mg, 44%) as a pale oil. MS m/z 509(M+H)⁺.

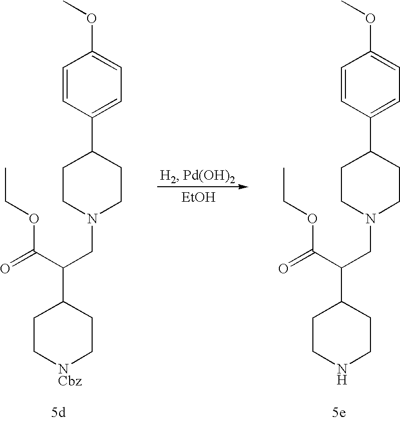

A solution of Compound 5d (236 mg, 0.46 mmol, 1 eq) and palladium hydroxide (156 mg, 0.11 mmol, 0.2 eq) in methanol (10 mL) was purged with nitrogen, then pressurized with hydrogen (60 psi) and shaken for 5 hrs. The reaction was purged with nitrogen and filtered through celite, then the filtrate was evaporated to provide 3-[4-(4-methoxy-phenyl)-piperidin-1-yl]-2-piperidin-4-yl-propionic acid ethyl ester Compound 5e (167 mg, 97%) as a clear, viscous oil that was used without further purification. MS m/z 375 (M+H)⁺.

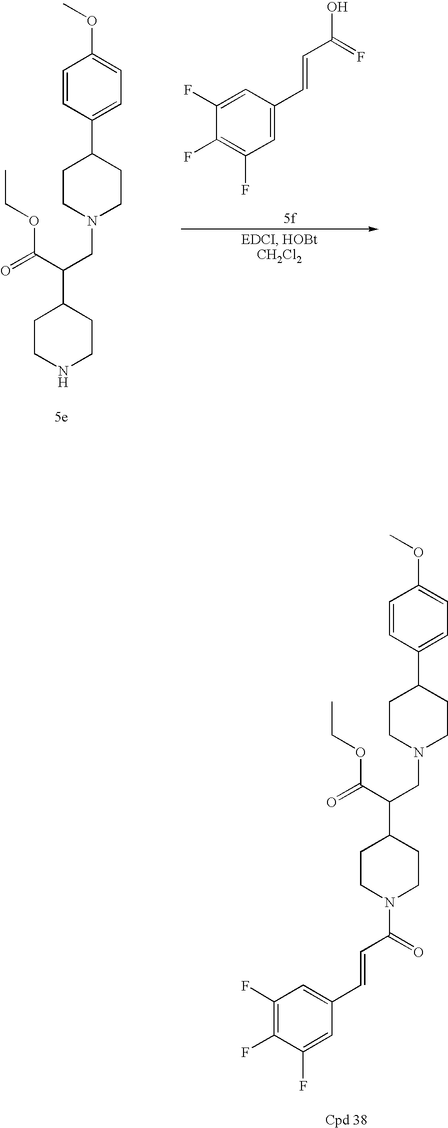

A solution of Compound 5e (56 mg, 0.15 mmol, 1 eq), 3-(3,4,5-trifluoro-phenyl)-acrylic acid Compound 5f (33 mg, 0.17 mmol, 1.1 eq), and HOBt (22 mg, 0.17 mmol, 1.1 eq) in DCM (1 mL) was treated with EDCl (32 mg, 0.17 mmol, 1.1 eq) and stirred for 16 h. The reaction was then diluted with DCM and partitioned with saturated sodium bicarbonate. The organic layer was removed and dried over anhydrous sodium sulfate, then filtered and evaporated to provide a crude residue that was subjected to silica gel chromatography (gradient 1:1 EtOAc:hexanes to 3:1 EtOAc:hexanes) to provide Compound 38 (71 mg, 85%) as a foam. MS m/z 559 (M+H)⁺.

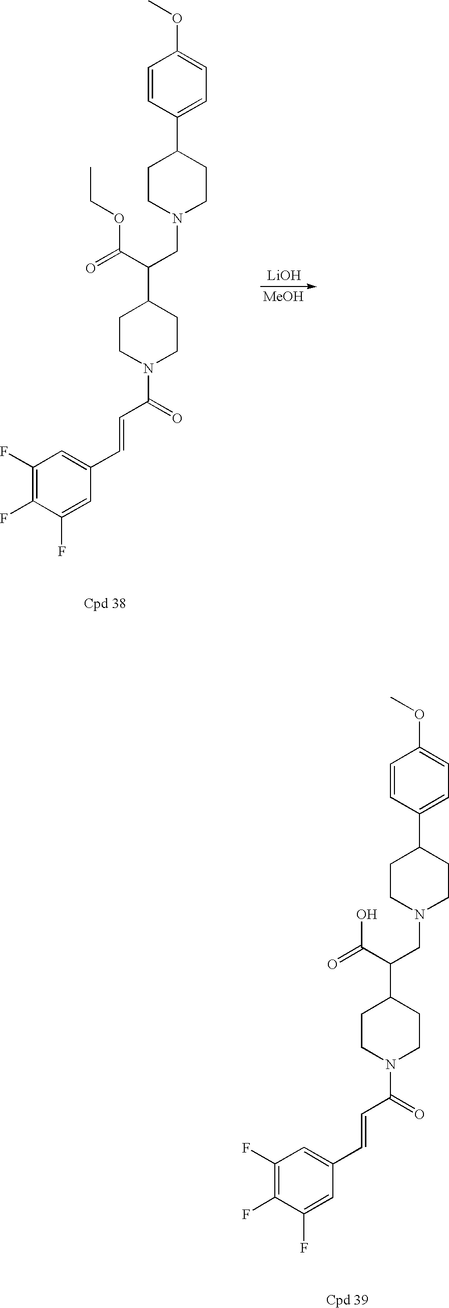

1M LiOH (0.55 mL, 0.55 mmol, 5 eq) was added to a solution of Compound 38 (63 mg, 0.11 mmol, 1 eq) in methanol (3 mL). The reaction was heated at reflux for 4 hrs, then cooled to RT and neutralized with 1M HCl. The volatile solvents were removed in vacuo and the resulting residue was triturated with water, then a minimal amount of methanol was added and the solution sonicated. The resulting white solid was collected by filtration and resuspended in methanol. 2M HCl in diethyl ether was added and the solid was dissolved. The volatile solvents were removed in vacuo to afford Compound 39 (22 mg, 35%) as a HCl salt. MS m/z 531 (M+H)⁺.

### EXAMPLE 6

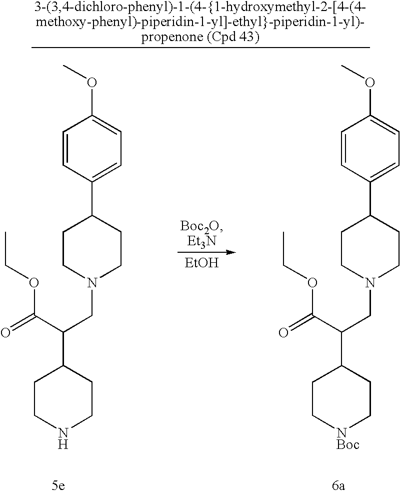

Di-tert-butyl dicarbonate (70 mg, 0.32 mmol, 1.1 eq) and 3-[4-(4-methoxy-phenyl)-piperidin-1-yl]-2-piperidin-4-yl-propionic acid ethyl ester Compound 5e (111 mg, 0.30 mmol, 1 eq) were dissolved in ethanol and heated at 65° C. for 4 hrs. The volatile solvents were removed in vacuo to provide 4-{1-ethoxycarbonyl-2-[4-(4-methoxy-phenyl)-piperidin-1-yl]-ethyl}-piperidine-1-carboxylic acid tert-butyl ester Compound 6a (142 mg, quant), which was used in the next step without any further purification. MS m/z 474 (M+H)⁺.

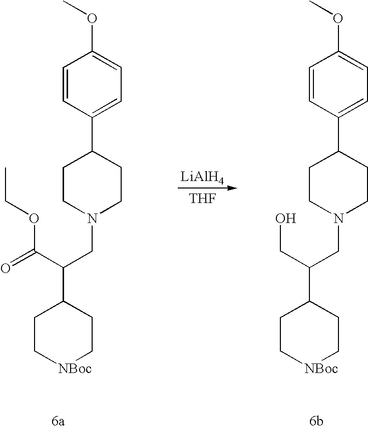

Compound 6a (142 mg, 0.30 mmol, 1 eq) was dissolved in a mixture of THF (2.3 mL) and DCM (2 mL) and cooled to 0° C. Lithium aluminum hydride (1M in THF, 0.45 mL, 0.45 mmol, 1.5 eq) was added dropwise and the mixture was stirred at 0° C. for 20 min and RT for 2 hrs. The reaction was quenched by careful addition of water (20 μL), 15% aqueous NaOH (20 μL) and an additional amount of water (60 μL). After stirring for 30 min, the solids were removed by filtration through Celite and the filter cake washed with additional portions of EtOAc. The combined filtrates were removed in vacuo to provide 4-{1-hydroxymethyl-2-[4-(4-methoxy-phenyl)-piperidin-1-yl]-ethyl}-piperidine-1-carboxylic acid tert-butyl ester Compound 6b (127 mg, 98%) as a solid. MS m/z 433 (M+H)⁺.

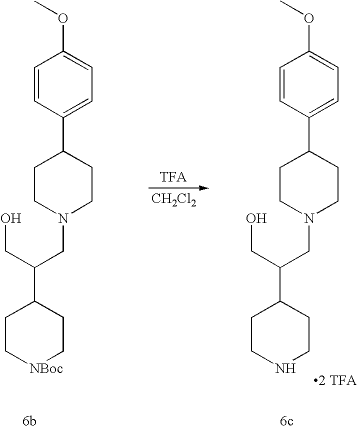

TFA (0.6 mL) was added dropwise to a solution of Compound 6b (127 mg, 0.29 mmol, 1 eq) in DCM (3.4 mL) and the mixture was stirred for 3 hrs. The volatile solvents were removed in vacuo to provide the bis-trifluoroacetate salt of 3-[4-(4-methoxy-phenyl)-piperidin-1-yl]-2-piperidin-4-yl-propan-1-ol Compound 6c as a viscous oil that was used in the next step without further purification. MS m/z 333 (M+H)⁺.

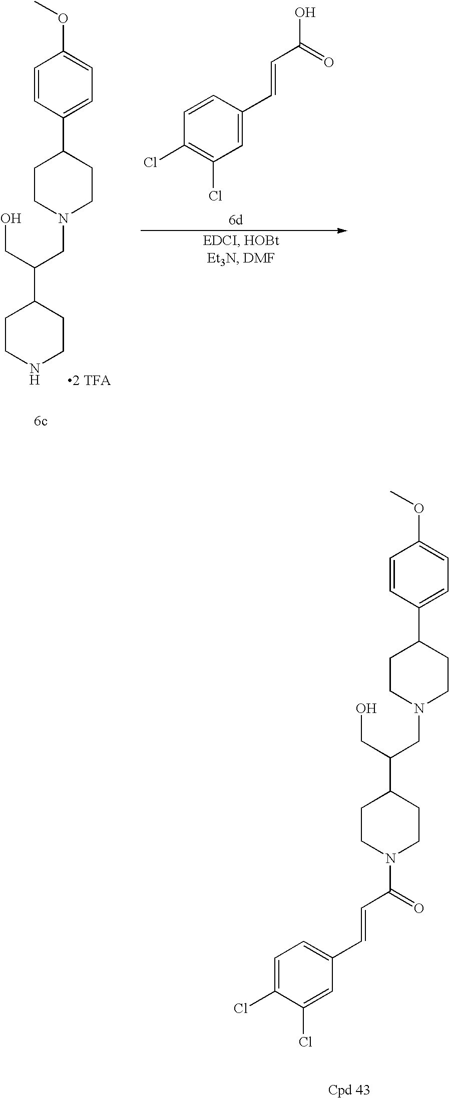

Compound 6c (41 mg, 0.073 mmol, 1 eq), 3-(3,4-dichloro-phenyl)-acrylic acid Compound 6d (16 mg, 0.073 mmol, 1 eq), HOBt (11 mg, 0.080 mmol, 1.1 eq) and Et₃N (31 μL, 0.22 mmol, 3 eq) were dissolved in DMF (1 mL). EDCl (15 mg, 0.80 mmol, 1.1 eq) was added and the mixture was stirred for 16 hrs. The volatile solvents were removed in vacuo and the resulting residue dissolved in CH₂Cl₂. The solution washed with saturated aqueous NaHCO₃ and dried over anhydrous Na₂SO₄, then filtered and evaporated to provide a crude residue that was subjected to silica gel chromatography (gradient 2% to 10% 2M MeOH.NH₃ in CH₂Cl₂) to provide Compound 43 as a pale foam. MS m/z 532 (M+H)⁺.

Using the procedure of Example 6 and known appropriate reagents and starting materials, the following compounds of the invention were prepared:

| Cpd | Name | MS |
|---|---|---|
| 42 | 1-(4-{1-hydroxymethyl-2-[4-(4-methoxy-phenyl)- | 477 |
| | piperidin-1-yl]-ethyl}-piperidin-1-yl)-3-m-tolyl-propenone | |
| 45 | 3-(3,5-difluoro-phenyl)-1-(4-{1-hydroxymethyl-2-[4-(4- | 499 |
| | methoxy-phenyl)-piperidin-1-yl]-ethyl}- | |
| | piperidin-1-yl)-propenone | |

### EXAMPLE 7

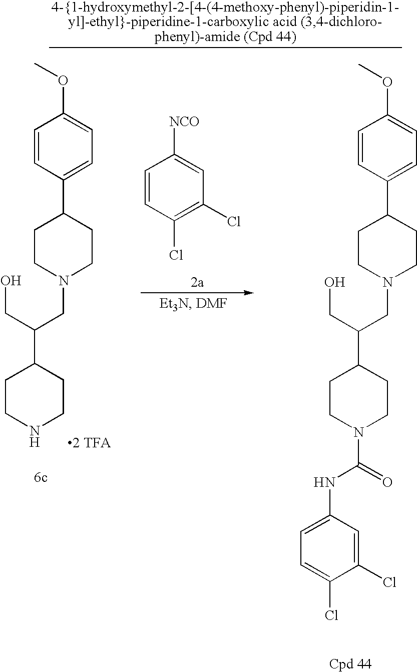

1,2-dichloro-4-isocyanato-benzene Compound 2a (14 mg, 0.073 mmol, 1 eq) was added to a solution of 3-[4-(4-methoxy-phenyl)-piperidin-1-yl]-2-piperidin-4-yl-propan-1-ol bis-trifluoroacetate salt Compound 6c (41 mg, 0.073 mmol, 1 eq) and triethylamine (31 μL, 0.22 mmol, 3 eq) in DMF (1 mL) and the mixture was stirred overnight. The volatile solvents were removed in vacuo and the resulting residue dissolved in CH₂Cl₂. The solution washed with saturated aqueous NaHCO₃ and dried over anhydrous Na₂SO₄, then filtered and evaporated to provide a crude residue that was subjected to silica gel chromatography (gradient 2% to 10% 2M MeOH.NH₃ in CH₂Cl₂) to provide Compound 44 as an off-white solid. MS m/z 520 (M+H)⁺.

### EXAMPLE 8

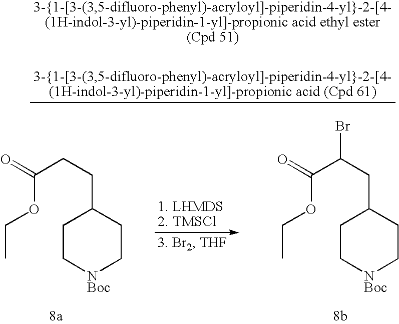

A solution of 4-(2-ethoxycarbonyl-ethyl)-piperidine-1-carboxylic acid tert-butyl ester Compound 8a (4 g, 14.0 mmol, 1 eq) in THF (19.2 mL) was cooled to −78° C. and treated with LHMDS (1M in THF, 18.2 mL, 18.2 mmol, 1.3 eq). The mixture was stirred for 30 min, then TMSCl (2.3 mL, 18.2 mmol, 1.3 eq) was added and the mixture was stirred for 1 hr. Bromine (0.7 mL, 14.0 mmol, 1 eq) was added dropwise and the mixture was stirred for 2 hrs at −78° C. and 30 min at 0° C. The reaction mixture was partitioned between EtOAc and a mixture of aqueous sodium thiosulfate and saturated sodium bicarbonate. The organic layer was removed and washed sequentially with water and brine and dried over anhydrous sodium sulfate, then filtered and evaporated to provide a crude oil that was subjected to silica gel chromatography (gradient 20% to 50% EtOAc in hexanes) to provide 4-(2-bromo-2-ethoxycarbonyl-ethyl)-piperidine-1-carboxylic acid tert-butyl ester Compound 8b in an inseparable mixture with Compound 8a, which was used in the next step without further purification.

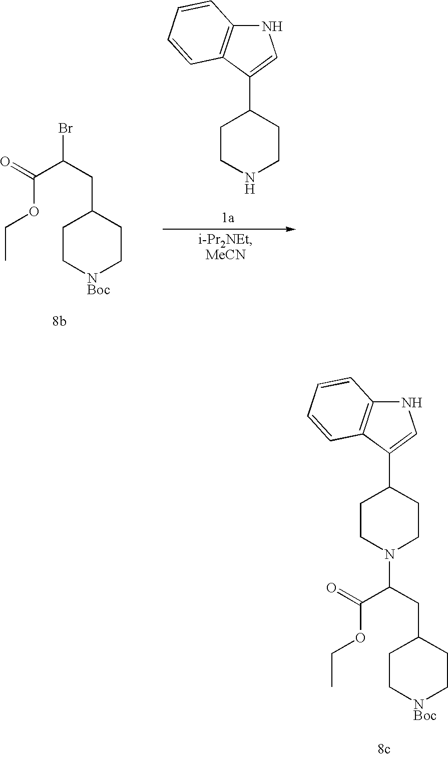

Compound 8b (1 g, 2.75 mmol, 1 eq), 3-piperidin-4-yl-1H-indole Compound 1a (550 mg, 2.75 mmol, 1 eq) and DIPEA (0.96 mL, 5.50 mmol, 2 eq) were added to MeCN (10 mL) and heated at reflux for 8 hrs. The reaction mixture was cooled, the solvents were removed in vacuo and the resulting residue was subjected to silica gel chromatography (gradient 3:1:0.5 CH₂Cl₂:hexanes:EtOAc to 3:1:2 CH₂Cl₂:hexanes:EtOAc) to provide 4-{2-ethoxycarbonyl-2-[4-(1H-indol-3-yl)-piperidin-1-yl]-ethyl}-piperidine-1-carboxylic acid tert-butyl ester Compound 8c (886 mg, 67%) as a pale oil. MS m/z 484 (M+H)⁺.

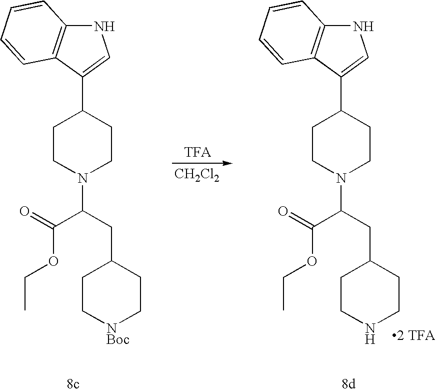

A solution of Compound 8c (266 mg, 0.55 mmol, 1 eq) in DCM (9 mL) was treated with TFA (1 mL) and stirred for 4 hrs. The solvents were removed in vacuo to provide 2-[4-(1H-indol-3-yl)-piperidin-1-yl]-3-piperidin-4-yl-propionic acid ethyl ester Compound 8d as a bis-trifluoroacetate salt that was used in the next step without further purification. MS m/z 384 (M+H)⁺.

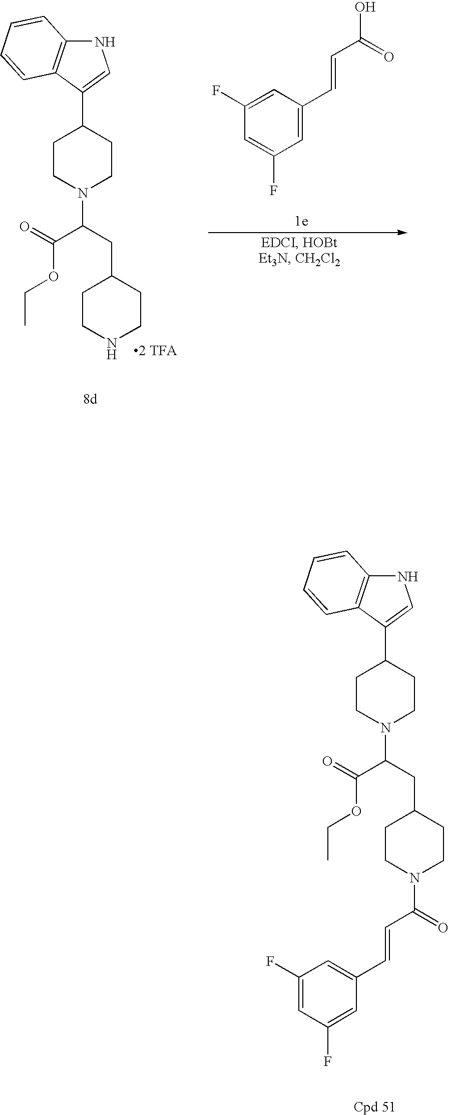

Compound 8d (0.11 mmol, 1 eq), 3-(3,5-difluoro-phenyl)-acrylic acid Compound 1e (22 mg, 0.12 mmol, 1.1 eq), HOBt (16 mg, 0.12 mmol, 1.1 eq) and triethylamine (46 mL, 0.33 mmol, 3 eq) were dissolved in DCM (1 mL). EDCl (23 mg, 0.12 mmol, 1.1 eq) was added and the mixture was stirred for 16 hrs. The reaction mixture was diluted with DCM and partitioned with saturated sodium bicarbonate. The organic layer was removed and the aqueous layer was extracted with DCM. The combined organic extracts were dried over anhydrous sodium sulfate, then filtered and evaporated to provide a crude residue that was subjected to silica gel chromatography (gradient 2% to 10% 2M MeOH.NH₃ in CH₂Cl₂) to provide Compound 51 (50 mg, 83%) as a pale foam. MS m/z 550 (M+H)⁺.

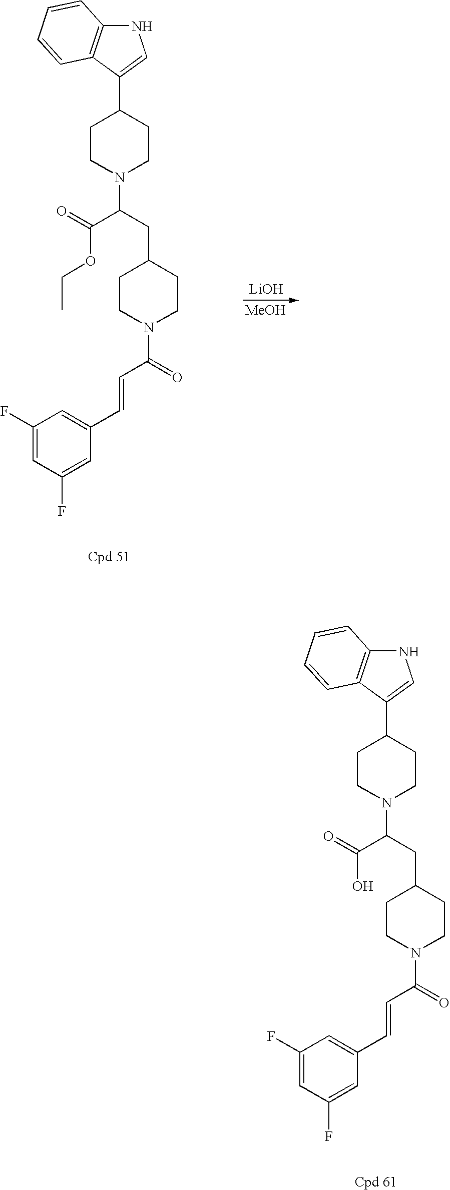

A solution of Compound 51 (40 mg, 0.073 mmol, 1 eq) in methanol (4 mL) was treated with 1M LiOH (0.8 mL, 0.8 mmol, 11 eq) and heated at reflux for 8 hrs. The reaction mixture was cooled, adjusted to pH 7 with aqueous HCl, and evaporated to provide a white solid. The solid was triturated with water and filtered to provide Compound 61 (23 mg, 60%) as a white solid that was approximately 80% pure by LCMS. MS m/z 522 (M+H)⁺.

Using the procedure of Example 8 and known appropriate reagents and starting materials, the following compounds of the invention were prepared:

| Cpd | Name | MS |
|---|---|---|
| 48 | 2-[4-(1H-indol-3-yl)-piperidin-1-yl]-3-[1-(3-m-tolyl- | 528 |
| | acryloyl)-piperidin-4-yl]-propionic acid ethyl ester | |
| 49 | 3-{1-[3-(3,4-dichloro-phenyl)-acryloyl]-piperidin-4-yl}-2- | 582 |
| | [4-(1H-indol-3-yl)-piperidin-1-yl]-propionic | |
| | acid ethyl ester | |
| 50 | 2-[4-(1H-indol-3-yl)-piperidin-1-yl]-3-{1-[3- | 568 |
| | (3,4,5-trifluoro-phenyl)-acryloyl]-piperidin- | |
| | 4-yl}-propionic acid ethyl ester | |
| 53 | 2-[4-(1H-indol-3-yl)-piperidin-1-yl]-3-[1-(3-m- | 500 |
| | tolyl-acryloyl)-piperidin-4-yl]-propionic acid | |
| 60 | 3-{1-[3-(3,4-dichloro-phenyl)-acryloyl]-piperidin-4- | 554 |
| | yl}-2-[4-(1H-indol-3-yl)-piperidin-1-yl]-propionic acid | |

### EXAMPLE 9

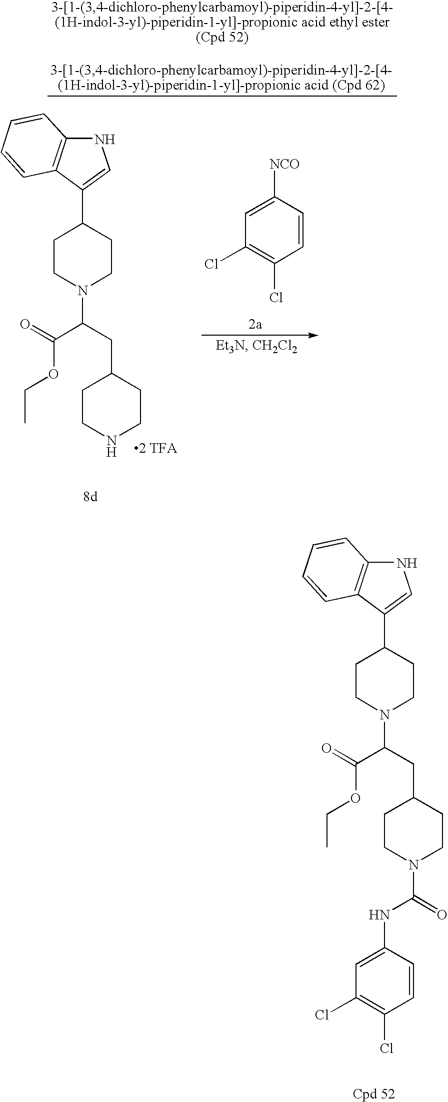

2-[4-(1H-indol-3-yl)-piperidin-1-yl]-3-piperidin-4-yl-propionic acid ethyl ester bis-trifluoroacetate salt Compound 8d (0.11 mmol, 1 eq) and triethylamine (46 mL, 0.33 mmol, 3 eq) were dissolved in DCM (1 mL). 1,2-dichloro-4-isocyanato-benzene Compound 2a (26 mg, 0.14 mmol, 1.3 eq) was added and the mixture was stirred for 16 hrs. The reaction mixture was diluted with DCM and partitioned with saturated sodium bicarbonate. The organic layer was removed and the aqueous layer was extracted with DCM. The combined organic extracts were dried over anhydrous sodium sulfate, filtered and evaporated to provide a crude residue that was subjected to silica gel chromatography (gradient 2% to 10% 2M MeOH.NH₃ in CH₂Cl₂) to provide Compound 52 (59 mg, 94%) as a pale foam. MS m/z 571 (M+H)⁺.

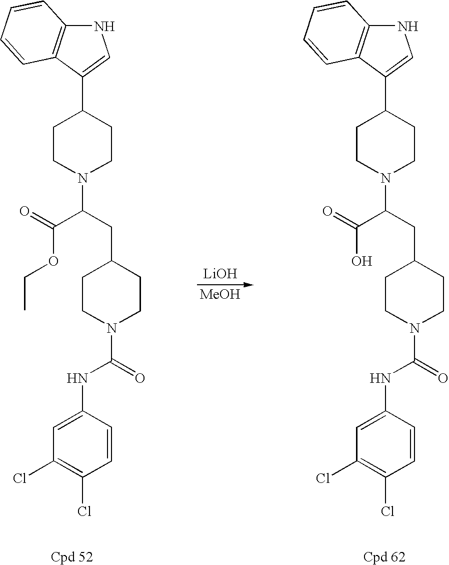

A solution of Compound 52 (49 mg, 0.086 mmol, 1 eq) in methanol (4 mL) was treated with 1M LiOH (0.8 mL, 0.8 mmol, 11 eq) and heated at reflux for 8 hrs. The reaction mixture was cooled, adjusted to pH 7 with aqueous HCl and evaporated to provide a white solid. The solid was triturated with water and filtered to provide Compound 62 (26 mg, 56%) as a white solid. MS m/z 544 (M+H)⁺.

### EXAMPLE 10

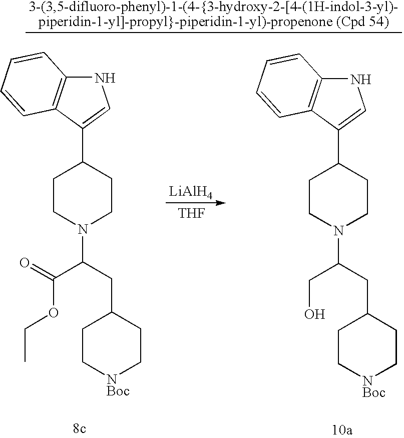

4-{2-ethoxycarbonyl-2-[4-(1H-indol-3-yl)-piperidin-1-yl]-ethyl}-piperidine-1-carboxylic acid tert-butyl ester Compound 8c (620 mg, 1.28 mmol, 1 eq) was dissolved in THF (9.6 mL) and cooled to 0° C. Lithium aluminum hydride (1M in THF, 1.9 mL, 1.92 mmol, 1.5 eq) was added dropwise, then the mixture was stirred for 3 hrs and allowed to warm to RT. The reaction was quenched by careful addition of water (77 μL), 15% aqueous NaOH (77 μL) and an additional amount of water (232 μL). The mixture was stirred for 30 min, the solids were removed by filtration through Celite and the filter cake washed with additional portions of EtOAc. The combined filtrates were evaporated to provide a crude residue that was subjected to silica gel chromatography (gradient 2% to 10% 2M MeOH.NH₃ in CH₂Cl₂) to provide 4-{3-hydroxy-2-[4-(1H-indol-3-yl)-piperidin-1-yl]-propyl}-piperidine-1-carboxylic acid tert-butyl ester Compound 10a (373 mg, 66%) as a white foam. MS m/z 442 (M+H)⁺.

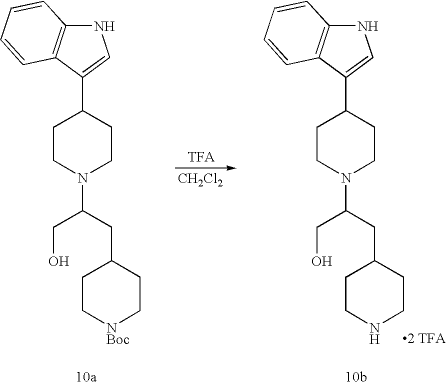

A solution of Compound 10a (344 mg, 0.78 mmol, 1 eq) in DCM (6 mL) was treated with TFA (2 mL) and stirred for 1 hr. The solvents were removed in vacuo to provide 2-[4-(1H-indol-3-yl)-piperidin-1-yl]-3-piperidin-4-yl-propan-1-ol Compound 10b as a bis-trifluoroacetate salt that was used in the next step without further purification. MS m/z 341 (M+H)⁺.

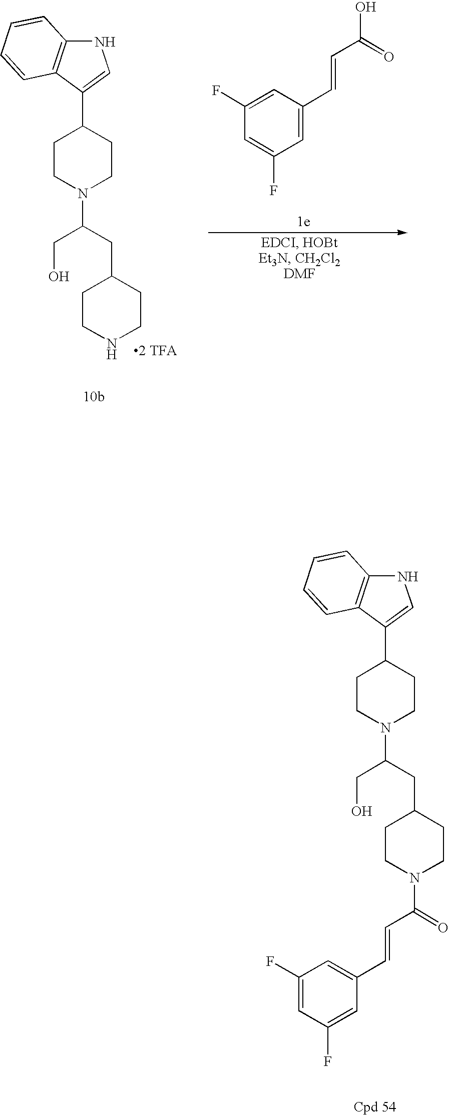

Compound 10b (0.11 mmol, 1 eq), 3-(3,5-difluoro-phenyl)-acrylic acid Compound 1e (20 mg, 0.11 mmol, 1 eq), HOBt (16 mg, 0.12 mmol, 1.1 eq), and triethylamine (46 mL, 0.33 mmol, 3 eq) were dissolved in a mixture of DCM (1 mL) and DMF (0.5 mL). EDCl (23 mg, 0.12 mmol, 1.1 eq) was added and the mixture was stirred for 16 hrs. The reaction mixture was evaporated, diluted with DCM and partitioned with saturated sodium bicarbonate. The organic layer was removed and the aqueous layer was extracted with DCM. The combined organic extracts were dried over anhydrous sodium sulfate, then filtered and evaporated to provide a crude residue that was subjected to silica gel chromatography (gradient 2% to 10% 2M MeOH.NH₃ in CH₂Cl₂) to provide Compound 54 (34 mg, 61%) as a tan foam. MS m/z 508 (M+H)⁺.

Using the procedure of Example 10 and known appropriate reagents and starting materials, the following compounds of the invention were prepared:

| Cpd | Name | MS |
|---|---|---|
| 55 | 1-(4-{3-hydroxy-2-[4-(1H-indol-3-yl)-piperidin-1-yl]- | 526 |
| | propyl}-piperidin-1-yl)-3-(3,4,5- | |
| | trifluoro-phenyl)-propenone | |
| 56 | 3-(3,4-dichloro-phenyl)-1-(4-{3-hydroxy-2-[4-(1H-indol-3- | 540 |
| | yl)-piperidin-1-yl]-propyl}-piperidin-1-yl)-propenone | |
| 57 | 1-(4-{3-hydroxy-2-[4-(1H-indol-3-yl)-piperidin-1-yl]- | 486 |
| | propyl}-piperidin-1-yl)-3-m-tolyl-propenone | |

### EXAMPLE 11

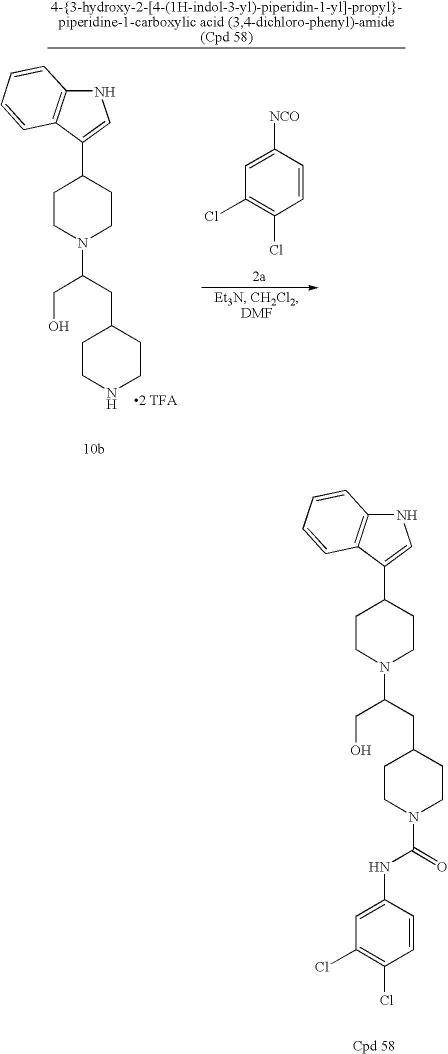

2-[4-(1H-indol-3-yl)-piperidin-1-yl]-3-piperidin-4-yl-propan-1-ol bis-trifluoroacetate salt Compound 10b (0.11 mmol, 1 eq) and triethylamine (46 mL, 0.33 mmol, 3 eq) were dissolved in a mixture of DCM (1 mL) and DMF (0.5 mL). 1,2-dichloro-4-isocyanato-benzene Compound 2a (21 mg, 0.11 mmol, 1 eq) was added and the mixture was stirred for 16 hrs. The reaction mixture was evaporated to dryness, the residue diluted with DCM and partitioned with saturated sodium bicarbonate. The organic layer was removed and the aqueous layer was extracted with DCM. The combined organic extracts were dried over anhydrous sodium sulfate, then filtered and evaporated to provide a crude residue that was subjected to silica gel chromatography (gradient 2% to 10% 2M MeOH.NH₃ in CH₂Cl₂) to provide Compound 58 (41 mg, 70%) as a tan foam. MS m/z 529 (M+H)⁺.

### EXAMPLE 12

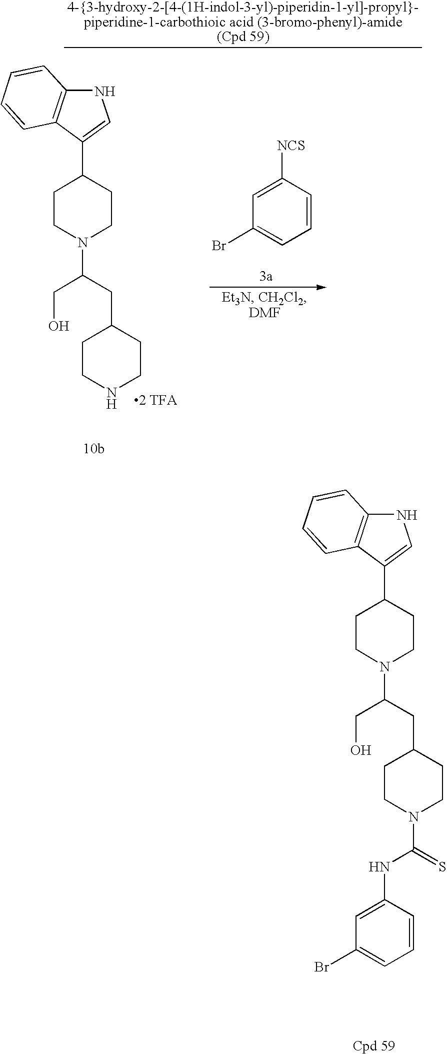

2-[4-(1H-indol-3-yl)-piperidin-1-yl]-3-piperidin-4-yl-propan-1-ol bis-trifluoroacetate salt Compound 10b (0.11 mmol, 1 eq) and triethylamine (46 mL, 0.33 mmol, 3 eq) were dissolved in a mixture of DCM (1 mL) and DMF (0.5 mL). 1-bromo-3-isothiocyanato-benzene Compound 3a (24 mg, 0.11 mmol, 1 eq) was added and the mixture was stirred for 16 hrs. The reaction mixture was evaporated to dryness, the residue diluted with DCM and partitioned with saturated sodium bicarbonate. The organic layer was removed and the aqueous layer was extracted with DCM. The combined organic extracts were dried over anhydrous sodium sulfate, then filtered and evaporated to provide a crude residue that was subjected to silica gel chromatography (gradient 2% to 10% 2M MeOH.NH₃ in CH₂Cl₂) to provide Compound 59 (61 mg, quant.) as a pink foam. MS m/z 556 (M+H)⁺.

### EXAMPLE 13

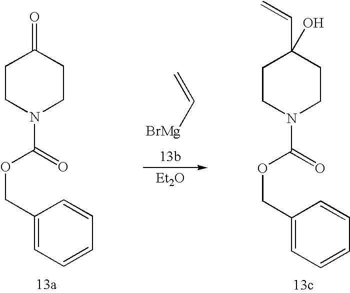

A solution of vinylmagnesium bromide Compound 13b (1.0M solution in THF, 300 mL, 0.3 mol, 1.3 eq) was added via syringe over 10-20 min to a solution of 4-oxo-piperidine-1-carboxylic acid benzyl ester Compound 13a (53.83 g, 0.23 mol, 1 eq) in diethyl ether (500 mL) under a nitrogen atmosphere at 0° C. The mixture was stirred for 2 hrs at 0° C. A saturated aqueous solution of NH₄Cl (300 mL) was added and the reaction mixture was warmed to RT and stirred for 5 min. The quenched reaction was diluted with H₂O (225 mL) and diethyl ether (500 mL). The organic layer was separated and dried over anhydrous Na₂SO₄, then filtered and concentrated in vacuo to give 4-hydroxy-4-vinyl-piperidine-1-carboxylic acid benzyl ester Compound 13c (58.7 g, 97%) as a light yellow oil.

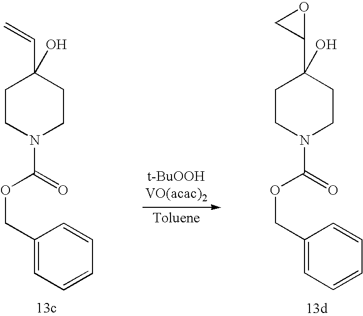

VO(acac)₂ (0.225 g, 0.85 mmol, 0.015 eq) was added to a solution of Compound 13c (15.0 g, 0.0574 mol, 1 eq) in toluene (150 mL). A 70% aqueous solution of tert-butyl-hydroxy-peroxide (16.5 mL, 0.1153, 2 eq) was added via syringe over 3-5 min and the mixture was stirred at ambient temperature for 18 hrs. Additional amounts of VO(acac)₂ (0.1125 g, 0.0075 eq) and tert-butyl-hydroxy-peroxide (8.25 mL, 1 eq) were added until the reaction was shown to be complete via TLC (30% EtOAc—CH₂Cl₂). The reaction mixture was stirred at ambient temperature for 12 hrs. The reaction was quenched by the slow addition of a saturated solution of Na₂SO₃ (110 mL). The reaction mixture was diluted with diethyl ether (300 mL) and the separated organic layer was dried over anhydrous Na₂SO₄, then filtered and concentrated in vacuo to give 4-hydroxy-4-oxiranyl-piperidine-1-carboxylic acid benzyl ester Compound 13d (15.9 g, 100%) as a clear yellow-orange oil.

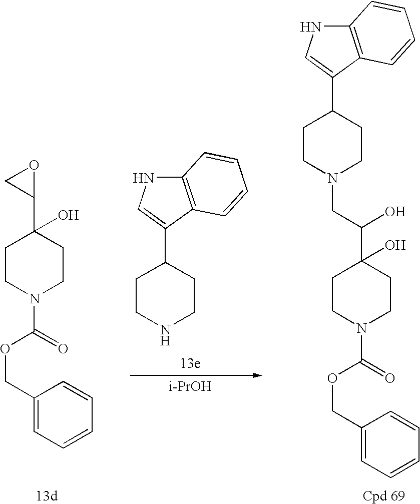

Compound 13d (9.2 g, 0.0332 mol, 1 eq) and 3-piperidin-4-yl-1H-indole Compound 13e (7 g, 0.035 mol, 1.05 eq) were dissolved in isopropanol (75 mL) and heated at 85° C. (bath temperature) for 24 hrs. The reaction mixture was cooled to RT and concentrated in vacuo. The residue was loaded (using EtOAc) onto a sintered glass funnel (8.5 cm diameter) filled with silica (5 cm) that was equilibrated with EtOAc. A slight vacuum was applied and EtOAc (500 mL) was passed through the funnel and collected in a first receiving flask. The first receiving flask was replaced with a second receiving flask and 10% CH₃OH—CH₂Cl₂ (1000 mL) was passed through the funnel under vacuum to collect a second fraction. The second fraction was concentrated in vacuo to give Compound 69 (9.8 g, 62%) as a tan foam.

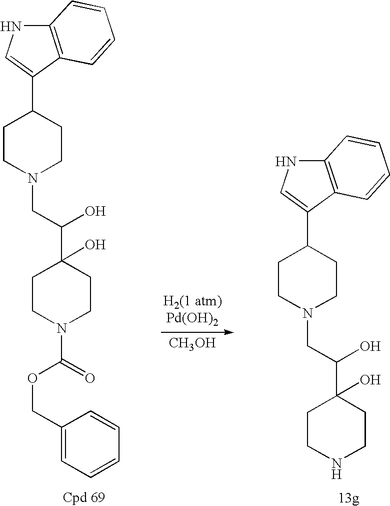

Compound 69 (11.4 g, 0.0239 mol, 1 eq) and palladium hydroxide (1.2 g, 20 wt %) were placed in a flask and methanol (120 mL) was added. The reaction vessel was purged with hydrogen gas two times, then the mixture was stirred under 1 atmosphere of hydrogen (balloon) for 24 hrs. The reaction mixture was purged with nitrogen and then filtered though Celite. The filter cake washed with methanol (200 mL) and the filtrate concentrated in vacuo to give 4-{1-hydroxy-2-[4-(1H-indol-3-yl)-piperidin-1-yl]-ethyl}-piperidin-4-ol Compound 13g (6.7 g, 82%) as a tan foam.

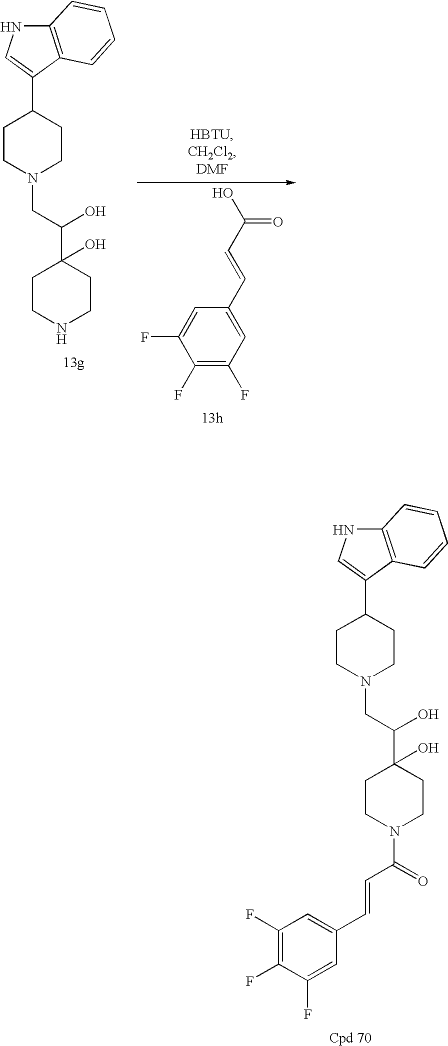

Compound 13g (0675 g, 1.96 mmol, 1 eq), HBTU (0.885 g, 2.36 mmol, 1.2 eq), and 3-(3,4,5-trifluoro-phenyl)-acrylic acid Compound 13h (0.44 g, 2.16 mmol, 1.1 eq) were dissolved in a 5:1 mixture of CH₂Cl₂:DMF (12 mL total; 10 mL:2 mL). The solution was stirred for 12 hrs at RT. The reaction mixture was then diluted with CH₂Cl₂ (100 mL) and washed with saturated aqueous NaHCO₃ (25 mL). The organic layer was dried over anhydrous Na₂SO₄, then filtered and concentrated in vacuo. The residue was purified by silica gel chromatography (10% 2M N₃—CH₃OH in CH₂Cl₂) to give Compound 70 (0.55 g, 53%) as a brown foam.

Using the procedures of Example 13 and known appropriate reagents and starting materials, the following compounds of the invention were prepared:

| Cpd | Name | MS |
|---|---|---|
| 1 | 3-(3,4-dichloro-phenyl)-1-(4-hydroxy-4-{1-hydroxy-2-[4-(4- | 533 |
| | methoxy-phenyl)-piperidin-1-yl]-ethyl}-piperidin-1-yl)- | |
| | propenone | |
| 71 | 3-(3,5-difluoro-phenyl)-1-(4-hydroxy-4-{1-hydroxy-2-[4-(1H- | 510 |
| | indol-3-yl)-piperidin-1-yl]-ethyl}-piperidin-1-yl)-propenone | |
| 72 | 3-(3,5-bis-trifluoromethyl-phenyl)-1-(4-hydroxy-4-{1-hydroxy-2-[4-(1H- | 610 |
| | indol-3-yl)-piperidin-1-yl]-ethyl}-piperidin-1-yl)- | |
| | propenone | |
| 73 | 3-(3,5-dichloro-phenyl)-1-(4-hydroxy-4-{1-hydroxy-2-[4-(1H- | 542 |
| | indol-3-yl)-piperidin-1-yl]-ethyl}-piperidin-1-yl)-propenone | |
| 74 | 3-(3-chloro-5-fluoro-phenyl)-1-(4-hydroxy-4-{1-hydroxy-2-[4- | 526 |
| | (1H-indol-3-yl)-piperidin-1-yl]-ethyl}-piperidin-1-yl)-propenone | |
| 75 | 3-(3,4-dichloro-phenyl)-1-(4-hydroxy-4-{1-hydroxy-2-[4-(1H- | 542 |
| | indol-3-yl)-piperidin-1-yl]-ethyl}-piperidin-1-yl)-propenone | |
| 76 | 3-(3-fluoro-phenyl)-1-(4-hydroxy-4-{1-hydroxy-2-[4-(1H-indol- | 492 |
| | 3-yl)-piperidin-1-yl]-ethyl}-piperidin-1-yl)-propenone | |
| 77 | 1-(4-hydroxy-4-{1-hydroxy-2-[4-(1H-indol-3-yl)-piperidin-1-yl]- | 542 |
| | ethyl}-piperidin-1-yl)-3-(3-trifluoromethyl-phenyl)-propenone | |
| 78 | 3-(3,4-difluoro-phenyl)-1-(4-hydroxy-4-{1-hydroxy-2-[4-(1H- | 510 |
| | indol-3-yl)-piperidin-1-yl]-ethyl}-piperidin-1-yl)-propenone | |
| 79 | 1-(4-hydroxy-4-{1-hydroxy-2-[4-(1H-indol-3-yl)-piperidin-1-yl]- | 480 |
| | ethyl}-piperidin-1-yl)-3-thiophen-3-yl-propenone | |
| 80 | 3-(3-bromo-4-fluoro-phenyl)-1-(4-hydroxy-4-{1-hydroxy-2-[4- | 570 |
| | (1H-indol-3-yl)-piperidin-1-yl]-ethyl}-piperidin-1-yl)-propenone | |
| 87 | 1-(4-{2-[4-(1H-benzoimidazol-2-yl)-piperidin-1-yl]-1-hydroxy- | 529 |
| | ethyl}-4-hydroxy-piperidin-1-yl)-3-(3,4,5-trifluoro-phenyl)-propenone | |
| 88 | 1-(4-hydroxy-4-{1-hydroxy-2-[4-(4-methoxy-phenyl)-piperidin- | 519 |
| | 1-yl]-ethyl}-piperidin-1-yl)-3-(3,4,5-trifluoro-phenyl)-propenone | |
| 92 | 3-(4-chloro-phenyl)-1-(4-hydroxy-4-{1-hydroxy-2-[4-(4- | 499 |
| | methoxy-phenyl)-piperidin-1-yl]-ethyl}-piperidin-1-yl)- | |
| | propenone | |
| 93 | 3-(3,4-difluoro-phenyl)-1-(4-hydroxy-4-{1-hydroxy-2-[4-(4- | 501 |
| | methoxy-phenyl)-piperidin-1-yl]-ethyl}-piperidin-1-yl)- | |
| | propenone | |
| 94 | 3-(3,5-difluoro-phenyl)-1-(4-hydroxy-4-{1-hydroxy-2-[4-(4- | 501 |
| | methoxy-phenyl)-piperidin-1-yl]-ethyl}-piperidin-1-yl)- | |
| | propenone | |
| 95 | 1-(4-hydroxy-4-{1-hydroxy-2-[4-(4-methoxy-phenyl)-piperidin- | 479 |
| | 1-yl]-ethyl}-piperidin-1-yl)-3-m-tolyl-propenone | |
| 112 | 4-{2-[4-(4-chloro-phenyl)-piperidin-1-yl]-1-hydroxy-ethyl}-4- | 473 |
| | hydroxy-piperidine-1-carboxylic acid benzyl ester | |
| 113 | 1-(4-hydroxy-4-{1-hydroxy-2-[4-(4-methoxy-phenyl)-piperidin- | 471 |
| | 1-yl]-ethyl}-piperidin-1-yl)-3-thiophen-3-yl-propenone | |
| 115 | 4-hydroxy-4-{1-hydroxy-2-[4-(4-methoxy-phenyl)-piperidin-1- | 490 |
| | yl]-ethyl}-piperidine-1-carboxylic acid (3,5-difluoro-phenyl)- | |
| | amide | |
| 125 | 4-{2-[4-(4-fluoro-phenyl)-piperidin-1-yl]-1-hydroxy-ethyl}-4- | 457 |
| | hydroxy-piperidine-1-carboxylic acid benzyl ester | |
| 126 | 4-hydroxy-4-{1-hydroxy-2-[4-(4-trifluoromethyl-phenyl)- | 507 |
| | piperidin-1-yl]-ethyl}-piperidine-1-carboxylic acid benzyl ester | |
| 128 | 1-(4-{2-[4-(4-fluoro-phenyl)-piperidin-1-yl]-1-hydroxy-ethyl}-4- | 507 |
| | hydroxy-piperidin-1-yl)-3-(3,4,5-trifluoro-phenyl)-propenone | |
| 129 | 3-(3,4-difluoro-phenyl)-1-(4-{2-[4-(4-fluoro-phenyl)-piperidin-1- | 489 |
| | yl]-1-hydroxy-ethyl}-4-hydroxy-piperidin-1-yl)-propenone | |
| 130 | 3-(3,5-difluoro-phenyl)-1-(4-{2-[4-(4-fluoro-phenyl)-piperidin-1- | 489 |
| | yl]-1-hydroxy-ethyl}-4-hydroxy-piperidin-1-yl)-propenone | |
| 131 | 3-(3,4-dichloro-phenyl)-1-(4-{2-[4-(4-fluoro-phenyl)-piperidin-1- | 521 |
| | yl]-1-hydroxy-ethyl}-4-hydroxy-piperidin-1-yl)-propenone | |
| 136 | 1-(4-hydroxy-4-{1-hydroxy-2-[4-(4-trifluoromethyl-phenyl)- | 557 |
| | piperidin-1-yl]-ethyl}-piperidin-1-yl)-3-(3,4,5-trifluoro-phenyl)- | |
| | propenone | |
| 137 | 3-(3,4-difluoro-phenyl)-1-(4-hydroxy-4-{1-hydroxy-2-[4-(4- | 539 |
| | trifluoromethyl-phenyl)-piperidin-1-yl]-ethyl}-piperidin-1-yl)- | |
| | propenone | |
| 138 | 3-(3,5-difluoro-phenyl)-1-(4-hydroxy-4-{1-hydroxy-2-[4-(4- | 539 |
| | trifluoromethyl-phenyl)-piperidin-1-yl]-ethyl}-piperidin-1-yl)- | |
| | propenone | |
| 139 | 3-(3,4-dichloro-phenyl)-1-(4-hydroxy-4-{1-hydroxy-2-[4-(4- | 571 |
| | trifluoromethyl-phenyl)-piperidin-1-yl]-ethyl}-piperidin-1-yl)- | |
| | propenone | |
| 140 | 3-(3,4-dimethoxy-phenyl)-1-(4-hydroxy-4-{1-hydroxy-2-[4-(4- | 563 |
| | trifluoromethyl-phenyl)-piperidin-1-yl]-ethyl}-piperidin-1-yl)- | |
| | propenone | |
| 143 | 4-hydroxy-4-{1-hydroxy-2-[4-(4-trifluoromethyl-phenyl)- | 560 |
| | piperidin-1-yl]-ethyl}-piperidine-1-carboxylic acid (3,4-dichloro- | |
| | phenyl)-amide | |
| 146 | 4-{2-[4-(4-chloro-phenyl)-piperidin-1-yl]-1-hydroxy-ethyl}-4- | 439 |
| | hydroxy-piperidine-1-carboxylic acid tert-butyl ester | |
| 148 | 1-(4-hydroxy-4-{1-hydroxy-2-[4-(5-morpholin-4-yl-1H-indol-3- | 613 |
| | yl)-piperidin-1-yl]-ethyl}-piperidin-1-yl)-3-(3,4,5-trifluoro- | |
| | phenyl)-propenone | |
| 149 | 3-(3,5-difluoro-phenyl)-1-(4-hydroxy-4-{1-hydroxy-2-[4-(5- | 595 |
| | morpholin-4-yl-1H-indol-3-yl)-piperidin-1-yl]-ethyl}-piperidin- | |
| | 1-yl)-propenone | |
| 150 | 3-(3,5-bis-trifluoromethyl-phenyl)-1-(4-hydroxy-4-{1-hydroxy-2- | 695 |
| | [4-(5-morpholin-4-yl-1H-indol-3-yl)-piperidin-1-yl]-ethyl}- | |
| | piperidin-1-yl)-propenone | |
| 151 | 3-(3,5-dichloro-phenyl)-1-(4-hydroxy-4-{1-hydroxy-2-[4-(5- | 627 |
| | morpholin-4-yl-1H-indol-3-yl)-piperidin-1-yl]-ethyl}-piperidin- | |
| | 1-yl)-propenone | |
| 152 | 1-(4-{2-[4-(4-fluoro-phenyl)-4-hydroxy-piperidin-1-yl]-1- | 523 |
| | hydroxy-ethyl}-4-hydroxy-piperidin-1-yl)-3-(3,4,5-trifluoro- | |
| | phenyl)-propenone | |
| 153 | 3-(3,4-difluoro-phenyl)-1-(4-{2-[4-(4-fluoro-phenyl)-4-hydroxy- | 505 |
| | piperidin-1-yl]-1-hydroxy-ethyl}-4-hydroxy-piperidin-1-yl)- | |
| | propenone | |
| 154 | 3-(3,5-difluoro-phenyl)-1-(4-{2-[4-(4-fluoro-phenyl)-4-hydroxy- | 505 |
| | piperidin-1-yl]-1-hydroxy-ethyl}-4-hydroxy-piperidin-1-yl)- | |
| | propenone | |
| 155 | 1-(4-{2-[4-(5-fluoro-1H-indol-3-yl)-piperidin-1-yl]-1-hydroxy- | 546 |
| | ethyl}-4-hydroxy-piperidin-1-yl)-3-(3,4,5-trifluoro-phenyl)- | |
| | propenone | |
| 156 | 3-(3,4-difluoro-phenyl)-1-(4-{2-[4-(5-fluoro-1H-indol-3-yl)- | 528 |
| | piperidin-1-yl]-1-hydroxy-ethyl}-4-hydroxy-piperidin-1-yl)- | |
| | propenone | |
| 157 | 3-(3,5-difluoro-phenyl)-1-(4-{2-[4-(5-fluoro-1H-indol-3-yl)- | 528 |
| | piperidin-1-yl]-1-hydroxy-ethyl}-4-hydroxy-piperidin-1-yl)- | |
| | propenone | |
| 158 | 3-(3,4-dichloro-phenyl)-1-(4-{2-[4-(5-fluoro-1H-indol-3-yl)- | 560 |
| | piperidin-1-yl]-1-hydroxy-ethyl}-4-hydroxy-piperidin-1-yl)- | |
| | propenone | |
| 159 | 3-(3-bromo-4-fluoro-phenyl)-1-(4-{2-[4-(5-fluoro-1H-indol-3- | 588 |
| | yl)-piperidin-1-yl]-1-hydroxy-ethyl}-4-hydroxy-piperidin-1-yl)- | |
| | propenone | |
| 160 | 3-(4-chloro-phenyl)-1-(4-{2-[4-(5-fluoro-1H-indol-3-yl)- | 528 |
| | piperidin-1-yl]-1-hydroxy-ethyl}-4-hydroxy-piperidin-1-yl)- | |
| | propan-1-one | |
| 163 | 1-(4-{2-[4-(4-chloro-phenyl)-piperidin-1-yl]-1-hydroxy-ethyl}-4- | 523 |
| | hydroxy-piperidin-1-yl)-3-(3,4,5-trifluoro-phenyl)-propenone | |
| 164 | 1-(4-{2-[4-(4-chloro-phenyl)-piperidin-1-yl]-1-hydroxy-ethyl}-4- | 505 |
| | hydroxy-piperidin-1-yl)-3-(3,5-difluoro-phenyl)-propenone | |
| 168 | 3-(3,4-dichloro-phenyl)-1-(4-{2-[4-(5-fluoro-1H-indol-3-yl)- | 562 |
| | piperidin-1-yl]-1-hydroxy-ethyl}-4-hydroxy-piperidin-1-yl)- | |
| | propan-1-one | |
| 169 | 3-(3,4-dichloro-phenyl)-1-(4-hydroxy-4-{1-hydroxy-2-[4-(5- | 627 |
| | morpholin-4-yl-1H-indol-3-yl)-piperidin-1-yl]-ethyl}-piperidin- | |
| | 1-yl)-propenone | |
| 170 | 3-(4-chloro-phenyl)-1-(4-hydroxy-4-{1-hydroxy-2-[4-(5- | 593 |
| | morpholin-4-yl-1H-indol-3-yl)-piperidin-1-yl]-ethyl}-piperidin- | |
| | 1-yl)-propenone | |
| 171 | 3-(4-fluoro-phenyl)-1-(4-hydroxy-4-{1-hydroxy-2-[4-(5- | 577 |
| | morpholin-4-yl-1H-indol-3-yl)-piperidin-1-yl]-ethyl}-piperidin- | |
| | 1-yl)-propenone | |
| 172 | 3-(3,4-difluoro-phenyl)-1-(4-hydroxy-4-{1-hydroxy-2-[4-(5- | 595 |
| | morpholin-4-yl-1H-indol-3-yl)-piperidin-1-yl]-ethyl}-piperidin- | |
| | 1-yl)-propenone | |
| 173 | 3-(3-bromo-4-fluoro-phenyl)-1-(4-hydroxy-4-{1-hydroxy-2-[4- | 655 |
| | (5-morpholin-4-yl-1H-indol-3-yl)-piperidin-1-yl]-ethyl}- | |
| | piperidin-1-yl)-propenone | |
| 174 | 1-(4-hydroxy-4-{1-hydroxy-2-[4-(5-morpholin-4-yl-1H-indol-3- | 573 |
| | yl)-piperidin-1-yl]-ethyl}-piperidin-1-yl)-3-m-tolyl-propenone | |
| 175 | 1-(4-hydroxy-4-{1-hydroxy-2-[4-(5-morpholin-4-yl-1H-indol-3- | 613 |
| | yl)-piperidin-1-yl]-ethyl}-piperidin-1-yl)-3-(2,4,5-trifluoro- | |
| | phenyl)-propenone | |
| 176 | 1-(4-hydroxy-4-{1-hydroxy-2-[4-(5-morpholin-4-yl-1H-indol-3- | 592 |
| | yl)-piperidin-1-yl]-ethyl}-piperidin-1-yl)-2-(3-nitro-phenyl)- | |
| | ethanone | |
| 177 | 1-(4-hydroxy-4-{1-hydroxy-2-[4-(5-morpholin-4-yl-1H-indol-3- | 589 |
| | yl)-piperidin-1-yl]-ethyl}-piperidin-1-yl)-3-(3-methoxy-phenyl)- | |
| | propenone | |
| 178 | 1-(4-hydroxy-4-{1-hydroxy-2-[4-(5-morpholin-4-yl-1H-indol-3- | 565 |
| | yl)-piperidin-1-yl]-ethyl}-piperidin-1-yl)-3-thiophen-3-yl- | |
| | propenone | |

### EXAMPLE 14

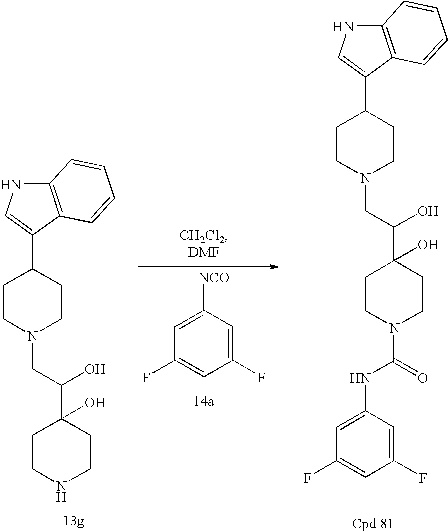

4-{1-hydroxy-2-[4-(1H-indol-3-yl)-piperidin-1-yl]-ethyl}-piperidin-4-ol Compound 13g (0.05 g, 0.146 mmol, 1 eq) and 1,3-difluoro-5-isocyanato-benzene Compound 14a were dissolved in a 10:1 mixture of CH₂Cl₂:DMF (2.2 mL total, 2.0 mL:0.2 mL) and stirred at RT for 18 hrs. The reaction mixture was purified via silica gel chromatography (10% 2M N₃—CH₃OH in CH₂Cl₂) to give Compound 81 (0.0158 g, 22%) as a white solid.

Using the procedures of Example 14 and known appropriate reagents and starting materials, the following compounds of the invention were prepared:

| Cpd | Name | MS |
|---|---|---|
| 82 | 4-hydroxy-4-{1-hydroxy-2-[4-(1H-indol-3-yl)-piperidin-1-yl]- | 499 |
| | ethyl}-piperidine-1-carboxylic acid (3,4-difluoro-phenyl)-amide | |
| 114 | 4-hydroxy-4-{1-hydroxy-2-[4-(4-methoxy-phenyl)-piperidin-1- | 490 |
| | yl]-ethyl}-piperidine-1-carboxylic acid (3,4-difluoro-phenyl)- | |
| | amide | |
| 116 | 4-hydroxy-4-{1-hydroxy-2-[4-(4-methoxy-phenyl)-piperidin-1- | 522 |
| | yl]-ethyl}-piperidine-1-carboxylic acid (3,4-dichloro-phenyl)- | |
| | amide | |
| 132 | 4-{2-[4-(4-fluoro-phenyl)-piperidin-1-yl]-1-hydroxy-ethyl}-4- | 478 |
| | hydroxy-piperidine-1-carboxylic acid (3,4-difluoro-phenyl)- | |
| | amide | |
| 133 | 4-{2-[4-(4-fluoro-phenyl)-piperidin-1-yl]-1-hydroxy-ethyl}-4- | 478 |
| | hydroxy-piperidine-1-carboxylic acid (3,5-difluoro-phenyl)- | |
| | amide | |
| 134 | 4-{2-[4-(4-fluoro-phenyl)-piperidin-1-yl]-1-hydroxy-ethyl}-4- | 510 |
| | hydroxy-piperidine-1-carboxylic acid (3,4-dichloro-phenyl)- | |
| | amide | |
| 141 | 4-hydroxy-4-{1-hydroxy-2-[4-(4-trifluoromethyl-phenyl)- | 528 |
| | piperidin-1-yl]-ethyl}-piperidine-1-carboxylic acid (3,4-difluoro- | |
| | phenyl)-amide | |
| 142 | 4-hydroxy-4-{1-hydroxy-2-[4-(4-trifluoromethyl-phenyl)- | 528 |
| | piperidin-1-yl]-ethyl}-piperidine-1-carboxylic acid (3,5-difluoro- | |
| | phenyl)-amide | |
| 161 | 4-{2-[4-(5-fluoro-1H-indol-3-yl)-piperidin-1-yl]-1-hydroxy- | 533 |
| | ethyl}-4-hydroxy-piperidine-1-carboxylic acid (2-chloro-5- | |
| | fluoro-phenyl)-amide | |
| 165 | 4-{2-[4-(4-chloro-phenyl)-piperidin-1-yl]-1-hydroxy-ethyl}-4- | 526 |
| | hydroxy-piperidine-1-carboxylic acid (3,4-dichloro-phenyl)- | |
| | amide | |
| 179 | 4-hydroxy-4-{1-hydroxy-2-[4-(5-morpholin-4-yl-1H-indol-3-yl)- | 584 |
| | piperidin-1-yl]-ethyl}-piperidine-1-carboxylic acid (3,4-difluoro-phenyl)- | |
| | amide | |
| 180 | 4-hydroxy-4-{1-hydroxy-2-[4-(5-morpholin-4-yl-1H-indol-3-yl)- | 634 |
| | piperidin-1-yl]-ethyl}-piperidine-1-carboxylic acid (3-fluoro-5- | |
| | trifluoromethyl-phenyl)-amide | |
| 181 | 4-hydroxy-4-{1-hydroxy-2-[4-(5-morpholin-4-yl-1H-indol-3-yl)- | 616 |
| | piperidin-1-yl]-ethyl}-piperidine-1-carboxylic acid (3,4-dichloro- | |
| | phenyl)-amide | |

### EXAMPLE 15

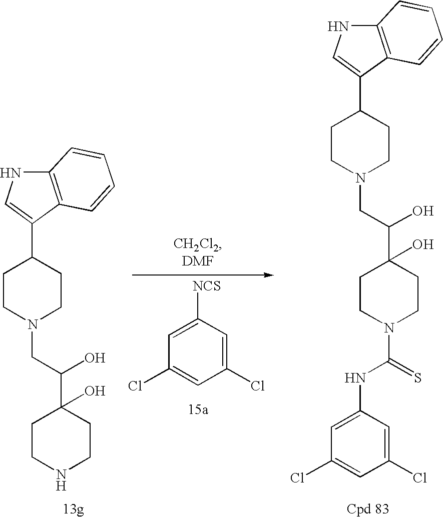

Compound 13g (0.05 g, 0.146 mmol, 1 eq) and 1,3-dichloro-5-isothiocyanato-benzene Compound 15a were dissolved in 10:1 CH₂Cl₂:DMF (2.2 mL, 2.0 mL:0.2 mL) and stirred at RT for 18 hrs. The reaction mixture was purified via silica gel chromatography (10% 2M N₃—CH₃OH in CH₂Cl₂) to give Compound 83 (0.0312 g, 39%) as a white solid. MS m/z 547, 549, 551 (M+H)⁺.

Using the procedure of Example 15 and known appropriate reagents and starting materials, the following compounds of the invention were prepared:

| Cpd | Name | MS |
|---|---|---|
| 84 | 4-hydroxy-4-{1-hydroxy-2-[4-(1H-indol-3-yl)-piperidin-1-yl]- | 547 |
| | ethyl}-piperidine-1-carbothioic acid (3,4-dichloro-phenyl)-amide | |
| 117 | 4-hydroxy-4-{1-hydroxy-2-[4-(4-methoxy-phenyl)-piperidin-1- | 538 |
| | yl]-ethyl}-piperidine-1-carbothioic acid (3,4-dichloro-phenyl)- | |
| | amide | |
| 118 | 4-hydroxy-4-{1-hydroxy-2-[4-(4-methoxy-phenyl)-piperidin-1- | 538 |
| | yl]-ethyl}-piperidine-1-carbothioic acid (3,5-dichloro-phenyl)- | |
| | amide | |
| 119 | 4-hydroxy-4-{1-hydroxy-2-[4-(4-methoxy-phenyl)-piperidin-1- | 528 |
| | yl]-ethyl}-piperidine-1-carbothioic acid (3,5-dimethoxy-phenyl)-amide | |
| 135 | 4-{2-[4-(4-fluoro-phenyl)-piperidin-1-yl]-1-hydroxy-ethyl}-4- | 526 |
| | hydroxy-piperidine-1-carbothioic acid (3,4-dichloro-phenyl)- | |
| | amide | |
| 144 | 4-hydroxy-4-{1-hydroxy-2-[4-(4-trifluoromethyl-phenyl)- | 576 |
| | piperidin-1-yl]-ethyl}-piperidine-1-carbothioic acid (3,4- | |
| | dichloro-phenyl)-amid | |
| 145 | 4-hydroxy-4-{1-hydroxy-2-[4-(4-trifluoromethyl-phenyl)- | 576 |
| | piperidin-1-yl]-ethyl}-piperidine-1-carbothioic acid (3,5- | |
| | dichloro-phenyl)-amide | |
| 162 | 4-{2-[4-(5-fluoro-1H-indol-3-yl)-piperidin-1-yl]-1-hydroxy- | 565 |
| | ethyl}-4-hydroxy-piperidine-1-carbothioic acid (3,4-dichloro- | |
| | phenyl)-amide | |
| 166 | 4-{2-[4-(4-chloro-phenyl)-piperidin-1-yl]-1-hydroxy-ethyl}-4- | 528 |
| | hydroxy-piperidine-1-carbothioic acid (2,3,5-trifluoro-phenyl)- | |
| | amide | |
| 167 | 4-{2-[4-(5-fluoro-1H-indol-3-yl)-piperidin-1-yl]-1-hydroxy- | 549 |
| | ethyl}-4-hydroxy-piperidine-1-carboxylic acid (3,4-dichloro- | |
| | phenyl)-amide | |
| 182 | 4-hydroxy-4-{1-hydroxy-2-[4-(5-morpholin-4-yl-1H-indol-3-yl)- | 632 |
| | piperidin-1-yl]-ethyl}-piperidine-1-carbothioic acid (3,5- | |
| | dichloro-phenyl)-amide | |
| 183 | 4-hydroxy-4-{1-hydroxy-2-[4-(5-morpholin-4-yl-1H-indol-3-yl)- | 600 |
| | piperidin-1-yl]-ethyl}-piperidine-1-carbothioic acid (3,5-difluoro-phenyl)- | |
| | amide | |

### EXAMPLE 16

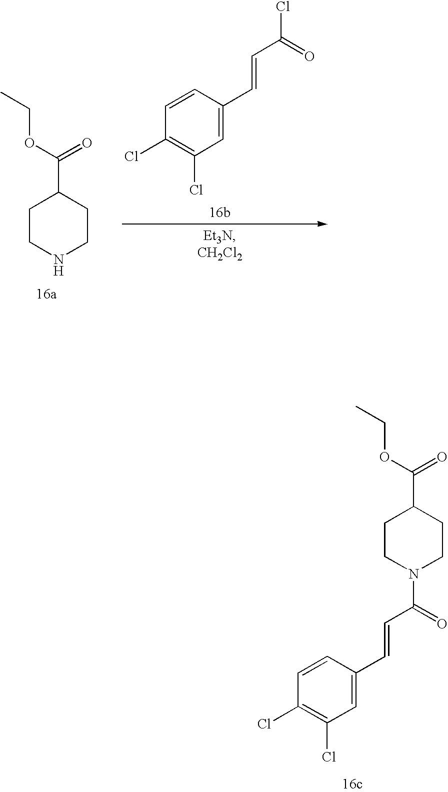

A mixture of piperidine-4-carboxylic acid ethyl ester Compound 16a (1.57 g, 10 mmol), 3-(3,4-dichloro-phenyl)-acryloyl chloride Compound 16b and triethyl amine (2 mL) in DCM (50 mL) was stirred overnight. The reaction mixture washed sequentially with 2N HCl (10 mL×3), 1N NaOH (10 mL) and brine (10 mL×2). The organic layer was dried over Na₂SO₄. Evaporation of DCM gave the product 1-[3-(3,4-dichloro-phenyl)-acryloyl]-piperidine-4-carboxylic acid ethyl ester Compound 16c (3.4 g, 96%).

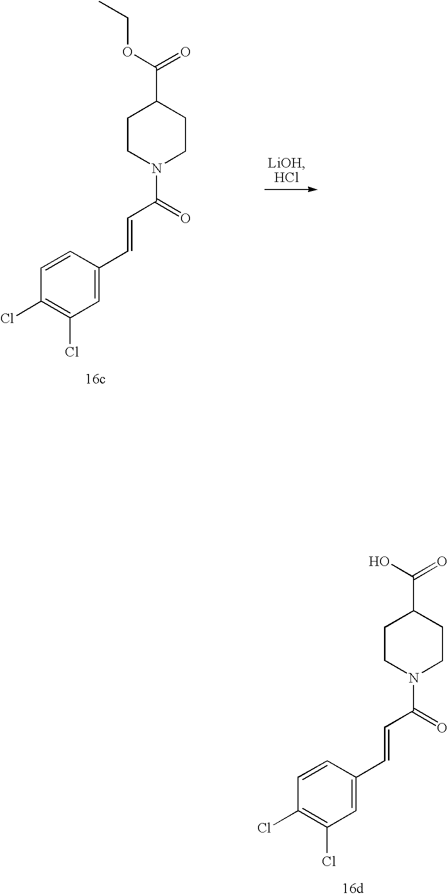

A solution of lithium hydroxide monohydrate (0.8 g) in water (10 mL) was added to the solution of Compound 16c (3.4 g, 9.6 mmol) in THF (30 mL) and methanol (10 mL). The mixture was stirred for 3 hrs, then the reaction was quenched with HCl and concentrated in vacuo. The product was extracted with DCM (20 mL×2). Evaporation of DCM provided 1-[3-(3,4-dichlorophenyl)-acryloyl]-piperidine-4-carboxylic acid Compound 16d (2.9 g, 92%).

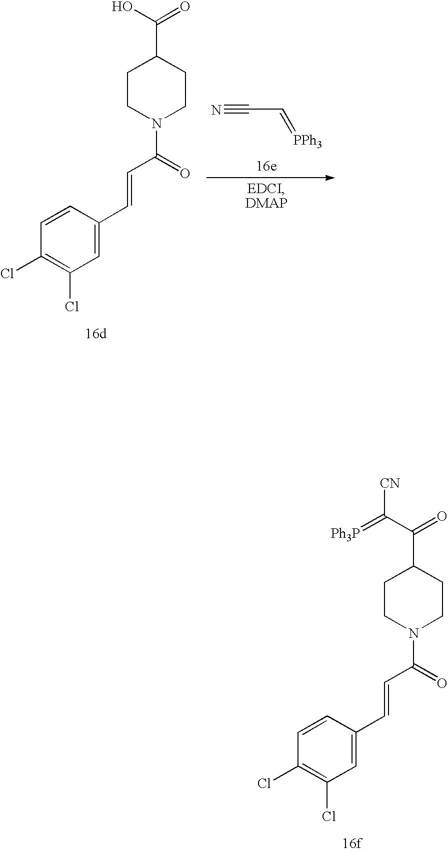

EDCl (1.87 g, 9.76 mmol) was added to a solution of Compound 16d (2.9 g, 8.87 mmol) in DCM (30 mL) and the mixture stirred for 0.5 hrs. (Triphenyl-λ⁵-phosphanylidene)-acetonitrile Compound 16e (2.94 g, 9.76 mmol) and DMAP (0.11 g, 0.89 mmol) were added and the mixture was stirred overnight under nitrogen. The reaction mixture was diluted to 50 mL with DCM and sequentially washed with 1N HCl (20 mL) and brine (20 mL). The crude product was purified via column chromatography (70% EtOAc in hexane) to give 3-{1-[3-(3,4-dichloro-phenyl)-acryloyl]-piperidin-4-yl}-3-oxo-2-(triphenyl-λ⁵-phosphanylidene)-propionitrile Compound 16f (4.0 g, 74%).

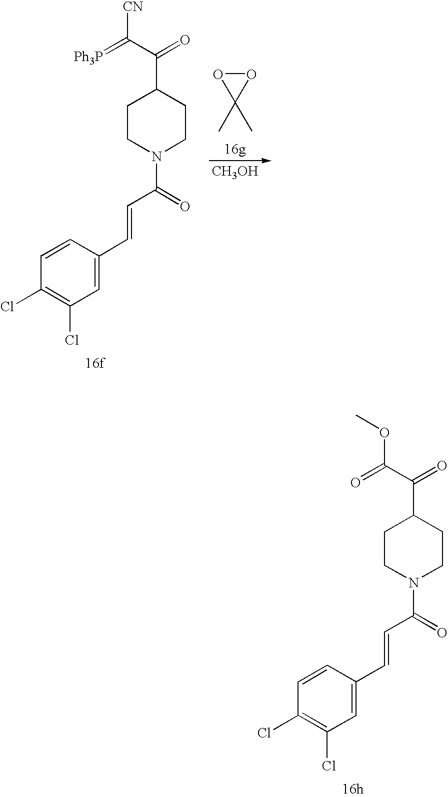

A solution of Compound 16f (0.31 g, 0.5 mmol) in methanol (10 mL) was cooled to −78° C. and then treated with a solution of 3,3-dimethyl-dioxirane Compound 16g in acetone (11 mL, 0.1 M). The mixture was stirred for 4 hrs at −78° C., then warmed to RT. The solvent was evaporated and the resulting crude product was purified with PTLC to give {1-[3-(3,4-dichloro-phenyl)-acryloyl]-piperidin-4-yl}-oxo-acetic acid methyl ester Compound 16h (0.16 g, 85%).

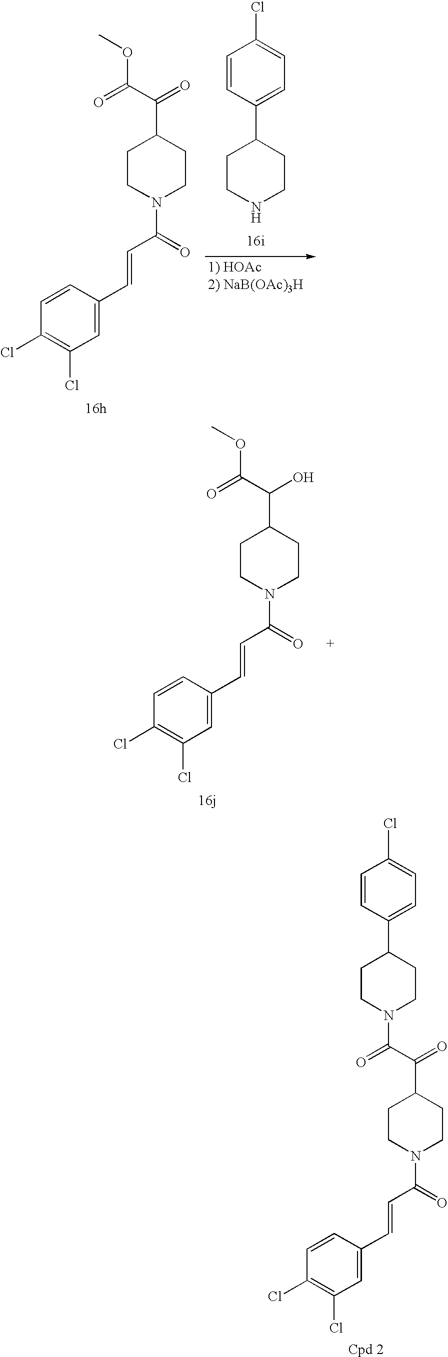

A solution of Compound 16h (0.11 g, 0.3 mmol) and 4-(4-chloro-phenyl)-piperidine Compound 161 (0.06 g, 0.3 mmol) in DCM/acetic acid (10 mL, pH 3-4) was stirred for 1 hr, then sodium triacetoxyborohydride (0.1 g, 0.5 mmol) was added. The mixture was stirred overnight, then neutralized to pH 10. The resulting crude product was purified with PTLC (10% methanol in DCM) to give a mixture of {1-[3-(3,4-dichloro-phenyl)-acryloyl]-piperidin-4-yl}-hydroxy-acetic acid methyl ester Compound 16j (0.08 g, 72%) and Compound 2 (0.02 g, 12%). MS m/z 533, 535, 537 (M+H)⁺.

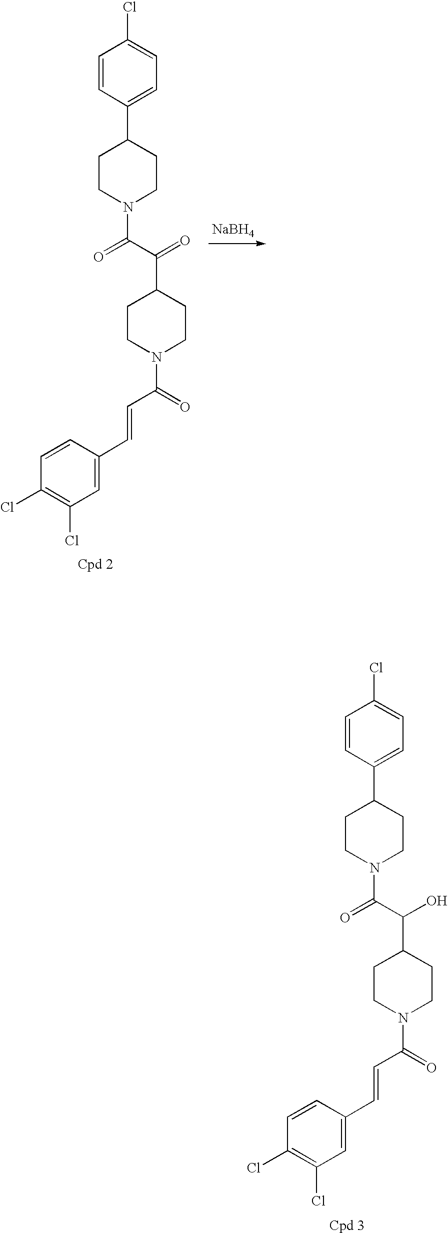

Sodium borohydride (3 mg) was added to a solution of Compound 2 (5 mg, 0.01 mmol) in DCM (5 mL). The mixture was stirred for 1 hr and diluted to 10 mL, then sequentially washed with water (5 mL) and brine (5 mL) and dried over Na₂SO₄. The solvent was evaporated to provide Compound 3 (3 mg, 60%). MS m/z 535, 537, 539 (M+H)⁺.

### EXAMPLE 17

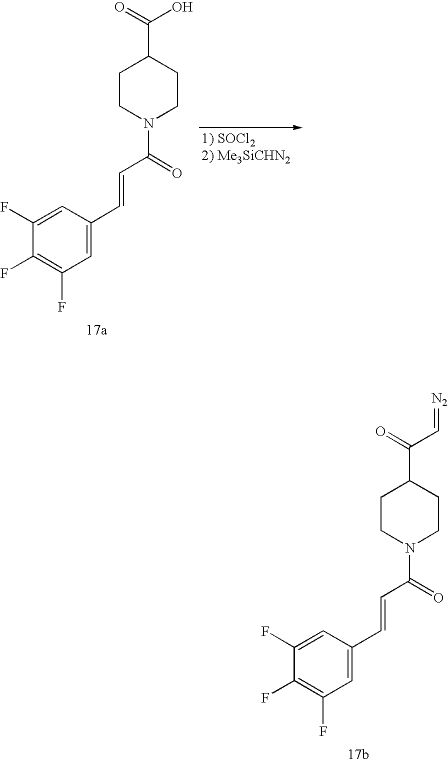

Thionyl chloride (5 mL) was added to 1-[3-(3,4,5-trifluoro-phenyl)-acryloyl]-piperidine-4-carboxylic acid Compound 17a (1 g, 3.2 mmol) in DCM (5 mL). The solution was refluxed for 1 hr and then concentrated in vacuo. The resulting residue was dissolved in CH₃CN (4 mL) and THF (4 mL). The mixture was added under nitrogen to an ice-cooled solution of trimethylsilyl-diazomethane in hexane (2 M, 4 mL). The mixture was stirred over an ice-bath for 5 hrs, then concentrated in vacuo. The resulting residue was chromatographed with 70% EtOAc in hexane to give 1-[4-(2-diazo-acetyl)-piperidin-1-yl]-3-(3,4,5-trifluoro-phenyl)-propenone Compound 17b (0.58 g, 54%).

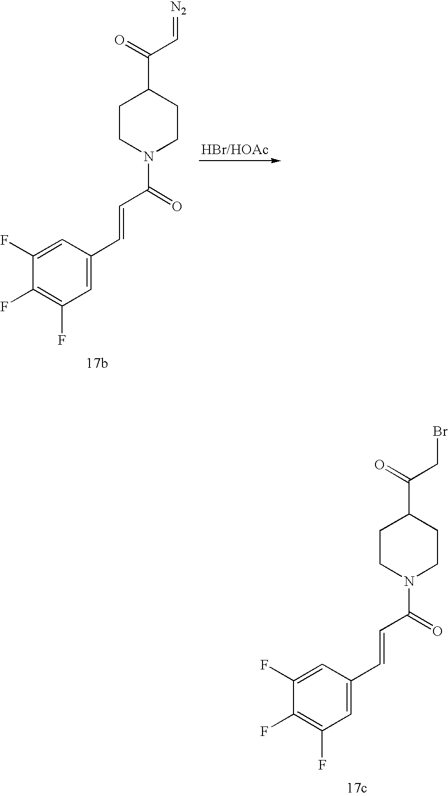

Hydrobromic acid (48%, 0.5 mL) was added to a solution of Compound 17b (0.14 g, 0.42 mmol) in acetic acid (1.5 mL). The mixture was stirred for 1 hr, diluted with DCM (20 mL) and sequentially washed with water (3 mL) and brine (3 mL). The organic layer was dried over Na₂SO₄. The solvents were evaporated to provide 1-[4-(2-bromo-acetyl)-piperidin-1-yl]-3-(3,4,5-trifluoro-phenyl)-propenone Compound 17c (0.16 g, 99%).

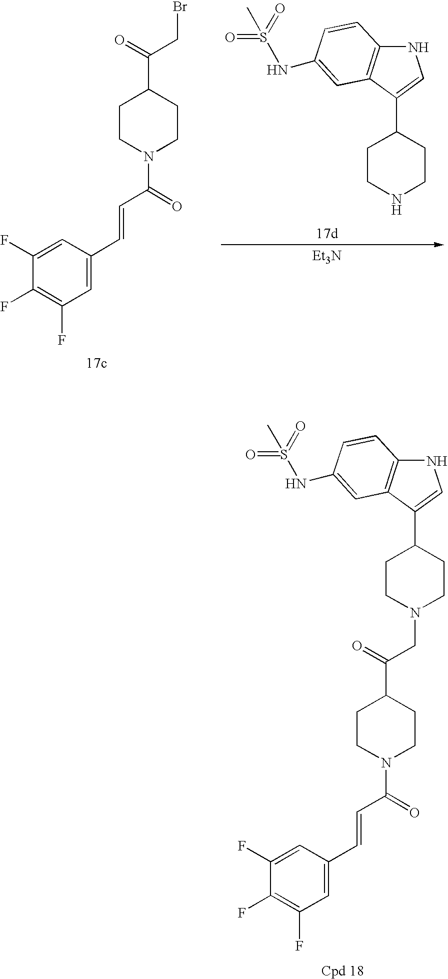

Compound 17c (28 mg, 0.07 mmol) was added to a solution of N-(3-piperidin-4-yl-1H-indol-5-yl)-methanesulfonamide Compound 17d (22 mg, 0.07 mmol) in DCM (10 mL) and triethylamine (0.5 mL). The mixture was stirred overnight and the reaction was quenched with 2N HCl. The crude product was purified with column (10% methanol in DCM) to give Compound 18 (15 mg, 35%). MS m/z 603 (M+H)⁺.

Using the procedure of Example 17 and known appropriate reagents and starting materials, the following compounds of the invention were prepared:

| Cpd | Name | MS |
|---|---|---|
| 17 | 1-(4-{2-[4-(1H-pyrrolo[2,3-b]pyridin-3-yl)-piperidin-1-yl]- | 511 |
| | acetyl}-piperidin-1-yl)-3-(3,4,5-trifluoro-phenyl)-propenone | |
| 64 | 1-(4-{2-[4-(4-methoxy-phenyl)-piperidin-1-yl]-acetyl}- | 501 |
| | piperidin-1-yl)-3-(3,4,5-trifluoro-phenyl)-propenone | |

### EXAMPLE 18

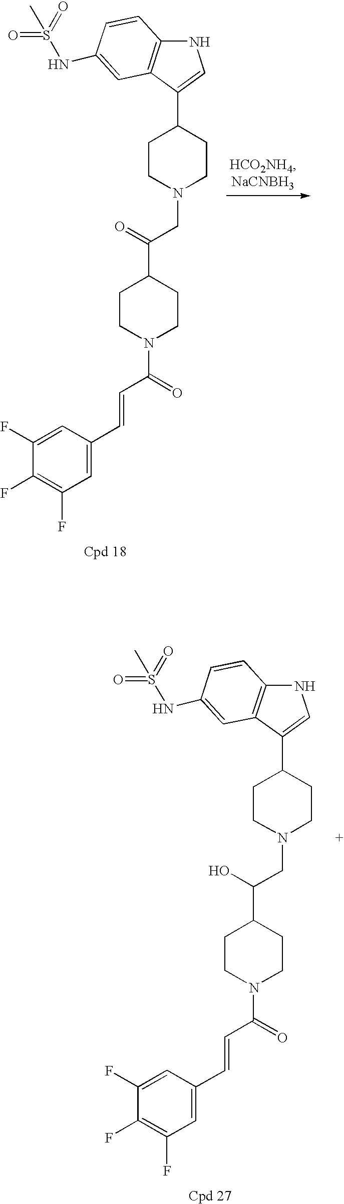

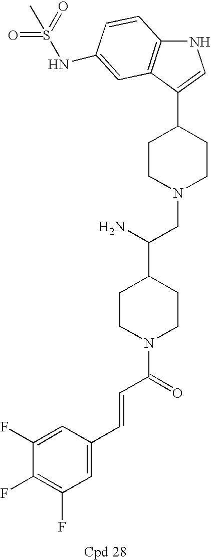

Sodium cyanoborohydride (15 mg) was added to a solution of Compound 18 (10 mg, 0.017 mmol) and ammonium formate (15 mg) in methanol (10 mL) and the mixture was stirred overnight. The reaction was quenched with 1N NaOH. The methanol was evaporated and DCM (10 mL) was added. The organic layer was dried over Na₂SO₄. Evaporation of DCM gave a crude product that was purified via PTLC (15% methanol in DCM) to give a mixture of Compound 27 (2 mg, 20%) and Compound 28 (5 mg, 50%).

Compound 27 MS m/z 605 (M+H)⁺ and Compound 28 MS m/z 604 (M+H)⁺.

Using the procedure of Example 18 and known appropriate reagents and starting materials, the following compounds of the invention were prepared:

| Cpd | Name | MS |
|---|---|---|
| 65 | 1-(4-{1-amino-2-[4-(4-methoxy-phenyl)-piperidin-1-yl]- | 502 |
| | ethyl}-piperidin-1-yl)-3-(3,4,5-trifluoro-phenyl)-propenone | |

### EXAMPLE 19

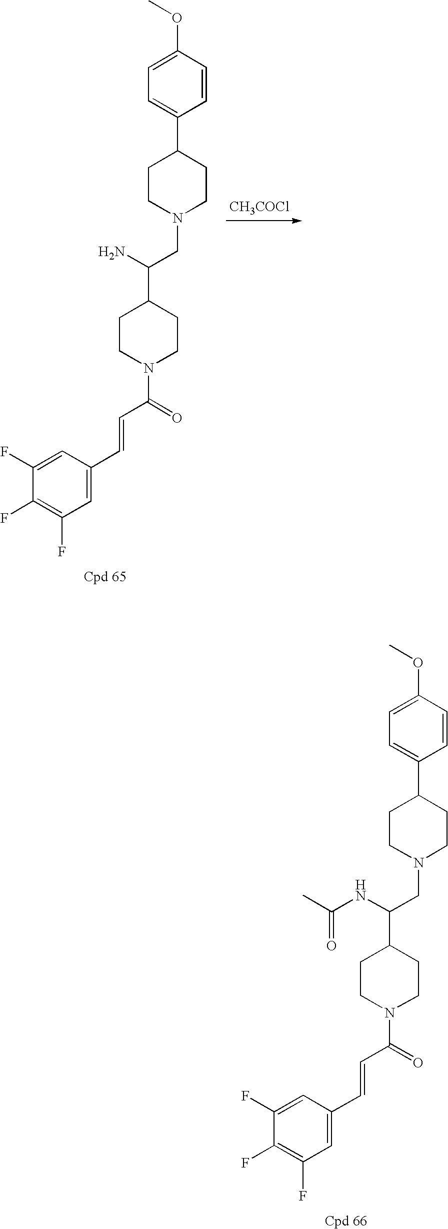

Acetyl chloride (2 mg) was added to a solution of Compound 65 (10 mg, 0.02 mmol) in DCM (10 mL) and triethylamine (0.5 mL). The solution was stirred for 1 hr and then concentrated in vacuo. The resulting residue was purified via PTLC (10% methanol in DCM) to give Compound 66 (7.5 mg, 69%). MS m/z 544 (M+H)⁺.

Using the procedure of Example 19 and known appropriate reagents and starting materials, the following compounds of the invention were prepared:

| Cpd | Name | MS |
|---|---|---|
| 67 | (2-[4-(4-methoxy-phenyl)-piperidin-1-yl]-1-{1-[3-(3,4,5- | 560 |
| | trifluoro-phenyl)-acryloyl]-piperidin-4-yl}-ethyl)- | |
| | carbamic acid methyl ester | |
| 68 | 3-(2-[4-(4-methoxy-phenyl)-piperidin-1-yl]-1-{1-[3-(3,4,5- | 573 |
| | trifluoro-phenyl)-acryloyl]-piperidin-4-yl}-ethyl)-1,1- | |
| | dimethyl-urea | |

### EXAMPLE 20

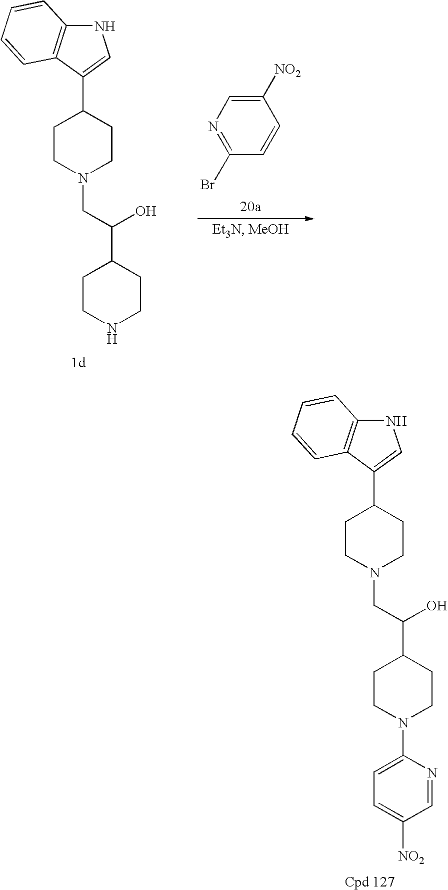

Triethylamine (350 mg, 3.49 mmol, 1.1 eq) and 2-bromo-5-nitro-pyridine Compound 20a (709 mg, 3.49 mmol, 1.1 eq) were added to a suspension of 2-[4-(1H-indol-3-yl)-piperidin-1-yl]-1-piperidin-4-yl-ethanol Compound 1d (1 g, 3.17 mmol, 1 eq) in MeOH (31 mL) at RT. The mixture was heated to reflux with stirring for 16 hrs, cooled to room temperature and the resulting precipitate was filtered and washed with MeOH to provide Compound 127 (1.1 g, 78%) as a bright yellow solid. MS m/z 441 (M+H⁺).

### EXAMPLE 21

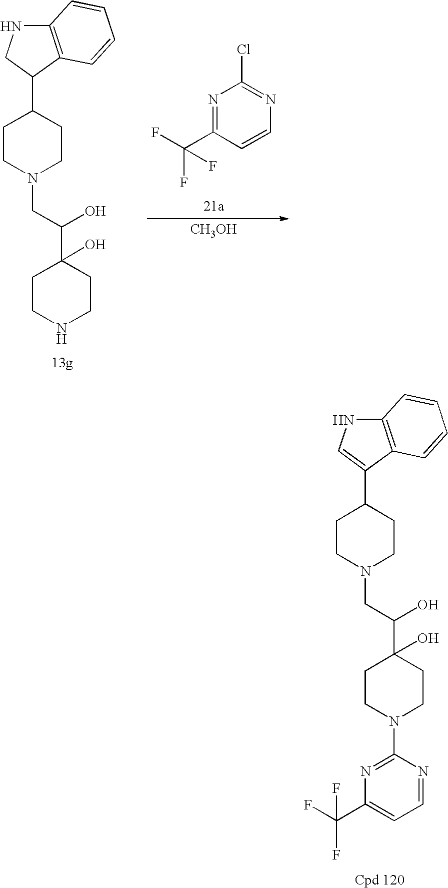

4-{1-hydroxy-2-[4-(1H-indol-3-yl)-piperidin-1-yl]-ethyl}-piperidin-4-ol Compound 13g (0.075 g, 2.18 mmol, 1 eq) and 2-chloro-4-trifluoromethyl-pyrimidine Compound 21b (0.06 g, 3.27 mmol, 1.5 eq) were dissolved in CH₃OH (2 mL, anhydrous) and placed in a 5 mL CEM microwave reaction vessel. The solution was then heated in a CEM microwave system at 140° C. for 45 min. The reaction mixture was cooled, concentrated and purified by silica gel chromatography (10% 2N N₃/CH₃OH in CH₂Cl₂) to give Compound 120 (0.0665 g, 62%) as a white solid. MS m/z 490 (M+H)⁺.

Using the procedure of Example 21 and known appropriate reagents and starting materials, the following compounds of the invention were prepared:

| Cpd | Name | MS |
|---|---|---|
| 121 | 4-{2-[4-(1H-benzoimidazol-2-yl)-piperidin-1-yl]-1- | 491 |
| | hydroxy-ethyl}-1-(4-trifluoromethyl-pyrimidin-2- | |
| | yl)-piperidin-4-ol | |
| 122 | 4-{1-hydroxy-2-[4-(5-morpholin-4-yl-1H-indol-3-yl)- | 575 |
| | piperidin-1-yl]-ethyl}-1-(4-trifluoromethyl-pyrimidin-2- | |
| | yl)-piperidin-4-ol | |
| 123 | 4-{1-hydroxy-2-[4-(5-methyl-1H-indol-3-yl)-piperidin- | 504 |
| | 1-yl]-ethyl}-1-(4-trifluoromethyl-pyrimidin-2-yl)- | |
| | piperidin-4-ol | |
| 124 | 3-(1-{2-hydroxy-2-[4-hydroxy-1-(4-trifluoromethyl- | 515 |
| | pyrimidin-2-yl)-piperidin-4-yl]-ethyl}-piperidin-4- | |
| | yl)-1H-indole-5-carbonitrile | |

Biological Activity

Compounds of the present invention were subjected to various representative biological tests to demonstrate their usefulness for preventing, treating or ameliorating a CCR2 mediated inflammatory syndrome, disorder or disease. The results of these tests are intended to illustrate the invention in a non-limiting fashion.

Examples 4-8 are intended as prophetic examples and are expected to demonstrate that said compounds are useful in preventing, treating or ameliorating such examples of a CCR2 mediated inflammatory syndrome, disorder or disease.

### EXAMPLE 1

MCP-1 Receptor Binding Assay in THP-1 Cells

THP-1 cells were obtained from American Type Culture Collection (Manassas, Va., USA). The THP-1 cells were grown in RPMI-1640 supplemented with 10% fetal bovine serum in a humidified 5% CO₂ atmosphere at 37° C. The cell density was maintained between 0.5×10⁶ cells/mL.

THP-1 cells were incubated with 0.5 nM ¹²⁵, labeled MCP-1 (Perkin-Elmer Life Sciences, Inc. Boston, Mass.) in the presence of varying concentrations of either unlabeled MCP-1 (R & D Systems, Minneapolis, Minn.) or test compound for 2 hours at 30° C. in a 96 well plate. Cells were then harvested onto a filter plate, dried, and 20 μL of Microscint 20 was added to each well. Plates were counted in a TopCount NXT, Microplate Scintillation & Luminescence Counter (Perkin-Elmer Life Sciences, Inc. Boston, Mass.). Blank values (buffer only) were subtracted from all values and drug treated values were compared to vehicle treated values. 1 μM cold MCP-1 was used for nonspecific binding.

Table 1 lists IC₅₀ values for inhibition of MCP-1 binding to CCR2 obtained for test compounds of the invention. Where an IC₅₀ value was not obtained for a particular compound, the percent inhibition is provided at a test concentration of 25 μM.

TABLE 1
Inhibition of MCP-1 Binding IC₅₀ (μM)

| Cpd | IC₅₀ |
|---|---|
| 1 | 0.25 |
| 2 | 4.4 |
| 3 | 3.4 |
| 4 | 0.03 |
| 5 | 0.16 |
| 6 | 0.04 |
| 7 | 0.03 |
| 8 | 6.4 |
| 9 | 0.05 |
| 10 | 0.15 |
| 11 | 0.01 |
| 12 | 0.02 |
| 13 | 0.01 |
| 14 | 0.14 |
| 15 | 0.03 |
| 16 | 0.13 |
| 17 | 9.4 |
| 18 | 0.68 |
| 19 | 0.13 |
| 20 | 0.05 |
| 21 | 0.05 |
| 22 | 0.19 |
| 23 | 0.02 |
| 24 | 0.07 |
| 25 | 0.81 |
| 26 | 0.07 |
| 27 | 0.01 |
| 28 | 0.25 |
| 29 | 0.07 |
| 30 | 0.05 |
| 31 | 1.6 |
| 32 | 0.07 |
| 33 | 1.4 |
| 34 | 0.57 |
| 35 | 1.2 |
| 36 | 1.1 |
| 37 | 8.6 |
| 38 | 4.7 |
| 39 | 52% |
| 40 | 0.08 |
| 41 | 0.04 |
| 42 | 2.2 |
| 43 | 0.59 |
| 44 | 1.8 |
| 45 | 0.12 |
| 46 | 2.1 |
| 47 | 3.9 |
| 48 | 48% |
| 49 | 40% |
| 50 | 44% |
| 51 | 48% |
| 52 | 53% |
| 53 | 48% |
| 54 | 5.6 |
| 55 | 3.5 |
| 56 | 5.7 |
| 57 | 2.6 |
| 58 | 5 |
| 59 | 6.1 |
| 60 | 0.31 |
| 61 | 0.83 |
| 62 | 55% |
| 63 | 46% |
| 64 | 1.3 |
| 65 | 0.08 |
| 66 | 0.42 |
| 67 | 0.24 |
| 68 | 2.7 |
| 69 | 0.95 |
| 70 | 0.18 |
| 71 | 0.04 |
| 72 | 0.7 |
| 73 | 0.13 |
| 74 | 0.04 |
| 75 | 0.35 |
| 76 | 0.47 |
| 77 | 0.5 |
| 78 | 0.37 |
| 79 | 0.81 |
| 80 | 0.2 |
| 81 | 2.6 |
| 82 | 1.3 |
| 83 | 0.86 |
| 84 | 0.55 |
| 85 | 0.15 |
| 86 | 0.08 |
| 87 | 0.47 |
| 88 | 0.17 |
| 89 | 0.55 |
| 90 | 87% |
| 91 | 0.81 |
| 92 | 0.48 |
| 93 | 0.31 |
| 94 | 0.35 |
| 95 | 1.4 |
| 100 | 50% |
| 101 | 51% |
| 102 | 0.48 |
| 103 | 0.5 |
| 104 | 2.7 |
| 105 | 0.54 |
| 106 | 0.76 |
| 107 | 0.29 |
| 108 | 0.21 |
| 109 | 4.2 |
| 110 | 0.09 |
| 111 | 2.6 |
| 112 | 3.7 |
| 113 | 1.6 |
| 114 | 1.1 |
| 115 | 0.91 |
| 116 | 0.53 |
| 117 | 1.3 |
| 118 | 1.3 |
| 119 | 43% |
| 120 | 1.9 |
| 121 | 50% |
| 122 | 51% |
| 123 | 9.4 |
| 124 | 8.3 |
| 125 | 51% |
| 126 | 55% |
| 127 | 59% |
| 128 | 0.11 |
| 129 | 0.5 |
| 130 | 0.1 |
| 131 | 0.18 |
| 132 | 2.4 |
| 133 | 3 |
| 134 | 1.9 |
| 135 | 0.73 |
| 136 | 0.14 |
| 137 | 0.255 |
| 138 | 0.21 |
| 139 | 0.92 |
| 140 | 15.4 |
| 141 | 2.8 |
| 142 | 0.26 |
| 143 | 1 |
| 144 | 3.4 |
| 145 | 1.9 |
| 146 | 41% |
| 147 | 3 |
| 148 | 1.1 |
| 149 | 1.3 |
| 150 | 8.7 |
| 151 | 0.04 |
| 152 | 3.1 |
| 153 | 0.73 |
| 154 | 1.8 |
| 155 | 0.1 |
| 156 | 0.12 |
| 157 | 0.15 |
| 158 | 0.35 |
| 159 | 0.73 |
| 160 | 0.84 |
| 161 | 8.3 |
| 162 | 8.1 |
| 163 | 0.0002 |
| 164 | 0.05 |
| 165 | 0.05 |
| 166 | 3.1 |
| 167 | 0.46 |
| 168 | 2.7 |
| 169 | 0.79 |
| 170 | 4.4 |
| 171 | 12.8 |
| 172 | 3.7 |
| 173 | 0.91 |
| 174 | 3.2 |
| 175 | 70% |
| 176 | 42% |
| 177 | 53% |
| 178 | 59% |
| 179 | 5.8 |
| 180 | 4.2 |
| 181 | 0.27 |
| 182 | 4.9 |
| 183 | 47% |
| 184 | 61% |
| 185 | 54% |

### EXAMPLE 2

MCP-1 Induced Calcium Mobilization in THP-1 Cells

THP-1 cells were plated at a density of 8×10⁵ cells/mL (100 μL/well) into poly-D lysine coated clear bottom, black 96 well plates. The cells were loaded with 5 μM fluo-3 for 45 minutes. The fluo-3 washed off and cells were incubated with varying concentrations of test compound for 15 minutes. The change in calcium ion concentration upon addition of 0.2 μM MCP-1 was determined using FLIPR and compared to vehicle.

Table 2 lists IC₅₀ values for inhibition of MCP-1 induced influx of calcium ions. Where an IC₅₀ value was not obtained for a particular compound, the percent inhibition is provided at a test concentration of 25 μM.

TABLE 2
Inhibition of MCP-1 Induced Calcium Ion Influx IC₅₀ (μM)

| Cpd | IC₅₀ |
|---|---|
| 1 | 0.07 |
| 4 | 0.02 |
| 5 | 0.08 |
| 6 | 0.007 |
| 7 | 0.007 |
| 9 | 0.12 |
| 10 | 0.4 |
| 11 | 0.35 |
| 12 | 0.65 |
| 13 | 0.24 |
| 14 | 0.003 |
| 15 | 0.11 |
| 16 | 0.98 |
| 18 | 1.7 |
| 19 | 0.03 |
| 20 | 0.23 |
| 21 | 0.48 |
| 22 | 0.03 |
| 23 | 0.007 |
| 24 | 0.12 |
| 25 | 1.5 |
| 26 | 0.37 |
| 27 | 0.008 |
| 28 | 0.31 |
| 29 | 0.09 |
| 30 | 0.22 |
| 32 | 0.08 |
| 34 | 10.9 |
| 40 | 0.17 |
| 41 | 0.09 |
| 42 | 3.7 |
| 43 | 0.18 |
| 44 | 0.72 |
| 45 | 0.15 |
| 58 | 42% |
| 60 | 0.38 |
| 61 | 2.8 |
| 65 | 0.04 |
| 66 | 1.1 |
| 67 | 0.3 |
| 70 | 0.08 |
| 71 | 0.2 |
| 72 | 4.3, 0.76 |
| 73 | 0.001 |
| 74 | 0.002 |
| 75 | 0.005 |
| 76 | 0.165 |
| 77 | 0.085 |
| 78 | 0.04 |
| 79 | 7.4 |
| 80 | 0.006 |
| 81 | 1.8 |
| 82 | 2.6 |
| 84 | 0.03 |
| 85 | 0.006 |
| 86 | 0.07 |
| 87 | 3.3 |
| 88 | 0.98 |
| 89 | 1.8 |
| 91 | 8.1, 1.7 |
| 92 | 7.7, 1.5 |
| 93 | 9.2 |
| 94 | 0.90 |
| 95 | 53% |
| 96 | 13.1 |
| 97 | 12.9 |
| 98 | 0.8 |
| 99 | 3.5 |
| 102 | 2.5 |
| 103 | 2.2 |
| 105 | 3.4 |
| 106 | 2.6 |
| 107 | 4.3 |
| 108 | 0.48 |
| 110 | 4.3 |
| 113 | 4.9 |
| 114 | 55% |
| 115 | 57% |
| 116 | 0.84 |
| 117 | 1.2 |
| 118 | 3.1 |
| 120 | 13% |
| 121 | 65% |
| 123 | 68% |
| 124 | 43% |
| 128 | 1.9 |
| 129 | 8 |
| 130 | 2.4 |
| 131 | 0.38 |
| 132 | 26% |
| 134 | 52% |
| 135 | 9.2 |
| 136 | 0.21 |
| 137 | 1.3 |
| 138 | 0.81 |
| 139 | 0.31 |
| 142 | 68% |
| 151 | 0.51 |
| 152 | 43% |
| 153 | 12% |
| 155 | 0.66 |
| 156 | 4 |
| 157 | 1.9 |
| 158 | 2.1 |
| 159 | 1.5 |
| 160 | 3.6 |
| 163 | 0.12 |
| 164 | 0.06 |
| 165 | 0.9 |
| 167 | 0.94 |
| 169 | 5.4 |
| 170 | 17.2 |
| 171 | 22% |
| 172 | 46% |
| 173 | 1.2 |
| 174 | 44% |
| 175 | 24% |
| 177 | 27% |
| 178 | 2% |
| 181 | 2.1 |
| 182 | 5.4 |
| 184 | 13% |

### EXAMPLE 3

MCP-1 Induced Chemotaxis in THP-1 Cells

MCP-1 induced chemotaxis was run in a 24-well chemotaxis chamber. MCP-1 (0.01 μg/mL) was added to the lower chamber and 100 μL of THP-1 cells (1×10⁷ cell/mL) was added to the top chamber. Varying concentrations of test compound were added to the top and bottom chambers. Cells were allowed to chemotax for 3 hours at 37° C. and 5% CO₂. An aliquot of the cells that had migrated to the bottom chamber was taken and counted then compared to vehicle.

Table 3 lists IC₅₀ values for inhibition of MCP-1 induced chemotaxis. Where an IC₅₀ value was not obtained for a particular compound, the percent inhibition is provided at a test concentration of 25 μM.

TABLE 3
Inhibition of MCP-1 Induced Chemotaxis IC₅₀ (μM)

| Cpd | IC₅₀ |
|---|---|
| 1 | 0.09 |
| 4 | 0.004 |
| 5 | 0.12 |
| 6 | 0.02 |
| 7 | 0.03 |
| 9 | 0.02 |
| 10 | 0.27 |
| 11 | 0.19 |
| 12 | 0.007 |
| 13 | 0.06 |
| 14 | 0.15 |
| 15 | 0.02 |
| 16 | 0.3 |
| 18 | 0.42 |
| 19 | 0.19 |
| 20 | 0.02 |
| 21 | 0.02 |
| 22 | 0.36 |
| 23 | 0.02 |
| 24 | 0.07 |
| 25 | 0.64 |
| 26 | 0.02 |
| 27 | 0.01 |
| 28 | 0.37 |
| 29 | 0.07 |
| 30 | 0.06 |
| 32 | 0.1 |
| 34 | 0.84 |
| 40 | 0.03 |
| 41 | 0.02 |
| 43 | 0.09 |
| 45 | 0.17 |
| 60 | 0.29 |
| 61 | 0.24 |
| 65 | 0.25 |
| 67 | 0.7 |
| 70 | 0.01 |
| 73 | 0.03 |
| 74 | 0.08 |
| 75 | 0.03 |
| 78 | 0.21 |
| 80 | 0.09 |
| 85 | 0.04 |
| 86 | 0.05 |
| 88 | 0.03 |
| 93 | 0.12 |
| 94 | 0.1 |
| 107 | 0.04 |
| 110 | 0.07 |
| 128 | 0.3 |
| 130 | 0.19 |
| 136 | 0.15 |
| 137 | 0.17 |
| 138 | 0.06 |
| 142 | 0.26 |
| 151 | 0.16 |
| 155 | 0.18 |
| 156 | 0.36 |
| 157 | 0.12 |
| 163 | 0.005 |
| 164 | 0.002 |
| 165 | 0.12 |
| 167 | 0.004 |
| 181 | 0.18 |

### EXAMPLE 4

Diet Induced Obesity Model

In a diet induced obesity model in mice, mice in three treatment groups (treated, untreated and vehicle control) are fed either regular chow or a high fat diet for 37 days. The treated groups are dosed (ip, bid) with a test compound at 100 mg/kg from day 1 to day 37. Body weight is monitored twice per week and on Day 37. Blood glucose, body weight, body mass, serum MCP-1 and insulin levels are also recorded on Day 37.

One or more compounds of the present invention are expected to show that either weight gain is inhibited or weight loss is induced. In the alternative, an improvement in insulin sensitivity is expected. Accordingly, said compounds are useful in preventing, treating or ameliorating obesity.

### EXAMPLE 5

Collagen-Induced Arthritis Model

In a collagen-induced arthritis model in mice, DBA1 mice are immunized with bovine type II collagen on day 0, injected (sc) with lipopolysaccharide (LPS) on day 21, and dosed (ip, bid) with a test compound at either 25, 50 or 100 mg/kg from day 20 to day 35. Body weight is monitored and the clinical disease score is recorded every 2-3 days starting on day 20.

Test compound is dosed in one of two vehicles:

1) 10% Pharmasolve:20% PEG-400:70% of a 1% solution of Tween-80 in water; or,

2) 30% PEG-400:20% Solutol:50% of a 0.1 N solution of NaHCO₃.

One or more compounds of the present invention are expected to demonstrate that said compounds are useful in preventing, treating or ameliorating arthritis by showing that the compounds significantly inhibit infiltration of monocytes and lymphocytes into the joints, but do not significantly affect infiltration by leukocytes.

### EXAMPLE 6

Adjuvant-Induced Arthritis Model (Dosing from Day 0-14)

In the adjuvant-induced arthritis model, 7-week old male Lewis rats are injected in the right hind footpad with a mixture of heat-killed Mycobacterium Butyricum (0.5 mg) in liquid paraffin oil (50 μL). An increase in volume of the contralateral (non-injected) hind paw is a measure of arthritis severity.

Body weight and hind paw volume (as measured by mercury plethysmography volume displacement) are typically recorded on days 0, 3, 7, 10, 12, 14, and 16. Animals are dosed with a test compound (ip, bid, 100 mg/kg) from days 0-14, or with a vehicle control. As a positive control for inhibition, a separate group of rats is injected with a non-steroidal antiinflammatory drug (orally, once per day, 3 mg/kg) from days 10-14.

One or more compounds of the present invention are expected to demonstrate that said compounds are prophylactically and therapeutically useful in preventing, treating or ameliorating arthritis by showing that the compounds inhibit or decrease swelling volume in the contralateral paws.

### EXAMPLE 7

Mouse Model of Allergic Asthma:

An allergic asthma model in mice is used to test compounds of the invention for therapeutic effect on asthmatic response as a function of airway inflammation and hyperresponsiveness (Malaviya, et al., J. Phar. Exp. Ther., 2000, 295: 912-926). Airway hyperresponsiveness in asthmatic patients is a cardinal feature of allergic asthma and is maintained as a result of persistent airway inflammation. Eosinophils are the prominent cells involved in airway inflammation and are found in large numbers in sputum and bronchoalveolar lavage fluids.

Airway responsiveness is measured in unrestrained mice by noninvasive whole body plethysmography using a BioSystem plethysmography instrument (BUXCO, Troy, N.Y.). Each animal is individually placed in the plethysmography instrument chamber and chamber pressure is used as a measure of the difference between thoracic volume expansion or contraction and air volume removed or added to the chamber during breathing. The differential of this function with respect to time produces a pseudo flow value that is proportionate to the difference between the rate of the thoracic volume expansion and nasal air flow (Hamelmann, et al., J. Respir. Crit. Care Med., 1997, 156: 766-775).

Animals and Method:

Three treatment groups of BALB/c female mice (6-8 weeks old) are tested in the 32 day study:
Group 1: vehicle control phosphate buffered saline (PBS)-sensitized and PBS-challenged mice;
Group 2: positive control ovalbumin (OVA)-sensitized and OVA-challenged mice; and,
Group 3: OVA-sensitized and OVA-challenged mice treated with a test compound in a suitable vehicle.
   Day 0 and 14:
Group 1 mice are sensitized by injection (ip) with PBS; and,
Group 2 mice are OVA sensitized by injection (ip) with OVA (20 μg) dissolved in PBS adsorbed on 2.25 mg alum.
   Day 28, 29 and 30:
   Challenge Phase
Group 1 mice are challenged with PBS by ultrasonic nebulization for 20 min.
A first subset of Group 2 mice is OVA-challenged by ultrasonic nebulization of OVA (5 mg/mL) for 20 min.
A second subset of Group 2 mice is also OVA-challenged by ultrasonic nebulization of OVA (5 mg/mL) for 20 min.
   Treatment Phase
Group 1 mice are treated by injection (ip) with vehicle at 30 min before and at 6 hr after the PBS challenge.
Group 2 (first subset) mice are treated by injection (ip) with vehicle at 30 min before and at 6 hr after the OVA challenge.
Group 2 (second subset) mice are treated by injection (ip) with a test compound (100 mg/kg) at 30 min before and at 6 hr after the OVA challenge. The second subset is then designated as treatment Group 3.
   Day 31:
Group 1 and Group 2 (first subset) mice are dosed twice with vehicle alone, the second dose for each group is administered 6 hr after the first dose; and,
Group 3 mice are dosed twice with a test compound (100 mg/kg), the second dose is administered 6 hr after the first dose.
   Day 32:

The three treatment groups are challenged by means of administering methacholine via airway inhalation; asthmatic response is measured as a function of airway hyper-responsiveness.

Baseline Phase

A baseline reading over a 5 min period for each of the mice in the three treatment groups is taken in the plethysmography instrument, then the baseline readings are averaged.

Challenge Phase

Group 1 mice are nebulized with saline at increasing doses (1-30 mg/mL) over a 2 min period.
Group 2 (first subset) and Group 3 mice are nebulized with methacholine at increasing doses (1-30 mg/ml) over a 2 min period.
   Post-Challenge Phase

A 5 min post-challenge reading for each of the mice is taken and the readings are averaged.

Reduction in airway hyperresponsiveness is calculated according to the following formula:

(Treated Reading^{Avg}−Veh. Control Reading^{Avg}) (100%)×1−(Positive Control Reading^{Avg}−Veh. Control Reading^{Avg})

Airway inflammation is measured by eosinophil cell count in bronchoalveolar saline lavage samples (1 mL) of the mice from the three groups. The lavage fluid is centrifuged and the supernatant is removed. The cell pellet is resuspended in saline containing 0.1% BSA, then cytospin smears are made from the cell suspension and stained with Giemsa. The number of eosinophils is counted and the cell concentration adjusted to 0.1×10⁶/mL.

One or more compounds of the present invention are expected to demonstrate that said compounds are prophylactically and therapeutically useful in preventing, treating or ameliorating asthma by showing that the compounds reduce airway hyperresponsiveness and reduce airway inflammation by inhibiting eosinophil infiltration in treated mice compared to non-treated mice.

### EXAMPLE 8

Inhibition of Ovalbumin-Induced Allergic Rhinitis in Mice

BALB/c mice are sensitized by i.p. injection of OVA emulsified in alum (Day 0, 5, 14, 21). Groups of mice are each challenged by intranasal injection of OVA (Day 22-35, 38). Control group mice receive an equal volume of vehicle by intranasal injection. Nasal symptoms (number of sneezes and episodes of nose rubbing by the front paws) are counted during the 5 min period following the last intranasal injection (Day 38).

Prophylactic Effect

A test compound (in PBS) is administered by intranasal injection (10 and 30 μg/nostril) to both nostrils twice daily 1 hr and 6 hrs prior to intranasal challenge (Days 22-35), once per day prior to intranasal challenge (Days 36, 37) then 1 hr and 6 hrs prior to intranasal challenge (Day 38). One or more suitable anti-allergen agents are used as a positive control.

Therapeutic Effect

The dosing of test compound is delayed until the symptoms of rhinitis have appeared (Day 29). A test compound (in PBS) is then administered by intranasal injection (10 μg/nostril) to both nostrils four times per day prior to intranasal challenge (Days 29-38). One or more suitable anti-allergen agents are used as a positive control.

One or more compounds of the present invention are expected to demonstrate that said compounds are prophylactically and therapeutically useful in preventing, treating or ameliorating allergic rhinitis by showing that the compounds inhibits nasal symptoms (sneezing/rubbing) in treated mice compared to non-treated mice.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound of Formula (I): 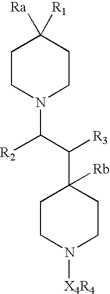
or a form thereof, wherein
R₁ is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino (optionally substituted with one or more of alkyl, alkylcarbonyl, alkoxycarbonyl, alkylsulfonyl, carboxyalkyl or alkoxycarbonylalkyl), carboxyalkyl, alkoxycarbonylalkyl, carboxyalkoxy, alkoxycarbonylalkoxy, aminoalkyl, alkylaminoalkyl, aminosulfonyl, alkylaminosulfonyl, alkylsulfonylamino, aminosulfonylalkyl, alkylaminosulfonylalkyl, carboxy, alkylcarbonyl, alkoxycarbonyl, aminocarbonyl, alkylaminocarbonyl, aryl or heterocyclyl;
Ra and Rb is each hydrogen or hydroxy;
R₂ is hydrogen or is oxo, hydroxyalkyl, haloalkyl, alkylamino, cyano, alkoxy, carboxy or alkoxycarbonyl;
R₃ is hydrogen or is oxo, hydroxy, hydroxyalkyl, halogen, haloalkyl, amino (optionally substituted with one or more of alkyl, formyl, alkylcarbonyl or alkoxycarbonyl), cyano, nitro, alkoxy, carboxy, alkoxycarbonyl, aminocarbonyl, alkylaminocarbonyl, alkoxycarbonylamino, aminocarbonylamino or alkylaminocarbonylamino, with the proviso that R₂ and R₃ are not simultaneously hydrogen;
X₄ is absent or is carbonyl, carboxyl, alkylcarbonyl, alkylcarbonyloxy, acrylyl, alkoxycarbonyl, aminocarbonyl, alkylaminocarbonyl, alkylcarbonylamino, alkylcarbonylaminoalkyl, thiocarbonyl, aminothiocarbonyl, alkylthiocarbonyl or carbonylaminoiminomethyl; and
R₄ is hydrogen or is cycloalkyl, aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl;
R₅ is hydrogen or is one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio or haloalkylthio.

2. The compound of claim 1, wherein R₁ is aryl or heterocyclyl each optionally substituted with one or more of alkyl, alkoxy, cyano, halogen, haloalkyl, amino, alkylamino, alkylcarbonylamino, alkylsulfonylamino, aryl or heterocyclyl.

3. The compound of claim 1, wherein R₁ is aryl or heterocyclyl each optionally substituted with one or more of alkyl, alkoxy, cyano, halogen, haloalkyl, amino, alkylcarbonylamino, alkylsulfonylamino or heterocyclyl.

4. The compound of claim 1, wherein Ra and Rb is each hydroxy.

5. The compound of claim 1, wherein Ra is hydrogen or hydroxy.

6. The compound of claim 1, wherein Rb is hydrogen or hydroxy.

7. The compound of claim 1, wherein R₂ is hydrogen or is oxo, hydroxyalkyl, haloalkyl, cyano, carboxy or alkoxycarbonyl.

8. The compound of claim 1, wherein R₂ is hydrogen or is oxo, hydroxyalkyl, carboxy or alkoxycarbonyl.

9. The compound of claim 1, wherein R₃ is hydrogen or is oxo, hydroxy, hydroxyalkyl, halogen, haloalkyl, amino, alkylamino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen.

10. The compound of claim 1, wherein R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen.

11. The compound of claim 1, wherein X₄ is absent or is carbonyl, alkylcarbonyl, acrylyl, alkoxycarbonyl, aminocarbonyl, alkylaminocarbonyl, thiocarbonyl, aminothiocarbonyl or carbonylaminoiminomethyl.

12. The compound of claim 1, wherein X₄ is absent or is carbonyl, alkylcarbonyl, acrylyl, alkoxycarbonyl, aminocarbonyl, aminothiocarbonyl or carbonylaminoiminomethyl.

13. The compound of claim 1, wherein R₄ is hydrogen or is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl.

14. The compound of claim 1, wherein R₄ is hydrogen or is aryl or heterocyclyl each optionally substituted with one or more of alkyl, alkoxy, cyano, halogen, haloalkyl, nitro, alkoxycarbonyl or alkylthio.

15. The compound of claim 1, wherein R₅ is hydrogen or is one or more of alkyl, alkoxy, cyano, halogen, haloalkyl, nitro, alkoxycarbonyl or alkylthio.

16. The compound of claim 1, wherein
X₄ is absent or is carbonyl, alkylcarbonyl, acrylyl, alkoxycarbonyl, aminocarbonyl, alkylaminocarbonyl, thiocarbonyl, aminothiocarbonyl or carbonylaminoiminomethyl;
R₄ is hydrogen or is aryl or heterocyclyl each optionally substituted with one or more of alkyl, alkoxy, cyano, halogen, haloalkyl, haloalkoxy, hydroxy, hydroxyalkyl, hydroxyalkoxy, nitro, amino, alkylamino, aminoalkyl, alkylaminoalkyl, carboxy, alkylcarbonyl, alkoxycarbonyl, aminocarbonyl, alkylaminocarbonyl, alkylthio, haloalkylthio, R₅-aryl or R₅-heteroaryl; and
R₅ is hydrogen or is one or more of alkyl, alkoxy, cyano, halogen, haloalkyl, nitro, alkoxycarbonyl or alkylthio.

17. The compound of claim 1, wherein
X₄ is absent or is carbonyl, alkylcarbonyl, acrylyl, alkoxycarbonyl, aminocarbonyl, aminothiocarbonyl or carbonylaminoiminomethyl; and
R₄ is hydrogen or is aryl or heterocyclyl each optionally substituted with one or more of alkyl, alkoxy, cyano, halogen, haloalkyl, nitro, alkoxycarbonyl or alkylthio.

18. The compound of claim 1, wherein
R₁ is aryl or heterocyclyl each optionally substituted with one or more of alkyl, alkoxy, cyano, halogen, haloalkyl, amino, alkylcarbonylamino, alkylsulfonylamino or heterocyclyl;
Ra and Rb is each hydrogen or hydroxy;
R₂ is hydrogen or is oxo, hydroxyalkyl, carboxy or alkoxycarbonyl;
R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen;
X₄ is absent or is carbonyl, alkylcarbonyl, acrylyl, alkoxycarbonyl, aminocarbonyl, aminothiocarbonyl or carbonylaminoiminomethyl;
R₄ is hydrogen or is aryl or heterocyclyl each optionally substituted with one or more of alkyl, alkoxy, cyano, halogen, haloalkyl, nitro, alkoxycarbonyl, alkylthio, R₅-aryl or R₅-heteroaryl; and
R₅ is hydrogen or is one or more of alkyl, alkoxy, cyano, halogen, haloalkyl, nitro, alkoxycarbonyl or alkylthio.

19. The compound of claim 1, wherein
R₁ is aryl or heterocyclyl each optionally substituted with one or more of alkyl, alkoxy, cyano, halogen, haloalkyl, amino, alkylcarbonylamino, alkylsulfonylamino or heterocyclyl;
Ra and Rb is each hydrogen or hydroxy;
R₂ is hydrogen or is oxo, hydroxyalkyl, carboxy or alkoxycarbonyl;
R₃ is hydrogen or is oxo, hydroxy, hydroxyalkyl, amino, alkylcarbonylamino, alkoxycarbonylamino, carboxy, alkoxycarbonyl, alkoxycarbonylamino or alkylaminocarbonylamino, with the proviso that R₂ and R₃ are not simultaneously hydrogen;
X₄ is absent or is carbonyl, alkylcarbonyl, acrylyl, alkoxycarbonyl, aminocarbonyl, aminothiocarbonyl or carbonylaminoiminomethyl; and
R₄ is hydrogen or is aryl or heterocyclyl each optionally substituted with one or more of alkyl, alkoxy, cyano, halogen, haloalkyl, nitro, alkoxycarbonyl or alkylthio.

20. The compound of claim 1, wherein
R₁ is selected from (4-Cl)-phenyl, (4-OCH₃)-phenyl, (4-F)-phenyl, (4-CF₃)-phenyl, indol-3-yl, (5-CH₃)-indol-3-yl, (5-OCH₃)-indol-3-yl, (5-CN)-indol-3-yl, (5-NH₂)-indol-3-yl, (5-F)-indol-3-yl, (5-NHC(O)—CH₃)-indol-3-yl, (5-NHSO₂CH₃)-indol-3-yl, (5-morpholin-4-yl)-indol-3-yl, 1H-pyrazol-3-yl, benzoxazolyl-2-yl, 1H-benzoimidazol-2-yl, or 1H-pyrrolo[2,3-b]pyridin-3-yl;
Ra is selected from hydrogen or hydroxy;
Rb is selected from hydrogen or hydroxy;
R₂ is selected from hydrogen, oxo, CO₂H, CO₂H₂CH₃ or CH₂OH;
R₃ is selected from hydrogen, oxo, hydroxy, NH₂, CO₂H, CO₂CH₂CH₃, CH₂OH, NHC(O)CH₃, NHC(O)OCH₃ or NHC(O)N(CH₃)₂, with the proviso that R₂ and R₃ are not simultaneously hydrogen; and
X₄R₄ is selected from hydrogen, C(O)CH═CH-3,4-Cl₂-phenyl, C(O)CH═CH-3,5-Cl₂-phenyl, C(O)CH═CH-3,4-F₂-phenyl, C(O)CH═CH-3,5-F₂-phenyl, C(O)CH═CH-3-CH₃-phenyl, C(O)CH═CH-3-OCH₃-phenyl, C(O)CH═CH-3-CF₃-phenyl, C(O)CH═CH-3-F-phenyl, C(O)CH═CH-4-F-phenyl, C(O)CH═CH-4-Cl-phenyl, C(O)CH═CH-3,5-(CF₃)-2-phenyl, C(O)CH═CH-3-F-4-Cl-phenyl, C(O)CH═CH-3,4-(OCH₃)-2-phenyl, C(O)CH═CH-3,5-CF₃-phenyl, C(O)CH═CH-2,4,5-F₃-phenyl, C(O)CH═CH-3,4,5-F₃-phenyl, C(O)CH═CH-3-Br-4-F-phenyl, C(O)CH═CH-thien-3-yl, C(O)NH-3,4-Cl₂-phenyl, C(O)NH-3,5-Cl₂-phenyl, C(O)NH-3,4-F₂-phenyl, C(O)NH-3,5-F₂-phenyl, C(O)NH-3-SCH₃-phenyl, C(O)NH-4-SCH₃-phenyl, C(O)NH-2-F-4,5-Cl₂-phenyl, C(O)NH-2-Cl₅-F-phenyl, C(O)NH-3-CF₃-5-F-phenyl, C(O)NH-3-F-5-CF₃-phenyl, C(O)NH-2-Cl₄-CF₃-phenyl, C(O)NH-benzo[1,3]dioxol-5-yl, C(S)NH-3-Br-phenyl, C(S)NH-3,5-Cl₂-phenyl, C(S)NH-3,4-Cl₂-phenyl, C(S)NH-3-OCH₃-phenyl, C(S)NH-3-CF₃-phenyl, C(S)NH-3-F-4-Br-phenyl, C(S)NH-3-Cl-phenyl, C(S)NH-3-CN-phenyl, C(S)NH-4-CF₃-phenyl, C(S)NH-3,5-F₂-phenyl, C(S)NH-2,3,5-F₃-phenyl, C(S)NH-3,5-(OCH₃)-2-phenyl, C(O)-3,4,5-F₃-phenyl, C(NH)NHC(O)-3,5-F₂-phenyl, C(O)OC(CH₃)₃, C(O)O-benzyl, (4-CF₃)-pyrimidin-2-yl, (5-NO₂)-pyridin-2-yl, C(O)(CH₂)₂-4-Cl-phenyl, C(O)(CH₂)₂-3,4-Cl₂-phenyl or C(O)-3-NO₂-benzyl.

21. A compound selected from the group consisting of:
3-(3,4-dichloro-phenyl)-1-(4-hydroxy-4-{1-hydroxy-2-[4-(4-methoxy-phenyl)-piperidin-1-yl]-ethyl}-piperidin-1-yl)-propenone,
1-[4-(4-chloro-phenyl)-piperidin-1-yl]-2-{1-[3-(3,4-dichloro-phenyl)-acryloyl]-piperidin-4-yl}-ethane-1,2-dione,
1-(4-{2-[4-(4-chloro-phenyl)-piperidin-1-yl]-1-hydroxy-2-oxo-ethyl}-piperidin-1-yl)-3-(3,4-dichloro-phenyl)-propenone,
4-{1-hydroxy-2-[4-(1H-indol-3-yl)-piperidin-1-yl]-ethyl}-piperidine-1-carboxylic acid (3,4-dichloro-phenyl)-amide,
4-{1-hydroxy-2-[4-(1H-indol-3-yl)-piperidin-1-yl]-ethyl}-piperidine-1-carboxylic acid (3,5-difluoro-phenyl)-amide,
3-(3,4-dichloro-phenyl)-1-(4-{1-hydroxy-2-[4-(1H-indol-3-yl)-piperidin-1-yl]-ethyl}-piperidin-1-yl)-prop enone,
3-(3,5-difluoro-phenyl)-1-(4-{1-hydroxy-2-[4-(1H-indol-3-yl)-piperidin-1-yl]-ethyl}-piperidin-1-yl)-prop enone,
(4-{1-hydroxy-2-[4-(1H-indol-3-yl)-piperidin-1-yl]-ethyl}-piperidin-1-yl)-(3,4,5-trifluoro-phenyl)-methanone,
4-{1-hydroxy-2-[4-(1H-indol-3-yl)-piperidin-1-yl]-ethyl}-piperidine-1-carbothioic acid (3-bromo-phenyl)-amide,
3,5-difluoro-N-[(4-{1-hydroxy-2-[4-(1H-indol-3-yl)-piperidin-1-yl]-ethyl}-piperidin-1-yl)-imino-methyl]-benzamide,
1-(4-{1-hydroxy-2-[4-(1H-indol-3-yl)-piperidin-1-yl]-ethyl}-piperidin-1-yl)-3-m-tolyl-propenone,
3-(3,5-difluoro-phenyl)-1-(4-{1-hydroxy-2-[4-(4-methoxy-phenyl)-piperidin-1-yl]-ethyl}-piperidin-1-yl)-propenone,
1-(4-{1-hydroxy-2-[4-(4-methoxy-phenyl)-piperidin-1-yl]-ethyl}-piperidin-1-yl)-3-(3,4,5-trifluoro-phenyl)-propenone,
3-(3,4-dichloro-phenyl)-1-(4-{1-hydroxy-2-[4-(4-methoxy-phenyl)-piperidin-1-yl]-ethyl}-piperidin-1-yl)-propenone,
4-{1-hydroxy-2-[4-(4-methoxy-phenyl)-piperidin-1-yl]-ethyl}-piperidine-1-carboxylic acid (3,4-dichloro-phenyl)-amide,
4-{1-hydroxy-2-[4-(4-methoxy-phenyl)-piperidin-1-yl]-ethyl}-piperidine-1-carbothioic acid (3-bromo-phenyl)-amide,
1-(4-{2-[4-(1H-pyrrolo[2,3-b]pyridin-3-yl)-piperidin-1-yl]-acetyl}-piperidin-1-yl)-3-(3,4,5-trifluoro-phenyl)-propenone,
N-{3-[1-(2-oxo-2-{1-[3-(3,4,5-trifluoro-phenyl)-acryloyl]-piperidin-4-yl}-ethyl)-piperidin-4-yl]-1H-indol-5-yl}-methanesulfonamide,
1-(4-{1-hydroxy-2-[4-(1H-pyrrolo[2,3-b]pyridin-3-yl)-piperidin-1-yl]-ethyl}-piperidin-1-yl)-3-m-tolyl-propenone,
3-(3,5-difluoro-phenyl)-1-(4-{1-hydroxy-2-[4-(1H-pyrrolo[2,3-b]pyridin-3-yl)-piperidin-1-yl]-ethyl}-piperidin-1-yl)-propenone,
1-(4-{1-hydroxy-2-[4-(1H-pyrrolo[2,3-b]pyridin-3-yl)-piperidin-1-yl]-ethyl}-piperidin-1-yl)-3-(3,4,5-trifluoro-phenyl)-propenone,
1-(4-{1-hydroxy-2-[4-(1H-pyrrolo[2,3-b]pyridin-3-yl)-piperidin-1-yl]-ethyl}-piperidin-1-yl)-3-(3-trifluoromethyl-phenyl)-propenone,
3-(3,4-dichloro-phenyl)-1-(4-{1-hydroxy-2-[4-(1H-pyrrolo[2,3-b]pyridin-3-yl)-piperidin-1-yl]-ethyl}-piperidin-1-yl)-propenone,
4-{1-hydroxy-2-[4-(1H-pyrrolo[2,3-b]pyridin-3-yl)-piperidin-1-yl]-ethyl}-piperidine-1-carboxylic acid (3,4-dichloro-phenyl)-amide,
4-{1-hydroxy-2-[4-(1H-pyrrolo[2,3-b]pyridin-3-yl)-piperidin-1-yl]-ethyl}-piperidine-1-carboxylic acid (4-methylsulfanyl-phenyl)-amide,
3-(3,5-difluoro-phenyl)-1-(4-{1-hydroxy-2-[4-(5-methoxy-1H-indol-3-yl)-piperidin-1-yl]-ethyl}-piperidin-1-yl)-propenone,
N-{3-[1-(2-hydroxy-2-{1-[3-(3,4,5-trifluoro-phenyl)-acryloyl]-piperidin-4-yl}-ethyl)-piperidin-4-yl]-1H-indol-5-yl}-methanesulfonamide,
N-{3-[1-(2-amino-2-{1-[3-(3,4,5-trifluoro-phenyl)-acryloyl]-piperidin-4-yl}-ethyl)-piperidin-4-yl]-1H-indol-5-yl}-methanesulfonamide,
1-(4-{1-hydroxy-2-[4-(4-methoxy-phenyl)-piperidin-1-yl]-ethyl}-piperidin-1-yl)-3-m-tolyl-propenone,
1-(4-{1-hydroxy-2-[4-(4-methoxy-phenyl)-piperidin-1-yl]-ethyl}-piperidin-1-yl)-3-(3-trifluoromethyl-phenyl)-propenone,
4-{1-hydroxy-2-[4-(4-methoxy-phenyl)-piperidin-1-yl]-ethyl}-piperidine-1-carboxylic acid (4-methylsulfanyl-phenyl)-amide,
3-(3,4-difluoro-phenyl)-1-(4-{1-hydroxy-2-[4-(4-methoxy-phenyl)-piperidin-1-yl]-ethyl}-piperidin-1-yl)-propenone,
1-(4-{1-hydroxy-2-[4-(1H-pyrazol-3-yl)-piperidin-1-yl]-ethyl}-piperidin-1-yl)-3-m-tolyl-propenone,
3-(3,5-difluoro-phenyl)-1-(4-{1-hydroxy-2-[4-(1H-pyrazol-3-yl)-piperidin-1-yl]-ethyl}-piperidin-1-yl)-prop enone,
1-(4-{1-hydroxy-2-[4-(1H-pyrazol-3-yl)-piperidin-1-yl]-ethyl}-piperidin-1-yl)-3-(3,4,5-trifluoro-phenyl)-propenone,
4-{1-hydroxy-2-[4-(1H-pyrazol-3-yl)-piperidin-1-yl]-ethyl}-piperidine-1-carboxylic acid (3,4-dichloro-phenyl)-amide,
4-{1-hydroxy-2-[4-(1H-pyrazol-3-yl)-piperidin-1-yl]-ethyl}-piperidine-1-carboxylic acid (3,4-difluoro-phenyl)-amide,
3-[4-(4-methoxy-phenyl)-piperidin-1-yl]-2-{1-[3-(3,4,5-trifluoro-phenyl)-acryloyl]-piperidin-4-yl}-propionic acid ethyl ester,
3-[4-(4-methoxy-phenyl)-piperidin-1-yl]-2-{1-[3-(3,4,5-trifluoro-phenyl)-acryloyl]-piperidin-4-yl}-propionic acid,
N-[3-(1-{2-hydroxy-2-[1-(3-m-tolyl-acryloyl)-piperidin-4-yl]-ethyl}-piperidin-4-yl)-1H-indol-5-yl]-methanesulfonamide,
N-{3-[1-(2-{1-[3-(3,5-difluoro-phenyl)-acryloyl]-piperidin-4-yl}-2-hydroxy-ethyl)-piperidin-4-yl]-1H-indol-5-yl}-methanesulfonamide,
1-(4-{1-hydroxymethyl-2-[4-(4-methoxy-phenyl)-piperidin-1-yl]-ethyl}-piperidin-1-yl)-3-m-tolyl-propenone,
3-(3,4-dichloro-phenyl)-1-(4-{1-hydroxymethyl-2-[4-(4-methoxy-phenyl)-piperidin-1-yl]-ethyl}-piperidin-1-yl)-propenone,
4-{1-hydroxymethyl-2-[4-(4-methoxy-phenyl)-piperidin-1-yl]-ethyl}-piperidine-1-carboxylic acid (3,4-dichloro-phenyl)-amide,
3-(3,5-difluoro-phenyl)-1-(4-{1-hydroxymethyl-2-[4-(4-methoxy-phenyl)-piperidin-1-yl]-ethyl}-piperidin-1-yl)-propenone,
1-{4-[2-(4-benzoxazol-2-yl-piperidin-1-yl)-1-hydroxy-ethyl]-piperidin-1-yl}-3-(3,5-difluoro-phenyl)-propenone,
1-{4-[2-(4-benzoxazol-2-yl-piperidin-1-yl)-1-hydroxy-ethyl]-piperidin-1-yl}-3-m-tolyl-propenone,
2-[4-(1H-indol-3-yl)-piperidin-1-yl]-3-[1-(3-m-tolyl-acryloyl)-piperidin-4-yl]-propionic acid ethyl ester,
3-{1-[3-(3,4-dichloro-phenyl)-acryloyl]-piperidin-4-yl}-2-[4-(1H-indol-3-yl)-piperidin-1-yl]-propionic acid ethyl ester,
2-[4-(1H-indol-3-yl)-piperidin-1-yl]-3-{1-[3-(3,4,5-trifluoro-phenyl)-acryloyl]-piperidin-4-yl}-propionic acid ethyl ester,
3-{1-[3-(3,5-difluoro-phenyl)-acryloyl]-piperidin-4-yl}-2-[4-(1H-indol-3-yl)-piperidin-1-yl]-propionic acid ethyl ester,
3-[1-(3,4-dichloro-phenylcarbamoyl)-piperidin-4-yl]-2-[4-(1H-indol-3-yl)-piperidin-1-yl]-propionic acid ethyl ester,
2-[4-(1H-indol-3-yl)-piperidin-1-yl]-3-[1-(3-m-tolyl-acryloyl)-piperidin-4-yl]-propionic acid,
3-(3,5-difluoro-phenyl)-1-(4-{3-hydroxy-2-[4-(1H-indol-3-yl)-piperidin-1-yl]-propyl}-piperidin-1-yl)-prop enone,
1-(4-{3-hydroxy-2-[4-(1H-indol-3-yl)-piperidin-1-yl]-propyl}-piperidin-1-yl)-3-(3,4,5-trifluoro-phenyl)-propenone,
3-(3,4-dichloro-phenyl)-1-(4-{3-hydroxy-2-[4-(1H-indol-3-yl)-piperidin-1-yl]-propyl}-piperidin-1-yl)-prop enone,
1-(4-{3-hydroxy-2-[4-(1H-indol-3-yl)-piperidin-1-yl]-propyl}-piperidin-1-yl)-3-m-tolyl-propenone,
4-{3-hydroxy-2-[4-(1H-indol-3-yl)-piperidin-1-yl]-propyl}-piperidine-1-carboxylic acid (3,4-dichloro-phenyl)-amide,
4-{3-hydroxy-2-[4-(1H-indol-3-yl)-piperidin-1-yl]-propyl}-piperidine-1-carbothioic acid (3-bromo-phenyl)-amide,
3-{1-[3-(3,4-dichloro-phenyl)-acryloyl]-piperidin-4-yl}-2-[4-(1H-indol-3-yl)-piperidin-1-yl]-propionic acid,
3-{1-[3-(3,5-difluoro-phenyl)-acryloyl]-piperidin-4-yl}-2-[4-(1H-indol-3-yl)-piperidin-1-yl]-propionic acid,
3-[1-(3,4-dichloro-phenylcarbamoyl)-piperidin-4-yl]-2-[4-(1H-indol-3-yl)-piperidin-1-yl]-propionic acid,
4-{1-hydroxy-2-[4-(5-methanesulfonylamino-1H-indol-3-yl)-piperidin-1-yl]-ethyl}-piperidine-1-carboxylic acid tert-butyl ester,
1-(4-{2-[4-(4-methoxy-phenyl)-piperidin-1-yl]-acetyl}-piperidin-1-yl)-3-(3,4,5-trifluoro-phenyl)-propenone,
1-(4-{1-amino-2-[4-(4-methoxy-phenyl)-piperidin-1-yl]-ethyl}-piperidin-1-yl)-3-(3,4,5-trifluoro-phenyl)-propenone,
N-(2-[4-(4-methoxy-phenyl)-piperidin-1-yl]-1-{1-[3-(3,4,5-trifluoro-phenyl)-acryloyl]-piperidin-4-yl}-ethyl)-acetamide,
(2-[4-(4-methoxy-phenyl)-piperidin-1-yl]-1-{1-[3-(3,4,5-trifluoro-phenyl)-acryloyl]-piperidin-4-yl}-ethyl)-carbamic acid methyl ester,
3-(2-[4-(4-methoxy-phenyl)-piperidin-1-yl]-1-{1-[3-(3,4,5-trifluoro-phenyl)-acryloyl]-piperidin-4-yl}-ethyl)-1,1-dimethyl-urea,
4-hydroxy-4-{1-hydroxy-2-[4-(1H-indol-3-yl)-piperidin-1-yl]-ethyl}-piperidine-1-carboxylic acid benzyl ester,
1-(4-hydroxy-4-{1-hydroxy-2-[4-(1H-indol-3-yl)-piperidin-1-yl]-ethyl}-piperidin-1-yl)-3-(3,4,5-trifluoro-phenyl)-propenone,
3-(3,5-difluoro-phenyl)-1-(4-hydroxy-4-{1-hydroxy-2-[4-(1H-indol-3-yl)-piperidin-1-yl]-ethyl}-piperidin-1-yl)-propenone,
3-(3,5-bis-trifluoromethyl-phenyl)-1-(4-hydroxy-4-{1-hydroxy-2-[4-(1H-indol-3-yl)-piperidin-1-yl]-ethyl}-piperidin-1-yl)-propenone,
3-(3,5-dichloro-phenyl)-1-(4-hydroxy-4-{1-hydroxy-2-[4-(1H-indol-3-yl)-piperidin-1-yl]-ethyl}-piperidin-1-yl)-propenone,
3-(3-chloro-5-fluoro-phenyl)-1-(4-hydroxy-4-{1-hydroxy-2-[4-(1H-indol-3-yl)-piperidin-1-yl]-ethyl}-piperidin-1-yl)-propenone,
3-(3,4-dichloro-phenyl)-1-(4-hydroxy-4-{1-hydroxy-2-[4-(1H-indol-3-yl)-piperidin-1-yl]-ethyl}-piperidin-1-yl)-propenone,
3-(3-fluoro-phenyl)-1-(4-hydroxy-4-{1-hydroxy-2-[4-(1H-indol-3-yl)-piperidin-1-yl]-ethyl}-piperidin-1-yl)-propenone,
1-(4-hydroxy-4-{1-hydroxy-2-[4-(1H-indol-3-yl)-piperidin-1-yl]-ethyl}-piperidin-1-yl)-3-(3-trifluoromethyl-phenyl)-propenone,
3-(3,4-difluoro-phenyl)-1-(4-hydroxy-4-{1-hydroxy-2-[4-(1H-indol-3-yl)-piperidin-1-yl]-ethyl}-piperidin-1-yl)-propenone,
1-(4-hydroxy-4-{1-hydroxy-2-[4-(1H-indol-3-yl)-piperidin-1-yl]-ethyl}-piperidin-1-yl)-3-thiophen-3-yl-propenone,
3-(3-bromo-4-fluoro-phenyl)-1-(4-hydroxy-4-{1-hydroxy-2-[4-(1H-indol-3-yl)-piperidin-1-yl]-ethyl}-piperidin-1-yl)-propenone,
4-hydroxy-4-{1-hydroxy-2-[4-(1H-indol-3-yl)-piperidin-1-yl]-ethyl}-piperidine-1-carboxylic acid (3,5-difluoro-phenyl)-amide,
4-hydroxy-4-{1-hydroxy-2-[4-(1H-indol-3-yl)-piperidin-1-yl]-ethyl}-piperidine-1-carboxylic acid (3,4-difluoro-phenyl)-amide,
4-hydroxy-4-{1-hydroxy-2-[4-(1H-indol-3-yl)-piperidin-1-yl]-ethyl}-piperidine-1-carbothioic acid (3,5-dichloro-phenyl)-amide,
4-hydroxy-4-{1-hydroxy-2-[4-(1H-indol-3-yl)-piperidin-1-yl]-ethyl}-piperidine-1-carbothioic acid (3,4-dichloro-phenyl)-amide,
1-(4-{2-[4-(1H-benzoimidazol-2-yl)-piperidin-1-yl]-1-hydroxy-ethyl}-piperidin-1-yl)-3-(3,4-dichloro-phenyl)-propenone,
1-(4-{2-[4-(1H-benzoimidazol-2-yl)-piperidin-1-yl]-1-hydroxy-ethyl}-piperidin-1-yl)-3-(3,4,5-trifluoro-phenyl)-propenone,
1-(4-{2-[4-(1H-benzoimidazol-2-yl)-piperidin-1-yl]-1-hydroxy-ethyl}-4-hydroxy-piperidin-1-yl)-3-(3,4,5-trifluoro-phenyl)-propenone,
1-(4-hydroxy-4-{1-hydroxy-2-[4-(4-methoxy-phenyl)-piperidin-1-yl]-ethyl}-piperidin-1-yl)-3-(3,4,5-trifluoro-phenyl)-propenone,
4-{1-hydroxy-2-[4-(4-methoxy-phenyl)-piperidin-1-yl]-ethyl}-piperidine-1-carbothioic acid (3,5-dichloro-phenyl)-amide,
4-{1-hydroxy-2-[4-(4-methoxy-phenyl)-piperidin-1-yl]-ethyl}-piperidine-1-carboxylic acid (4,5-dichloro-2-fluoro-phenyl)-amide,
4-{1-hydroxy-2-[4-(4-methoxy-phenyl)-piperidin-1-yl]-ethyl}-piperidine-1-carboxylic acid (3-fluoro-5-trifluoromethyl-phenyl)-amide,
3-(4-chloro-phenyl)-1-(4-hydroxy-4-{1-hydroxy-2-[4-(4-methoxy-phenyl)-piperidin-1-yl]-ethyl}-piperidin-1-yl)-propenone,
3-(3,4-difluoro-phenyl)-1-(4-hydroxy-4-{1-hydroxy-2-[4-(4-methoxy-phenyl)-piperidin-1-yl]-ethyl}-piperidin-1-yl)-propenone,
3-(3,5-difluoro-phenyl)-1-(4-hydroxy-4-{1-hydroxy-2-[4-(4-methoxy-phenyl)-piperidin-1-yl]-ethyl}-piperidin-1-yl)-propenone,
1-(4-hydroxy-4-{1-hydroxy-2-[4-(4-methoxy-phenyl)-piperidin-1-yl]-ethyl}piperidin-1-yl)-3-m-tolyl-propenone,
4-{1-hydroxy-2-[4-(4-methoxy-phenyl)-piperidin-1-yl]-ethyl}-piperidine-1-carbothioic acid (3-methoxy-phenyl)-amide,
4-{1-hydroxy-2-[4-(4-methoxy-phenyl)-piperidin-1-yl]-ethyl}-piperidine-1-carbothioic acid (3-trifluoromethyl-phenyl)-amide,
4-{1-hydroxy-2-[4-(4-methoxy-phenyl)-piperidin-1-yl]-ethyl}-piperidine-1-carbothioic acid (3,4-dichloro-phenyl)-amide,
4-{1-hydroxy-2-[4-(4-methoxy-phenyl)-piperidin-1-yl]-ethyl}-piperidine-1-carbothioic acid (4-bromo-3-fluoro-phenyl)-amide,
4-{1-hydroxy-2-[4-(4-methoxy-phenyl)-piperidin-1-yl]-ethyl}-piperidine-1-carboxylic acid benzo[1,3]dioxol-5-ylamide,
4-{1-hydroxy-2-[4-(4-methoxy-phenyl)-piperidin-1-yl]-ethyl}-piperidine-1-carboxylic acid (2-chloro-4-trifluoromethyl-phenyl)-amide,
4-{1-hydroxy-2-[4-(4-methoxy-phenyl)-piperidin-1-yl]-ethyl}-piperidine-1-carbothioic acid (3-chloro-phenyl)-amide,
4-{1-hydroxy-2-[4-(4-methoxy-phenyl)-piperidin-1-yl]-ethyl}-piperidine-1-carbothioic acid (3-methoxy-phenyl)-amide,
4-{1-hydroxy-2-[4-(4-methoxy-phenyl)-piperidin-1-yl]-ethyl}-piperidine-1-carbothioic acid (3-cyano-phenyl)-amide,
4-{1-hydroxy-2-[4-(4-methoxy-phenyl)-piperidin-1-yl]-ethyl}-piperidine-1-carbothioic acid (3-trifluoromethyl-phenyl)-amide,
4-{1-hydroxy-2-[4-(4-methoxy-phenyl)-piperidin-1-yl]-ethyl}-piperidine-1-carbothioic acid (4-trifluoromethyl-phenyl)-amide,
4-{1-hydroxy-2-[4-(4-methoxy-phenyl)-piperidin-1-yl]-ethyl}-piperidine-1-carbothioic acid (3,5-difluoro-phenyl)-amide,
4-{1-hydroxy-2-[4-(4-methoxy-phenyl)-piperidin-1-yl]-ethyl}-piperidine-1-carbothioic acid (3,4-dichloro-phenyl)-amide,
4-{1-hydroxy-2-[4-(4-methoxy-phenyl)-piperidin-1-yl]-ethyl}-piperidine-1-carbothioic acid (2,3,5-trifluoro-phenyl)-amide,
4-{1-hydroxy-2-[4-(4-methoxy-phenyl)-piperidin-1-yl]-ethyl}-piperidine-1-carbothioic acid (4-bromo-3-fluoro-phenyl)-amide,
4-{1-hydroxy-2-[4-(4-methoxy-phenyl)-piperidin-1-yl]-ethyl}-piperidine-1-carbothioic acid (3,5-dimethoxy-phenyl)-amide,
4-{2-[4-(4-chloro-phenyl)-piperidin-1-yl]-1-hydroxy-ethyl}-4-hydroxy-piperidine-1-carboxylic acid benzyl ester,
1-(4-hydroxy-4-{1-hydroxy-2-[4-(4-methoxy-phenyl)-piperidin-1-yl]-ethyl}piperidin-1-yl)-3-thiophen-3-yl-propenone,
4-hydroxy-4-{1-hydroxy-2-[4-(4-methoxy-phenyl)-piperidin-1-yl]-ethyl}-piperidine-1-carboxylic acid (3,4-difluoro-phenyl)-amide,
4-hydroxy-4-{1-hydroxy-2-[4-(4-methoxy-phenyl)-piperidin-1-yl]-ethyl}-piperidine-1-carboxylic acid (3,5-difluoro-phenyl)-amide,
4-hydroxy-4-{1-hydroxy-2-[4-(4-methoxy-phenyl)-piperidin-1-yl]-ethyl}-piperidine-1-carboxylic acid (3,4-dichloro-phenyl)-amide,
4-hydroxy-4-{1-hydroxy-2-[4-(4-methoxy-phenyl)-piperidin-1-yl]-ethyl}-piperidine-1-carbothioic acid (3,4-dichloro-phenyl)-amide,
4-hydroxy-4-{1-hydroxy-2-[4-(4-methoxy-phenyl)-piperidin-1-yl]-ethyl}-piperidine-1-carbothioic acid (3,5-dichloro-phenyl)-amide,
4-hydroxy-4-{1-hydroxy-2-[4-(4-methoxy-phenyl)-piperidin-1-yl]-ethyl}-piperidine-1-carbothioic acid (3,5-dimethoxy-phenyl)-amide,
4-{1-hydroxy-2-[4-(1H-indol-3-yl)-piperidin-1-yl]-ethyl}-1-(4-trifluoromethyl-pyrimidin-2-yl)-piperidin-4-ol,
4-{2-[4-(1H-benzoimidazol-2-yl)-piperidin-1-yl]-1-hydroxy-ethyl}-1-(4-trifluoromethyl-pyrimidin-2-yl)-piperidin-4-ol,
4-{1-hydroxy-2-[4-(5-morpholin-4-yl-1H-indol-3-yl)-piperidin-1-yl]-ethyl}-1-(4-trifluoromethyl-pyrimidin-2-yl)-piperidin-4-ol,
4-{1-hydroxy-2-[4-(5-methyl-1H-indol-3-yl)-piperidin-1-yl]-ethyl}-1-(4-trifluoromethyl-pyrimidin-2-yl)-piperidin-4-ol,
3-(1-{2-hydroxy-2-[4-hydroxy-1-(4-trifluoromethyl-pyrimidin-2-yl)-piperidin-4-yl]-ethyl}-piperidin-4-yl)-1H-indole-5-carbonitrile,
4-{2-[4-(4-fluoro-phenyl)-piperidin-1-yl]-1-hydroxy-ethyl}-4-hydroxy-piperidine-1-carboxylic acid benzyl ester,
4-hydroxy-4-{1-hydroxy-2-[4-(4-trifluoromethyl-phenyl)-piperidin-1-yl]-ethyl}-piperidine-1-carboxylic acid benzyl ester,
2-[4-(4-methoxy-phenyl)-piperidin-1-yl]-1-(5′-nitro-3,4,5,6-tetrahydro-2H-[1,2′]bipyridinyl-4-yl)-ethanol,
1-(4-{2-[4-(4-fluoro-phenyl)-piperidin-1-yl]-1-hydroxy-ethyl}-4-hydroxy-piperidin-1-yl)-3-(3,4,5-trifluoro-phenyl)-propenone,
3-(3,4-difluoro-phenyl)-1-(4-{2-[4-(4-fluoro-phenyl)-piperidin-1-yl]-1-hydroxy-ethyl}-4-hydroxy-piperidin-1-yl)-propenone,
3-(3,5-difluoro-phenyl)-1-(4-{2-[4-(4-fluoro-phenyl)-piperidin-1-yl]-1-hydroxy-ethyl}-4-hydroxy-piperidin-1-yl)-propenone,
3-(3,4-dichloro-phenyl)-1-(4-{2-[4-(4-fluoro-phenyl)-piperidin-1-yl]-1-hydroxy-ethyl}-4-hydroxy-piperidin-1-yl)-propenone,
4-{2-[4-(4-fluoro-phenyl)-piperidin-1-yl]-1-hydroxy-ethyl}-4-hydroxy-piperidine-1-carboxylic acid (3,4-difluoro-phenyl)-amide,
4-{2-[4-(4-fluoro-phenyl)-piperidin-1-yl]-1-hydroxy-ethyl}-4-hydroxy-piperidine-1-carboxylic acid (3,5-difluoro-phenyl)-amide,
4-{2-[4-(4-fluoro-phenyl)-piperidin-1-yl]-1-hydroxy-ethyl}-4-hydroxy-piperidine-1-carboxylic acid (3,4-dichloro-phenyl)-amide,
4-{2-[4-(4-fluoro-phenyl)-piperidin-1-yl]-1-hydroxy-ethyl}-4-hydroxy-piperidine-1-carbothioic acid (3,4-dichloro-phenyl)-amide,
1-(4-hydroxy-4-{1-hydroxy-2-[4-(4-trifluoromethyl-phenyl)-piperidin-1-yl]-ethyl}-piperidin-1-yl)-3-(3,4,5-trifluoro-phenyl)-propenone,
3-(3,4-difluoro-phenyl)-1-(4-hydroxy-4-{1-hydroxy-2-[4-(4-trifluoromethyl-phenyl)-piperidin-1-yl]-ethyl}-piperidin-1-yl)-propenone,
3-(3,5-difluoro-phenyl)-1-(4-hydroxy-4-{1-hydroxy-2-[4-(4-trifluoromethyl-phenyl)-piperidin-1-yl]-ethyl}-piperidin-1-yl)-propenone,
3-(3,4-dichloro-phenyl)-1-(4-hydroxy-4-{1-hydroxy-2-[4-(4-trifluoromethyl-phenyl)-piperidin-1-yl]-ethyl}-piperidin-1-yl)-propenone,
3-(3,4-dimethoxy-phenyl)-1-(4-hydroxy-4-{1-hydroxy-2-[4-(4-trifluoromethyl-phenyl)-piperidin-1-yl]-ethyl}-piperidin-1-yl)-propenone,
4-hydroxy-4-{1-hydroxy-2-[4-(4-trifluoromethyl-phenyl)-piperidin-1-yl]-ethyl}-piperidine-1-carboxylic acid (3,4-difluoro-phenyl)-amide,
4-hydroxy-4-{1-hydroxy-2-[4-(4-trifluoromethyl-phenyl)-piperidin-1-yl]-ethyl}-piperidine-1-carboxylic acid (3,5-difluoro-phenyl)-amide,
4-hydroxy-4-{1-hydroxy-2-[4-(4-trifluoromethyl-phenyl)-piperidin-1-yl]-ethyl}-piperidine-1-carboxylic acid (3,4-dichloro-phenyl)-amide,
4-hydroxy-4-{1-hydroxy-2-[4-(4-trifluoromethyl-phenyl)-piperidin-1-yl]-ethyl}-piperidine-1-carbothioic acid (3,4-dichloro-phenyl)-amide,
4-hydroxy-4-{1-hydroxy-2-[4-(4-trifluoromethyl-phenyl)-piperidin-1-yl]-ethyl}-piperidine-1-carbothioic acid (3,5-dichloro-phenyl)-amide,
4-{2-[4-(4-chloro-phenyl)-piperidin-1-yl]-1-hydroxy-ethyl}-4-hydroxy-piperidine-1-carboxylic acid tert-butyl ester,
4-{2-[4-(5-amino-1H-indol-3-yl)-piperidin-1-yl]-1-hydroxy-ethyl}-piperidine-1-carboxylic acid benzyl ester,
1-(4-hydroxy-4-{1-hydroxy-2-[4-(5-morpholin-4-yl-1H-indol-3-yl)-piperidin-1-yl]-ethyl}-piperidin-1-yl)-3-(3,4,5-trifluoro-phenyl)-propenone,
3-(3,5-difluoro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(3,5-bis-trifluoromethyl-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(3,5-dichloro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
1-(4-{2-[4-(4-fluoro-phenyl)-4-hydroxy-piperidin-1-yl]-1-hydroxy-ethyl}-4-hydroxy-piperidin-1-yl)-3-(3,4,5-trifluoro-phenyl)-propenone,
3-(3,4-difluoro-phenyl)-1-(4-{2-[4-(4-fluoro-phenyl)-4-hydroxy-piperidin-1-yl]-1-hydroxy-ethyl}-4-hydroxy-piperidin-1-yl)-propenone,
3-(3,5-difluoro-phenyl)-1-(4-{2-[4-(4-fluoro-phenyl)-4-hydroxy-piperidin-1-yl]-1-hydroxy-ethyl}-4-hydroxy-piperidin-1-yl)-propenone,
1-(4-{2-[4-(5-fluoro-1H-indol-3-yl)-piperidin-1-yl]-1-hydroxy-ethyl}-4-hydroxy-piperidin-1-yl)-3-(3,4,5-trifluoro-phenyl)-propenone,
3-(3,4-difluoro-phenyl)-1-(4-{2-[4-(5-fluoro-1H-indol-3-yl)-piperidin-1-yl]-1-hydroxy-ethyl}-4-hydroxy-piperidin-1-yl)-propenone,
3-(3,5-difluoro-phenyl)-1-(4-{2-[4-(5-fluoro-1H-indol-3-yl)-piperidin-1-yl]-1-hydroxy-ethyl}-4-hydroxy-piperidin-1-yl)-propenone,
3-(3,4-dichloro-phenyl)-1-(4-{2-[4-(5-fluoro-1H-indol-3-yl)-piperidin-1-yl]-1-hydroxy-ethyl}-4-hydroxy-piperidin-1-yl)-propenone,
3-(3-bromo-4-fluoro-phenyl)-1-(4-{2-[4-(5-fluoro-1H-indol-3-yl)-piperidin-1-yl]-1-hydroxy-ethyl}-4-hydroxy-piperidin-1-yl)-propenone,
3-(4-chloro-phenyl)-1-(4-{2-[4-(5-fluoro-1H-indol-3-yl)-piperidin-1-yl]-1-hydroxy-ethyl}-4-hydroxy-piperidin-1-yl)-propan-1-one,
2-(2-chloro-5-fluoro-phenyl)-1-(4-{2-[4-(5-fluoro-1H-indol-3-yl)-piperidin-1-yl]-1-hydroxy-ethyl}-4-hydroxy-piperidin-1-yl)-ethanone,
2-(3,4-dichloro-phenyl)-1-(4-{2-[4-(5-fluoro-1H-indol-3-yl)-piperidin-1-yl]-1-hydroxy-ethyl}-4-hydroxy-piperidin-1-yl)-ethanethione,
1-(4-{2-[4-(4-chloro-phenyl)-piperidin-1-yl]-1-hydroxy-ethyl}-4-hydroxy-piperidin-1-yl)-3-(3,4,5-trifluoro-phenyl)-propenone,
1-(4-{2-[4-(4-chloro-phenyl)-piperidin-1-yl]-1-hydroxy-ethyl}-4-hydroxy-piperidin-1-yl)-3-(3,5-difluoro-phenyl)-propenone,
4-{2-[4-(4-chloro-phenyl)-piperidin-1-yl]-1-hydroxy-ethyl}-4-hydroxy-piperidine-1-carboxylic acid (3,4-dichloro-phenyl)-amide,
4-{2-[4-(4-chloro-phenyl)-piperidin-1-yl]-1-hydroxy-ethyl}-4-hydroxy-piperidine-1-carbothioic acid (2,3,5-trifluoro-phenyl)-amide,
4-{2-[4-(5-fluoro-1H-indol-3-yl)-piperidin-1-yl]-1-hydroxy-ethyl}-4-hydroxy-piperidine-1-carboxylic acid (3,4-dichloro-phenyl)-amide,
3-(3,4-dichloro-phenyl)-1-(4-{2-[4-(5-fluoro-1H-indol-3-yl)-piperidin-1-yl]-1-hydroxy-ethyl}-4-hydroxy-piperidin-1-yl)-propan-1-one,
3-(3,4-dichloro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(4-chloro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(4-fluoro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(3,4-difluoro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
3-(3-bromo-4-fluoro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
1-(4-hydroxy-4-{1-hydroxy-2-[4-(5-morpholin-4-yl-1H-indol-3-yl)-piperidin-1-yl]-ethyl}-piperidin-1-yl)-3-m-tolyl-propenone,
1-(4-hydroxy-4-{1-hydroxy-2-[4-(5-morpholin-4-yl-1H-indol-3-yl)-piperidin-1-yl]-ethyl}-piperidin-1-yl)-3-(2,4,5-trifluoro-phenyl)-propenone,
1-(4-hydroxy-4-{1-hydroxy-2-[4-(5-morpholin-4-yl-1H-indol-3-yl)-piperidin-1-yl]-ethyl}-piperidin-1-yl)-2-(3-nitro-phenyl)-ethanone,
1-(4-hydroxy-4-{1-hydroxy-2-[4-(5-morpholin-4-yl-1H-indol-3-yl)-piperidin-1-yl]-ethyl}-piperidin-1-yl)-3-(3-methoxy-phenyl)-propenone,
1-(4-hydroxy-4-{1-hydroxy-2-[4-(5-morpholin-4-yl-1H-indol-3-yl)-piperidin-1-yl]-ethyl}-piperidin-1-yl)-3-thiophen-3-yl-propenone,
4-hydroxy-4-{1-hydroxy-2-[4-(5-morpholin-4-yl-1H-indol-3-yl)-piperidin-1-yl]-ethyl}-piperidine-1-carboxylic acid (3,4-difluoro-phenyl)-amide,
4-hydroxy-4-{1-hydroxy-2-[4-(5-morpholin-4-yl-1H-indol-3-yl)-piperidin-1-yl]-ethyl}-piperidine-1-carboxylic acid (3-fluoro-5-trifluoromethyl-phenyl)-amide,
4-hydroxy-4-{1-hydroxy-2-[4-(5-morpholin-4-yl-1H-indol-3-yl)-piperidin-1-yl]-ethyl}-piperidine-1-carboxylic acid (3,4-dichloro-phenyl)-amide,
4-hydroxy-4-{1-hydroxy-2-[4-(5-morpholin-4-yl-1H-indol-3-yl)-piperidin-1-yl]-ethyl}-piperidine-1-carbothioic acid (3,5-dichloro-phenyl)-amide,
4-hydroxy-4-{1-hydroxy-2-[4-(5-morpholin-4-yl-1H-indol-3-yl)-piperidin-1-yl]-ethyl}-piperidine-1-carbothioic acid (3,5-difluoro-phenyl)-amide,
4-{2-[4-(5-acetylamino-1H-indol-3-yl)-piperidin-1-yl]-1-hydroxy-ethyl}-piperidine-1-carboxylic acid benzyl ester, and
N-{3-[1-(2-hydroxy-2-piperidin-4-yl-ethyl)-piperidin-4-yl]-1H-indol-5-yl}-acetamide.

22. The compound of claim 21 selected from the group consisting of:
4-{1-hydroxy-2-[4-(1H-indol-3-yl)-piperidin-1-yl]-ethyl}-piperidine-1-carboxylic acid (3,4-dichloro-phenyl)-amide,
4-{1-hydroxy-2-[4-(1H-indol-3-yl)-piperidin-1-yl]-ethyl}-piperidine-1-carboxylic acid (3,5-difluoro-phenyl)-amide,
3-(3,4-dichloro-phenyl)-1-(4-{1-hydroxy-2-[4-(1H-indol-3-yl)-piperidin-1-yl]-ethyl}-piperidin-1-yl)-prop enone,
3-(3,5-difluoro-phenyl)-1-(4-{1-hydroxy-2-[4-(1H-indol-3-yl)-piperidin-1-yl]-ethyl}-piperidin-1-yl)-prop enone,
4-{1-hydroxy-2-[4-(1H-indol-3-yl)-piperidin-1-yl]-ethyl}-piperidine-1-carbothioic acid (3-bromo-phenyl)-amide,
3,5-difluoro-N-[(4-{1-hydroxy-2-[4-(1H-indol-3-yl)-piperidin-1-yl]-ethyl}-piperidin-1-yl)-imino-methyl]-benzamide,
1-(4-{1-hydroxy-2-[4-(1H-indol-3-yl)-piperidin-1-yl]-ethyl}-piperidin-1-yl)-3-m-tolyl-propenone,
3-(3,5-difluoro-phenyl)-1-(4-{1-hydroxy-2-[4-(4-methoxy-phenyl)-piperidin-1-yl]-ethyl}-piperidin-1-yl)-propenone,
1-(4-{1-hydroxy-2-[4-(4-methoxy-phenyl)-piperidin-1-yl]-ethyl}-piperidin-1-yl)-3-(3,4,5-trifluoro-phenyl)-propenone,
3-(3,4-dichloro-phenyl)-1-(4-{1-hydroxy-2-[4-(4-methoxy-phenyl)-piperidin-1-yl]-ethyl}-piperidin-1-yl)-propenone,
4-{1-hydroxy-2-[4-(4-methoxy-phenyl)-piperidin-1-yl]-ethyl}-piperidine-1-carboxylic acid (3,4-dichloro-phenyl)-amide,
4-{1-hydroxy-2-[4-(4-methoxy-phenyl)-piperidin-1-yl]-ethyl}-piperidine-1-carbothioic acid (3-bromo-phenyl)-amide,
1-(4-{1-hydroxy-2-[4-(1H-pyrrolo[2,3-b]pyridin-3-yl)-piperidin-1-yl]-ethyl}-piperidin-1-yl)-3-m-tolyl-propenone,
3-(3,5-difluoro-phenyl)-1-(4-{1-hydroxy-2-[4-(1H-pyrrolo[2,3-b]pyridin-3-yl)-piperidin-1-yl]-ethyl}-piperidin-1-yl)-propenone,
1-(4-{1-hydroxy-2-[4-(1H-pyrrolo[2,3-b]pyridin-3-yl)-piperidin-1-yl]-ethyl}-piperidin-1-yl)-3-(3,4,5-trifluoro-phenyl)-propenone,
1-(4-{1-hydroxy-2-[4-(1H-pyrrolo[2,3-b]pyridin-3-yl)-piperidin-1-yl]-ethyl}-piperidin-1-yl)-3-(3-trifluoromethyl-phenyl)-propenone,
3-(3,4-dichloro-phenyl)-1-(4-{1-hydroxy-2-[4-(1H-pyrrolo[2,3-b]pyridin-3-yl)-piperidin-1-yl]-ethyl}-piperidin-1-yl)-propenone,
4-{1-hydroxy-2-[4-(1H-pyrrolo[2,3-b]pyridin-3-yl)-piperidin-1-yl]-ethyl}-piperidine-1-carboxylic acid (3,4-dichloro-phenyl)-amide,
3-(3,5-difluoro-phenyl)-1-(4-{1-hydroxy-2-[4-(5-methoxy-1H-indol-3-yl)-piperidin-1-yl]-ethyl}-piperidin-1-yl)-propenone,
N-{3-[1-(2-hydroxy-2-{1-[3-(3,4,5-trifluoro-phenyl)-acryloyl]-piperidin-4-yl}-ethyl)-piperidin-4-yl]-1H-indol-5-yl}-methanesulfonamide,
1-(4-{1-hydroxy-2-[4-(4-methoxy-phenyl)-piperidin-1-yl]-ethyl}-piperidin-1-yl)-3-m-tolyl-propenone,
1-(4-{1-hydroxy-2-[4-(4-methoxy-phenyl)-piperidin-1-yl]-ethyl}-piperidin-1-yl)-3-(3-trifluoromethyl-phenyl)-propenone,
3-(3,4-difluoro-phenyl)-1-(4-{1-hydroxy-2-[4-(4-methoxy-phenyl)-piperidin-1-yl]-ethyl}-piperidin-1-yl)-propenone,
3-[4-(4-methoxy-phenyl)-piperidin-1-yl]-2-{1-[3-(3,4,5-trifluoro-phenyl)-acryloyl]-piperidin-4-yl}-propionic acid,
N-[3-(1-{2-hydroxy-2-[1-(3-m-tolyl-acryloyl)-piperidin-4-yl]-ethyl}-piperidin-4-yl)-1H-indol-5-yl]-methanesulfonamide,
N-{3-[1-(2-{1-[3-(3,5-difluoro-phenyl)-acryloyl]-piperidin-4-yl}-2-hydroxy-ethyl)-piperidin-4-yl]-1H-indol-5-yl}-methanesulfonamide,
3-(3,4-dichloro-phenyl)-1-(4-{1-hydroxymethyl-2-[4-(4-methoxy-phenyl)-piperidin-1-yl]-ethyl}-piperidin-1-yl)-propenone,
3-(3,5-difluoro-phenyl)-1-(4-{1-hydroxymethyl-2-[4-(4-methoxy-phenyl)-piperidin-1-yl]-ethyl}-piperidin-1-yl)-propenone,
2-[4-(1H-indol-3-yl)-piperidin-1-yl]-3-[1-(3-m-tolyl-acryloyl)-piperidin-4-yl]-propionic acid ethyl ester,
3-{1-[3-(3,4-dichloro-phenyl)-acryloyl]-piperidin-4-yl}-2-[4-(1H-indol-3-yl)-piperidin-1-yl]-propionic acid ethyl ester,
2-[4-(1H-indol-3-yl)-piperidin-1-yl]-3-{1-[3-(3,4,5-trifluoro-phenyl)-acryloyl]-piperidin-4-yl}-propionic acid ethyl ester,
3-{1-[3-(3,5-difluoro-phenyl)-acryloyl]-piperidin-4-yl}-2-[4-(1H-indol-3-yl)-piperidin-1-yl]-propionic acid ethyl ester,
3-[1-(3,4-dichloro-phenylcarbamoyl)-piperidin-4-yl]-2-[4-(1H-indol-3-yl)-piperidin-1-yl]-propionic acid ethyl ester,
2-[4-(1H-indol-3-yl)-piperidin-1-yl]-3-[1-(3-m-tolyl-acryloyl)-piperidin-4-yl]-propionic acid,
4-{3-hydroxy-2-[4-(1H-indol-3-yl)-piperidin-1-yl]-propyl}-piperidine-1-carboxylic acid (3,4-dichloro-phenyl)-amide,
3-[1-(3,4-dichloro-phenylcarbamoyl)-piperidin-4-yl]-2-[4-(1H-indol-3-yl)-piperidin-1-yl]-propionic acid,
4-{1-hydroxy-2-[4-(5-methanesulfonylamino-1H-indol-3-yl)-piperidin-1-yl]-ethyl}-piperidine-1-carboxylic acid tert-butyl ester,
1-(4-{1-amino-2-[4-(4-methoxy-phenyl)-piperidin-1-yl]-ethyl}-piperidin-1-yl)-3-(3,4,5-trifluoro-phenyl)-propenone,
1-(4-hydroxy-4-{1-hydroxy-2-[4-(1H-indol-3-yl)-piperidin-1-yl]-ethyl}-piperidin-1-yl)-3-(3,4,5-trifluoro-phenyl)-propenone,
3-(3,5-difluoro-phenyl)-1-(4-hydroxy-4-{1-hydroxy-2-[4-(1H-indol-3-yl)-piperidin-1-yl]-ethyl}-piperidin-1-yl)-propenone,
3-(3,5-dichloro-phenyl)-1-(4-hydroxy-4-{1-hydroxy-2-[4-(1H-indol-3-yl)-piperidin-1-yl]-ethyl}-piperidin-1-yl)-propenone,
3-(3-chloro-5-fluoro-phenyl)-1-(4-hydroxy-4-{1-hydroxy-2-[4-(1H-indol-3-yl)-piperidin-1-yl]-ethyl}-piperidin-1-yl)-propenone,
3-(3,4-dichloro-phenyl)-1-(4-hydroxy-4-{1-hydroxy-2-[4-(1H-indol-3-yl)-piperidin-1-yl]-ethyl}-piperidin-1-yl)-propenone,
1-(4-hydroxy-4-{1-hydroxy-2-[4-(1H-indol-3-yl)-piperidin-1-yl]-ethyl}-piperidin-1-yl)-3-(3-trifluoromethyl-phenyl)-propenone,
3-(3,4-difluoro-phenyl)-1-(4-hydroxy-4-{1-hydroxy-2-[4-(1H-indol-3-yl)-piperidin-1-yl]-ethyl}-piperidin-1-yl)-propenone,
3-(3-bromo-4-fluoro-phenyl)-1-(4-hydroxy-4-{1-hydroxy-2-[4-(1H-indol-3-yl)-piperidin-1-yl]-ethyl}-piperidin-1-yl)-propenone,
4-hydroxy-4-{1-hydroxy-2-[4-(1H-indol-3-yl)-piperidin-1-yl]-ethyl}-piperidine-1-carbothioic acid (3,4-dichloro-phenyl)-amide,
1-(4-{2-[4-(1H-benzoimidazol-2-yl)-piperidin-1-yl]-1-hydroxy-ethyl}-piperidin-1-yl)-3-(3,4-dichloro-phenyl)-propenone,
1-(4-{2-[4-(1H-benzoimidazol-2-yl)-piperidin-1-yl]-1-hydroxy-ethyl}-piperidin-1-yl)-3-(3,4,5-trifluoro-phenyl)-propenone,
1-(4-hydroxy-4-{1-hydroxy-2-[4-(4-methoxy-phenyl)-piperidin-1-yl]-ethyl}-piperidin-1-yl)-3-(3,4,5-trifluoro-phenyl)-propenone,
4-{1-hydroxy-2-[4-(4-methoxy-phenyl)-piperidin-1-yl]-ethyl}-piperidine-1-carboxylic acid (4,5-dichloro-2-fluoro-phenyl)-amide,
3-(3,4-difluoro-phenyl)-1-(4-hydroxy-4-{1-hydroxy-2-[4-(4-methoxy-phenyl)-piperidin-1-yl]-ethyl}-piperidin-1-yl)-propenone,
3-(3,5-difluoro-phenyl)-1-(4-hydroxy-4-{1-hydroxy-2-[4-(4-methoxy-phenyl)-piperidin-1-yl]-ethyl}-piperidin-1-yl)-propenone,
1-(4-hydroxy-4-{1-hydroxy-2-[4-(4-methoxy-phenyl)-piperidin-1-yl]-ethyl}-piperidin-1-yl)-3-m-tolyl-propenone,
4-{1-hydroxy-2-[4-(4-methoxy-phenyl)-piperidin-1-yl]-ethyl}-piperidine-1-carboxylic acid benzo[1,3]dioxol-5-ylamide,
4-{1-hydroxy-2-[4-(4-methoxy-phenyl)-piperidin-1-yl]-ethyl}-piperidine-1-carboxylic acid (2-chloro-4-trifluoromethyl-phenyl)-amide,
4-{1-hydroxy-2-[4-(4-methoxy-phenyl)-piperidin-1-yl]-ethyl}-piperidine-1-carbothioic acid (3,5-difluoro-phenyl)-amide,
4-{1-hydroxy-2-[4-(4-methoxy-phenyl)-piperidin-1-yl]-ethyl}-piperidine-1-carbothioic acid (4-bromo-3-fluoro-phenyl)-amide,
4-hydroxy-4-{1-hydroxy-2-[4-(4-methoxy-phenyl)-piperidin-1-yl]-ethyl}-piperidine-1-carboxylic acid (3,4-difluoro-phenyl)-amide,
4-hydroxy-4-{1-hydroxy-2-[4-(4-methoxy-phenyl)-piperidin-1-yl]-ethyl}-piperidine-1-carboxylic acid (3,5-difluoro-phenyl)-amide,
4-hydroxy-4-{1-hydroxy-2-[4-(4-methoxy-phenyl)-piperidin-1-yl]-ethyl}-piperidine-1-carbothioic acid (3,5-dimethoxy-phenyl)-amide,
4-{2-[4-(1H-benzoimidazol-2-yl)-piperidin-1-yl]-1-hydroxy-ethyl}-1-(4-trifluoromethyl-pyrimidin-2-yl)-piperidin-4-ol,
4-{1-hydroxy-2-[4-(5-morpholin-4-yl-1H-indol-3-yl)-piperidin-1-yl]-ethyl}-1-(4-trifluoromethyl-pyrimidin-2-yl)-piperidin-4-ol,
4-{1-hydroxy-2-[4-(5-methyl-1H-indol-3-yl)-piperidin-1-yl]-ethyl}-1-(4-trifluoromethyl-pyrimidin-2-yl)-piperidin-4-ol,
3-(1-{2-hydroxy-2-[4-hydroxy-1-(4-trifluoromethyl-pyrimidin-2-yl)-piperidin-4-yl]-ethyl}-piperidin-4-yl)-1H-indole-5-carbonitrile,
4-{2-[4-(4-fluoro-phenyl)-piperidin-1-yl]-1-hydroxy-ethyl}-4-hydroxy-piperidine-1-carboxylic acid benzyl ester,
4-hydroxy-4-{1-hydroxy-2-[4-(4-trifluoromethyl-phenyl)-piperidin-1-yl]-ethyl}-piperidine-1-carboxylic acid benzyl ester,
2-[4-(4-methoxy-phenyl)-piperidin-1-yl]-1-(5′-nitro-3,4,5,6-tetrahydro-2H-[1,2′]bipyridinyl-4-yl)-ethanol,
1-(4-{2-[4-(4-fluoro-phenyl)-piperidin-1-yl]-1-hydroxy-ethyl}-4-hydroxy-piperidin-1-yl)-3-(3,4,5-trifluoro-phenyl)-propenone,
3-(3,5-difluoro-phenyl)-1-(4-{2-[4-(4-fluoro-phenyl)-piperidin-1-yl]-1-hydroxy-ethyl}-4-hydroxy-piperidin-1-yl)-propenone,
3-(3,4-dichloro-phenyl)-1-(4-{2-[4-(4-fluoro-phenyl)-piperidin-1-yl]-1-hydroxy-ethyl}-4-hydroxy-piperidin-1-yl)-propenone,
4-{2-[4-(4-fluoro-phenyl)-piperidin-1-yl]-1-hydroxy-ethyl}-4-hydroxy-piperidine-1-carboxylic acid (3,4-dichloro-phenyl)-amide,
1-(4-hydroxy-4-{1-hydroxy-2-[4-(4-trifluoromethyl-phenyl)-piperidin-1-yl]-ethyl}-piperidin-1-yl)-3-(3,4,5-trifluoro-phenyl)-propenone,
3-(3,4-difluoro-phenyl)-1-(4-hydroxy-4-{1-hydroxy-2-[4-(4-trifluoromethyl-phenyl)-piperidin-1-yl]-ethyl}-piperidin-1-yl)-propenone,
3-(3,5-difluoro-phenyl)-1-(4-hydroxy-4-{1-hydroxy-2-[4-(4-trifluoromethyl-phenyl)-piperidin-1-yl]-ethyl}-piperidin-1-yl)-propenone,
4-hydroxy-4-{1-hydroxy-2-[4-(4-trifluoromethyl-phenyl)-piperidin-1-yl]-ethyl}-piperidine-1-carboxylic acid (3,5-difluoro-phenyl)-amide,
4-{2-[4-(4-chloro-phenyl)-piperidin-1-yl]-1-hydroxy-ethyl}-4-hydroxy-piperidine-1-carboxylic acid tert-butyl ester,
3-(3,5-dichloro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
1-(4-{2-[4-(4-fluoro-phenyl)-4-hydroxy-piperidin-1-yl]-1-hydroxy-ethyl}-4-hydroxy-piperidin-1-yl)-3-(3,4,5-trifluoro-phenyl)-propenone,
3-(3,4-difluoro-phenyl)-1-(4-{2-[4-(4-fluoro-phenyl)-4-hydroxy-piperidin-1-yl]-1-hydroxy-ethyl}-4-hydroxy-piperidin-1-yl)-propenone,
1-(4-{2-[4-(5-fluoro-1H-indol-3-yl)-piperidin-1-yl]-1-hydroxy-ethyl}-4-hydroxy-piperidin-1-yl)-3-(3,4,5-trifluoro-phenyl)-propenone,
3-(3,4-difluoro-phenyl)-1-(4-{2-[4-(5-fluoro-1H-indol-3-yl)-piperidin-1-yl]-1-hydroxy-ethyl}-4-hydroxy-piperidin-1-yl)-propenone,
3-(3,5-difluoro-phenyl)-1-(4-{2-[4-(5-fluoro-1H-indol-3-yl)-piperidin-1-yl]-1-hydroxy-ethyl}-4-hydroxy-piperidin-1-yl)-propenone,
1-(4-{2-[4-(4-chloro-phenyl)-piperidin-1-yl]-1-hydroxy-ethyl}-4-hydroxy-piperidin-1-yl)-3-(3,4,5-trifluoro-phenyl)-propenone,
1-(4-{2-[4-(4-chloro-phenyl)-piperidin-1-yl]-1-hydroxy-ethyl}-4-hydroxy-piperidin-1-yl)-3-(3,5-difluoro-phenyl)-propenone,
4-{2-[4-(4-chloro-phenyl)-piperidin-1-yl]-1-hydroxy-ethyl}-4-hydroxy-piperidine-1-carboxylic acid (3,4-dichloro-phenyl)-amide,
4-{2-[4-(5-fluoro-1H-indol-3-yl)-piperidin-1-yl]-1-hydroxy-ethyl}-4-hydroxy-piperidine-1-carboxylic acid (3,4-dichloro-phenyl)-amide,
3-(3,4-difluoro-phenyl)-1-(4-hydroxy-4-{1-hydroxy-2-[4-(5-morpholin-4-yl-1H-indol-3-yl)-piperidin-1-yl]-ethyl}-piperidin-1-yl)-propenone,
1-(4-hydroxy-4-{1-hydroxy-2-[4-(5-morpholin-4-yl-1H-indol-3-yl)-piperidin-1-yl]-ethyl}-piperidin-1-yl)-3-m-tolyl-propenone,
1-(4-hydroxy-4-{1-hydroxy-2-[4-(5-morpholin-4-yl-1H-indol-3-yl)-piperidin-1-yl]-ethyl}-piperidin-1-yl)-3-(2,4,5-trifluoro-phenyl)-propenone,
1-(4-hydroxy-4-{1-hydroxy-2-[4-(5-morpholin-4-yl-1H-indol-3-yl)-piperidin-1-yl]-ethyl}-piperidin-1-yl)-2-(3-nitro-phenyl)-ethanone,
1-(4-hydroxy-4-{1-hydroxy-2-[4-(5-morpholin-4-yl-1H-indol-3-yl)-piperidin-1-yl]-ethyl}-piperidin-1-yl)-3-(3-methoxy-phenyl)-propenone,
1-(4-hydroxy-4-{1-hydroxy-2-[4-(5-morpholin-4-yl-1H-indol-3-yl)-piperidin-1-yl]-ethyl}-piperidin-1-yl)-3-thiophen-3-yl-propenone,
4-hydroxy-4-{1-hydroxy-2-[4-(5-morpholin-4-yl-1H-indol-3-yl)-piperidin-1-yl]-ethyl}-piperidine-1-carboxylic acid (3,4-dichloro-phenyl)-amide,
4-hydroxy-4-{1-hydroxy-2-[4-(5-morpholin-4-yl-1H-indol-3-yl)-piperidin-1-yl]-ethyl}-piperidine-1-carbothioic acid (3,5-difluoro-phenyl)-amide,
4-{2-[4-(5-acetylamino-1H-indol-3-yl)-piperidin-1-yl]-1-hydroxy-ethyl}-piperidine-1-carboxylic acid benzyl ester, and
N-{3-[1-(2-hydroxy-2-piperidin-4-yl-ethyl)-piperidin-4-yl]-1H-indol-5-yl}-acetamide.

23. The compound of claim 1, wherein the compound is a CCR2 antagonist.

24. The compound of claim 23, wherein the compound is a prodrug form thereof.

25. The compound of claim 23, wherein the compound is an isolated form thereof.

26. The compound of claim 24, wherein the compound is a metabolite form thereof.

27. The compound of claim 25, wherein the compound is labeled with a ligand for use as a marker, and wherein the ligand is a radioligand selected from deuterium or tritium.

28. A pharmaceutical composition comprising an effective amount of the compound of claim 25.

29. The pharmaceutical composition of claim 28, further comprising a pharmaceutically acceptable carrier.

30. The pharmaceutical composition of claim 28, wherein the effective amount of the compound is in a range of from about 0.001 mg/kg to about 300 mg/kg of body weight per day.

31. A process for preparing a pharmaceutical composition comprising the step of admixing the compound of claim 1 and a pharmaceutically acceptable carrier.

32. A medicament comprising an effective amount of the compound of claim 25.

33. The medicament of claim 32, wherein the effective amount of the compound is in a range of from about 0.001 mg/kg to about 300 mg/kg of body weight per day.

34. Use of the compound of claim 25 as a CCR2 antagonist comprising contacting the receptor with the compound.

35. The use of claim 34, wherein the use further comprises use of the compound in a pharmaceutical composition, medicine or medicament for preventing, treating or ameliorating a CCR2 mediated inflammatory syndrome, disorder or disease.

36. Use of the compound of claim 25 in the manufacture of a medicament for preventing, treating or ameliorating a CCR2 mediated inflammatory syndrome, disorder or disease.

37. A method for preventing, treating or ameliorating a CCR2 mediated inflammatory syndrome, disorder or disease in a subject in need thereof comprising administering to the subject an effective amount of the compound of claim 1.

38. The method of claim 37, wherein the effective amount of the compound is in a range of from about 0.001 mg/kg to about 300 mg/kg of body weight per day.

39. The method of claim 37, further comprising administering to the subject an effective amount of the compound as a pharmaceutical composition, medicine or medicament thereof.

40. The method of claim 37, wherein the syndrome, disorder or disease is associated with elevated MCP-1 expression or MCP-1 overexpression, or is an inflammatory condition that accompanies syndromes, disorders or diseases associated with elevated MCP-1 expression or MCP-1 overexpression.

41. The method of claim 37, wherein the syndrome, disorder or disease is selected from ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, diabetes and diabetic complications, diabetic nephropathy, diabetic retinopathy, diabetic retinitis, diabetic microangiopathy, obesity, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, or carcinomas of the bladder, breast, cervix, colon, lung, prostate, or stomach.

42. The method of claim 37, wherein the method further comprises preventing, treating or ameliorating CCR2 mediated ophthalmic disorders, rheumatoid arthritis, psoriasis, psoriatic arthritis, atopic dermatitis, obesity, chronic obstructive pulmonary disease, allergic rhinitis, asthma, allergic asthma, periodontal diseases in a subject in need thereof comprising administering to the subject an effective amount of the compound of claim 1.

43. The method of claim 42, wherein the ophthalmic disorder is selected from uveitis or allergic conjunctivitis and the periodontal disease is selected from periodonitis, gingivitis or gum disease.

44. The method of claim 43, wherein uveitis is selected from acute, recurring or chronic uveitis.

45. The method of claim 43, wherein uveitis is selected from anterior uveitis, intermediate uveitis, posterior uveitis or panuveitis.

46. The method of claim 42, wherein the effective amount of the compound is in a range of from about 0.001 mg/kg to about 300 mg/kg of body weight per day.

47. The method of claim 42, further comprising administering to the subject an effective amount of the compound of claim 1 as a pharmaceutical composition, medicine or medicament thereof.

48. The method of claim 42, further comprising administering to the subject an effective amount of a combination product comprising the compound of claim 1 and one or more therapeutic agent.

49. The method of claim 48, wherein the therapeutic agent is selected from an anti-inflammatory agent, an anti-infective agent or an immunosuppressive agent.

50. A method for preventing, treating or ameliorating CCR2 mediated obesity in a subject in need thereof comprising administering to the subject an effective amount of the compound of claim 1.

51. The method of claim 50, wherein obesity in the subject is prevented, treated or ameliorated by the inhibition of weight gain, the inducement of weight loss or, in the alternative, an improvement in insulin sensitivity.

52. The method of claim 50, wherein the effective amount of the compound is in a range of from about 0.001 mg/kg to about 300 mg/kg of body weight per day.

53. The method of claim 50, further comprising administering to the subject an effective amount of the compound of claim 1 as a pharmaceutical composition, medicine or medicament thereof.
